(19) United States
(12) Patent Application Publication
(10) Pub. No.: US 2006/0194220 A1
(43) Pub. Date: Aug. 31, 2006

(54) Compositions and methods for detecting and treating tumors

(75) Inventors: Ramachandra Reddy, Pearland, TX (US); Parkash Gill, Agoura Hills, CA (US)

Correspondence Address: FISH & NEAVE IP GROUP;ROPES & GRAY LLP, ONE INTERNATIONAL PLACE, BOSTON, MA, 02110-2624, US

(73) Assignee: VasGene Therapeutics, Inc., Sharon Hills, PA (US)

(21) Appl. No.: 11/234,587

(22) Filed: Sep. 23, 2005

Related U.S. Application Data
(60) Provisional application No. 60/612,861, filed on Sep. 23, 2004.

(51) Int. Cl. C12Q 1/68

(52) U.S. Cl. 435/6

(57) ABSTRACT

In certain embodiments, this present disclosure provides compositions and methods for detecting EphB4 gene amplification in test cells. In certain embodiments, the present disclosure provides methods and compositions for evaluating tumor (cancer) status and prognosis in a subject having or suspected of having a tumor.

### RELATED APPLICATIONS

This application claims the benefit of U.S. Provisional Application No. 60/612,861, filed Sep. 23, 2004. The entire teachings of the referenced Application are incorporated herein by reference in their entirety.

### BACKGROUND OF THE INVENTION

Cancers are a significant cause of mortality in the adult American population. However, in many instances early stage cancers are treatable by surgical removal (resection). Surgical treatment can be combined with chemotherapeutic agents to achieve an even higher survival rate in certain cancers. In most cancers, survival rate drops precipitously in patients with metastatic (late stage) colon cancer.

Effective screening and early identification of affected patients coupled with appropriate therapeutic intervention is proven to reduce the number of mortalities in, for example, colon cancer. Additionally, diagnostic tests to monitor treatments and cancer progression are highly useful in developing therapeutic plans and adapting such plans to the status of the patient.

Modern molecular biology has made it possible to identify proteins and nucleic acids that are specifically associated with certain physiological states. These molecular markers have revolutionized diagnostics for a variety of health conditions ranging from pregnancy to viral infections, such as HIV.

It is a goal of the present disclosure to provide agents and methods for tumor detection and tumor treatment.

### SUMMARY OF THE INVENTION

In certain aspects, the disclosure relates to the discovery that indicators of heightened EphB4 activity are associated with cancerous states, and particularly with metastatic cancer. Such indicators include EphB4 mRNA and protein levels. Surprisingly, the EphB4 gene is amplified in many cancers, and particularly in metastatic cancers. EphB4 amplification shows correlation with EphB4 protein levels. Accordingly, EphB4 gene amplification (e.g., copy number) may also be used to detect and evaluate cancerous states.

In certain aspects, the disclosure provides methods for identifying a tumor that is suitable for treatment with an inhibitor of EphB4 expression or function. Such methods may include detecting in the tumor a cell having one or more of the following characteristics: (a) abnormally high expression of EphB4 protein; (b) abnormally high expression of EphB4 mRNA; and (c) gene amplification of the EphB4 gene. A tumor comprising cells having one or more of characteristics (a)-(c) is likely to be sensitive to treatment with an inhibitor of EphB4 expression or function. It should be noted that tumors that do not directly express EphB4 may also be sensitive to treatments targeted at these proteins, as these proteins are known to be expressed in the vascular endothelium and to participate in the formation of new capillaries that service growing tumors. An inhibitor of EphB4 expression or function may be, for example, (i) an EphB4-selective compound (e.g., a nucleic acid compound that hybridizes to an EphB4 transcript under physiological conditions and decreases the expression of EphB4 in a cell, or a polypeptide that inhibits a cellular function of EphB4).

In certain aspects, the disclosure provides methods for evaluating gene amplification of the EphB4 gene in a test cell. Such methods may comprise detecting the EphB4 gene copy number in a test cell, wherein an increase in the EphB4 gene copy number in the test cell relative to that in a control cell is indicative of gene amplification of the EphB4 gene in the test cell. The EphB4 gene copy number can be detected by hybridization-based assays (e.g., Southern blot, in situ hybridization (ISH), and comparative genomic hybridization (CGH)), or by amplification-based assays (e.g., quantative PCR). Optionally, the EphB4 gene copy number is detected by using a microarray-based platform. Preferably, test cells in these methods are mammalian cells such as human cells. In certain cases, the test cell is a tumor cell which includes, but is not limited to, a squamous cell carcinoma of the head and neck (HNSCC), a prostate tumor cell, a breast tumor cell, a colorectal carcinoma cell, a lung tumor cell, a bladder tumor cell, and a brain tumor cell. Test cells may be obtained from a subject suspected of having a tumor, or a subject that is known to have a tumor. In the latter case, a test cell can be obtained from a tumor tissue, a primary tumor, or a tissue that is suspected of harboring metastatic cells derived from the primary tumor. As a specific example, a test cell is obtained from lymph nodes or bone marrow. As another specific example, a test cell is obtained from (present in) a bodily fluid selected from the group consisting of blood, serum, plasma, a blood-derived fraction, lymph fluid, pleural fluid, stool, urine, and a colonic effluent. In a specific embodiment, a test cell is present in a pool of test cells. Thus, the methods can be used for identifying or screening for gene amplification of EphB4 in multiple test cells.

In certain aspects, the disclosure provides methods for evaluating the cancer status of a cell in a subject. Such methods comprise: (a) obtaining a test cell from a subject suspected of having or known to have a tumor; (b) detecting the EphB4 gene copy number in the test cell, wherein an increase in the EphB4 gene copy number in the test cell relative to that in a control cell indicates that the test cell is a tumor cell. The EphB4 gene copy number can be detected by hybridization-based assays (e.g., Southern blot, in situ hybridization (ISH), and comparative genomic hybridization (CGH)), or by amplification-based assays (e.g., quantative PCR). Optionally, the EphB4 gene copy number is detected by using a microarray-based platform. Preferably, test cells in these methods are mammalian cells such as human cells. In certain cases, the test cell is a tumor cell which includes, but is not limited to, a squamous cell carcinoma of the head and neck (HNSCC), a prostate tumor cell, a breast tumor cell, a colorectal carcinoma cell, a lung tumor cell, a bladder tumor cell, and a brain tumor cell. To illustrate, test cells can be obtained from a tumor tissue, a primary tumor, or a tissue that is suspected of harboring metastatic cells derived from the primary tumor. As a specific example, a test cell is obtained from lymph nodes or bone marrow. As another specific example, a test cell is obtained from or present in a bodily fluid selected from the group consisting of blood, serum, plasma, a blood-derived fraction, lymph fluid, pleural fluid, stool, urine, and a colonic effluent. In a specific embodiment, a test cell is present in a pool of test cells. Thus, the methods can be used for evaluating the cancer status of multiple cells in one or more subjects.

In certain aspects, the disclosure provides methods for evaluating the prognosis of a subject, comprising: (a) obtaining a test cell from a subject suspected of having or known to have a tumor; (b) detecting an indicator of elevated EphB4 activity in the test cell, wherein an increase in the indicator of EphB4 activity in the test cell relative to that in a control cell indicates that the subject is at increased risk for having or developing a metastatic cancer. As used herein, the indicator of EphB4 activity includes EphB4 mRNA, EphB4 protein, and EphB4 gene copy number. In a specific embodiment, the EphB4 mRNA can be detected by a hybridization-based assay using an EphB4 nucleotide probe or by an amplification-based assay using an EphB4 nucleotide primer. Optionally, the nucleotide probe or primer used in the methods is labeled. In another specific embodiment, the EphB4 protein can be detected by an immuno-assay using an EphB4 antibody, including but is not limited to, EphB4 antibody No. 1, 23, 35, 47, 57, 79, 85L, 85H, 91, 98, 121, 131, or 138. Optionally, the antibody used in the methods is labeled. In yet another specific embodiment, the EphB4 gene copy number can be detected by hybridization-based assays or by amplification-based assays. In certain cases, the indicator of EphB4 is detected by using a microarray-based platform. In certain embodiments, the increase of the indicator of EphB4 in the test cell is at least three fold relative to that in a control cell. Preferably, test cells in these methods are mammalian cells such as human cells. In certain cases, the test cell is a tumor cell which includes, but is not limited to, a squamous cell carcinoma of the head and neck (HNSCC), a prostate tumor cell, a breast tumor cell, a colorectal carcinoma cell, a lung tumor cell, a bladder tumor cell, and a brain tumor cell. To illustrate, test cells can be obtained from a tumor tissue (e.g., a primary tumor), or a tissue that is suspected of harboring metastatic cells derived from the primary tumor. As a specific example, a test cell is obtained from lymph nodes or bone marrow. As another specific example, a test cell is obtained from or present in a bodily fluid selected from the group consisting of blood, serum, plasma, a blood-derived fraction, lymph fluid, pleural fluid, stool, urine, and a colonic effluent. In a specific embodiment, a test cell is present in a pool of test cells. Thus, the methods can be used for evaluating the prognosis of more than two subjects.

In certain aspects, the disclosure provides methods for treating a patient suffering from a cancer, comprising: (a) identifying in the patient a tumor having a plurality of cancer cells having a gene amplification of the EphB4 gene; and (b) administering to the patient an EphB4-selective therapeutic compound (e.g., a nucleic acid compound that hybridizes to an EphB4 transcript under physiological conditions and decreases the expression of EphB4 in a cell, or a polypeptide that inhibits a cellular function of EphB4). Such methods may include, as a diagnostic part, identifying in the patient a tumor having a plurality of cancer cells having gene amplification of the EphB4. Gene amplifications may be detected in a variety of ways, including hybridization-based assays (e.g., in situ hybridization) and amplification-based assays (e.g., quantative PCR).

In certain aspects, the disclosure provides kits for detecting gene amplification of the EphB4 gene in a test cell. Such kits may comprise: (a) one or more nucleic acid capable of hybridizing to the EphB4 gene under high stringency conditions; and (b) a control nucleic acid comprising human genomic DNA having one copy of EphB4 at the normal position. For example, the nucleic acids of the kit may be used in hybridization-based assays as probes or in amplification-based assays as primers.

In certain aspects, the disclosure provides kits for detecting gene amplification of the EphB4 gene in a test cell. Such kits may comprise: (a) one or more nucleic acid capable of hybridizing to the EphB4 gene under high stringency conditions; and (b) at least one control cell line that exhibits a mean of about two copies of EphB4 gene. Optionally, the kits may further comprise at least one control cell line that exhibits a mean of about four copies of EphB4 gene.

### BRIEF DESCRIPTION OF THE DRAWINGS

FIG. 1 shows amino acid sequence of the B4ECv3 protein (predicted sequence of the precursor including uncleaved Eph B4 leader peptide is shown).

FIG. 2 shows amino acid sequence of the B4ECv3NT protein (predicted sequence of the precursor including uncleaved Eph B4 leader peptide is shown).

FIG. 3 shows amino acid sequence of the B2EC protein (predicted sequence of the precursor including uncleaved Ephrin B2 leader peptide is shown).

FIG. 4 shows amino acid sequence of the B4ECv3-FC protein (predicted sequence of the precursor including uncleaved Eph B4 leader peptide is shown).

FIG. 5 shows amino acid sequence of the B2EC-FC protein (predicted sequence of the precursor including uncleaved Ephrin B2 leader peptide is shown).

FIG. 6 shows the domain structure of the recombinant soluble EphB4EC proteins. Designation of the domains are as follows: L—leader peptide, G—globular (ligand-binding domain), C—Cys-rich domain, F1, F2—fibronectin type III repeats, H—6×His-tag.

FIG. 7 shows EphB4 expression and gene amplification in HNSCC primary tissues and metastases. A) Top: Immunohistochemistry of a representative archival section stained with EphB4 monoclonal antibody as described in the methods and visualized with DAB (brown color) localized to tumor cells. Bottom: Hematoxylin and Eosin (H&E) stain of an adjacent section. Dense purple staining indicates the presence of tumor cells. The right hand column are frozen sections of lymph node metastasis stained with EphB4 polyclonal antibody (top right) and visualized with DAB. Control (middle) was incubation with goat serum and H&E (bottom) reveals the location of the metastatic foci surrounded by stroma which does not stain. B) In situ hybridization of serial frozen sections of a HNSCC case probed with EphB4 (left column) and ephrin B2 (right column) DIG labeled antisense or sense probes generated by run-off transcription. Hybridization signal (dark blue) was detected using alkaline-phosphatase-conjugated anti-DIG antibodies and sections were counterstained with Nuclear Fast Red. A serial section stained with H&E is shown (bottom left) to illustrate tumor architecture. C) Western blot of protein extract of patient samples consisting of tumor (T), uninvolved normal tissue (N) and lymph node biopsies (LN). Samples were fractionated by polyacrylamide gel electrophoresis in 4-20% Tris-glycine gels and subsequently electroblotted onto nylon membranes. Membranes were sequentially probed with EphB4 monoclonal antibody and β-actin MoAb. Chemiluminescent signal was detected on autoradiography film. Shown is the EphB4 specific band which migrated at 120 kD and β-actin which migrated at 40 kD. The β-actin signal was used to control for loading and transfer of each sample. D) Survey of EphB4 gene amplification in the HNSCC primary tissues and metastases, as indicated by the increase in EphB4 gene copy number.

FIG. 8 shows EphB4 expression and gene amplification in HNSCC cell lines. A) Survey of EphB4 expression in SCC cell lines. Western blot of total cell lysates sequentially probed with EphB4 monoclonal antibody, stripped and reprobed with β-actin monoclonal antibody as described for FIG. 7C. B) Survey of EphB4 gene amplification in some of the SCC cell lines, as indicated by the increase in EphB4 gene copy number.

FIG. 9 shows expression of EphB4 in prostate cell lines. A) Western blot of total cell lysates of various prostate cancer cell lines, normal prostate gland derived cell line (MLC) and acute myeloblastic lymphoma cells (AML) probed with EphB4 monoclonal antibody. B) Phosphorylation of EphB4 in PC-3 cells determined by Western blot.

FIG. 10 shows expression of EphB4 in prostate cancer tissue. Representative prostate cancer frozen section stained with EphB4 monoclonal antibody (top left) or isotype specific control (bottom left). Adjacent BPH tissue stained with EphB4 monoclonal antibody (top right). Positive signal is brown color in the tumor cells. Stroma and the normal epithelia are negative. Note membrane localization of stain in the tumor tissue, consistent with trans-membrane localization of EphB4. Representative QRT-PCR of RNA extracted from cancer specimens and adjacent BPH tissues (lower right).

FIG. 11 shows that EphB4 and EphrinB2 are expressed in mesothelioma cell lines as shown by RT-PCR (A) and Western Blot (B).

FIG. 12 shows expression of ephrin B2 and EphB4 by in situ hybridization in mesothelioma cells. NCI H28 mesothelioma cell lines cultured in chamber slides hybridized with antisense probe to ephrin B2 or EphB4 (top row). Control for each hybridization was sense (bottom row). Positive reaction is dark blue cytoplasmic stain.

FIG. 13 shows cellular expression of EphB4 and ephrin B2 in mesothelioma cultures. Immunofluorescence staining of primary cell isolate derived from pleural effusion of a patient with malignant mesothelioma and cell lines NCI H28, NCI H2373, and NCI H2052 for ephrin B2 and EphB4. Green color is positive signal for FITC labeled secondary antibody. Specificity of immunofluorescence staining was demonstrated by lack of signal with no primary antibody (first row). Cell nuclei were counterstained with DAPI (blue color) to reveal location of all cells. Shown are merged images of DAPI and FITC fluorescence. Original magnification 200×.

FIG. 14 shows expression of ephrin B2 and EphB4 in mesothelioma tumor. Immunohistochemistry of malignant mesothelioma biopsy. H&E stained section to reveals tumor architecture; bottom left panel is background control with no primary antibody. EphB4 and ephrin B2 specific staining is brown color. Original magnification 200×.

FIG. 15 shows that Ephrin B2, but not EphB4 is expressed in KS biopsy tissue. (A) In situ hybridization with antisense probes for ephrin B2 and EphB4 with corresponding H&E stained section to show tumor architecture. Dark blue color in the ISH indicates positive reaction for ephrin B2. No signal for EphB4 was detected in the Kaposi's sarcoma biopsy. For contrast, ISH signal for EphB4 is strong in squamous cell carcinoma tumor cells. Ephrin B2 was also detected in KS using EphB4-AP fusion protein (bottom left). (B) Detection of ephrin B2 with EphB4/Fc fusion protein. Adjacent sections were stained with H&E (left) to show tumor architecture, black rectangle indicates the area shown in the EphB4/Fc treated section (middle) detected with FITC-labeled anti-human Fc antibody as described in the methods section. As a control an adjacent section was treated with human Fc fragment (right). Specific signal arising from EphB4/Fc binding to the section is seen only in areas of tumor cells. (C) Co-expression of ephrin B2 and the HHV8 latency protein LANA1. Double-label confocal immunofluorescence microscopy with antibodies to ephrin B2 (red) LANA1 (green), or EphB4 (red) of frozen KS biopsy material directly demonstrates co-expression of LANA1 and ephrin B2 in KS biopsy. Coexpression is seen as yellow color. Double label confocal image of biopsy with antibodies to PECAM-1 (green) in cells with nuclear propidium iodide stain (red), demonstrating the vascular nature of the tumor.

FIG. 16 shows that HHV-8 induces arterial marker expression in Kaposi's sarcoma cells. (A) Western blot for ephrin B2 on various cell lysates. SLK-vGPCR is a stable clone of SLK expressing the HHV-8 vGPCR, and SLK-pCEFL is control stable clone transfected with empty expression vector. SLK cells transfected with LANA or LANAΔ440 are SLK-LANA and SLK-Δ440 respectively. Quantity of protein loading and transfer was determined by reprobing the membranes with β-actin monoclonal antibody. (B) Transient transfection of KS-SLK cells with expression vector pvGPCR-CEFL resulted in the expression of ephrin B2 as shown by immunofluorescence staining with FITC (green), whereas the control vector pCEFL had no effect. KS-SLK cells (0.8×10⁵/well) were transfected with 0.8 μg DNA using Lipofectamine 2000. 24 hr later cells were fixed and stained with ephrin B2 polyclonal antibody and FITC conjugated secondary antibody. (C) Transient transfection of HUVEC with vGPCR induces transcription from ephrin B2 luciferase constructs. 8×10³ HUVEC in 24 well plates were transfected using Superfect with 0.8 μg/well ephrin B2 promoter constructs containing sequences from −2941 to −11 with respect to the translation start site, or two 5′-deletions as indicated, together with 80 ng/well pCEFL or pvGPCR-CEFL. Luciferase was determined 48 h post transfection and induction ratios are shown to the right of the graph. pGL3Basic is promoterless luciferase control vector. Luciferase was normalized to protein since GPCR induced expression of the cotransfected β-galactosidase. Graphed is mean+SEM of 6 replicates. Shown is one of three similar experiments.

FIG. 17 shows expression of EphB4 in bladder cancer cell lines (A), and regulation of EphB4 expression by EGFR signaling pathway (B).

FIG. 18 shows a genomic nucleotide sequence of human EphB4.

FIG. 19 shows a cDNA nucleotide sequence of human EphB4.

FIG. 20 shows a genomic nucleotide sequence of human Ephrin B2.

FIG. 21 shows a cDNA nucleotide sequence of human Ephrin B2.

FIG. 22 shows an amino acid sequence of human EphB4.

FIG. 23 shows an amino acid sequence of human Ephrin B2.

FIG. 24 shows examples of EphB4 antibodies and epitope mapping of these antibodies. The topology of the EphB4 extracellular domain is shown, including a globular domain (G), a cystein-rich domain (C), and two fibronectin type 3 domains (F1 and F2).

### DETAILED DESCRIPTION OF THE INVENTION

I. Overview

The current disclosure is based in part on the discovery that signaling through the Ephrin B2/EphB4 ligand/receptor pathway contributes to tumorigenesis. Applicants detected expression or elevated expression of Ephrin B2 or EphB4 in tumor tissues and developed anti-tumor therapeutic agents for blocking either expression or activity of Ephrin B2 or EphB4. In addition, Applicants found that EphB4 gene is amplified in some tumors, for example, squamous cell carcinoma of the head and neck (HNSCC), prostate cancer, breast cancer, colorectal carcinoma, lung cancer, bladder cancer, and brain cancer. Accordingly, in certain aspects, the disclosure provides detection methods and diagnostic methods that comprise assessing gene amplification of EphB4 in a test cell sample.

Further, the disclosure contemplates gene amplification of Ephrin B2 in tumors, and thus provides detection methods and diagnostic methods that comprise assessing gene amplification of Ephrin B2 in a test cell sample. For ease of reading, the discussion below will mostly refer to methods and compositions directed to EphB4. However, one of ordinary skill in the art will readily recognize that similar methods and compositions can be derived using Ephrin B2.

The work described herein, particularly in the examples, refers to Ephrin B2 and EphB4. However, the present invention contemplates any ephrin ligand and/or Eph receptor within their respective family, which is expressed in a tumor. The ephrins (ligands) are of two structural types, which can be further subdivided on the basis of sequence relationships and, functionally, on the basis of the preferential binding they exhibit for two corresponding receptor subgroups. Structurally, there are two types of ephrins: those which are membrane-anchored by a glycerophosphatidylinositol (GPI) linkage and those anchored through a transmembrane domain. Conventionally, the ligands are divided into the Ephrin-A subclass, which are GPI-linked proteins which bind preferentially to EphA receptors, and the Ephrin-B subclass, which are transmembrane proteins which generally bind preferentially to EphB receptors.

The Eph family receptors are a family of receptor protein-tyrosine kinases which are related to Eph, a receptor named for its expression in an erythropoietin-producing human hepatocellular carcinoma cell line. They are divided into two subgroups on the basis of the relatedness of their extracellular domain sequences and their ability to bind preferentially to Ephrin-A proteins or Ephrin-B proteins. Receptors which interact preferentially with Ephrin-A proteins are EphA receptors and those which interact preferentially with Ephrin-B proteins are EphB receptors.

EphB4 is specific for the membrane-bound ligand Ephrin B2 (Sakano, S. et al 1996; Brambilla R. et al 1995). Ephrin B2 belongs to the class of Eph ligands that have a transmembrane domain and cytoplasmic region with five conserved tyrosine residues and PDZ domain. Eph receptors are activated by binding of clustered, membrane attached ephrins (Davis S et al, 1994), indicating that contact between cells expressing the receptors and cells expressing the ligands is required for Eph activation. Upon ligand binding, an Eph receptor dimerizes and autophosphorylate the juxtamembrane tyrosine residues to acquire full activation (Kalo M S et al, 1999, Binns K S, 2000). In addition to forward signaling through the Eph receptor, reverse signaling can occur through the ephrin Bs. Eph engagement of ephrins results in rapid phosphorylation of the conserved intracellular tyrosines (Bruckner K, 1997) and somewhat slower recruitment of PDZ binding proteins (Palmer A 2002). Recently, several studies have shown that high expression of Eph/ephrins may be associated with increased potentials for tumor growth, tumorigenicity, and metastasis (Easty D J, 1999; Kiyokawa E, 1994; Tang X X, 1999; Vogt T, 1998; Liu W, 2002; Stephenson S A, 2001; Steube K G 1999; Berclaz G, 1996).

One aspect of the present disclosure provides detection methods for evaluating status of a tumor or tumor prognosis, wherein the tumor expresses Ephrin B2 and/or EphB4. Such methods comprise assessing gene amplification of Ephrin B2 or EphB4 in a test cell sample.

Another aspect of the present disclosure provides therapeutic methods for reducing the growth rate of a tumor expressing Ephrin B2 and/or EphB4. Such methods comprise administering an amount of a therapeutic agent that inhibits expression or activity of Ephrin B2, EphB4, or both.

II. Detection Reagents and Methods

In certain embodiments, the present invention is based at least in part on Applicants' discovery that EphB4 gene is amplified in some tumors, for example, squamous cell carcinoma of the head and neck (HNSCC), prostate cancer, breast cancer, colorectal carcinoma, lung cancer, bladder cancer, and brain cancer. In addition, expression of EphB4 was found to be correspondingly elevated in those tumors with the EphB4 gene amplification. Thus, EphB4 gene amplification can be diagnostic of neoplasia or the potential therefor. Alternatively, detecting the elevated expression of human EphB4-encoded products (e.g., mRNAs and proteins) is also diagnostic of neoplasia or the potential for neoplastic transformation. It is further contemplated that other genetic alterations leading to elevated EphB4 expression may also be involved in tumorigenesis, such as mutations in regulatory regions of the EphB4 gene.

In certain aspects, the present invention provides the methods for evaluating (assessing or measuring) tumor status, tumor prognosis, and survivability in a subject (individual or patient), which comprise detection of an indicator of EphB4 activity. As used herein, the indicator of EphB4 activity includes EphB4 gene copy number, EphB4 mRNA, and EphB4 protein. EphB4 mRNA and EphB4 protein are also referred to herein as gene expression products of EphB4.

In certain specific embodiments, the present invention provides methods of detecting EphB4 gene amplification. Detection of gene amplification can be evaluated by determining the copy number of a target gene. Generally, a normal diploid cell has two copies of a given autosomal gene. The copy number can be increased, however, by gene amplification or duplication, for example, in cancer cells. Methods of evaluating the copy number of a particular gene are well known in the art, and include, inter alia, hybridization based and amplification based assays.

For example, a number of hybridization based assays can be used to detect the copy number of a target gene in the cells of a biological sample. One such method is Southern blot (see Ausubel et al., or Sambrook et al.), where the genomic DNA is typically fragmented, separated electrophoretically, transferred to a membrane, and subsequently hybridized to a specific probe. Comparison of the intensity of the hybridization signal from a test cell sample with a signal from a control cell comprising normal non-amplified genomic DNA can provide an estimate of the relative EphB4 gene copy number. An increased signal compared to a control represents the presence of gene amplification.

Another methodology for determining the copy number of a gene in a sample is in situ hybridization, for example, fluorescence in situ hybridization (FISH) (see Angerer, 1987 Meth. Enzymol., 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

Another alternative methodology for determining the number of gene copies is comparative genomic hybridization (CGH). In comparative genomic hybridization methods, a “test” collection of nucleic acids is labeled with a first label, while a second collection (for example, from a normal cell or tissue) is labeled with a second label. The ratio of hybridization of the nucleic acids is determined by the ratio of the first and second labels binding to each fiber in an array. Difference in the ratio of the signals from the two labels (e.g., due to gene amplification in the test collection) is detected and the ratio provides a measure of the EphB4 gene copy number. A cytogenetic representation of gene copy number variation can be generated by CGH, which provides fluorescence ratios along the length of chromosomes from differentially labeled test and reference genomic DNAs.

Hybridization protocols suitable for use with the methods of the invention are described, for example, in Albertson (1984) EMBO J. 3:1227-1234; Pinkel (1988) Proc. Natl. Acad. Sci. USA, 85:9138-9142; EPO Pub. No. 430:402; Methods in Molecular Biology, Vol. 33: In Situ Hybridization Protocols, Choo, ed., Humana Press, Totowa, N.J. (1994).

Alternatively, amplification-based assays can also be used to measure the copy number of a target gene (e.g., EphB4). In such assays, a target nucleic acid sequence acts as a template in an amplification reaction (for example, Polymerase Chain Reaction or PCR). In a quantitative amplification, the amount of amplification product will be proportional to the amount of template in the original sample. Comparison to appropriate controls provides a measure of the copy number of the gene, according to the principles discussed above. Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994. A TaqMan-based assay can also be used to quantify EphB4 gene copy number. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase (e.g., AmpliTaq) results in the cleavage of the TaqMan probe. This cleavage separates the 5′ fluorescent dye and the 3′ quenching agent, thereby resulting in an increase in fluorescence as a function of amplification (see, for example, http://www2.perkin-elmer.com).

Examples of preferred primers for use in quantitative amplification reactions for determining EphB4 gene copy number are provided below:

| | |
|---|---|
| EphB4-forward: | 5′-TCC TGC AAG GAG ACC TTC AC-3′ |
| EphB4-reverse: | 5′-CAG AGG CCT CGC AAC TAC AT-3′ |

Predicted size of PCR product: 195 bp

These primers are preferably employed with a set of control primers directed to a gene that is not expected to have increased copy number, such as below:

| | |
|---|---|
| GAPDH-forward: | 5′-GAG GGG TGA TGT GGG GAG TA-3′ |
| GAPDH-reverse: | 5′-GAG CTT CCC GTT CAG CTC AG-3′ |

Predicted size: 206 bp

Annealing temperature for both sets of primers: 64° C.

Other suitable amplification based methods include, but are not limited to, ligase chain reaction (LCR) (see, Wu and Wallace, Genomics, 4: 560, 1989; Landegren et al., Science, 241: 1077, 1988; and Barringer et al., Gene, 89:117, 1990), transcription amplification (Kwoh et al., Proc. Natl. Acad. Sci. USA, 86:1173, 1989), self-sustained sequence replication (Guatelli et al., Proc Nat Acad Sci, USA 87:1874, 1990), dot PCR, and linker adapter PCR, for example.

Another powerful method for determining gene copy numbers employs microarray-based platforms. Microarray technology may be used because it offers high resolution. For example, the traditional CGH generally has a 20 Mb limited mapping resolution; whereas in microarray-based CGH, the fluorescence ratios of the differentially labeled test and reference genomic DNAs provide a locus-by-locus measure of DNA copy-number variation, thereby achieving increased mapping resolution. Details of various microarray methods can be found in the literature. See, for example, U.S. Pat. No. 6,232,068; Pollack et al., Nat. Genet., 23(1):41-6, (1999), and others.

In other specific embodiments, the present invention relates to detection of gene expression products (e.g., EphB4 mRNAs or proteins). mRNA transcription can be measured by a variety of techniques, including Northern blotting (Thomas (1980) Proc. Natl. Acad. Sci. USA 77:5201-5205), dot blots, and in situ hybridization. A variety of methods for measuring expression of the gene product exist, including Western blotting and immunohistochemical staining. Western blots are run by spreading a protein sample on a gel, usually an SDS gel, blotting the gel with a cellulose nitrate filter, and probing the filters with labeled antibodies. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al. (1980) Am. J. Clin. Path. 75:734-738.

In certain aspects, an increased level of the indicator of EphB4 activity (e.g., gene amplification) is directly related to the invasiveness of the tumor and the likelihood that the tumor has metastasized or will metastasize. For example, patients who test positively for EphB4 gene amplification are less likely to survive (poor prognosis) and will usually suffer a shorter time to relapse after surgical removal of the tumor than patients without such gene amplification. As another example, the patients displaying gene amplification may benefit from aggressive treatment regimens after surgical removal of their tumors. Conversely, patients who do not display gene amplification may be less likely to require such rigorous treatment.

In certain aspects, the present invention is useful for screening a wide variety of neoplastic diseases, including both solid tumors and hematopoietic cancers. Exemplary neoplastic diseases include, but are not limited to, carcinomas such as adenocarcinomas and melanomas; mesodermal tumors such as neuroblastomas and retinoblastomas; sarcomas such as osteosarcomas, Ewing's sarcoma, and various leukemias; and lymphomas. Of particular interest are carcinomas of the prostate, head and neck, breast, ovaries, colon and rectum, lung, stomach, brain, and liver.

According to one embodiment of the invention, a method of diagnosing a neoplastic tissue in a human is provided. Test samples (e.g., tissues or bodily fluids) are isolated from a human, and the copy number of human EphB4 gene is determined. Alternatively, levels of human EphB4 gene expression products can be determined. These include protein and mRNA.

Depending on the nature of the cancer (tumor), an appropriate patient sample is obtained. For example, in the case of solid tumors, a tissue sample from the surgically removed tumor will be obtained and prepared for testing by conventional techniques. In the case of lymphomas and leukemias, lymphocytes, leukemic cells, or lymph tissues will be obtained and appropriately prepared. In certain cases, test tissues or cells are obtained from a tumor (e.g., the primary tumor) or a tissue suspected of being neoplastic. Normally, the test tissues or cells are desirably separated from normal appearing tissue for analysis. This can be done by paraffin or cryostat sectioning or flow cytometry, as is known in the art. In other cases, test tissues or cells are obtained from a tissue that is suspected of having metastatic cells derived from the primary tumor, such as a lymph node or a tissue that is close to the primary tumor. Optionally, non-neoplastic tissue or any normal tissue can be used to determine a baseline level of gene expression or gene copy number, against which the amount of EphB4 gene expression or gene amplification can be compared.

In another specific embodiment, test samples such as bodily fluids will also find use with particular tumors. For example, a bodily fluid can be assayed to detect the elevated levels of the gene expression product or of gene amplification. Suitable body fluids include blood, serum, plasma, a blood-derived fraction, lymph fluid, saliva, sputum, stool, urine, a colonic effluent, breast exudate, and a wide variety of useful immunoassays are described in the patent and scientific literature. See, for example, U.S. Pat. Nos. 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876.

In certain specific embodiments, measurement of gene amplification will be performed quantitatively so that the number of EphB4 gene copies can be estimated. Status of the disease may correlate directly with the number of gene copies present in the tumor cells. For example, patients displaying gene amplification (e.g., three copies of the EphB4 gene) are at a higher risk of relapse than patients not displaying gene amplification (e.g., two copies of EphB4 gene). Moreover, as the number of EphB4 gene copies increases, the invasiveness and likelihood of metastasis also appears to increase. Optionally, the number of EphB4 gene copies should be taken as an important factor in assessing the tumor status along with the traditional factors, including lymph node status, estrogen receptor status, progesterone receptor status, and the like. With all this information available, the treating physician is best able to assess the tumor status and recommend the best treatment strategy to benefit the patient.

Generally, the human EphB4 gene is considered to be amplified if the cell contains more than the normal copy number (2) of this gene per genome. The various techniques for detecting gene amplification are well known in the art. Gene amplification can be determined, for example, by Southern blot analysis, wherein cellular DNA from a human tissue is digested, separated, and transferred to a filter where it is hybridized with a probe containing complementary nucleic acids. Alternatively, quantitative polymerase chain reaction (PCR) employing primers can be used to determine gene amplification. Appropriate primers will bind to sequences that bracket human EphB4 coding sequences. Other techniques for determining gene copy number as are known in the art can be used without limitation.

The gene product which is measured may be either mRNA or protein. The term “elevated expression” means an increase in mRNA production or protein production over that which is normally produced by non-cancerous cells. Although amplification has been observed in human tumors, other genetic alterations leading to elevated expression of EphB4 may be present in these or other tumors. Examples of tumors include, but are not limited to, HNSCC, lung, breast, brain, colorectal, bladder, prostate, brain, liver, skin, and stomach. Non-cancerous cells for use in determining baseline expression levels can be obtained from cells surrounding a tumor, from other humans or from human cell lines. Any increase can have diagnostic value, but generally the mRNA or protein expression will be elevated at least about 2-fold, 3-fold, 5-fold, 10-fold, 20-fold, and in some cases up to about 50-fold over that found in non-cancerous cells. The techniques employed for detecting mRNA or protein are well known and routine in the art. Increased production of mRNA or protein may be detected, for example, using the techniques of Northern blot analysis or Western blot analysis, respectively, or other known techniques such as ELISA, immunoprecipitation, RIA and the like. All these techniques are well known to the skilled artisan.

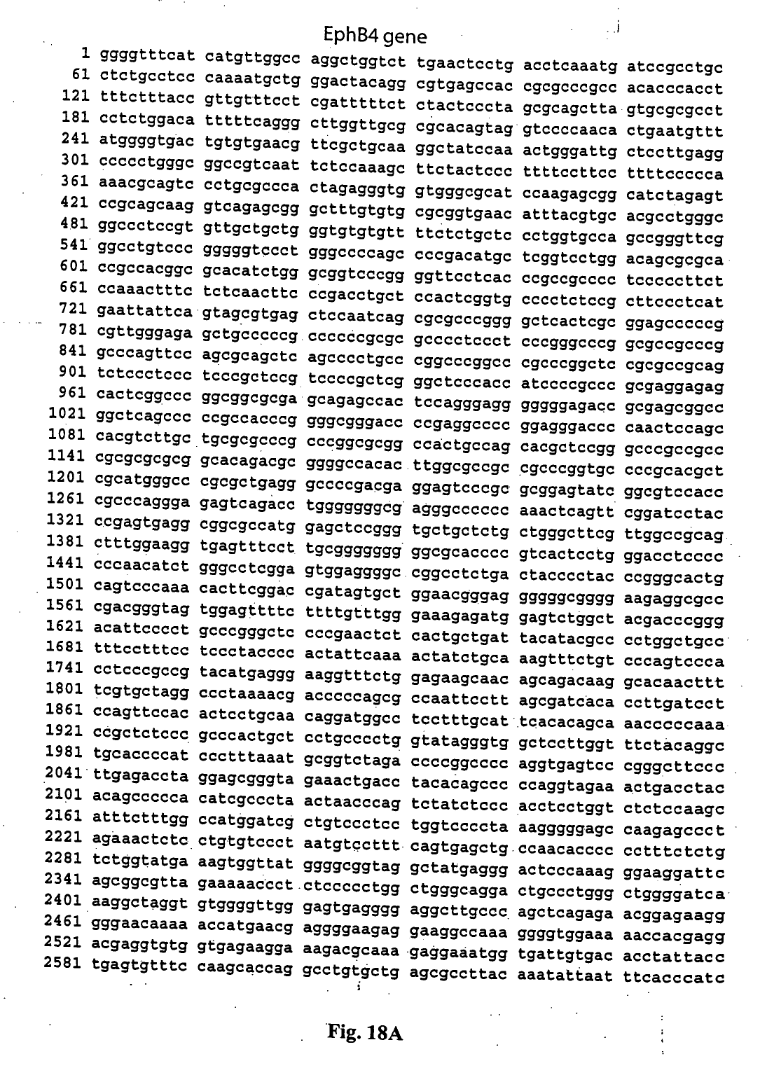

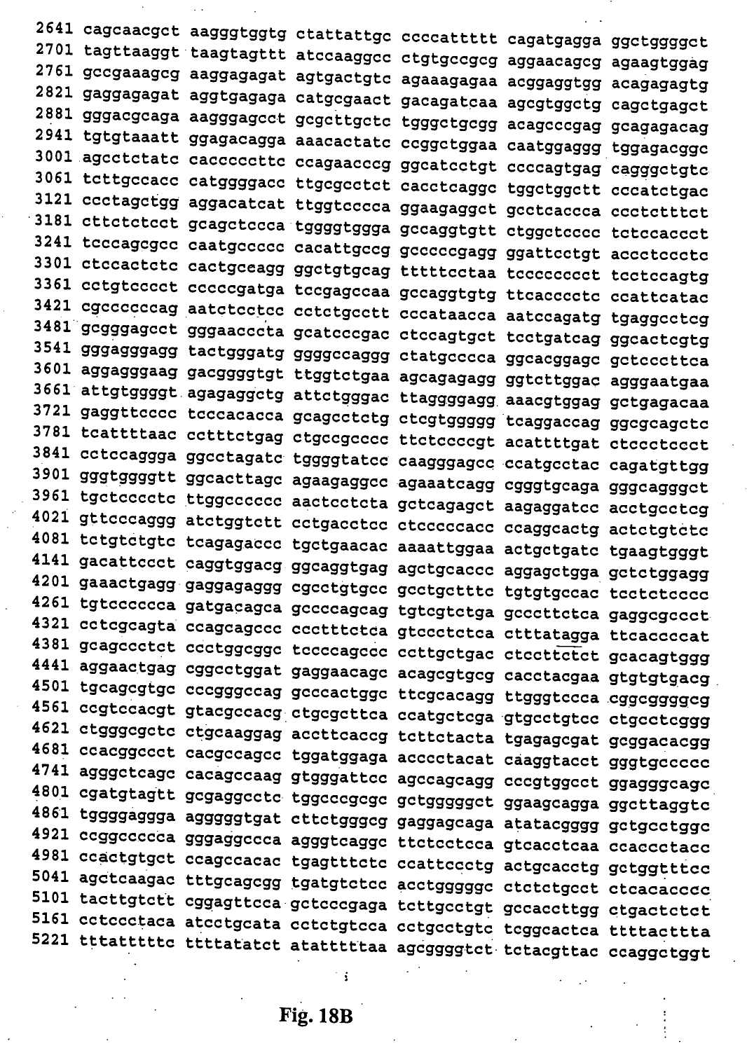

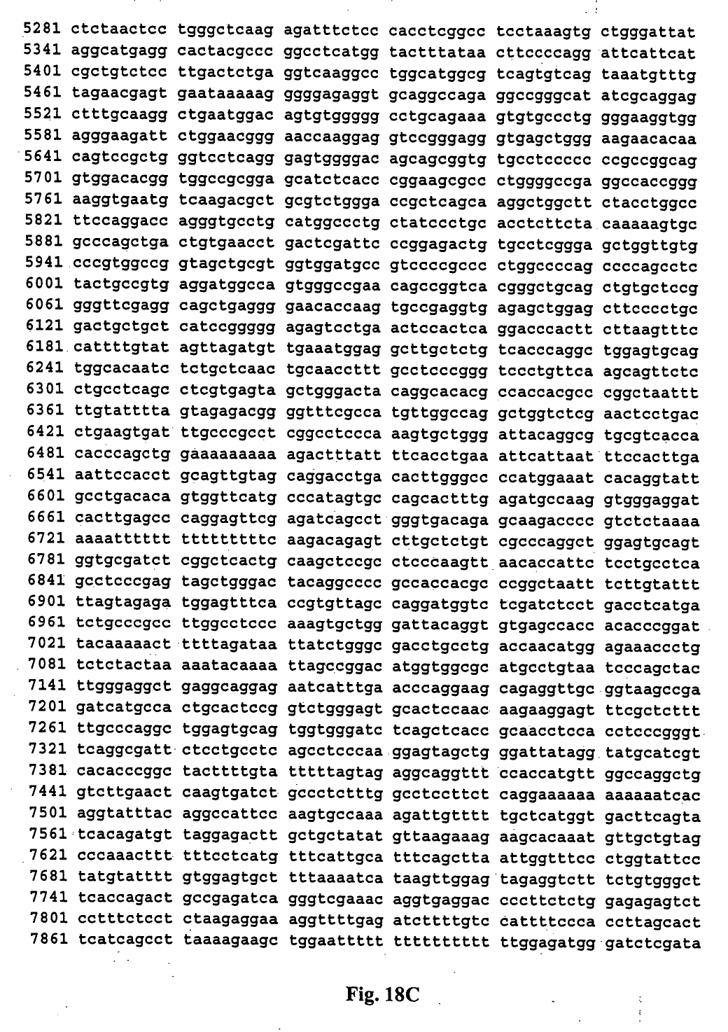

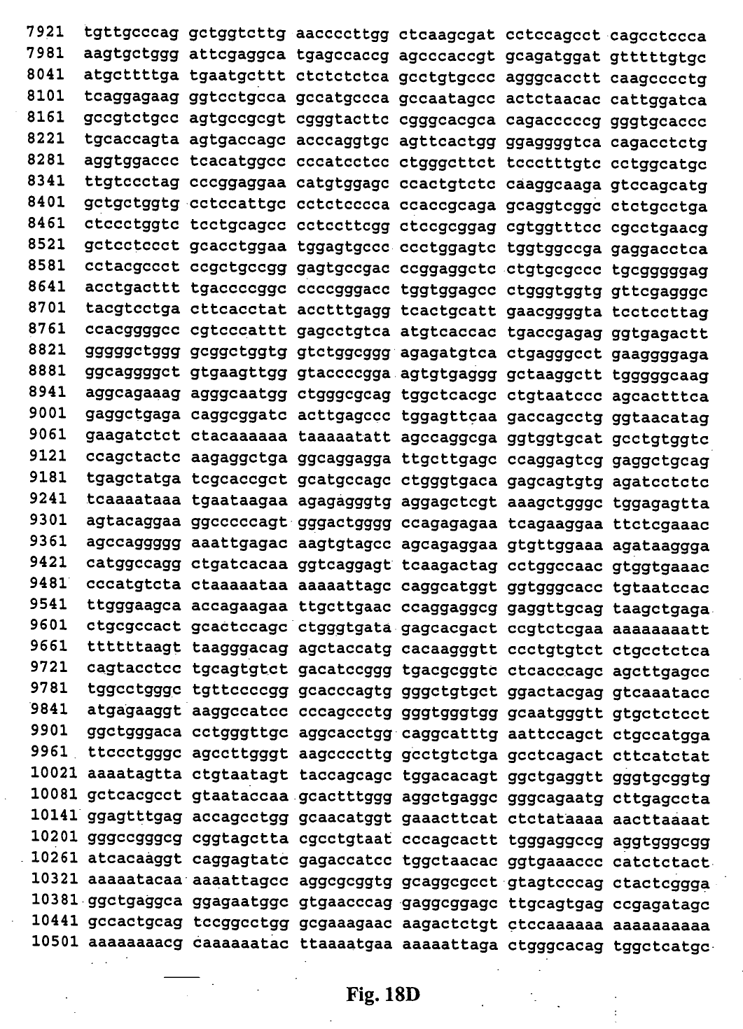

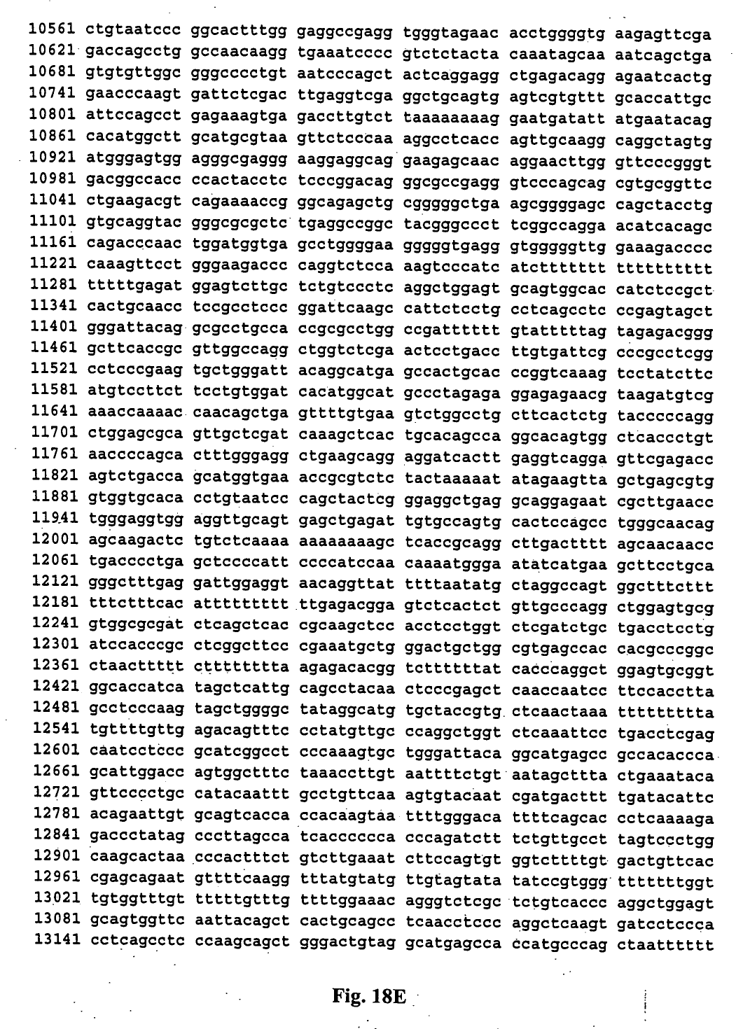

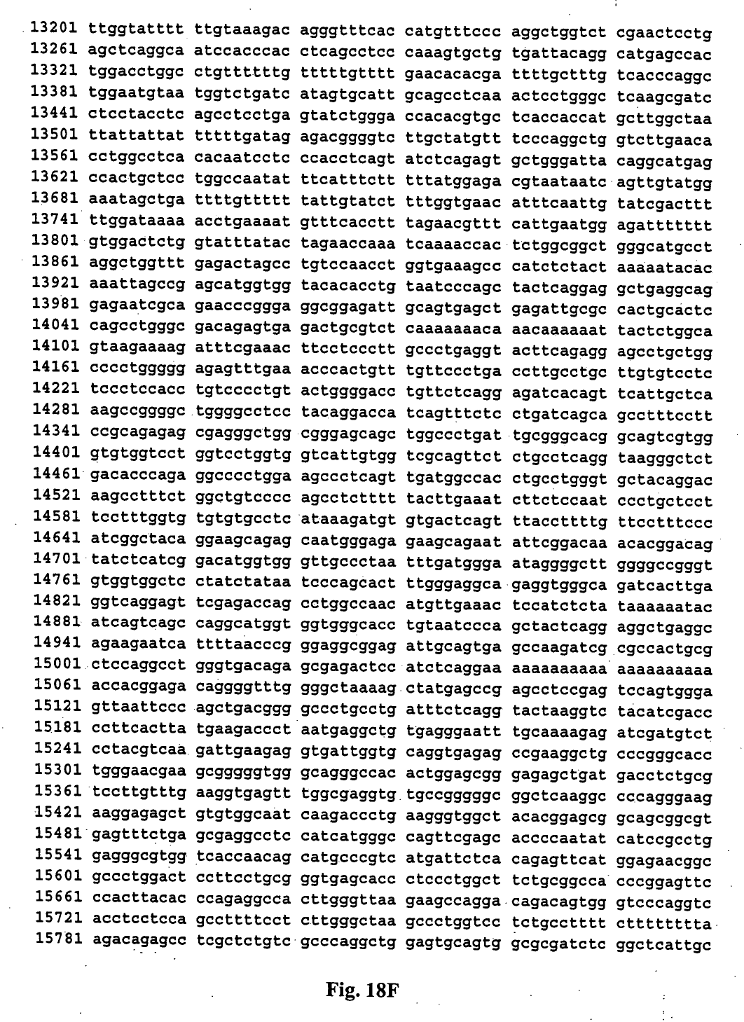

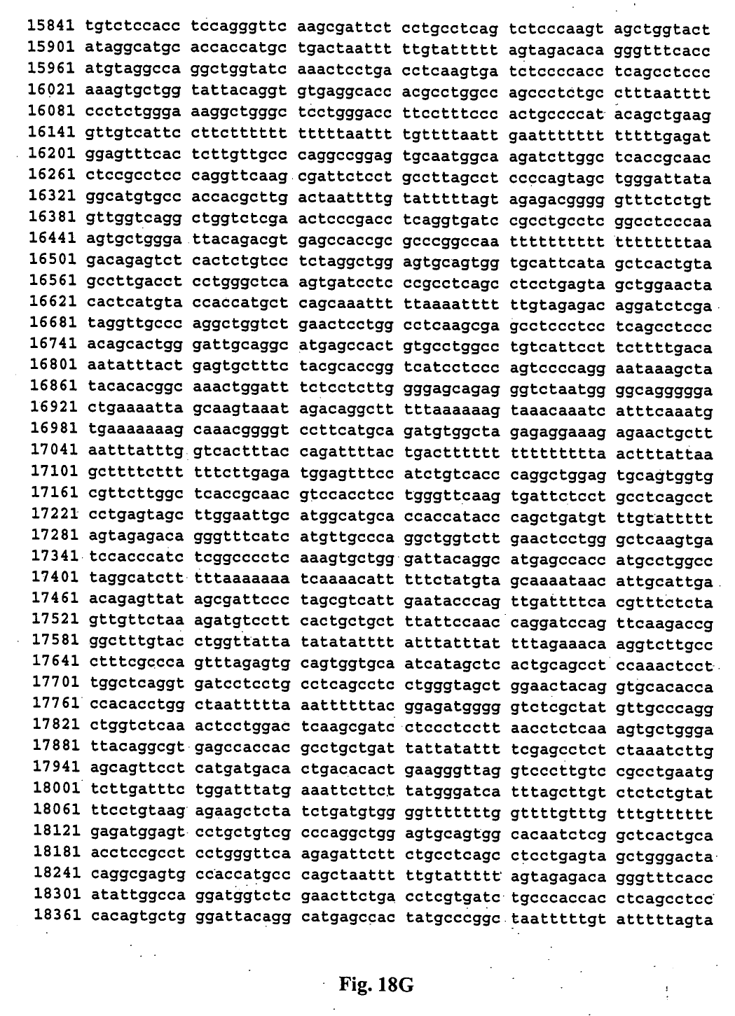

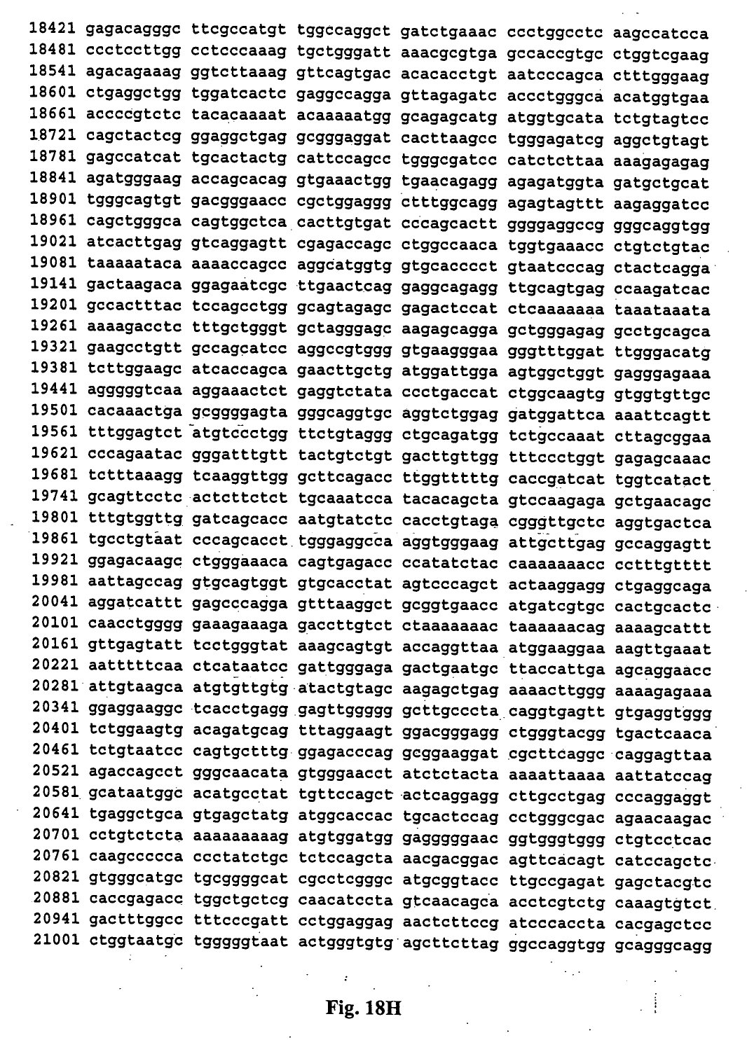

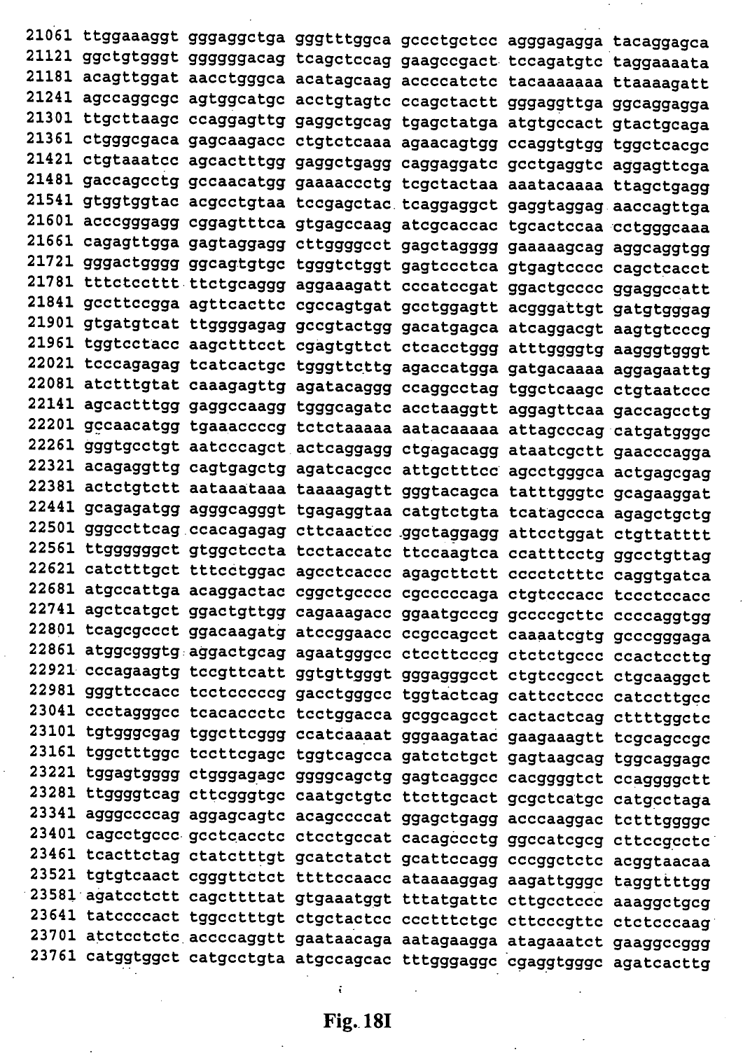

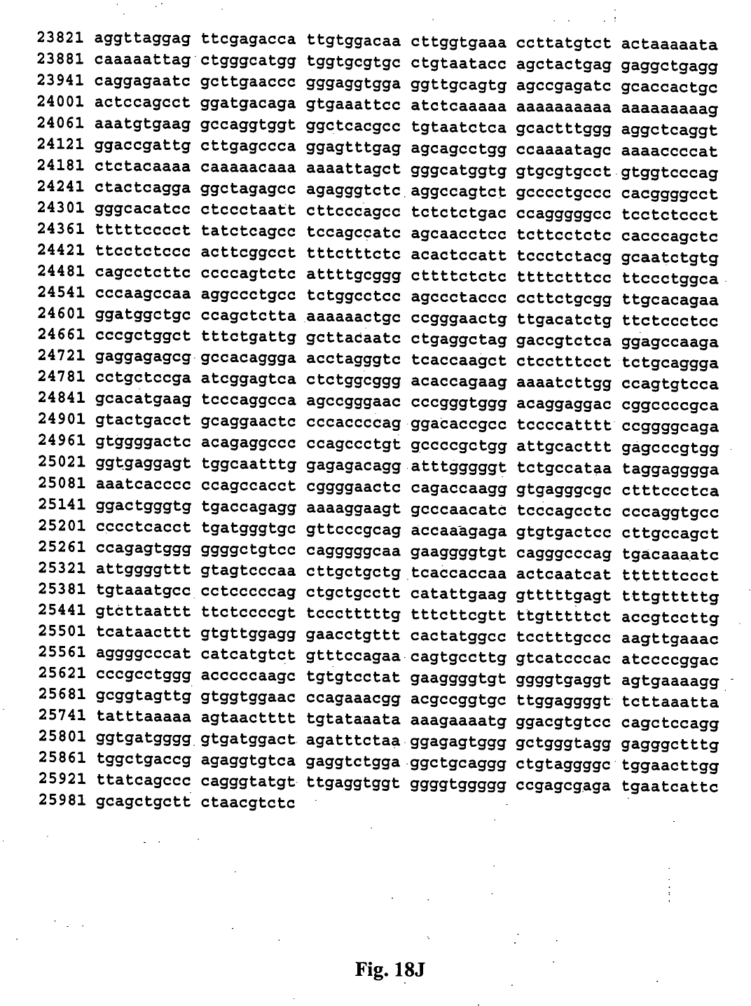

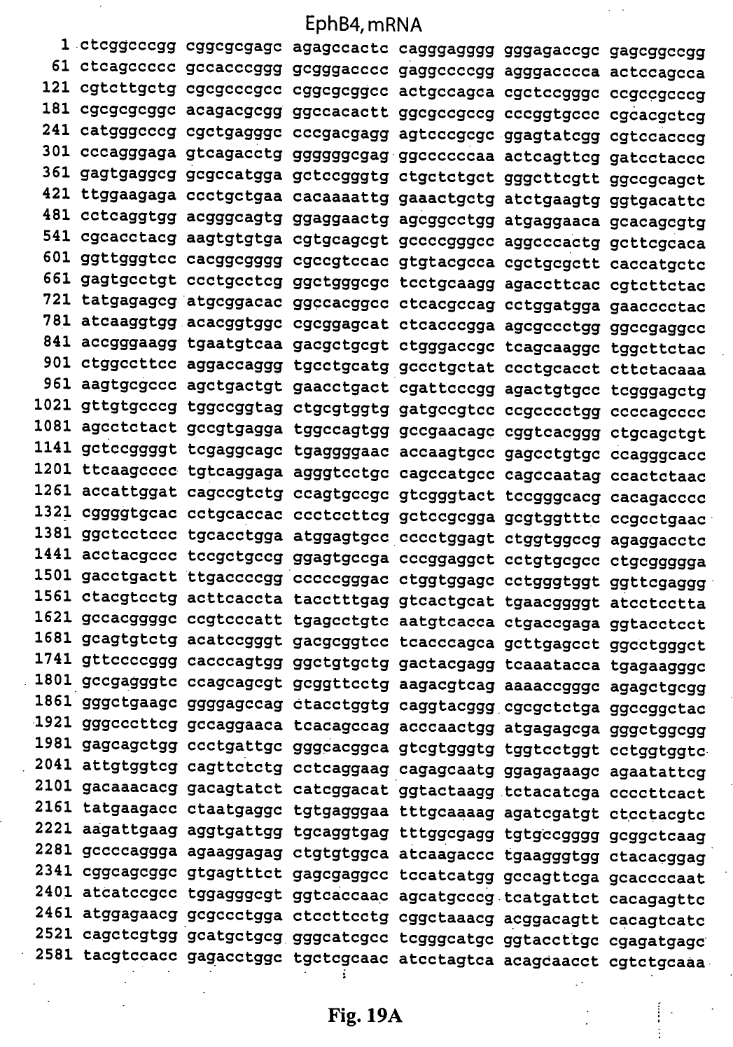

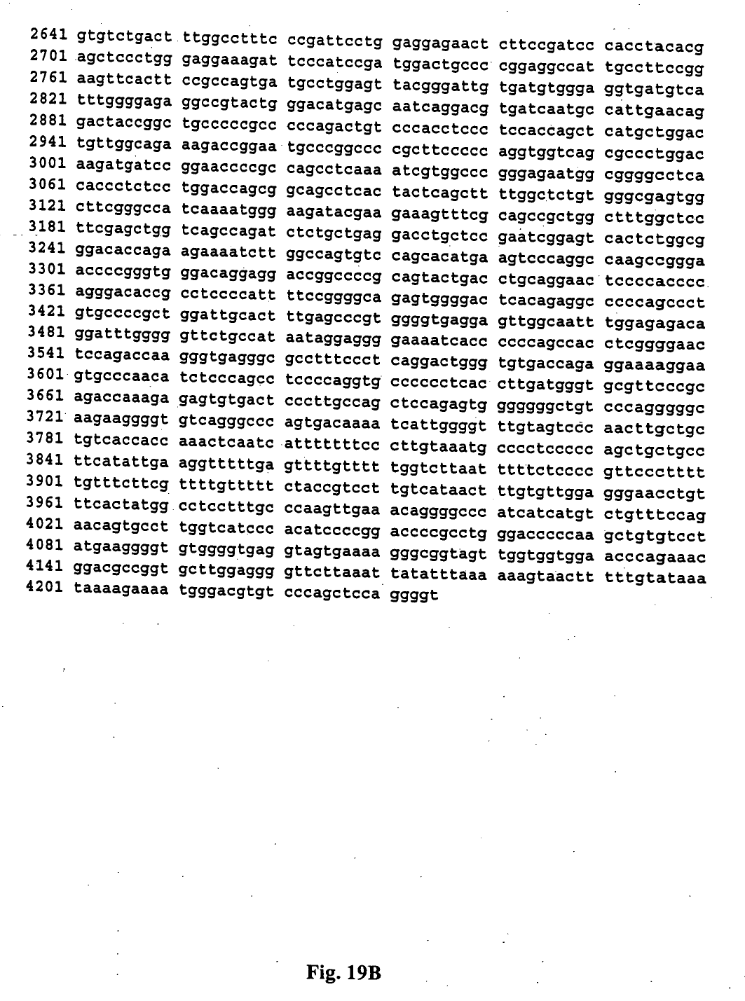

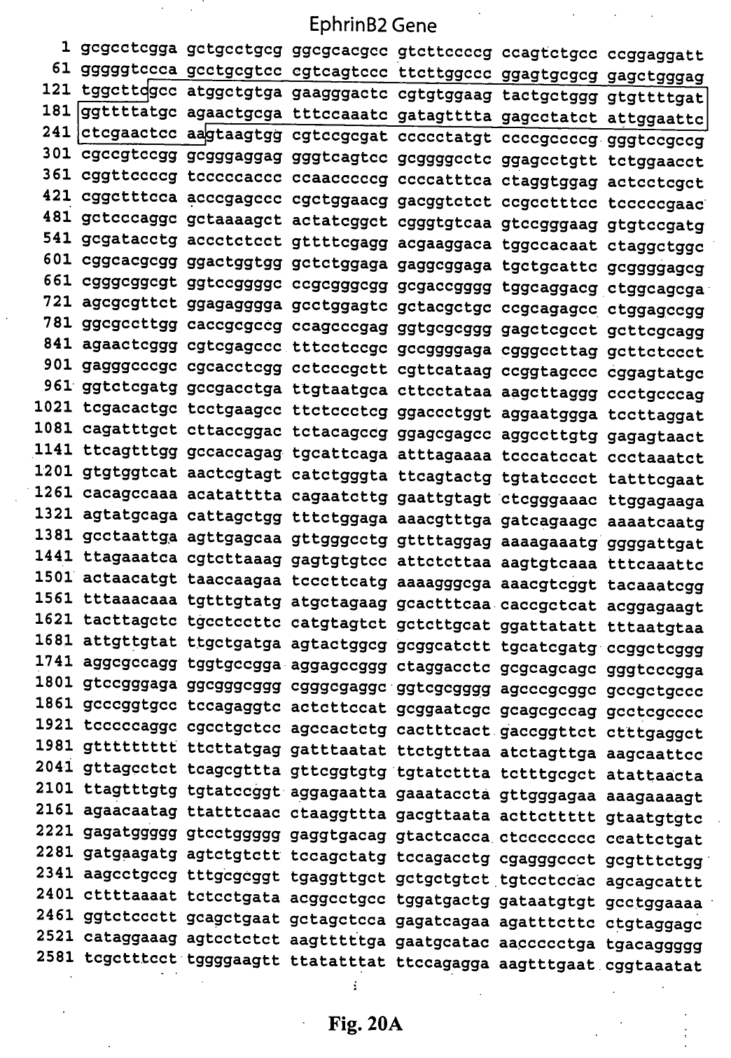

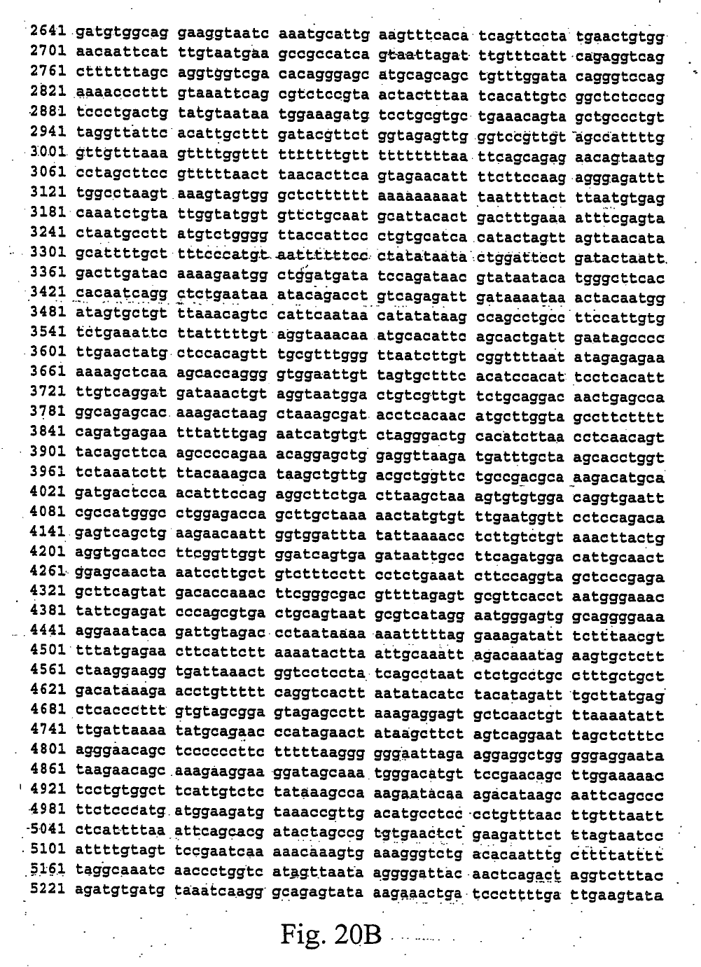

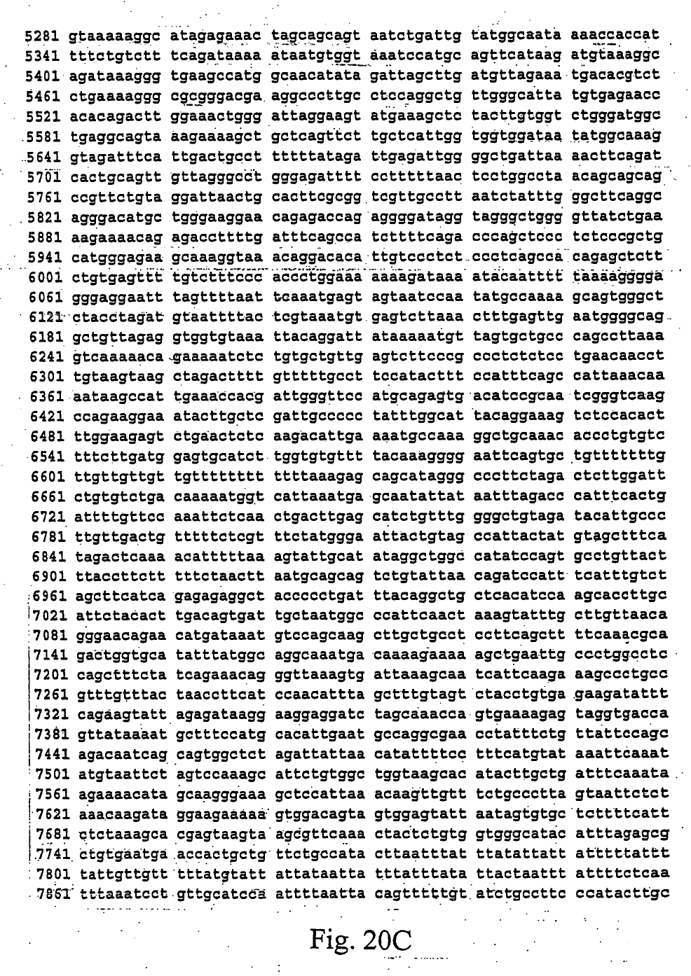

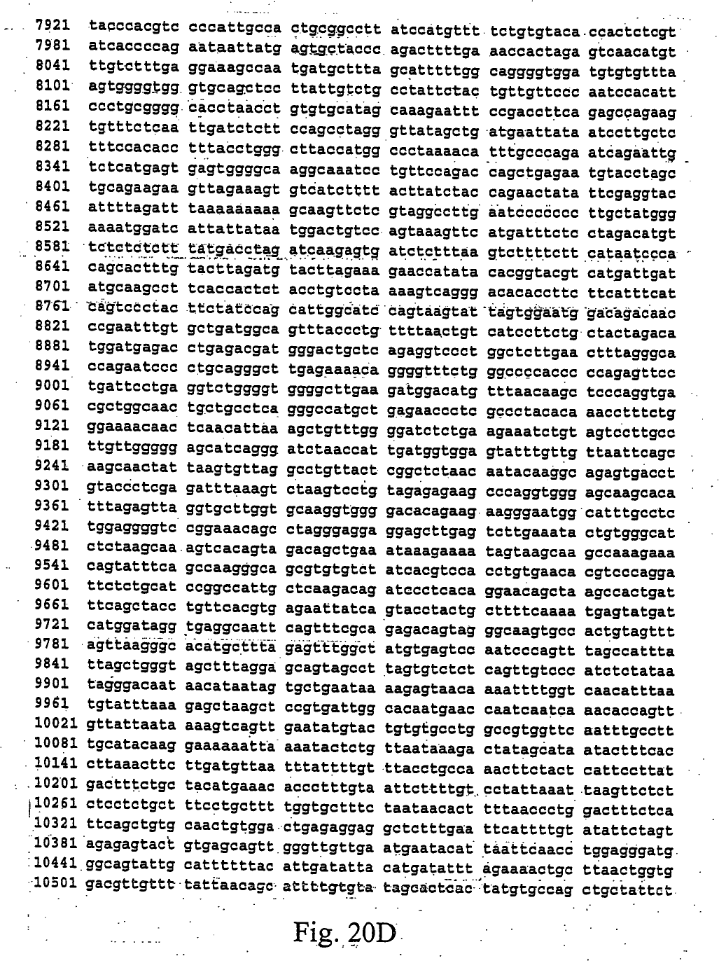

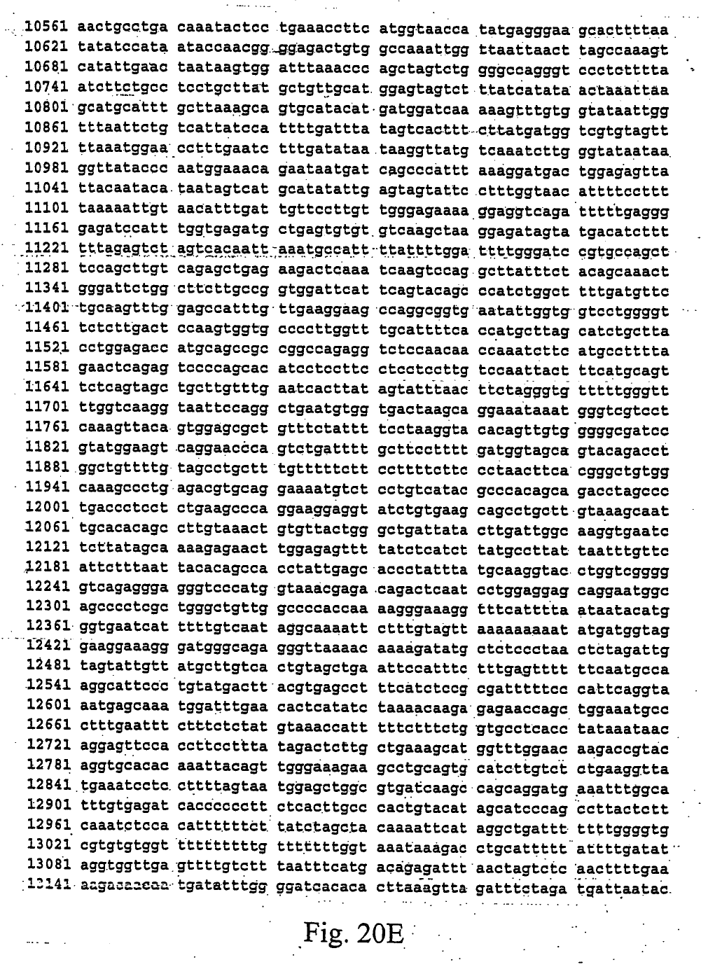

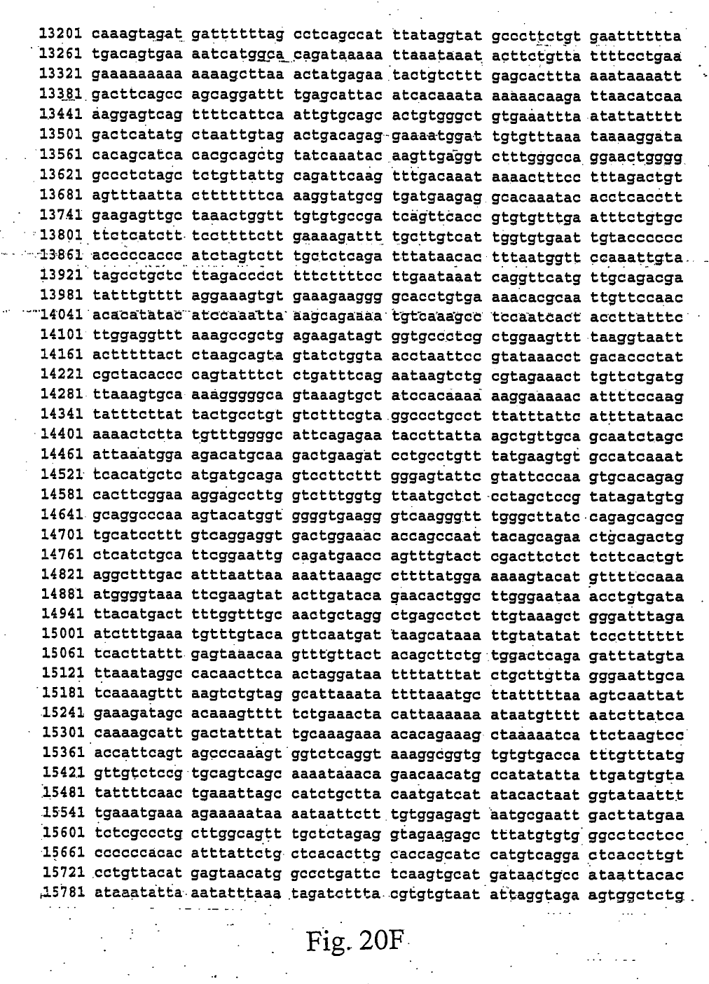

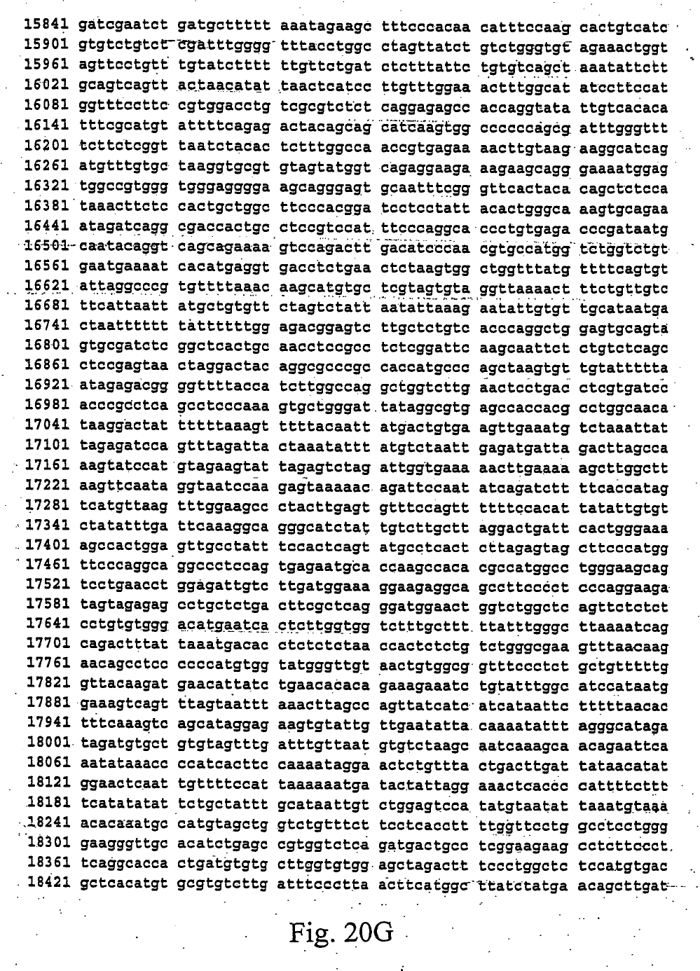

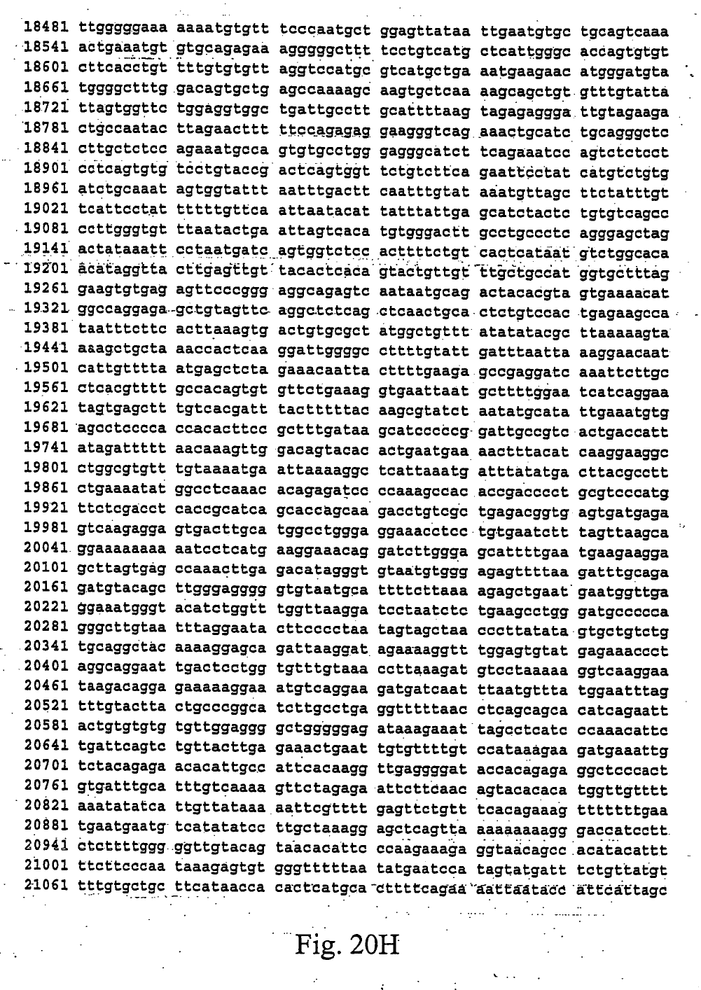

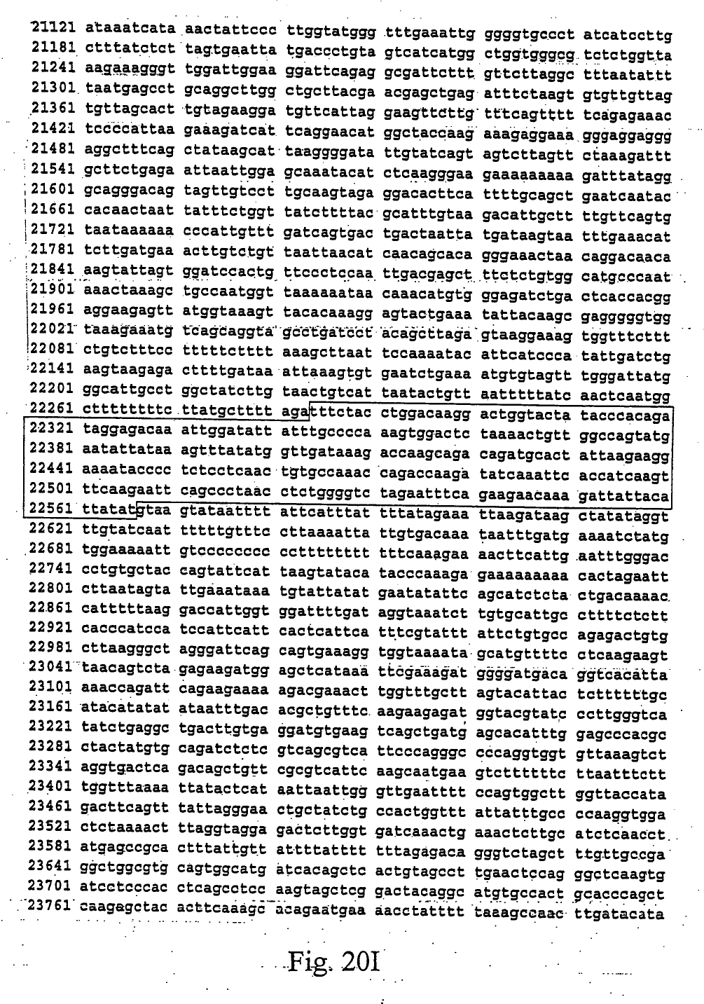

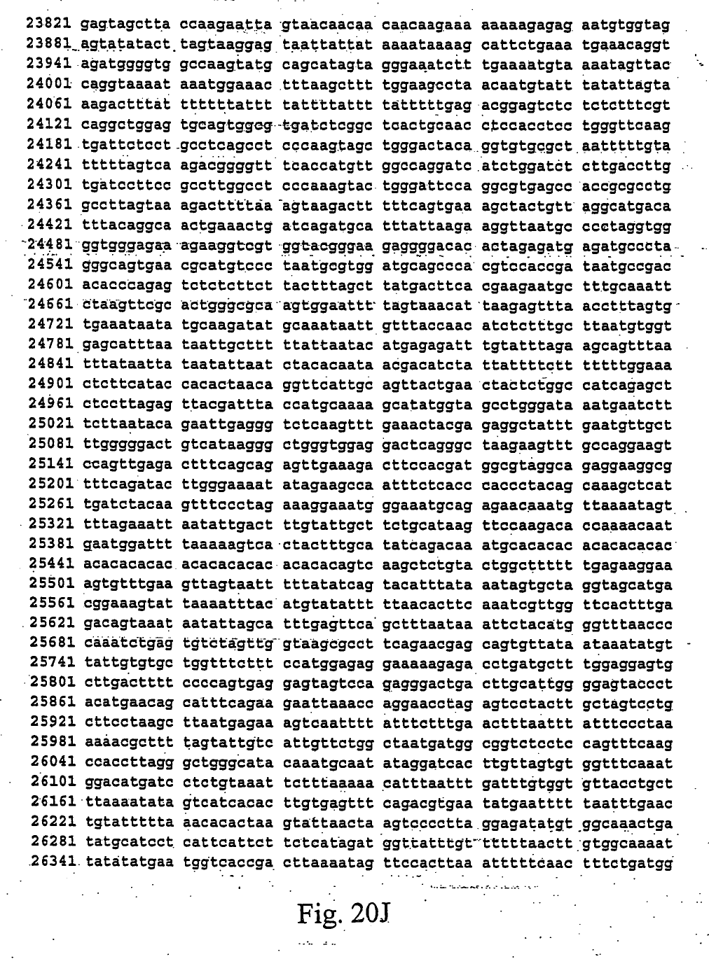

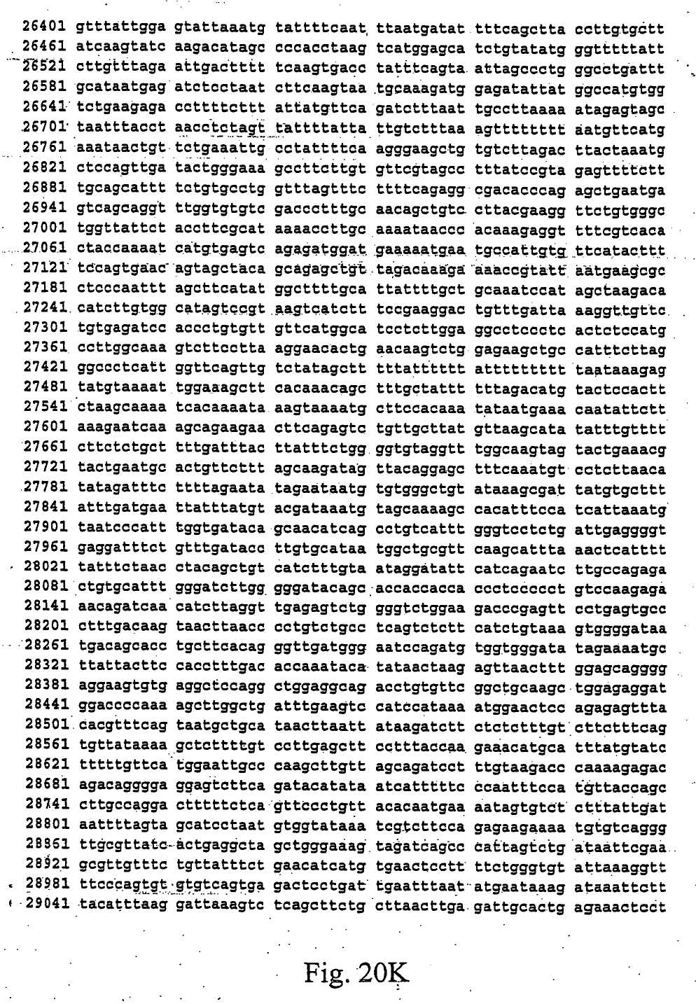

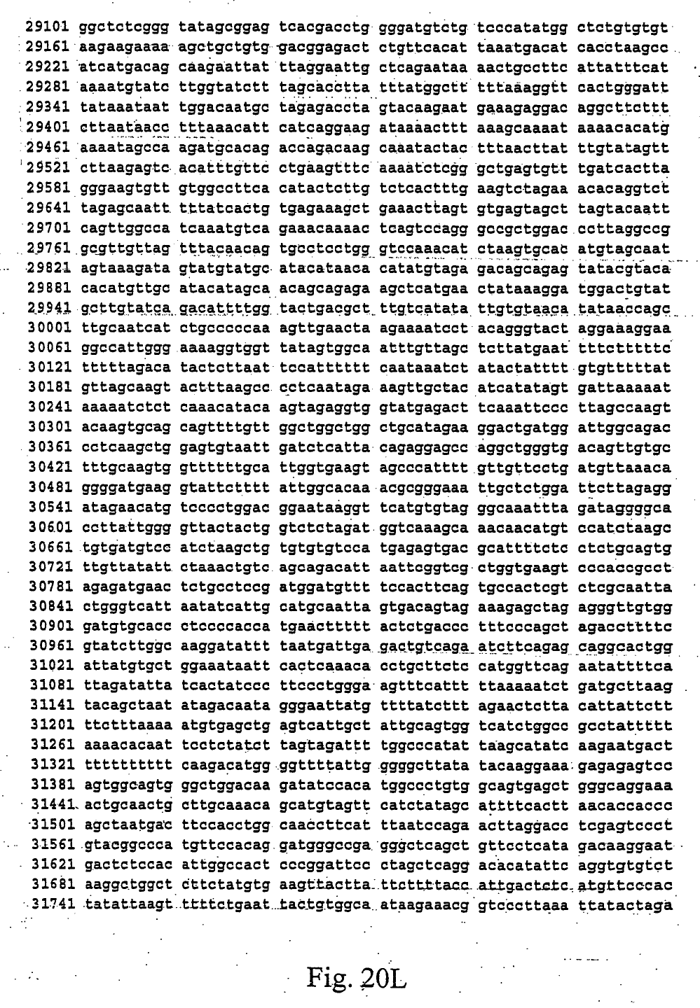

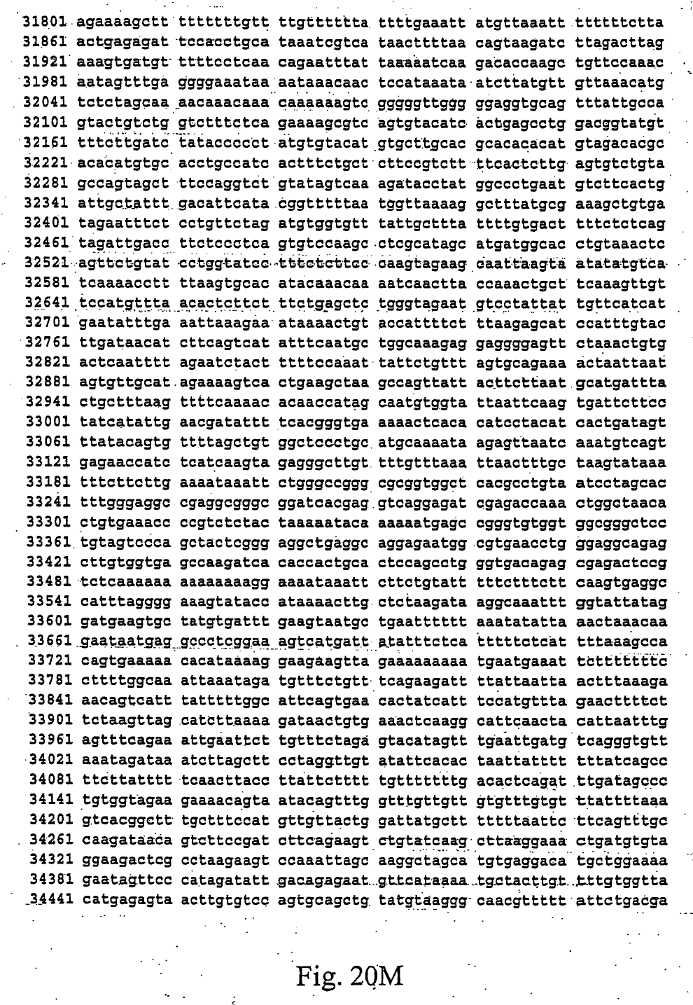

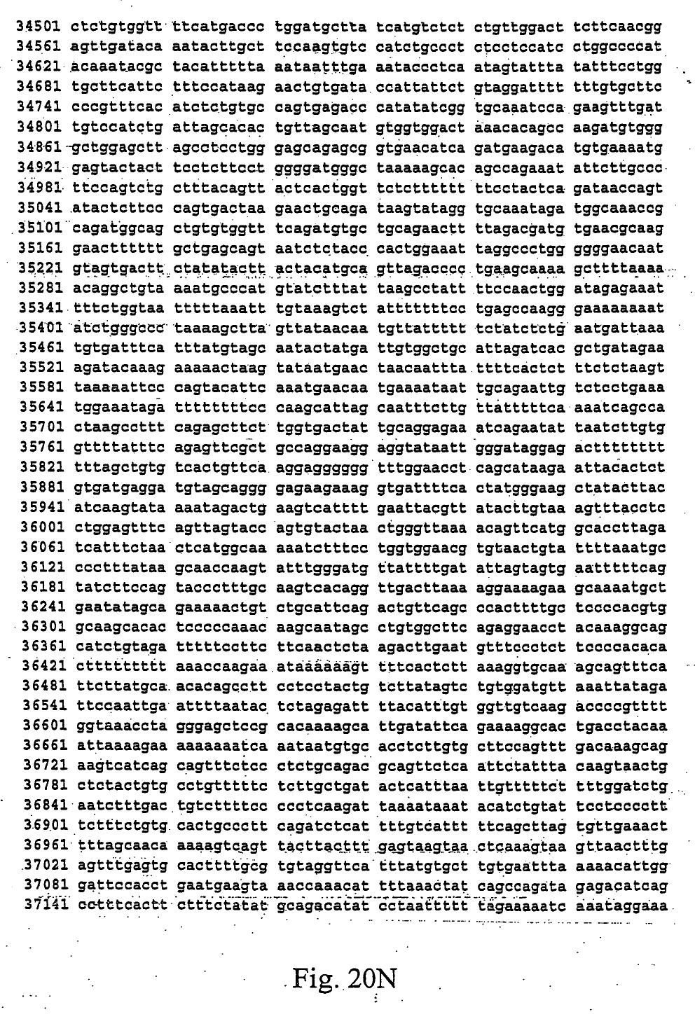

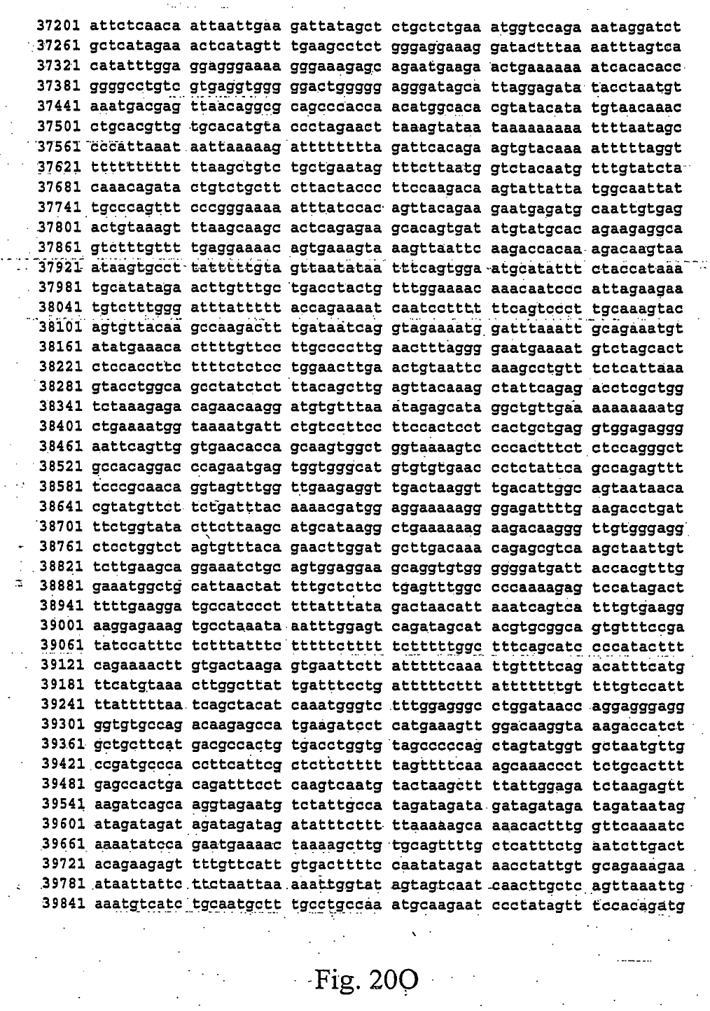

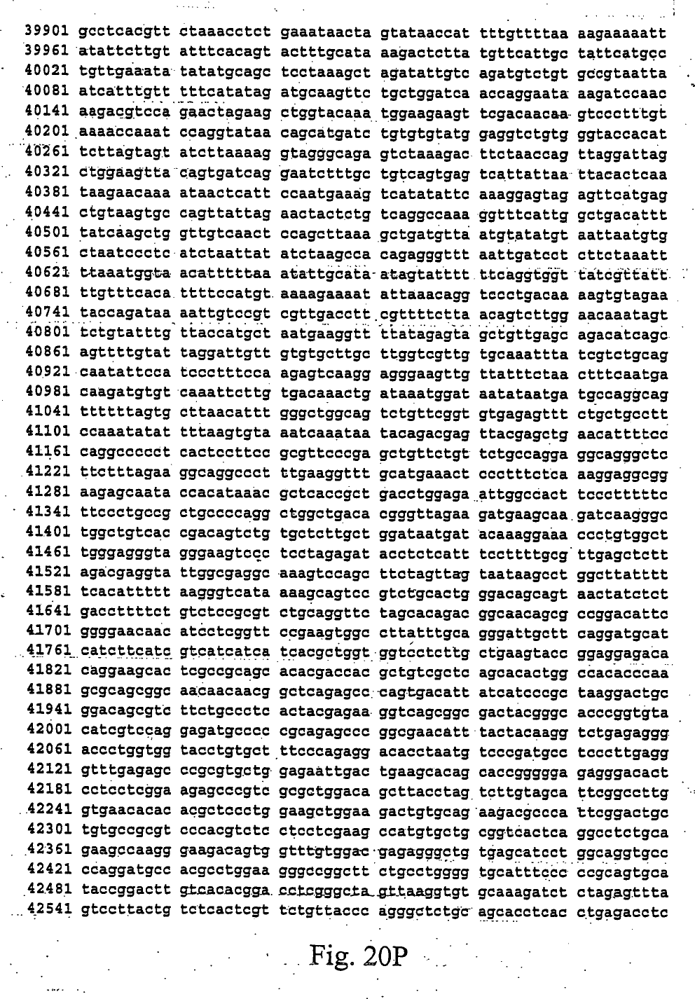

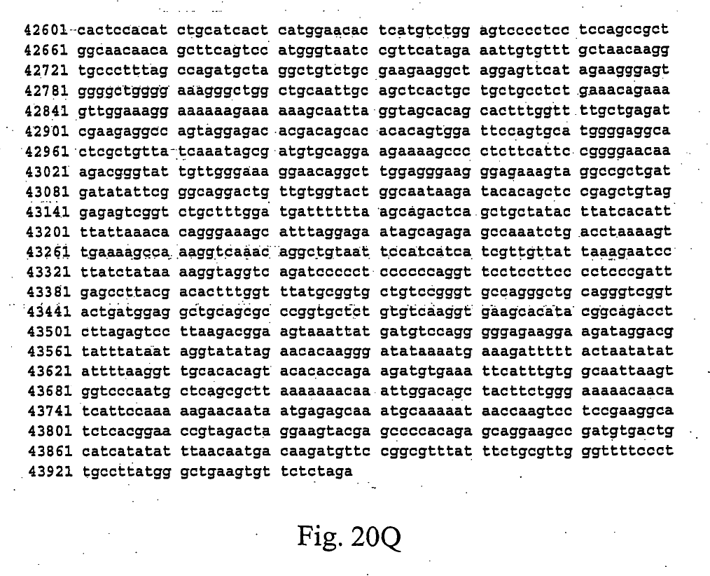

According to another embodiment of the invention, nucleic acid probes or primers for the determining of human EphB4 gene amplification or elevated expression of mRNA are provided. The probe may comprise ribo- or deoxyribonucleic acids, and may contain the entire human EphB4 coding nucleotide sequence, a sequence complementary thereto, or fragments thereof. A probe may contain, for example, nucleotides as shown in FIGS. 18 and 19. Generally, probes or primers will contain at least about 14 contiguous nucleotides of the human sequence but may desirably contain about 40, 50 or 100 nucleotides. Probes are typically labelled with a fluorescent tag, a radioisotope, or the like to render them easily detectable. Preferably the probes will hybridize under stringent hybridization conditions. The probes of the invention are complementary to the human EphB4 gene. This means that they share a high sequence identity (e.g., 95%, 98%, 99% or 100%) with the human EphB4 sequence.

EphB4 protein can be produced, according to the invention, substantially free of other human proteins. Provided with the EphB4 DNA sequence, those of skill in the art can express the cDNA in a non-human cell. Lysates of such cells provide proteins substantially free of other human proteins. The lysates can be further purified, for example, by immunoprecipitation, or by affinity chromatography.

The antibodies of the invention are specifically reactive with EphB4 protein. Preferably, they do not cross-react with EphB4 from other species. They can be polyclonal or monoclonal, and can be raised against a native EphB4, or an EphB4 fusion protein, or synthetic peptide. The antibodies are specifically immunoreactive with EphB4 epitopes which are not present on other human proteins. Optionally, some antibodies are reactive with epitopes unique to human EphB4 and not present on the mouse homolog. The antibodies are useful in conventional analyses, such as Western blot analysis, ELISA, immunohistochemistry, and other immunological assays for the detection of proteins. Techniques for raising and purifying polyclonal antibodies are well known in the art, as are techniques for preparing monoclonal antibodies. Antibody binding can be determined by methods known in the art, such as use of an enzyme-labelled secondary antibody, staphylococcal protein A, and the like.

Kits are provided which contain the necessary reagents for determining gene copy number, such as probes or primers specific for the EphB4 gene, as well as written instructions. The instructions can provide calibration curves to compare with the determined values. Kits are also provided to determine elevated expression of mRNA (e.g., containing probes) or EphB4 protein (e.g., containing antibodies). Instructions will allow one to determine whether the expression levels of EphB4 are elevated. Reaction vessels and auxiliary reagents such as chromogens, buffers, and enzymes, may also be included in the kits.

III. Therapeutic Agents

In certain embodiments, the present invention provides a therapeutic modality for interfering with the expression or activity of EphB4 and/or Ephrin B2. The method can be applied in vivo, in vitro, or ex vivo, and will be particularly useful in cancers having one or more indicators of elevated EphB4 activity. Therapeutic agents may include nucleic acids, polypeptides, antibodies, and small molecule compounds. Examples of these therapeutic agents have been described in U.S. patent application Ser. Nos. 10/800,077 and 10/800,350, and in PCT Application Nos. US04/07491 and US04/07755.

For example, the present invention provides nucleic acid therapeutic agents which can be single-, double-, or multiple-stranded, and can comprise modified or unmodified nucleotides or non-nucleotides or various mixtures, and combinations thereof. Examples of nucleic acid therapeutic agents include, but are not limited to, antisense nucleic acids, dsRNA, siRNA, and enzymatic nucleic acid compounds (e.g., riozyme or DNA enzyme). Optionally, the disclosure features one or more nucleic acid therapeutic agents that independently or in combination modulate expression of the Ephrin B2 gene encoding an Ephrin B2 protein (e.g., Genbank Accession No.: NP_{—}004084) or the EphB4 receptor gene which encodes an EphB4 protein (e.g., Genbank Accession No.: NP_{—}004435).

In a specific embodiment, exemplary EphB4 antisense nucleic acids are provided in Table 1 below, and exemplary EphB4 siRNAs are provided in Table 2 below. In another specific embodiment, exemplary Ephrin B2 antisense nucleic acids are provided in Table 3 below, and exemplary Ephrin B2 siRNAs are provided in Table 2 below.

TABLE 1
Examples of EphB4 antisense probes

| | | | | |
|---|---|---|---|---|
| | | | | Percent |
| | | | Inhibition | reduction |
| | | | of EphB4 | in |
| Name | Sequence 5′ → 3′ | position | Expression | viability |

| | | | | |
|---|---|---|---|---|
| Eph B4 169 | TCA GTA CTG CGG GGC CGG TCC | (2944-2963) | ++ | 36 |
| Eph B4 168 | TCC TGT CCC ACC CGG GGT TC | (2924-2943) | ++ | 51 |
| Eph B4 167 | CCG GCT TGG CCT GGG ACT TC | (2904-2923) | +++ | 66 |
| Eph B4 166 | ATG TGC TGG ACA CTG GCC AA | (2884-2903) | ++++ | 70 |
| Eph B4 165 | GAT TTT CTT CTG GTG TCC CG | (2864-2883) | ++++ | 75 |
| Eph B4 164 | CCA GAG TGA CTC CGA TTC GG | (2844-2863) | ++ | 40 |
| Eph B4 163 | AGC AGG TCC TCA GCA GAG AT | (2824-2843) | ++++ | 66 |
| Eph B4 162 | CTG GCT GAC CAG CTC GAA GG | (2804-2823) | | 25 |
| Eph B4 161 | AGC CPA AGC CAG CGG CTG CG | (2784-2803) | + | 33 |
| Eph B4 160 | AAA CTT TCT TCG TAT CTT CC | (2763-2783) | + | 25 |
| Eph B4 159 | CAT TTT GAT GGC CCG AAG CC | (2743-2762) | ++ | 40 |
| Eph B4 158 | ACT CGC CCA CAG AGC CAA AA | (2723-2742) | | 30 |
| Eph B4 157 | GCT GAG TAG TGA GGC TGC CG | (2703-2722) | + | 25 |
| Eph B4 156 | CTG GTC CAG GAG AGG GTG TG | (2683-2702) | ++ | 30 |
| Eph B4 155 | AGG CCC CGC CAT TCT CCC GG | (2663-2682) | | 25 |
| Eph B4 154 | GCC ACG ATT TTG AGG CTG GC | (2643-2662) | ++ | 40 |
| Eph B4 153 | GGG GTT CCG GAT CAT CTT GT | (2623-2642) | ++ | 35 |
| Eph B4 152 | CCA GGG CGC TGA CCA CCT GG | (2603-2622) | + | 30 |
| Eph B4 151 | GGG AAG CGG GGC CGG GCA TT | (2583-2602) | + | 25 |
| Eph B4 150 | CCG GTC TTT CTG CCA ACA GT | (2563-2582) | ++ | 25 |
| Eph B4 149 | CCA GCA TGA GCT GGT GGA GG | (2543-2562) | ++ | 20 |
| Eph B4 148 | GAG GTG GGA CAG TCT GGG GG | (2523-2542) | + | 30 |
| Eph B4 147 | CGG GGG CAG CCG GTA GTC CT | (2503-2522) | ++ | 40 |
| Eph B4 146 | GTT CAA TGG CAT TGA TCA CG | (2483-2502) | ++++ | 70 |
| Eph B4 145 | TCC TGA TTG CTC ATG TCC CA | (2463-2482) | ++++ | 80 |
| Eph B4 144 | GTA CGG CCT CTC CCC AAA TG | (2443-2462) | +++ | 60 |
| Eph B4 143 | ACA TCA CCT CCC ACA TCA CA | (2423-2442) | ++++ | 80 |
| Eph B4 142 | ATC CCG TAA CTC CAG GCA TC | (2403-2422) | ++ | 40 |
| Eph B4 141 | ACT GGC GGA AGT GAA CTT CC | (2383-2402) | +++ | 50 |
| Eph B4 140 | GGA AGG CAA TGG CCT CCG GG | (2363-2382) | ++ | 45 |
| Eph B4 139 | GCA GTC CAT CGG ATG GGA AT | (2343-2362) | ++++ | 70 |
| Eph B4 138 | CTT TCC TCC CAG GGA GCT CG | (2323-2342) | ++++ | 70 |
| Eph B4 137 | TGT AGG TGG GAT CGG AAG AG | (2303-2322) | ++ | 40 |
| Eph B4 136 | TTC TCC TCC AGG AAT CGG GA | (2283-2302) | ++ | 35 |
| Eph B4 135 | AAG GCC AAA GTC AGA CAC TT | (2263-2282) | ++++ | 60 |
| Eph B4 134 | GCA GAC GAG GTT GCT GTT GA | (2243-2262) | ++ | 50 |
| Eph B4 133 | CTA GGA TGT TGC GAG CAG CC | (2223-2242) | ++ | 40 |
| Eph B4 132 | AGG TCT CGG TGG ACG TAG CT | (2203-2222) | ++ | 40 |
| Eph B4 131 | CAT CTC GGC AAG GTA CCG CA | (2183-2202) | +++ | 50 |
| Eph B4 130 | TGC CCG AGG CGA TGC CCC GC | (2163-2182) | ++ | 50 |
| Eph B4 129 | AGC ATG CCC ACG AGC TGG AT | (2143-2162) | ++ | 50 |
| Eph B4 128 | GAC TGT GAA CTG TCC GTC GT | (2123-2142) | ++ | 50 |
| Eph B4 127 | TTA GCC GCA GGA AGG AGT CC | (2103-2122) | +++ | 60 |
| Eph B4 126 | AGG GCG CCG TTC TCC ATG AA | (2083-2102) | ++ | 50 |
| Eph B4 125 | CTC TGT GAG AAT CAT GAC GG | (2063-2082) | ++++ | 80 |
| Eph B4 124 | GCA TGC TGT TGG TGA CCA CG | (2043-2062) | ++++ | 70 |
| Eph B4 123 | CCC TCC AGG CGG ATG ATA TT | (2023-2042) | ++ | 50 |
| Eph B4 122 | GGG GTG CTC GAA CTG GCC CA | (2003-2022) | ++++ | 80 |
| Eph B4 121 | TGA TGG AGG CCT CGC TCA GA | (1983-2002) | ++ | 50 |
| Eph B4 120 | AAC TCA CGC CGC TGC CGC TC | (1963-1982) | ++ | 40 |
| Eph B4 119 | CGT GTA GCC ACC CTT CAG GG | (1943-1962) | ++++ | 75 |
| Eph B4 118 | TCT TGA TTG CCA CAC AGC TC | (1923-1942) | ++++ | 80 |
| Eph B4 117 | TCC TTC TTC CCT GGG GCC TT | (1903-1922) | ++++ | 70 |
| Eph B4 116 | GAG CCG CCC CCG GCA CAC CT | (1883-1902) | ++ | 50 |
| Eph B4 115 | CGC CAA ACT CAC CTG CAC CA | (1863-1882) | ++++ | 60 |
| Eph B4 114 | ATC ACC TCT TCA ATC TTG AC | (1843-1862) | ++++ | 65 |
| Eph B4 113 | GTA GGA GAC ATC GAT CTC TT | (1823-1842) | ++++ | 90 |
| Eph B4 112 | TTG CAA ATT CCC TCA CAG CC | (1803-1822) | ++++ | 70 |
| Eph B4 111 | TCA TTA GGG TCT TCA TAA GT | (1783-1802) | ++++ | 70 |
| Eph B4 110 | GAA GGG GTC GAT GTA GAC CT | (1763-1782) | ++++ | 80 |
| Eph B4 109 | TAG TAC CAT GTC CGA TGA GA | (1743-1762) | ++ | 50 |
| Eph B4 108 | TAC TGT CCG TGT TTG TCC GA | (1723-1742) | ++ | 45 |
| Eph B4 107 | ATA TTC TGC TTC TCT CCC AT | (1703-1722) | ++++ | 70 |
| Eph B4 106 | TGC TCT GCT TCC TGA GGC AG | (1683-1702) | ++++ | 70 |
| Eph B4 105 | AGA ACT GCG ACC ACA ATG AC | (1663-1682) | ++ | 40 |
| Eph B4 104 | CAC CAG GAC CAG GAC CAC AC | (1643-1662) | ++++ | 70 |
| Eph B4 103 | CCA CGA CTG CCG TGC CCG CA | (1623-1642) | ++ | 40 |
| Eph B4 102 | ATC AGG GCC AGC TGC TCC CG | (1603-1622) | +++ | 50 |
| Eph B4 101 | CCA GCC CTC GCT CTC ATC CA | (1583-1602) | ++++ | 80 |
| Eph B4 100 | GTT GGG TCT GGC TGT GAT GT | (1563-1582) | ++++ | 80 |
| Eph B4 99 | TCC TGG CCG AAG GGC CCG TA | (1543-1562) | ++ | 35 |
| Eph B4 98 | GCC GGC CTC AGA GCG CGC CC | (1523-1542) | ++ | 50 |
| Eph B4 97 | GTA CCT GCA CCA GGT AGC TG | (1503-1522) | ++++ | 80 |
| Eph B4 96 | GCT CCC CGC TTC AGC CCC CG | (1483-1502) | ++ | 50 |
| Eph B4 95 | CAG CTC TGC CCG GTT TTC TG | (1463-1482) | ++ | 50 |
| Eph B4 94 | ACG TCT TCA GGA ACC GCA CG | (1443-1462) | ++++ | 80 |
| Eph B4 93 | CTG CTG GGA CCC TCG GCG CC | (1423-1442) | ++ | 40 |
| Eph B4 92 | CTT CTC ATG GTA TTT GAC CT | (1403-1422) | ++++ | 80 |
| Eph B4 91 | CGT AGT CCA GCA CAG CCC CA | (1383-1402) | ++++ | 85 |
| Eph B4 90 | CTG GGT GCC CGG GGA ACA GC | (1363-1382) | +++ | 50 |
| Eph B4 89 | CCA GGC CAG GCT CAA GCT GC | (1343-1462) | ++++ | 70 |
| Eph B4 88 | TGG GTG AGG ACC GCG TCA CC | (1323-1342) | ++ | 40 |
| Eph B4 87 | CGG ATG TCA GAC ACT GCA GG | (1303-1322) | ++++ | 60 |
| Eph B4 86 | AGG TAC CTC TCG GTC AGT GG | (1283-1302) | ++ | 50 |
| Eph B4 85 | TGA CAT TGA CAG GCT CAA AT | (1263-1282) | ++++ | 80 |
| Eph B4 84 | GGG ACG GGC CCC GTG GCT AA | (1243-1262) | ++ | 50 |
| Eph B4 83 | GGA GGA TAC CCC GTT CAA TG | (1223-1242) | +++ | 60 |
| Eph B4 82 | CAG TGA CCT CAA AGG TAT AG | (1203-1222) | ++++ | 70 |
| Eph B4 81 | GTG AAG TCA GGA CGT AGC CC | (1183-1202) | +++ | 60 |
| Eph B4 80 | TCG AAC CAC CAC CCA GGG CT | (1163-1182) | +++ | 50 |
| Eph B4 79 | CCA CCA GGT CCC GGG GGC CG | (1143-1162) | ++ | 40 |
| Eph B4 78 | GGG TCA AAA GTC AGG TCT CC | (1123-1142) | ++++ | 70 |
| Eph B4 77 | CCC GCA GGG CGC ACA GGA GC | (1103-1122) | +++ | 60 |
| Eph B4 76 | CTC CGG GTC GGC ACT CCC GG | (1083-1102) | +++ | 60 |
| Eph B4 75 | CAG CGG AGG GCG TAG GTG AG | (1063-1082) | ++ | 40 |
| Eph B4 74 | GTC CTC TCG GCC ACC AGA CT | (1043-1062) | ++ | 50 |
| Eph B4 73 | CCA GGG GGG CAC TCC ATT CC | (1023-1042) | ++ | 50 |
| Eph B4 72 | AGG TGC AGG GAG GAG CCG TT | (1003-1022) | ++++ | 70 |
| Eph B4 71 | CAG GCG GGA AAC CAC GCT CC | (983-1002) | ++ | 40 |
| Eph B4 70 | GCG GAG CCG AAG GAG GGG TG | (963-982) | +++ | 50 |
| Eph B4 69 | GTG CAG GGT GCA CCC CGG GG | (943-962) | +++ | 50 |
| Eph B4 68 | GTC TGT GCG TGC CCG GAA GT | (923-942) | ++ | 40 |
| Eph B4 67 | ACC CGA CGC GGC ACT GGC AG | (903-922) | ++ | 40 |
| Eph B4 66 | ACG GCT GAT CCA ATG GTG TT | (883-902) | ++ | 50 |
| Eph B4 65 | AGA GTG GCT ATT GGC TGG GC | (863-882) | ++++ | 60 |
| Eph B4 64 | ATG GCT GGC AGG ACC CTT CT | (843-862) | ++++ | 80 |
| Eph B4 63 | CCT GAC AGG GGC TTG AAG GT | (823-842) | ++++ | 80 |
| Eph B4 62 | GCC CTG GGC ACA GGC TCG GC | (803-822) | +++ | 70 |
| Eph B4 61 | ACT TGG TGT TCC CCT CAG CT | (783-802) | ++++ | 80 |
| Eph B4 60 | GCC TCG AAC CCC GGA GCA CA | (763-782) | +++ | 50 |
| Eph B4 59 | GCT GCA GCC CGT GAC CGG CT | (743-762) | +++ | 50 |
| Eph B4 58 | GTT CGG CCC ACT GGC CAT CC | (723-742) | ++ | 45 |
| Eph B4 57 | TCA CGG CAG TAG AGG CTG GG | (703-722) | +++ | 70 |
| Eph B4 56 | GCT GGG GCC AGG GGC GGG GA | (683-702) | ++ | 50 |
| Eph B4 55 | CGG CAT CCA CCA CGC AGC TA | (663-682) | ++ | 50 |
| Eph B4 54 | CCG GCC ACG GGC ACA ACC AG | (643-662) | ++ | 50 |
| Eph B4 53 | CTC CCG AGG CAC AGT CTC CG | (623-642) | +++ | 50 |
| Eph B4 52 | GGA ATC GAG TCA GGT TCA CA | (603-622) | ++++ | 90 |
| Eph B4 51 | GTC AGC TGG GCG CAC TTT TT | (583-602) | +++ | 70 |
| Eph B4 50 | GTA GAA GAG GTG CAG GGA TA | (563-582) | ++++ | 80 |
| Eph B4 49 | GCA GGG CCA TGC AGG CAC CC | (543-562) | ++++ | 80 |
| Eph B4 48 | TGG TCC TGG AAG GCC AGG TA | (523-542) | ++++ | 90 |
| Eph B4 47 | GAA GCC AGC CTT GCT GAG CG | (503-522) | ++++ | 80 |
| Eph B4 46 | GTC CCA GAC GCA GCG TCT TG | (483-502) | ++ | 40 |
| Eph B4 45 | ACA TTC ACC TTC CCG GTG GC | (463-482) | +++ | 50 |
| Eph B4 44 | CTC GGC CCC AGG GCG CTT CC | (443-462) | ++ | 50 |
| Eph B4 43 | GGG TGA GAT GCT CCG CGG CC | (423-442) | +++ | 60 |
| Eph B4 42 | ACC GTG TCC ACC TTG ATG TA | (403-422) | ++++ | 80 |
| Eph B4 41 | GGG GTT CTC CAT CCA GGC TG | (383-402) | ++++ | 80 |
| Eph B4 40 | GCG TGA GGG CCG TGG CCG TG | (363-382) | ++ | 50 |
| Eph B4 39 | TCC GCA TCG CTC TCA TAG TA | (343-362) | +++ | 60 |
| Eph B4 38 | GAA GAC GGT GAA GGT CTC CT | (323-342) | ++++ | 80 |
| Eph B4 37 | TGC AGG AGC GCC CAG CCC GA | (303-322) | +++ | 50 |
| Eph B4 36 | GGC AGG GAC AGG CAC TCG AG | (283-302) | +++ | 45 |
| Eph B4 35 | CAT GGT GAA GCG CAG CGT GG | (263-282) | ++ | 50 |
| Eph B4 34 | CGT ACA CGT GGA CGG CGC CC | (243-262) | ++ | 40 |
| Eph B4 33 | CGC CGT GGG ACC CAA CCT GT | (223-242) | +++ | 60 |
| Eph B4 32 | GCG AAG CCA GTG GGC CTG GC | (203-222) | ++++ | 70 |
| Eph B4 31 | CCG GGG CAC GCT GCA CGT CA | (183-202) | +++ | 60 |
| Eph B4 30 | CAC ACT TCG TAG GTG CGC AC | (163-182) | +++ | 70 |
| Eph B4 29 | GCT GTG CTG TTC CTC ATC CA | (143-162) | ++++ | 80 |
| Eph B4 28 | GGC CGC TCA GTT CCT CCC AC | (123-142) | ++ | 40 |
| Eph B4 27 | TGC CCG TCC ACC TGA GGG AA | (103-122) | ++ | 50 |
| Eph B4 26 | TGT CAC CCA CTT CAG ATC AG | (83-102) | ++++ | 70 |
| Eph B4 25 | CAG TTT CCA ATT TTG TGT TC | (63-82) | ++++ | 70 |
| Eph B4 24 | AGC AGG GTC TCT TCC AAA GC | (43-62) | ++++ | 80 |
| Eph B4 23 | TGC GGC CAA CGA AGC CCA GC | (23-42) | ++ | 50 |
| Eph B4 22 | AGA GCA GCA CCC GGA GCT CC | (3-22) | +++ | 50 |
| Eph B4 21 | AGC AGC ACC CGG AGC TCC AT | (1-20) | +++ | 50 |

Additional antisense probes described in the specification

| | | |
|---|---|---|
| EphB4 AS-1 | GTG CAG GGA TAG CAG GGC CAT | (552-572) |
| EphB4 AS-2 | AAG GAG GGG TGG TGC ACG GTG | (952-972) |
| EphB4 AS-3 | TTC CAG GTG CAG GGA GGA GCC | (1007-1027) |
| EphB4 AS-4 | GTG GTG ACA TTG ACA GGC TCA | (1263-1285) |
| EphB4 AS-5 | TCT GGC TGT GAT GTT CCT GGC | (1555-1575) |
| EphB4 AS-6 | GCC GCT CAG TTC CTC CCA | (123-140) |
| EphB4 AS-7 | TGA AGG TCT CCT TGC AGG | (316-333) |
| EphB4 AS-8 | CGC GGC CAC CGT GTC CAC CTT | (408-428) |
| EphB4 AS-9 | CTT CAG GGT CTT GAT TGC CAC | (1929-1949) |
| EphB4 AS-10 | ATG GAG GCC TCG CTC AGA AA | (1980-1999) |
| Ephb4 AS-11 | CAT GCC CAC GAG CTG GAT GAC | (2138-2158) |

TABLE 2
Examples of EphB4 RNAi probes (siRNAs)

| | | | |
|---|---|---|---|
| | | | Percent |
| | | Inhibition | reduction |
| | | of EphB4 | in |
| RNAi | EphB4 RNAi sequence | Expression | viability |

| | | | | | |
|---|---|---|---|---|---|
| 1 | 446 | aaattggaaactgctgatctg | 466 | | |
| 2 | 447 | aattggaaactgctgatctga | 467 | +++ | 70 |
| 3 | 453 | aaactgctgatctgaagtggg | 473 | ++++ | 70 |
| 4 | 454 | aactgctgatctgaagtgggt | 474 | +++ | 80 |
| 5 | 854 | aatgtcaagacgctgcgtctg | 874 | +++ | 65 |
| 6 | 467 | aagtgggtgacattccctcag | 487 | + | 35 |
| 7 | 848 | aaggtgaatgtcaagacgctg | 868 | ++ | 50 |
| 8 | 698 | aaggagaccttcaccgtcttc | 718 | +++ | 75 |
| 9 | 959 | aaaaagtgcgcccagctgact | 979 | + | 40 |
| 10 | 1247 | aatagccactctaacaccatt | 1267 | ++ | 50 |
| 11 | 1259 | aacaccattggatcagccgtc | 1279 | ++ | 50 |
| 12 | 1652 | aatgtcaccactgaccgagag | 1672 | + | 35 |
| 13 | 1784 | aaataccatgagaagggcgcc | 1804 | +++ | 65 |
| 14 | 1832 | aagacgtcagaaaaccgggca | 1852 | + | 30 |
| 15 | 1938 | aacatcacagccagacccaac | 19 | ++ | 50 |
| 16 | 2069 | aagcagagcaatgggagagaa | 2089 | ++++ | 75 |
| 17 | 2078 | aatgggagagaagcagaatat | 2098 | +++ | 65 |
| 18 | 2088 | aagcagaatattcggacaaac | 2108 | +++ | 70 |
| 19 | 2094 | aatattcggacaaacacggac | 2114 | ++ | 40 |
| 20 | 2105 | aaacacggacagtatctcatc | 2125 | ++ | 50 |
| 21 | 2106 | aacacggacagtatctcatcg | 2126 | + | 35 |
| 22 | 2197 | aaaagagatcgatgtctccta | 2217 | +++ | 65 |
| 23 | 2174 | aatgaggctgtgagggaattt | 2194 | ++ | 50 |
| 24 | 2166 | aagaccctaatgaggctgtga | 2186 | ++ | 50 |
| 25 | 2198 | aaagagatcgatgtctcctac | 2218 | +++ | 55 |
| 26 | 2199 | aagagatcgatgtctcctacg | 2219 | +++ | 70 |
| 27 | 2229 | aagaggtgattggtgcaggtg | 2249 | + | 33 |
| 28 | 2222 | aagattgaagaggtgattggt | 2242 | + | 30 |
| 29 | 2429 | aacagcatgcccgtcatgatt | 2449 | ++ | 40 |
| 30 | 2291 | aagaaggagagctgtgtggca | 2311 | +++ | 50 |
| 31 | 2294 | aaggagagctgtgtggcaatc | 2314 | +++ | 60 |
| 32 | 2311 | aatcaagaccctgaagggtgg | 2331 | +++ | 70 |
| 33 | 2497 | aaacgacggacagttcacagt | 2517 | + | 35 |
| 34 | 2498 | aacgacggacagttcacagtc | 2518 | + | 40 |
| 35 | 2609 | aacatcctagtcaacagcaac | 2629 | ++ | 50 |
| 36 | 2621 | aacagcaacctcgtctgcaaa | 2641 | + | 35 |
| 37 | 2678 | aactcttccgatcccacctac | 2698 | ++ | 50 |
| 38 | 2640 | aagtgtctgactttggccttt | 2660 | +++ | 70 |
| 39 | 2627 | aacctcgtctgcaaagtgtct | 2647 | ++ | 50 |
| 40 | 2639 | aaagtgtctgactttggcctt | 2659 | + | 25 |
| 41 | 2852 | aatcaggacgtgatcaatgcc | 2872 | +++ | 75 |
| 42 | 2716 | aaagattcccatccgatggac | 2736 | ++ | 50 |
| 43 | 2717 | aagattcccatccgatggact | 2737 | ++ | 60 |
| 44 | 2762 | aagttcacttccgccagtgat | 2782 | +++ | 70 |
| 45 | 3142 | aagatacgaagaaagtttcgc | 3162 | ++ | 50 |
| 46 | 3136 | aatgggaagatacgaagaaag | 3156 | +++ | 66 |
| 47 | 2867 | aatgccattgaacaggactac | 2887 | | |
| 48 | 3029 | aaaatcgtggcccgggagaat | 3049 | + | 33 |
| 49 | 3254 | aaaatcttggccagtgtccag | 3274 | ++ | 50 |
| 50 | 3255 | aaatcttggccagtgtccagc | 3275 | +++ | 75 |
| 51 | 3150 | aagaaagtttcgcagccgctg | 3170 | +++ | 80 |
| 52 | 3251 | aagaaaatcttggccagtgtc | 3271 | ++ | 50 |
| 53 | 3256 | aatcttggccagtgtccagca | 3276 | ++ | 50 |

Additional RNAi probes described in the specification

| | |
|---|---|
| Eph B4 50 | gagacccugcugaacacaauu |
| Eph B4 472 | ggugaaugucaagacgcuguu |
| Eph B4 1562 | caucacagccagacccaacuu |
| siRNA 2303 | cucuuccgaucccaccuacuu |
| Eph B4 2302 | cucuuccgaucccaccuacuu |

TABLE 3
Examples of Ephrin B2 antisense probes

| | | | |
|---|---|---|---|
| | | Percent | Inhibition |
| | coding | reduction in | of Ephrin B2 |
| sequence | region | viability | Expression |

| | | | | |
|---|---|---|---|---|
| Ephrin AS-51 | TCA GAC CTT GTA GTA AAT GT | (983-1002) | 35 | ++ |
| Ephrin AS-50 | TCG CCG GGC TCT GCG GGG GC | (963-982) | 50 | +++ |
| Ephrin AS-49 | ATC TCC TGG ACG ATG TAC AC | (943-962) | 45 | ++ |
| Ephrin AS-48 | CGG GTG CCC GTA GTC CCC GC | (923-942) | 35 | ++ |
| Ephrin AS-47 | TGA CCT TCT CGT AGT GAG GG | (903-922) | 40 | +++ |
| Ephrin AS-46 | CAG AAG ACG CTG TCC GCA GT | (883-902) | 40 | ++ |
| Ephrin AS-45 | CCT TAG CGG GAT GAT AAT GT | (863-882) | 35 | ++ |
| Ephrin AS-44 | CAC TGG GCT CTG AGC CGT TG | (843-862) | 60 | +++ |
| Ephrin AS-43 | TTG TTG CCG CTG CGC TTG GG | (823-842) | 40 | ++ |
| Ephrin AS-42 | TGT GGC CAG TGT GCT GAG CG | (803-822) | 40 | ++ |
| Ephrin AS-41 | ACA GCG TGG TCG TGT GCT GC | (783-802) | 70 | +++ |
| Ephrin AS-40 | GGC GAG TGC TTC CTG TGT CT | (763-782) | 80 | ++++ |
| Ephrin AS-39 | CCT CCG GTA CTT CAG CAA GA | (743-762) | 50 | +++ |
| Ephrin AS-38 | GGA CCA CCA GCG TGA TGA TG | (723-742) | 60 | +++ |
| Ephrin AS-37 | ATG ACG ATG AAG ATG ATG CA | (703-722) | 70 | +++ |
| Ephrin AS-36 | TCC TGA AGC AAT CCC TGC AA | (683-702) | 60 | +++ |
| Ephrin AS-35 | ATA AGG CCA CTT CGG AAC CG | (663-682) | 45 | ++ |
| Ephrin AS-34 | AGG ATG TTG TTC CCC GAA TG | (643-662) | 50 | +++ |
| Ephrin AS-33 | TCC GGC GCT GTT GCC GTC TG | (623-642) | 75 | +++ |
| Ephrin AS-32 | TGC TAG AAC CTG GAT TTG GT | (603-622) | 60 | +++ |
| Ephrin AS-31 | TTT ACA AAG GGA CTT GTT GT | (583-602) | 66 | +++ |
| Ephrin AS-30 | CGA ACT TCT TCC ATT TGT AC | (563-582) | 50 | ++ |
| Ephrin AS-29 | CAG CTT CTA GTT CTG GAC GT | (543-562) | 50 | +++ |
| Ephrin AS-28 | CTT GTT GGA TCT TTA TTC CT | (523-542) | 70 | +++ |
| Ephrin AS-27 | GGT TGA TCC AGC AGA ACT TG | (503-S22) | 65 | +++ |
| Ephrin AS-26 | CAT CTT GTC CAA CTT TCA TG | (483-502) | 75 | +++ |
| Ephrin AS-25 | AGG ATC TTC ATG GCT CTT GT | (463-482) | 60 | +++ |
| Ephrin AS-24 | CTG GCA CAC CCC TCC CTC CT | (443-462) | 45 | ++ |
| Ephrin AS-23 | GGT TAT CCA GGC CCT CCA AA | (423-442) | 50 | +++ |
| Ephrin AS-22 | GAC CCA TTT GAT GTA GAT AT | (403-422) | 50 | +++ |
| Ephrin AS-21 | AAT GTA ATA ATC TTT GTT CT | (383-402) | 60 | +++ |
| Ephrin AS-20 | TCT GAA ATT CTA GAC CCC AG | (363-382) | 60 | +++ |
| Ephrin AS-19 | AGG TTA GGG CTG AAT TCT TG | (343-362) | 75 | +++ |
| Ephrin AS-18 | AAA CTT GAT GGT GAA TTT GA | (323-342) | 60 | +++ |
| Ephrin AS-17 | TAT CTT GGT CTG GTT TGG CA | (303-322) | 50 | ++ |
| Ephrin AS-16 | CAG TTG AGG AGA GGG GTA TT | (283-302) | 40 | ++ |
| Ephrin AS-15 | TTC CTT CTT AAT AGT GCA TC | (263-282) | 66 | +++ |
| Ephrin AS-14 | TGT CTG CTT GGT CTT TAT CA | (243-262) | 70 | ++++ |
| Ephrin AS-13 | ACC ATA TAA ACT TTA TAA TA | (223-242) | 50 | +++ |
| Ephrin AS-12 | TTC ATA CTG GCC AAC AGT TT | (203-222) | 50 | +++ |
| Ephrin AS-11 | TAG AGT CCA CTT TGG GGC AA | (183-202) | 70 | ++++ |
| Ephrin AS-10 | ATA ATA TCC AAT TTG TCT CC | (163-182) | 70 | ++++ |
| Ephrin AS-9 | TAT CTG TGG GTA TAG TAC CA | (143-162) | 80 | ++++ |
| Ephrin AS-8 | GTC CTT GTC CAG GTA GAA AT | (123-142) | 60 | +++ |
| Ephrin AS-7 | TTG GAG TTC GAG GAA TTC CA | (103-122) | 80 | ++++ |
| Ephrin AS-6 | ATA GAT AGG CTC TAA AAC TA | (83-102) | 70 | +++ |
| Ephrin AS-5 | TCG ATT TGG AAA TCG CAG TT | (63-82) | 50 | +++ |
| Ephrin AS-4 | CTG CAT AAA ACC ATC AAA AC | (43-62) | 80 | ++++ |
| Ephrin AS-3 | ACC CCA GCA GTA CTT CCA CA | (23-42) | 85 | ++++ |
| Ephrin AS-2 | CGG AGT CCC TTC TCA CAG CC | (3-22) | 70 | +++ |
| Ephrin AS-1 | GAG TCC CTT CTC ACA GCC AT | (1-20) | 80 | ++++ |

TABLE 4
Examples of Ephrin B2 RNAi probes (siRNAs).

| | | | |
|---|---|---|---|
| | Percent | Inhibition | |
| RNAi Sequence and homology | reduction in | of Ephrin B2 | RNAi |
| with other human genes. | viability | Expression | no. |

| | | | | | |
|---|---|---|---|---|---|
| 89 | aactgcgatttccaaatcgat | 109 | 80 | ++++ | 1 |
| 141 | aactccaaatttctacctgga | 161 | 70 | ++++ | 2 |
| 148 | aatttctacctggacaaggac | 168 | 75 | +++ | 3 |
| 147 | aaatttctacctggacaagga | 167 | 60 | +++ | 4 |
| 163 | aaggactggtactatacccac | 183 | 40 | ++ | 5 |
| 217 | aagtggactctaaaactgttg | 237 | 80 | ++++ | 6 |
| 229 | aaactgttggccagtatgaat | 249 | 50 | +++ | 7 |
| 228 | aaaactgttggccagtatgaa | 248 | 80 | ++++ | 8 |
| 274 | aagaccaagcagacagatgca | 294 | 80 | ++++ | 11 |
| 273 | aaagaccaagcagacagatgc | 293 | 60 | +++ | 12 |
| 363 | aagtttcaagaattcagccct | 383 | 66 | +++ | 13 |
| 370 | aagaattcagccctaacctct | 390 | 50 | +++ | 14 |
| 373 | aattcagccctaacctctggg | 393 | 50 | +++ | 15 |
| 324 | aactgtgccaaaccagaccaa | 344 | 90 | ++++ | 16 |
| 440 | aaatgggtctttggagggcct | 460 | 80 | ++++ | 17 |
| 501 | aagatcctcatgaaagttgga | 521 | 50 | +++ | 18 |
| 513 | aaagttggacaagatgcaagt | 533 | 50 | +++ | 19 |
| 491 | aagagccatgaagatcctcat | 511 | 50 | +++ | 20 |
| 514 | aagttggacaagatgcaagtt | 534 | 66 | +++ | 21 |
| 523 | aagatgcaagttctgctggat | 543 | 66 | +++ | 22 |
| 530 | aagttctgctggatcaaccag | 550 | 50 | +++ | 23 |
| 545 | aaccaggaataaagatccaac | 565 | 35 | ++ | 24 |
| 555 | aaagatccaacaagacgtcca | 575 | 40 | ++ | 25 |
| 556 | aagatccaacaagacgtccag | 576 | 60 | +++ | 26 |
| 563 | aacaagacgtccagaactaga | 583 | 60 | +++ | 27 |
| 566 | aagacgtccagaactagaagc | 586 | 70 | +++ | 28 |
| 593 | aaatggaagaagttcgacaac | 613 | 75 | ++++ | 29 |
| 577 | aactagaagctggtacaaatg | 597 | 66 | +++ | 30 |
| 594 | aatggaagaagttcgacaaca | 614 | 35 | ++ | 31 |
| 583 | aagctggtacaaatggaagaa | 603 | 50 | +++ | 32 |
| 611 | aacaagtccctttgtaaaacc | 631 | 70 | ++++ | 33 |
| 599 | aagaagttcgacaacaagtcc | 619 | 70 | ++++ | 34 |
| 602 | aagttcgacaacaagtccctt | 622 | 80 | ++++ | 35 |
| 626 | aaaaccaaatccaggttctag | 646 | 50 | +++ | 36 |
| 627 | aaaccaaatccaggttctagc | 647 | 25 | + | 37 |
| 628 | aaccaaatccaggttctagca | 648 | 30 | ++ | 38 |
| 632 | aaatccaggttctagcacaga | 652 | 60 | +++ | 39 |
| 633 | aatccaggttctagcacagac | 653 | 40 | ++ | 40 |
| 678 | aacaacatcctcggttccgaa | 698 | 30 | ++ | 41 |
| 681 | aacatcctcggttccgaagtg | 701 | 20 | + | 42 |
| 697 | aagtggccttatttgcaggga | 717 | 30 | ++ | 43 |

Additional Ephrin B2 RNAi probes described in the
specification

| | |
|---|---|
| GCAGACAGAUGCACUAUUAUU | ephrin |
| | B2 264 |
| CUGCGAUUUCCAAAUCGAUUU | ephrin |
| | B2 63 |
| GGACUGGUACUAUACCCACUU | ephrin |
| | B2 137 |

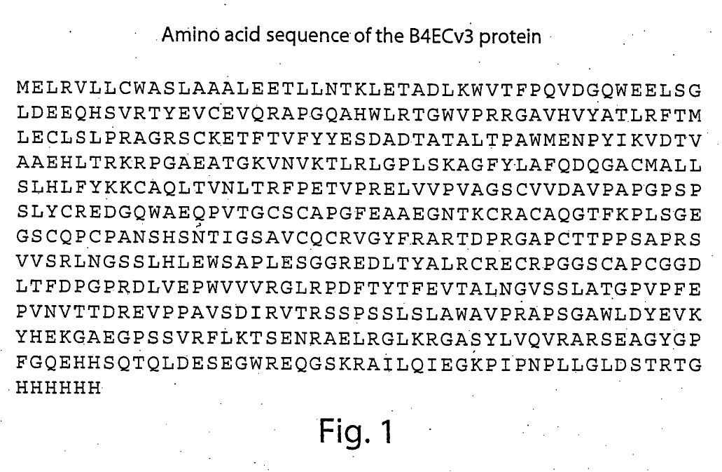

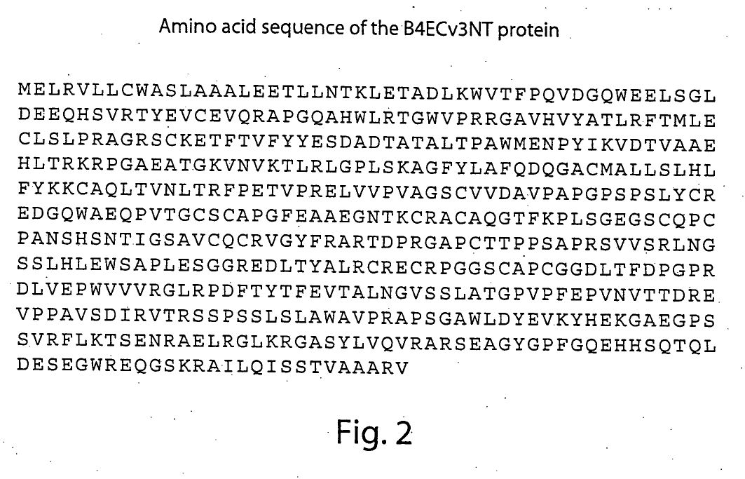

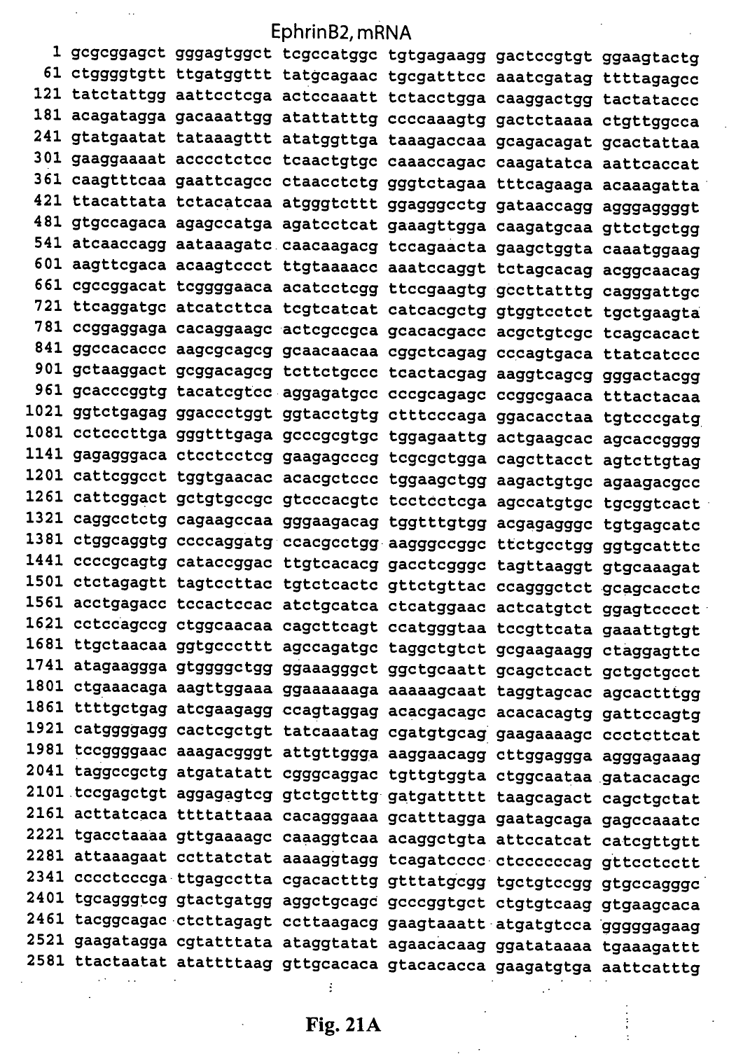

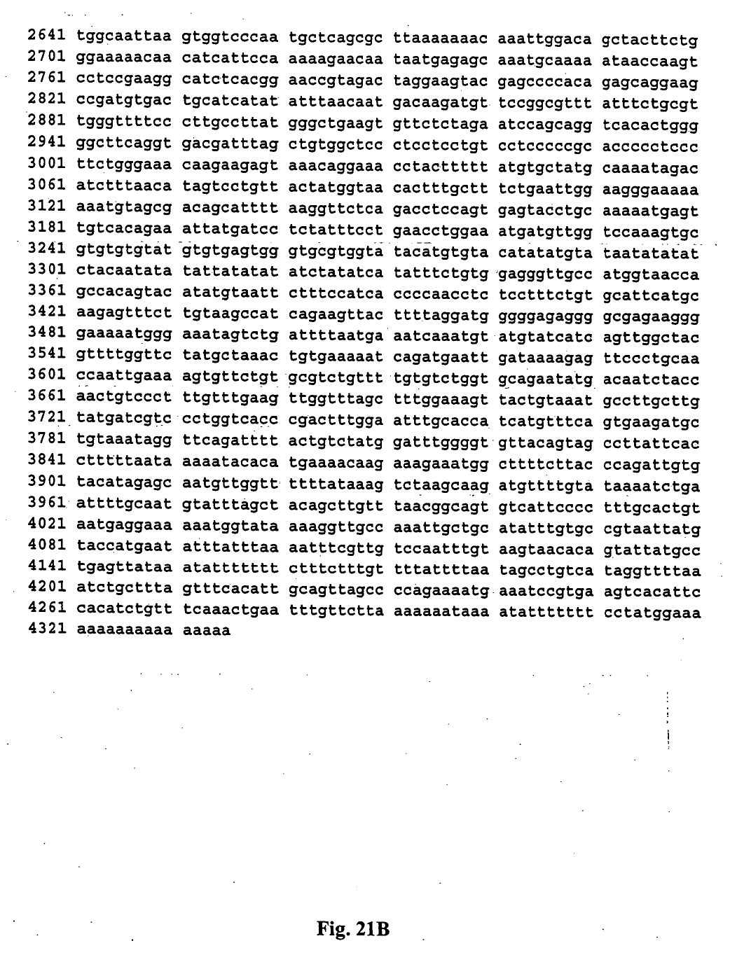

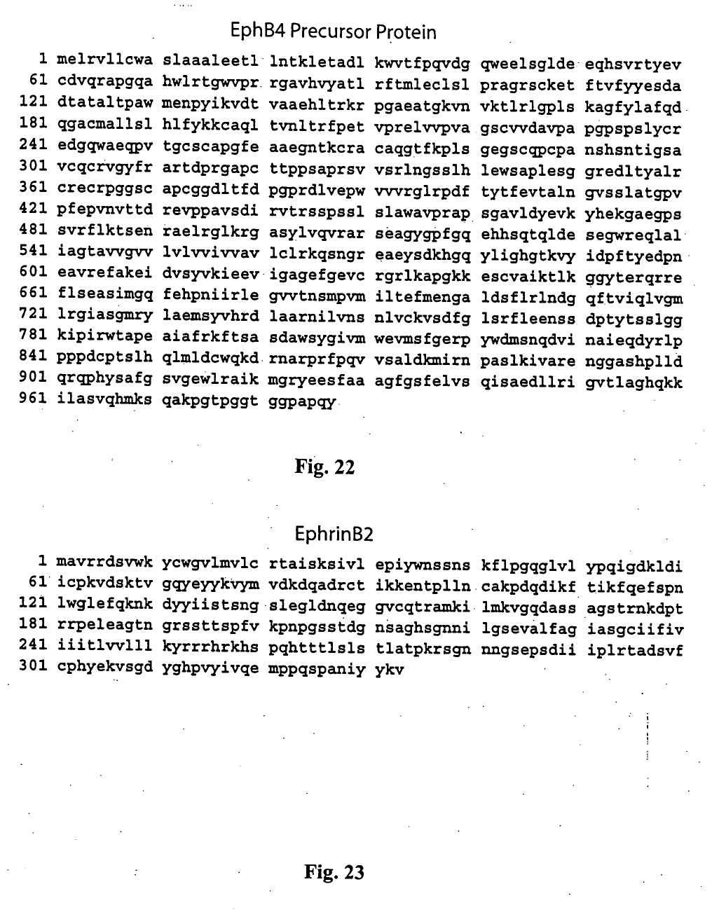

In other embodiments, the present invention provides polypeptide therapeutic agents which include soluble polypeptides, antibodies and antigen-binding portions of antibodies. In certain aspects, the disclosure provides soluble EphB4 polypeptides comprising an amino acid sequence of an extracellular domain of an EphB4 protein. The soluble EphB4 polypeptides bind specifically to an EphrinB2 polypeptide. The term “soluble” is used merely to indicate that these polypeptides do not contain a transmembrane domain or a portion of a transmembrane domain sufficient to compromise the solubility of the polypeptide in a physiological salt solution. Soluble polypeptides are preferably prepared as monomers that compete with EphB4 for binding to ligand such as EphrinB2 and inhibit the signaling that results from EphB4 activation. Optionally, a soluble polypeptide may be prepared in a multimeric form, by, for example, expressing as an Fc fusion protein or fusion with another multimerization domain. Such multimeric forms may have complex activities, having agonistic or antagonistic effects depending on the context. In certain embodiments the soluble EphB4 polypeptide comprises a globular domain of an EphB4 protein. A soluble EphB4 polypeptide may comprise a sequence at least 90% identical to residues 1-522 of the amino acid sequence defined by FIG. 22. A soluble EphB4 polypeptide may comprise a sequence at least 90% identical to residues 1-412 of the amino acid sequence defined by FIG. 22. A soluble EphB4 polypeptide may comprise a sequence at least 90% identical to residues 1-312 of the amino acid sequence defined by FIG. 22. A soluble EphB4 polypeptide may comprise a sequence encompassing the globular (G) domain (amino acids 29-197 of FIG. 22), and optionally additional domains, such as the cysteine-rich domain (amino acids 239-321 of FIG. 22), the first fibronectin type 3 domain (amino acids 324-429 of FIG. 22) and the second fibronectin type 3 domain (amino acids 434-526 of FIG. 22). Preferred polypeptides described herein and demonstrated as having ligand binding activity include polypeptides corresponding to 1-537, 1-427 and 1-326, respectively, of the amino acid sequence shown in FIG. 22. A soluble EphB4 polypeptide may comprise a sequence as set forth in FIG. 1 or 2. As is well known in the art, expression of such EphB4 polypeptides in a suitable cell, such as HEK293T cell line, will result in cleavage of a leader peptide. Although such cleavage is not always complete or perfectly consistent at a single site, it is known that EphB4 tends to be cleaved so as to remove the first 15 amino acids of the sequence shown in FIG. 22. Accordingly, as specific examples, the disclosure provides unprocessed soluble EphB4 polypeptides that bind to EphrinB2 and comprise an amino acid sequence selected from the following group (numbering is with respect to the sequence of FIG. 22): 1-197, 29-197, 1-312, 29-132, 1-321, 29-321, 1-326, 29-326, 1-412, 29-412, 1-427, 29-427, 1-429, 29-429, 1-526, 29-526, 1-537 and 29-537. Such polypeptides may be used in a processed form, such forms having a predicted amino acid sequence selected from the following group (numbering is with respect to the sequence of FIG. 22): 16-197, 16-312, 16-321, 16-326, 16-412, 16-427, 16-429, 16-526 and 16-537. Additionally, a soluble EphB4 polypeptide may be one that comprises an amino acid sequence at least 90%, and optionally 95% or 99% identical to any of the preceding amino acid sequences while retaining EphrinB2 binding activity. Preferably, any variations in the amino acid sequence from the sequence shown in FIG. 21 are conservative changes or deletions of no more than 1, 2, 3, 4 or 5 amino acids, particularly in a surface loop region. In certain embodiments, the soluble EphB4 polypeptide may inhibit the interaction between Ephrin B2 and EphB4. The soluble EphB4 polypeptide may inhibit clustering of or phosphorylation of Ephrin B2 or EphB4. Phosphorylation of EphrinB2 or EphB4 is generally considered to be one of the initial events in triggering intracellular signaling pathways regulated by these proteins. As noted above, the soluble EphB4 polypeptide may be prepared as a monomeric or multimeric fusion protein. The soluble polypeptide may include one or more modified amino acids. Such amino acids may contribute to desirable properties, such as increased resistance to protease digestion.

The present disclosure provides soluble EphB4 polypeptides having an additional component that confers increased serum half-life while still retaining EphrinB2 binding activity. In certain embodiments soluble EphB4 polypeptides are monomeric and are covalently linked to one or more polyethylene glycol (PEG) groups. The one or more PEG may have a molecular weight ranging from about 1 kDa to about 100 kDa, and will preferably have a molecular weight ranging from about 10 to about 60 kDa or about 20 to about 40 kDa. In a preferred embodiment, the soluble, monomeric EphB4 conjugate comprises an EphB4 polypeptide covalently linked to one PEG group of from about 20 to about 40 kDa (monoPEGylated EphB4), preferably via an ε-amino group of EphB4 lysine or the N-terminal amino group. Most preferably, EphB4 is randomly PEGylated at one amino group out of the group consisting of the ε-amino groups of EphB4 lysine and the N-terminal amino group. Surprisingly, it has been found that monoPEGylated EphB4 according to the invention has superior properties in regard to the therapeutic applicability of unmodified soluble EphB4 polypeptides and poly-PEGylated EphB4. Nonetheless, the disclosure also provides poly-PEGylated EphB4 having PEG at more than one position. Such polyPEGylated forms provide improved serum-half life relative to the unmodified form. In certain embodiments, a soluble EphB4 polypeptide is stably associated with a second stabilizing polypeptide that confers improved half-life without substantially diminishing EphrinB2 binding. A stabilizing polypeptide will preferably be immunocompatible with human patients (or animal patients, where veterinary uses are contemplated) and have little or no significant biological activity. In a preferred embodiment, the stabilizing polypeptide is a human serum albumin, or a portion thereof. A human serum albumin may be stably associated with the EphB4 polypeptide covalently or non-covalently. Covalent attachment may be achieved by expression of the EphB4 polypeptide as a co-translational fusion with human serum albumin. The albumin sequence may be fused at the N-terminus, the C-terminus or at a non-disruptive internal position in the soluble EphB4 polypeptide. Exposed loops of the EphB4 would be appropriate positions for insertion of an albumin sequence. Albumin may also be post-translationally attached to the EphB4 polypeptide by, for example, chemical cross-linking. An EphB4 polypeptide may also be stably associated with more than one albumin polypeptide.

Examples of soluble EphB4 polypeptides are provided in the Examples below.

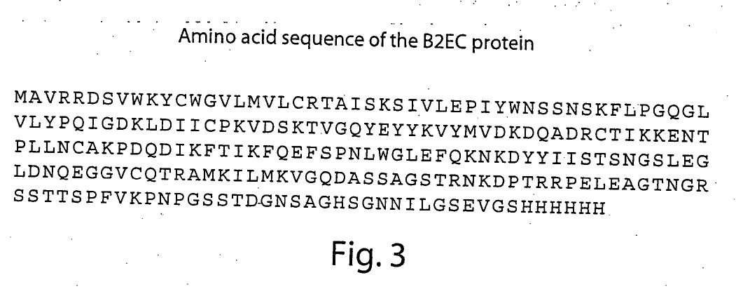

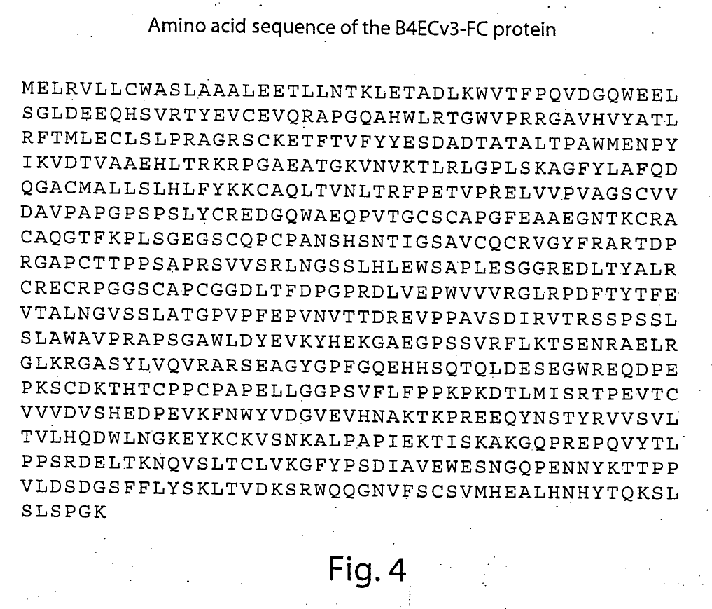

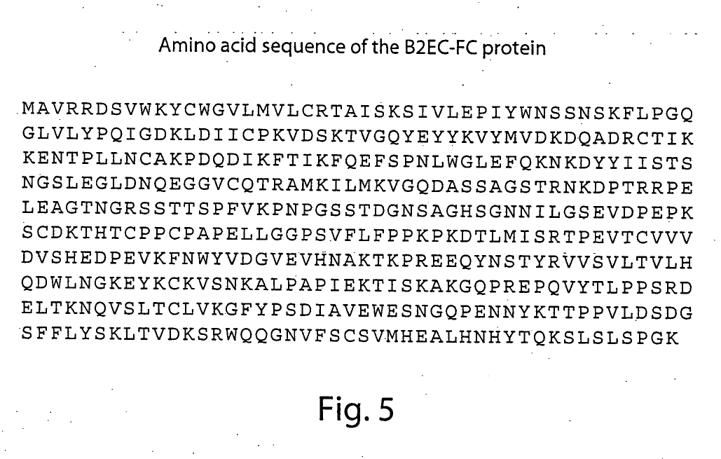

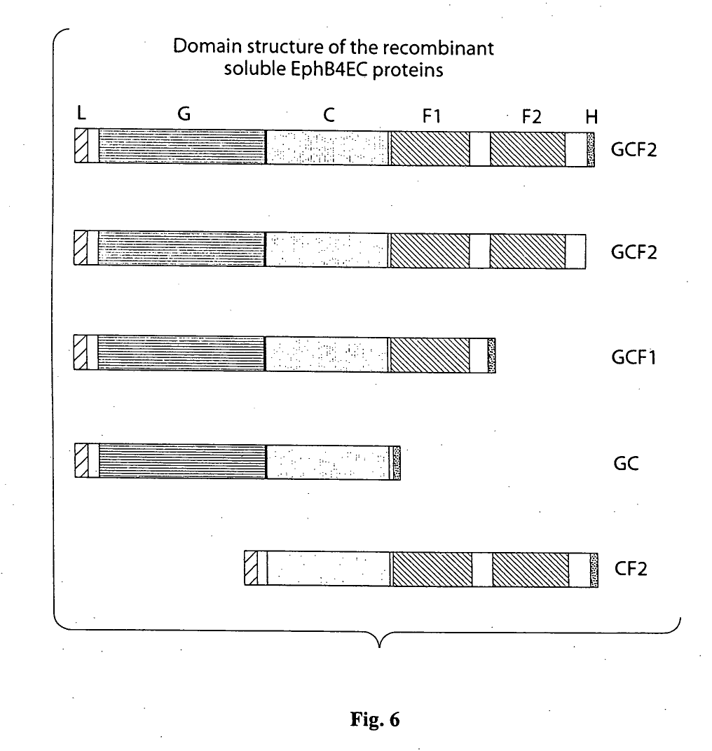

In certain aspects, the disclosure provides soluble EphrinB2 polypeptides comprising an amino acid sequence of an extracellular domain of an EphrinB2 protein. The soluble EphrinB2 polypeptides bind specifically to an EphB4 polypeptide. The term “soluble” is used merely to indicate that these polypeptides do not contain a transmembrane domain or a portion of a transmembrane domain sufficient to compromise the solubility of the polypeptide in a physiological salt solution. Soluble polypeptides are preferably prepared as monomers that compete with EphrinB2 for binding to ligand such as EphB4 and inhibit the signaling that results from EphrinB2 activation. Optionally, a soluble polypeptide may be prepared in a multimeric form, by, for example, expressing as an Fc fusion protein or fusion with another multimerization domain. Such multimeric forms may have complex activities, having agonistic or antagonistic effects depending on the context. A soluble EphrinB2 polypeptide may comprise residues 1-225 of the amino acid sequence defined by FIG. 22. A soluble EphrinB2 polypeptide may comprise a sequence defined by FIG. 3. As is well known in the art, expression of such EphrinB2 polypeptides in a suitable cell, such as HEK293T cell line, will result in cleavage of a leader peptide. Although such cleavage is not always complete or perfectly consistent at a single site, it is known that EphrinB2 tends to be cleaved so as to remove the first 26 amino acids of the sequence shown in FIG. 22. Accordingly, as specific examples, the disclosure provides unprocessed soluble EphrinB2 polypeptides that bind to EphB4 and comprise an amino acid sequence corresponding to amino acids 1-225 of FIG. 22. Such polypeptides may be used in a processed form, such forms having a predicted amino acid sequence selected from the following group (numbering is with respect to the sequence of FIG. 22): 26-225. In certain embodiments, the soluble EphrinB2 polypeptide may inhibit the interaction between Ephrin B2 and EphB4. The soluble EphrinB2 polypeptide may inhibit clustering of or phosphorylation of EphrinB2 or EphB4. As noted above, the soluble EphrinB2 polypeptide may be prepared as a monomeric or multimeric fusion protein. The soluble polypeptide may include one or more modified amino acids. Such amino acids may contribute to desirable properties, such as increased resistance to protease digestion.

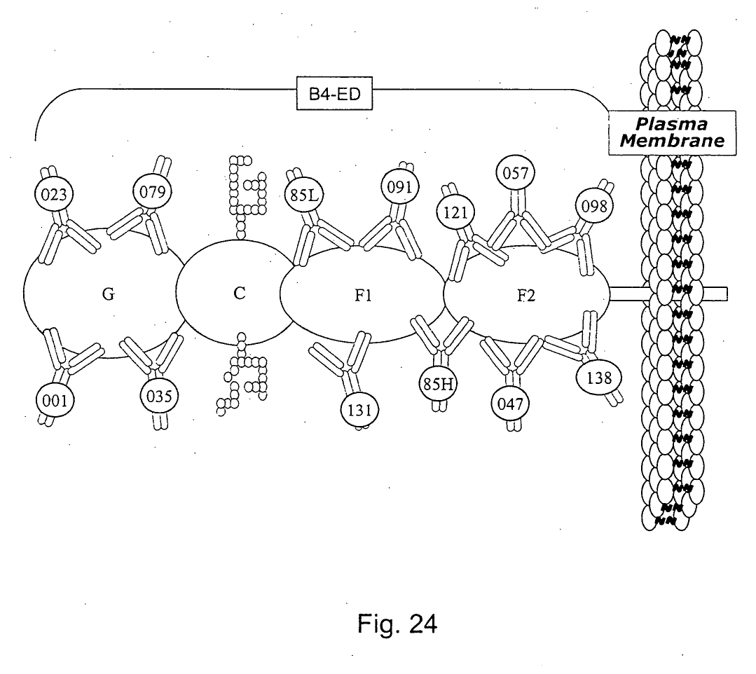

In another specific embodiment, the present invention provides antibodies against Ephrin B2 or EphB4. As described herein, the term “antagonist antibody” refers to an antibody that inhibits function of Ephrin B2 or EphB4. Preferably, the antagonist antibody binds to an extracellular domain of Ephrin B2 or EphB4. It is understood that antibodies of the invention may be polyclonal or monoclonal; intact or truncated, e.g., F(ab′)2, Fab, Fv; xenogeneic, allogeneic, syngeneic, or modified forms thereof, e.g., humanized, chimeric, etc. Examples of these antibodies include, but are not limited to, EphB4 antibody Nos. 1, 23, 35, 47, 57, 79, 85L, 85H, 91, 98, 121, 131, and 138 as shown in FIG. 24.

Hybridomas producing antibody No. 23 (epitope within amino acids 16-198), antibody No. 91 (kinase activating antibody; epitope within amino acids 324-429), antibody No. 98 (epitope within amino acids 430-537), antibody No. 131 (epitope within amino acids 324-429), and antibody No. 138 (epitope within amino acids 430-537) were deposited in the American Type Culture Collection (ATCC), 10801 University Boulevard, Manassas, Va. 20110-2209. The ATCC Deposit Designation Nos. for antibody No. 23, No. 91, No. 98, No. 131, and No. 138 are PTA-6208, PTA-6209, PTA-6210, PTA-6214, and PTA-6211, respectively. Therefore, certain specific aspects of the disclosure relate to a hybridoma cell having an ATCC Deposit Designation No. selected from the group consisting of PTA-6208, PTA-6209, PTA-6210, PTA-6214, and PTA-6211.

VI. Methods of Treatment

In certain embodiments, the present disclosure provides methods of inhibiting or reducing tumor growth and methods of treating an individual suffering from cancer. Optionally, one or more of these therapeutic methods is applied after gene amplification (EphB4 or Ephrin B2) has been detected by the methods as described above. These methods involve administering to the individual a therapeutically effective amount of one or more therapeutic agent as described above, or a conventional anti-tumor compounds as described below, or both. These methods are particularly aimed at therapeutic and prophylactic treatments of animals, and more particularly, humans.

As described herein, the tumor includes a tumor inside an individual, a tumor xenograft, or a tumor cultured in vitro. In particular, nucleic acid therapeutic agents of the present disclosure are useful for treating or preventing a cancer (tumor), including, but not limited to, colorectal carcinoma, breast cancer, ovary cancer, mesothelioma, prostate cancer, bladder cancer, lung cancer, brain cancer, stomach cancer, HNSCC, Kaposi sarcoma, and leukemia.

In certain embodiments of such methods, one or more therapeutic agents can be administered, together (simultaneously) or at different times (sequentially). In addition, therapeutic agents can be administered with more than two types of compounds for treating cancer. For example, a therapeutic agent of the present invention can be used in combination with one of the conventional anti-tumor therapeutic approaches. Such methods can be used in prophylactic cancer prevention, prevention of cancer recurrence and metastases after surgery, and as an adjuvant of other conventional cancer therapy. The present disclosure recognizes that the effectiveness of conventional cancer therapies (e.g., chemotherapy, radiation therapy, phototherapy, immunotherapy, and surgery) can be enhanced through the use of a subject nucleic acid therapeutic agent.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When a therapeutic agent of the present disclosure is administered in combination with another conventional anti-neoplastic (anti-tumor or chemotherapeutic) agent, either concomitantly or sequentially, such therapeutic agent is shown to enhance the therapeutic effect of the anti-tumor agent or overcome cellular resistance to such anti-tumor agent. This allows decrease of dosage of an anti-tumor agent, thereby reducing the undesirable side effects, or restores the effectiveness of an anti-neoplastic agent in resistant cells.

Conventional anti-tumor compounds include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

Depending on the nature of the combinatory therapy, administration of the therapeutic agents may be continued while the other therapy is being administered and/or thereafter. Administration of the therapeutic agents may be made in a single dose, or in multiple doses. In some instances, administration of the therapeutic agents is commenced at least several days prior to the conventional therapy. In other instances, administration is begun either immediately before or at the time of the administration of the conventional therapy.

VI. Methods of Administration and Pharmaceutical Compositions

In certain embodiments, the therapeutic agents (compounds) of the present disclosure are formulated with a pharmaceutically acceptable carrier. Such therapeutic agents can be administered alone or as a component of a pharmaceutical formulation (composition). The agents may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject agents include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of anti-tumor therapeutic agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject therapeutic agent as an active ingredient.

In solid dosage forms for oral administration (capsules, tablets, pills, dragees, powders, granules, and the like), one or more therapeutic agents may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, methods of the disclosure can be administered topically, either to skin or to mucosal membranes such as those on the cervix and vagina. This offers the greatest opportunity for direct delivery to tumor with the lowest chance of inducing side effects. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The subject therapeutic agents may be mixed under sterile conditions with a pharmaceutically acceptable carrier, and with any preservatives, buffers, or propellants which may be required. The ointments, pastes, creams and gels may contain, in addition to a subject nucleic acid molecule, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a therapeutic agent, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more therapeutic agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations for intravaginal or rectally administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

In certain embodiments, one or more therapeutic agents are formulated with a pharmaceutically acceptable agent that allows for the effective distribution of the agent in the physical location most suitable for their desired activity. Non-limiting examples of such pharmaceutically acceptable agents include: PEG, phospholipids, phosphorothioates, P-glycoprotein inhibitors (such as Pluronic P85) which can enhance entry of drugs into various tissues, biodegradable polymers, such as poly(DL-lactide-coglycolide) microspheres for sustained release delivery after implantation (Emerich, D F et al, 1999, Cell Transplant, 8, 47-58), and loaded nanoparticles such as those made of polybutylcyanoacrylate, which can deliver drugs across the blood brain barrier and can alter neuronal uptake mechanisms (Prog Neuropsychopharmacol Biol Psychiatry, 23, 941-949, 1999).

### EXEMPLIFICATION

The disclosure now being generally described, it will be more readily understood by reference to the following examples, which are included merely for purposes of illustration of certain aspects and embodiments of the present disclosure, and are not intended to limit the disclosure.

### Example 1

### EphB4 is Expressed in Squamous Cell Carcinoma of the Head and Neck (HNSCC)

A. HNSCC Tumors Express EphB4

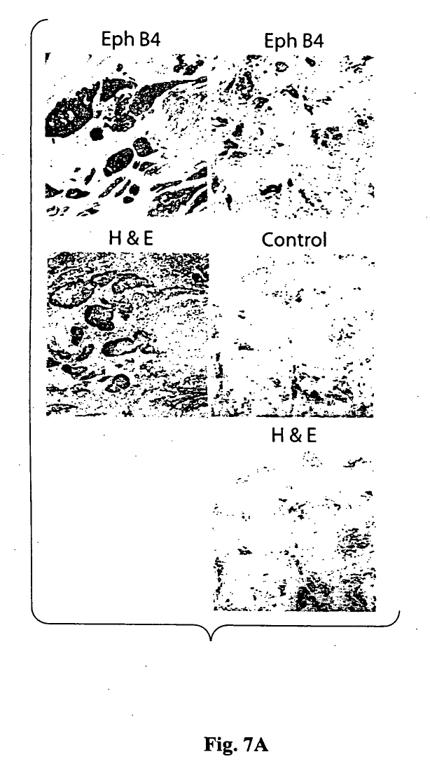

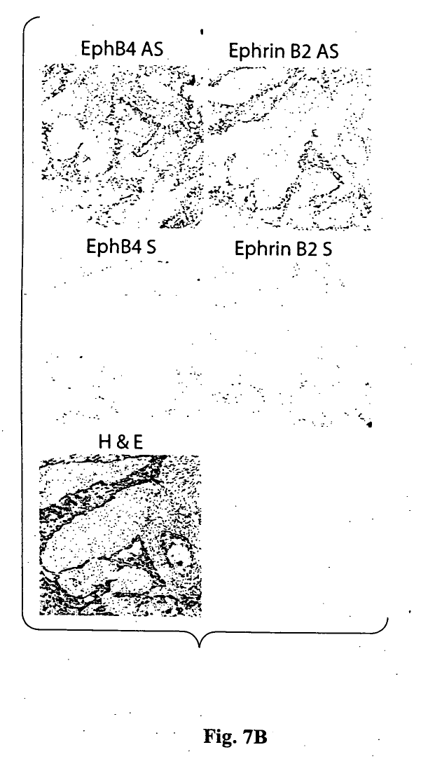

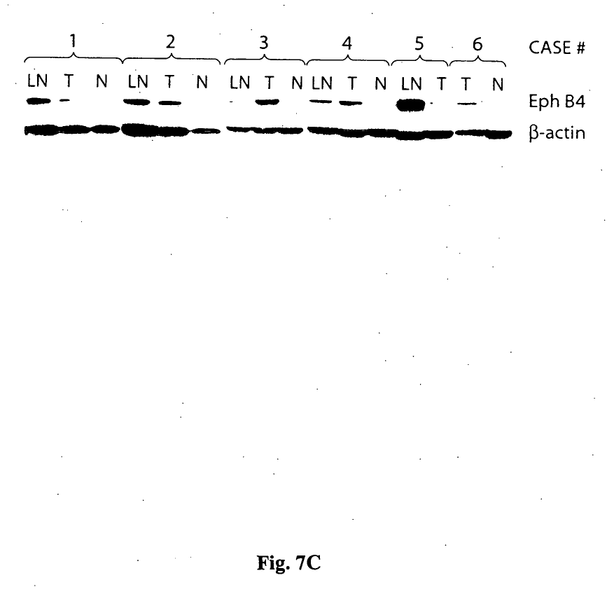

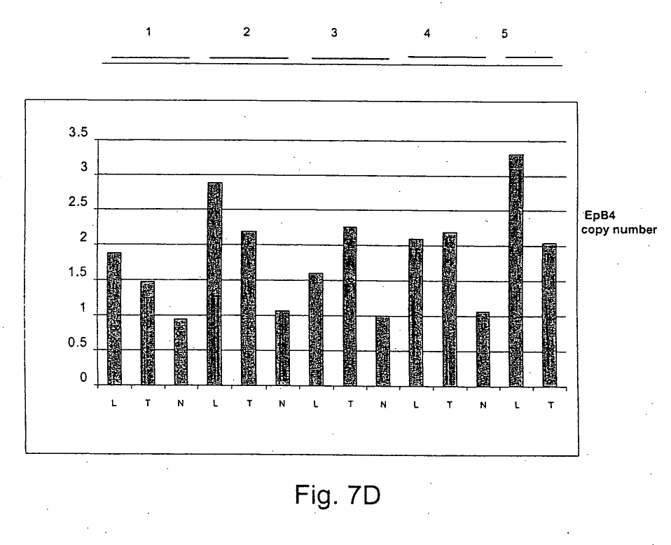

We studied the expression of EphB4 in human tumor tissues by immunohistochemistry, in situ hybridization, and Western blot. Twenty prospectively collected tumor tissues following IRB approval have been evaluated with specific EphB4 monoclonal antibody that does not react with other members of the EphB and EphA family. EphB4 expression is observed in alt cases, with varying intensity of staining. FIG. 7A (top left) illustrates a representative case, showing that EphB4 is expressed in the tumor regions only, as revealed by the H&E tumor architecture (FIG. 7A bottom left). Note the absence of staining for EphB4 in the stroma. Secondly, a metastatic tumor site in the lymph node shows positive staining while the remainder of the lymph node is negative (FIG. 7A, top right).

In situ hybridization was carried out to determine the presence and location of EphB4 transcripts in the tumor tissue. Strong signal for EphB4 specific antisense probe was detected indicating the presence of transcripts (FIG. 7B, top left). Comparison with the H&E stain (FIG. 7B, bottom left) to illustrate tumor architecture reveals that the signal was localized to the tumor cells, and was absent from the stromal areas. Ephrin B2 transcripts were also detected in tumor sample, and as with EphB4, the signal was localized to the tumor cells (FIG. 7B, top right). Neither EphB4 nor ephrin B2 sense probes hybridized to the sections, proving specificity of the signals.

B. Increased Expression and Gene Copy Number of EphB4 in Primary and Metastatic Sites of HNSCC

Western blots of tissue from primary tumor, lymph node metastases and uninvolved tissue were carried out to determine the relative levels of EphB4 expression in these sites. Tumor and normal adjacent tissues were collected on 20 cases, while lymph nodes positive for tumor were harvested in 9 of these 20 cases. Representative cases are shown in FIG. 7C. EphB4 expression is observed in each of the tumor samples. Similarly, all tumor positive lymph nodes show EphB4 expression that was equal to or greater than the primary tumor. No or minimal expression is observed in the normal adjacent tissue. Further, it was found that high expression of EphB4 was correlated with increased gene copy number in HNSCC primary tissues and metastasized tissues (FIG. 7D), suggesting that EphB4 gene amplification may be used a diagnostic marker for tumor status (in particular, tumor metastasis).

C. Increased Expression and Gene Copy Number of EphB4 in HNSCC Cell Lines

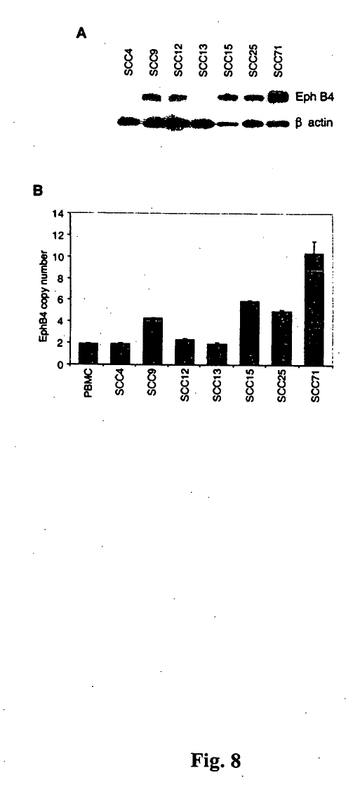

Having demonstrated the expression of EphB4 limited to tumor cells, we next sought to determine whether there was an in vitro model of EphB4 expression in HNSCC. Six HNSCC cell lines were surveyed for EphB4 protein expression by Western Blot (FIG. 8A). A majority of these showed strong EphB4 expression and thus established the basis for subsequent studies. A strong correlation between high expression of EphB4 and increased gene copy number of EphB4 was also found in the HNSCC cell lines (FIG. 8B). This result further supports that EphB4 gene amplification may be used a diagnostic marker for tumor status (in particular, tumor metastasis).

### Example 2

### EphB4 is Upregulated and Imparts Growth Advantage in Prostate Cancer

A. Expression of EphB4 in Prostate Cancer Cell Lines

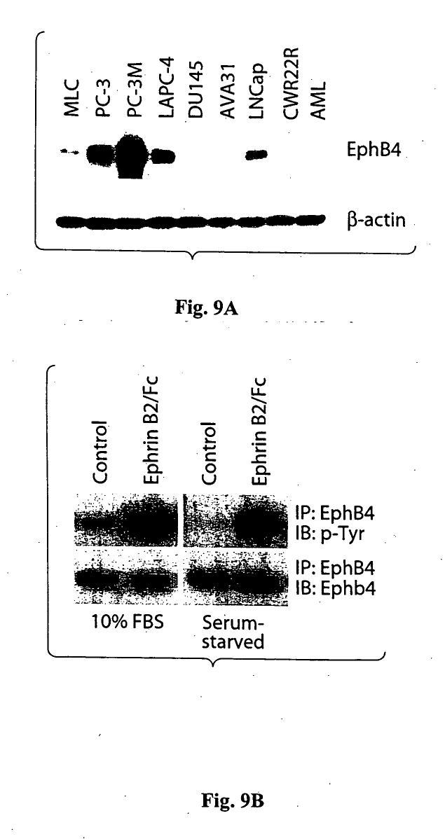

We first examined the expression of EphB4 protein in a variety of prostate cancer cell lines by Western blot. We found that prostate cancer cell lines show marked variation in the abundance of the 120 kD EphB4. The levels were relatively high in PC3 and even higher in PC3M, a metastatic clone of PC3, while normal prostate gland derived cell lines (MLC) showed low or no expression of EphB4 (FIG. 9A). We next checked the activation status of EphB4 in PC3 cells by phosphorylation study. We found that even under normal culture conditions, EphB4 is phosphorylated though it can be further induced by its ligand, ephrin B2 (FIG. 9B).

B. Expression of EphB4 in Clinical Prostate Cancer Samples

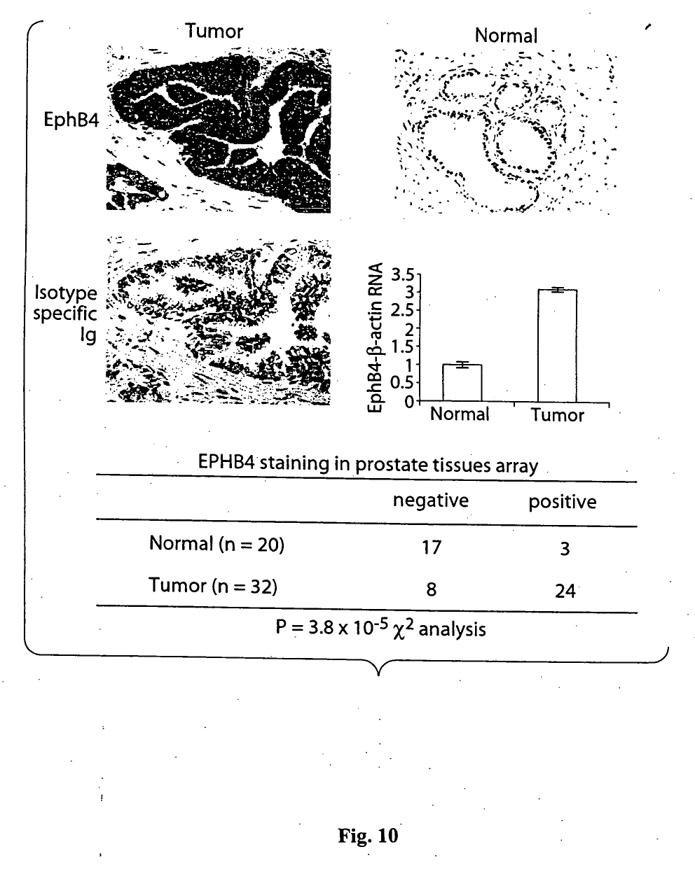

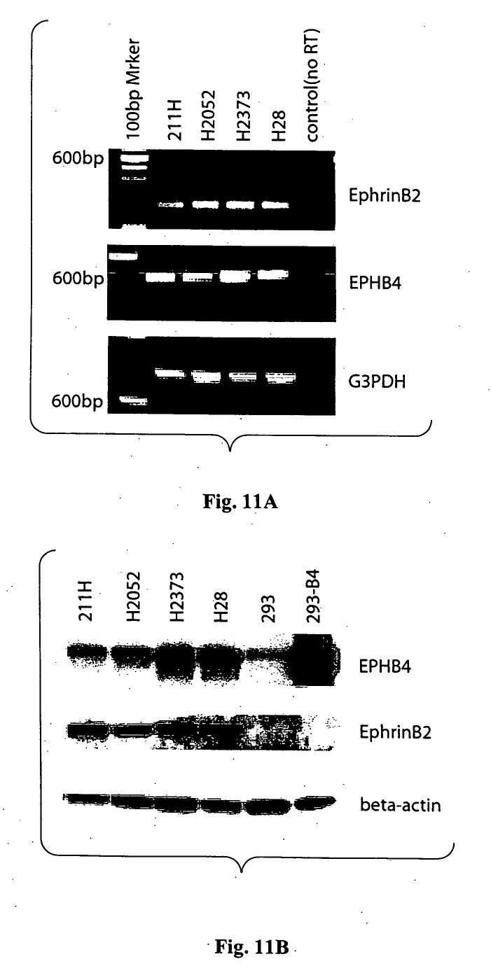

To determine whether EphB4 is expressed in clinical prostate samples, tumor tissues and adjacent normal tissue from prostate cancer surgical specimens were examined. The histological distribution of EphB4 in the prostate specimens was determined by immunohistochemistry. Clearly, EphB4 expression is confined to the neoplastic epithelium (FIG. 10, top left), and is absent in stromal and normal prostate epithelium (FIG. 10, top right). In prostate tissue array, 24 of the 32 prostate cancers examined were positive. We found EphB4 mRNA is expressed both in the normal and tumor tissues of clinical samples by quantitative RT-PCR. However, tumor EphB4 mRNA levels were at least 3 times higher than in the normal in this case (FIG. 10, lower right).

### Example 3

### Expression of EphB4 in Mesothelioma

A. EphB4 and EphrinB2 are Expressed in Mesothelioma Cell Lines

The expression of Ephrin B2 and EphB4 in malignant mesothelioma cell lines was determined at the RNA and protein level by a variety of methods. RT-PCR showed that all of the four cell lines express EphrinB2 and EphB4 (FIG. 11A). Protein expression was determined by Western blot in these cell lines. Specific bands for EphB4 were seen at 120 kD. In addition, Ephrin B2 was detected in all cell lines tested as a 37 kD band on Western blot (FIG. 11B). No specific band for Ephrin B2 was observed in 293 human embryonic kidney cells, which were included as a negative control.

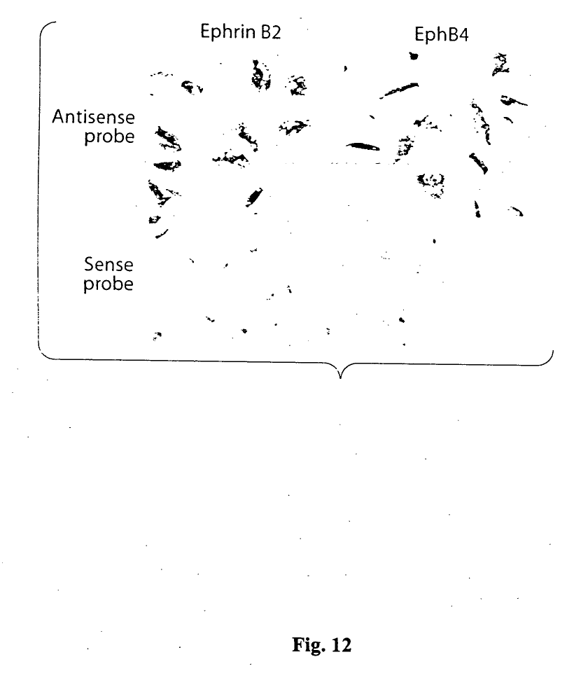

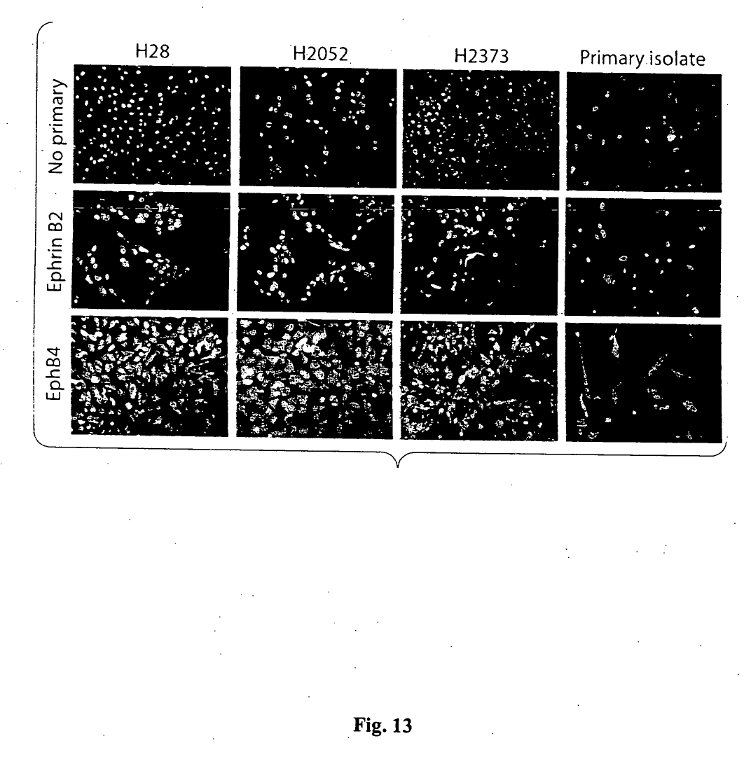

To confirm the presence of EphB4 transcription in mesothelioma cells, in situ hybridization was carried out on NCI H28 cell lines cultured on chamber slides. Specific signal for EphB4 was detected using antisense probe Ephrin B2 transcripts were also detected in the same cell line. Sense probes for both EphB4 and Ephrin B2 served as negative controls and did not hybridize to the cells (FIG. 12). Expression of EphB4 and Ephrin B2 proteins was confirmed in the cell lines by immunofluorescence analysis (FIG. 13). Three cell lines showed strong expression of EphB4, whereas expression of Ephrin B2 was present in H28 and H2052, and weakly detectable in H2373.

B. Evidence of Expression of EphB4 and EphrinB2 in Clinical Samples

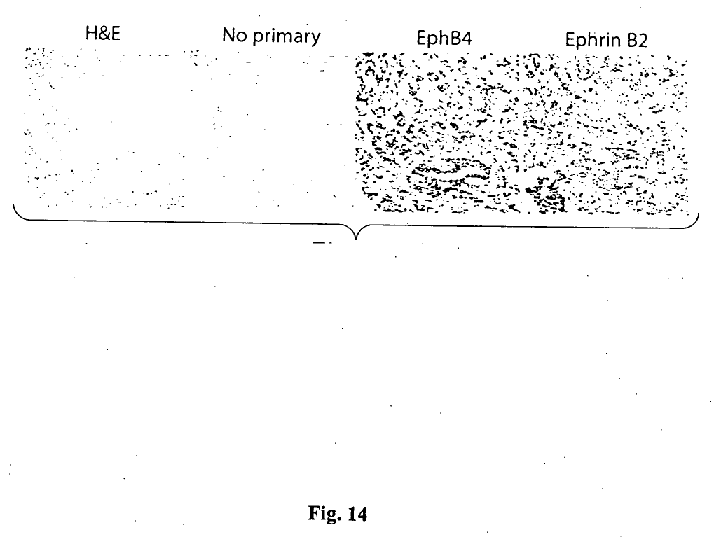

Tumor cells cultured from the pleural effusion of a patient diagnosed with pleural malignant mesothelioma were isolated and showed positive staining for both EphB4 and Ephrin B2 at passage 1 (FIG. 13, bottom row). These results confirm co-expression of EphB4 and Ephrin B2 in mesothelioma cell lines. To determine whether these results seen in tumor cell lines were a real reflection of expression in the disease state, tumor biopsy samples were subjected to immunohistochemical staining for EphB4 and Ephrin B2. Antibodies to both proteins revealed positive stain in the tumor cells. Representative data is shown in FIG. 14.

### Example 4

### Ephrin B2 Expression in Kaiposi's Sarcoma (KS) is Induced by Human Hermesvirus Type 8

A. KS Tumors Express Ephrin B2, but not EphB4

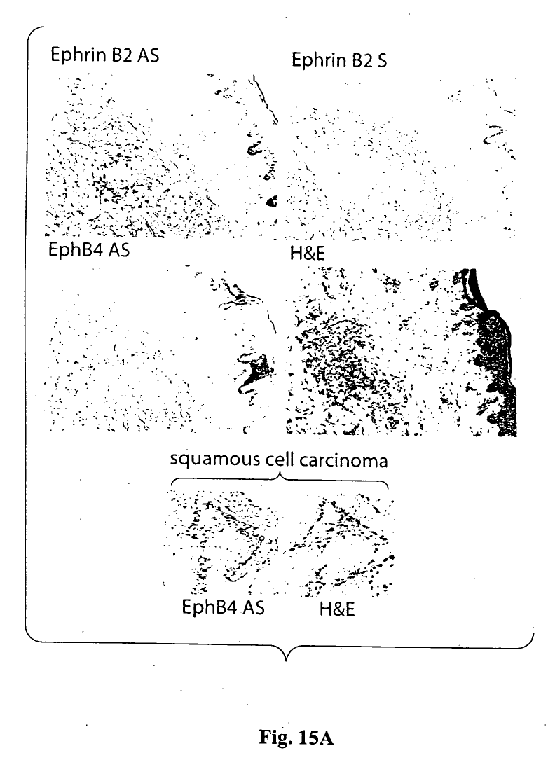

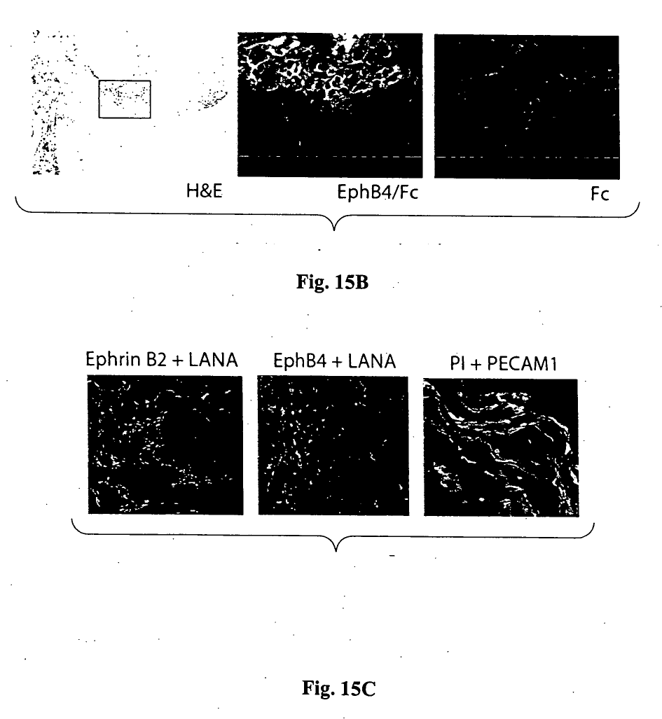

The highly vascular nature of KS lesions and the probable endothelial cell origin of the tumor cells prompted investigation of expression of EphB4 and ephrin B2 which are markers for venous and arterial endothelial cells, respectively. Ephrin B2, but not EphB4 transcripts were detected in tumor cells of KS biopsies by in situ hybridization (FIG. 15A). Comparison of the positive signal with ephrin B2 antisense probe and tumor cells as shown by H&E staining shows that ephrin B2 expression is limited to the areas of the biopsy that contain tumor cells. The lack of signal in KS with EphB4 antisense probe is not due to a defect in the probe, as it detected transcripts in squamous cell carcinoma, which we have shown expresses this protein. Additional evidence for the expression of ephrin B2 in KS tumor tissue is afforded by the localization of EphB4/Fc signal to tumor cells, detected by FITC conjugated anti human Fc antibody. Because ephrin B2 is the only ligand for EphB4 this reagent is specific for the expression of ephrin B2 (FIG. 15B, left). An adjacent section treated only with the secondary reagent shows no specific signal. Two-color confocal microscopy demonstrated the presence of the HHV-8 latency protein, LANA1 in the ephrin B2 positive cells (FIG. 15C, left), indicating that it is the tumor cells, not tumor vessels, which are expressing this arterial marker. Staining of tumor biopsy with PECAM-1 antibody revealed the highly vascular nature of this tumor (FIG. 15C, right). A pilot study of the prevalence of this pattern of ephrin B2 and EphB4 expression on KS biopsies was conducted by RT-PCR analysis. All six samples were positive for ephrin B2, while only 2 were weakly positive for EphB4 (data not shown).

B. HHV-8 vGPCR Induces Ephrin B2 Expression

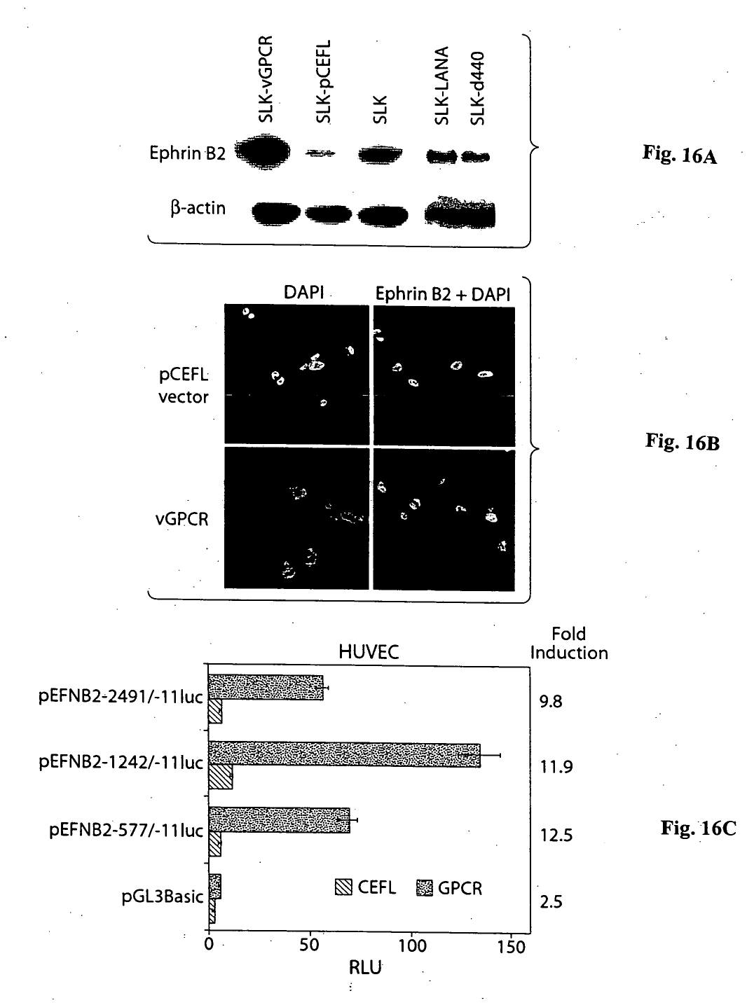

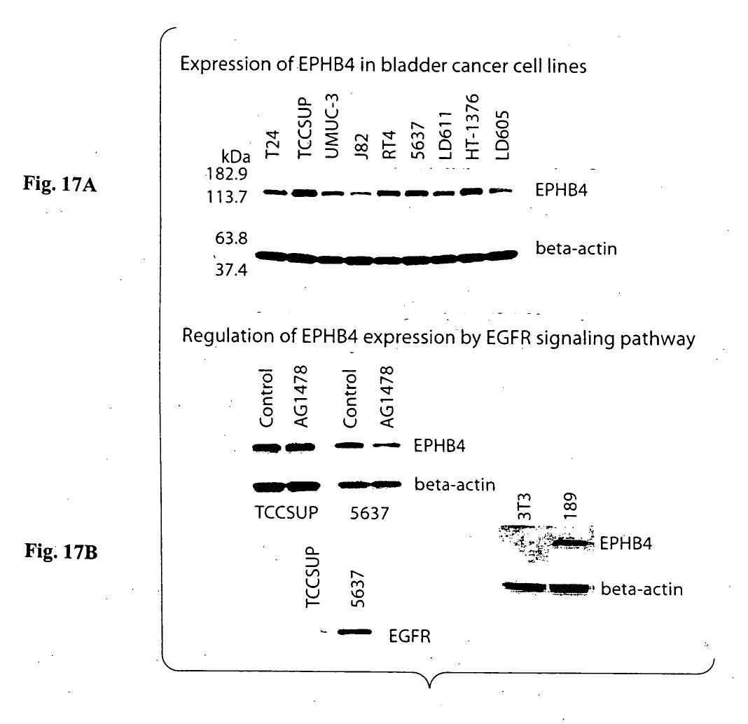

To test whether individual viral proteins could induce the expression of ephrin B2 seen with the whole virus KS-SLK cells were stably transfected with HHV-8 LANA, or LANAΔ440 or vGPCR. Western Blot of stable clones revealed a five-fold induction of ephrin B2 in KS-SLK transfected with vGPCR compared to SLK-LANA or SLK-LANAΔ440 (FIG. 16A). SLK transfected with vector alone (pCEFL) was used as a control. SLK-vGPCR and SLK-pCEFL cells were also examined for ephrin B2 and Eph B4 expression by immunofluorescence in transiently transfected KS-SLK cells. FIG. 16B shows higher expression of ephrin B2 in the SLK-vGPCR cells compared to SLK-pCEFL. No changes in EphB4 were observed in SLK-vGPCR compared to SLK-pCEFL. This clearly demonstrates that SLK-vGPCR cells expressed high levels of ephrin B2 compared to SLK-pCEFL cells. This suggests that vGPCR of HHV-8 is directly involved in the induction of Ephrin B2 and the arterial phenotype switch in KS. Since we had shown that HHV-8 induced expression of ephrin B2 in HUVEC, we next asked if this could be mediated by a transcriptional effect. Ephrin B2 5′-flanking DNA-luciferase reporter plasmids were constructed and transiently transfected into HUVECs. Ephrin B2 5′-flanking DNA sequences −2491/−11 have minimal activity in HUVEC cells (FIG. 16C). This is consistent with ephrin B2 being an arterial, not venous marker. However, we have noted that HUVEC in culture do express some ephrin B2 at the RNA level. Cotransfection of HHV-8 vGPCR induces ephrin B2 transcription approximately 10-fold compared to the control expression vector pCEFL. Roughly equal induction was seen with ephrin B2 sequences −2491/−11, −1242/−11, or −577/−11, which indicates that elements between −577 and −11 are sufficient to mediate the response to vGPCR, although maximal activity is seen with the −1242/−11 luciferase construct.

### Example 5

### Expression of EphB4 in Bladder Cancer

FIG. 17 shows expression of EphB4 in bladder cancer cell lines (A), and regulation of EphB4 expression by EGFR signaling pathway (B).

### Example 6

### Production of Soluble EphB4 Polypeptides

1) Mammalian Expression Vectors for Producing Recombinant Soluble Derivatives of Ephrin B2 and Eph B4.

A vector comprising a human EphB4 (hB4) cDNA comprising the full length ORF was amplified by PCR out with primers: GGATCCgccATGGAGCTCCGGGTGCTGCT (5Bam-hB4) and GCGGCCGCTCAGTACTGCGGGGCCGGT (3Not 1-B4), and cloned in BamHI-NotI cut pRK5 vector.

Sequence of BamHI-NotI-1 fragment with full length hB4 ORF

ggatccgccatggagctccgggtgctgctctgctgggcttcgttggccgcagctttggaagagaccctgctgaacacaaaattggaaactg
ctgatctgaagtgggtgacattccctcaggtggacgggcagtgggaggaactgagcggcctggatgaggaacagcacagcgtgcgcac
ctacgaagtgtgtgaagtgcagcgtgccccgggccaggcccactggcttcgcacaggttgggtcccacggcggggcgccgtccacgtgt
acgccacgctgcgcttcaccatgctcgagtgcctgtccctgcctcgggctgggcgctcctgcaaggagaccttcaccgtcttctactatgag
agcgatgcggacacggccacggccctcacgccagcctggatggagaacccctacatcaaggtggacacggtggccgcggagcatctca
cccggaagcgccctggggccgaggccaccgggaaggtgaatgtcaagacgctgcgtctgggaccgctcagcaaggctggcttctacct
ggccttccaggaccagggtgcctgcatggccctgctatccctgcacctcttctacaaaaagtgcgcccagctgactgtgaacctgactcgat
tcccggagactgtgcctcgggagctggttgtgcccgtggccggtagctgcgtggtggatgccgtccccgcccctggccccagccccagc
ctctactgccgtgaggatggccagtgggccgaacagccggtcacgggctgcagctgtgctccggggttcgaggcagctgaggggaaca
ccaagtgccgagcctgtgcccagggcaccttcaagcccctgtcaggagaagggtcctgccagccatgcccagccaatagccactctaac
accattggatcagccgtctgccagtgccgcgtcgggtacttccgggcacgcacagacccccggggtgcaccctgcaccacccctccttcg
gctccgcggagcgtggtttcccgcctgaacggctcctccctgcacctggaatggagtgcccccctggagtctggtggccgagaggacctc
acctacgccctccgctgccgggagtgccgacccggaggctcctgtgcgccctgcgggggagacctgacttttgaccccggcccccggg
acctggtggagccctgggtggtggttcgagggctacgtccggacttcacctatacctttgaggtcactgcattgaacggggtatcctccttag
ccacggggcccgtcccatttgagcctgtcaatgtcaccactgaccgagaggtacctcctgcagtgtctgacatccgggtgacgcggtcctc
acccagcagcttgagcctggcctgggctgttccccgggcacccagtggggcgtggctggactacgaggtcaaataccatgagaagggcg
ccgagggtcccagcagcgtgcggttcctgaagacgtcagaaaaccgggcagagctgcgggggctgaagcggggagccagctacctgg
tgcaggtacgggcgcgctctgaggccggctacgggcccttcggccaggaacatcacagccagacccaactggatgagagcgagggct
ggcgggagcagctggccctgattgcgggcacggcagtcgtgggtgtggtcctggtcctggtggtcattgtggtcgcagttctctgcctcag
gaagcagagcaatgggagagaagcagaatattcggacaaacacggacagtatctcatcggacatggtactaaggtctacatcgacccctt
cacttatgaagaccctaatgaggctgtgagggaatttgcaaaagagatcgatgtctcctacgtcaagattgaagaggtgattggtgcaggtg
agtttggcgaggtgtgccgggggcggctcaaggccccagggaagaaggagagctgtgtggcaatcaagaccctgaagggtggctacac
ggagcggcagcggcgtgagtttctgagcgaggcctccatcatgggccagttcgagcaccccaatatcatccgcctggagggcgtggtca
ccaacagcatgcccgtcatgattctcacagagttcatggagaacggcgccctggactccttcctgcggctaaacgacggacagttcacagt
catccagctcgtgggcatgctgcggggcatcgcctcgggcatgcggtaccttgccgagatgagctacgtccaccgagacctggctgctcg
caacatcctagtcaacagcaacctcgtctgcaaagtgtctgactttggcctttcccgattcctggaggagaactcttccgatcccacctacacg
agctccctgggaggaaagattcccatccgatggactgccccggaggccattgccttccggaagttcacttccgccagtgatgcctggagtta
cgggattgtgatgtgggaggtgatgtcatttggggagaggccgtactgggacatgagcaatcaggacgtgatcaatgccattgaacaggac
taccggctgcccccgcccccagactgtcccacctccctccaccagctcatgctggactgttggcagaaagaccggaatgcccggccccgc
ttcccccaggtggtcagcgccctggacaagatgatccggaaccccgccagcctcaaaatcgtggcccgggagaatggcggggcctcac
accctctcctggaccagcggcagcctcactactcagcttttggctctgtgggcgagtggcttcgggccatcaaaatgggaagatacgaaga
aagtttcgcagccgctggctttggctccttcgagctggtcagccagatctctgctgaggacctgctccgaatcggagtcactctggcgggac
accagaagaaaatcttggccagtgtccagcacatgaagtcccaggccaagccgggaaccccgggtgggacaggaggaccggccccgc
agtactgagcggccgc

Another version of BamHI-NotI full length (FL) human EphB4 was also cloned. The difference is the 3′-terminal PCR oligo primer used for cloning:

| | |
|---|---|
| 3Not1-B4 | GCGGCCGCTCAGTACTGCGGGGCCGGT |
| 3Not2-B4 | GCGGCCGCAGTTCCTGCAGGTCAAGTACT |

Plasmids vectors for expressing recombinant soluble derivatives of Ephrin B2 and EphB4 were based on pEF6/V5-His-TOPO vector (Invitrogen), pIG (Novagen) or pRK5. pEF6/V5-His-TOPO contains human elongation factor 1α enhancer/promoter and blasticidin resistance marker. pIG vector is designed for high-level expression of protein fusions with Fc portion of human IgG1 under CMV promoter control and pRK5 is a general purpose CMV promoter-containing mammalian expression vector. To generate plasmid construct pEF6-B4EC-NT, cDNA fragment of human EphB4 was amplified by PCR using oligo primers 5′-GGATCCGCC ATGGAGCTC CGGGTGCTGCT-3′ and 5′-TGGATCCCT GCTCCCGC CAGCCCTCG CTCTCATCCA-3′, and TOPO-cloned into pEF6NV5-His-TOPO vector. pEF6-hB4ECv3 was derived from pEF6-B4ECNT by digesting the plasmid DNA with EcoRV and BstBI, filling-in the ends with Klenow enzyme and religating the vector. Recombinant EphB4 derivative encoded by pEF6-B4EC-NT does not contain epitope- or purification tags, while the similar B4ECv3 protein encoded by pEF6-hB4ECv3 contains V5 epitope tag and 6×His tag on its C-terminus to facilitate purification from conditioned media. Plasmid construct pEF6-hB2EC was created by PCR amplification of Ephrin B2 cDNA using oligo primers 5′-TGGATCCAC CATGGCTGT GAGAAGGGAC-3′ plus 5′-ATTAATGGTGATGGT GAT GATGACTAC CCACTTCGG AACCGAGGAT GTTGTTC-3′ and TOPO-cloning into pEF6/V5-His-TOPO vector. Plasmid construct pIG-hB2EC-FC was created by PCR amplification of Ephrin B2 cDNA with oligo primers 5′-TAAAGCTTFCCGCCATGG CTGTGAGAAGGGAC-3′ and 5′-TAGGATCCACTTCGGA ACCGAGGATGTTGTT CCC-3′, followed by TOPO-cloning and sequencing the resulting PCR fragment with consecutive subcloning in pIG hIgG1 Fc fusion expression vector cut with Bam HI and Hind III. Similarly, pIG-hB2EC and pIG-hB4ECv3 were generated by PCR amplifying portions of EphB4 ECD cDNA using oligo primers 5′-ATAAGCTTCC GCCATGGAGC TCCGGGTGCTG-3′ plus 5′-TTGGATCCTGCTCCCG CCAGCCCTCGC TCTCATC-3′ with consecutive subcloning into pIG hIgG1 Fc fusion expression vector cut with Bam HI and Hind III. Predicted sequences of the proteins encoded by the vectors described above.

A construct encoding a truncated human EphB4 polypeptide comprising the globular (G) and cysteine-rich domains (C), the “GC” polypeptide, was prepared by PCR amplification using oligonucleotides:

5SpeB4
TACTAGTCCGCCATGGAGCTCCGGGTGCTGCT
3NotB4GC
gcggccgcttaatggtgatggtgatgatgAGCCGAAGGAGGGGTGGTGCA

The amplified portion was cloned by TA cloning into pEF6.

Sequence of the cloned fragment (SpeI-NotI fragment):

actagtccgccATGGAGCTCCGGGTGCTGCTCTGCTGGGCTTCGTTGGCCGCAGCTTTGGA
AGAGACCCTGCTGAACACAAAATTGGAAACTGCTGATCTGAAGTGGGTGACATTCC
CTCAGGTGGACGGGCAGTGGGAGGAACTGAGCGGCCTGGATGAGGAACAGCACAG
CGTGCGCACCTACGAAGTGTGTGAAGTGCAGCGTGCCCCGGGCCAGGCCCACTGGC
TTCGCACAGGTTGGGTCCCACGGCGGGGCGCCGTCCACGTGTACGCCACGCTGCGCT
TCACCATGCTCGAGTGCCTGTCCCTGCCTCGGGCTGGGCGCTCCTGCAAGGAGACCT
TCACCGTCTTCTACTATGAGAGCGATGCGGACACGGCCACGGCCCTCACGCCAGCCT
GGATGGAGAACCCCTACATCAAGGTGGACACGGTGGCCGCGGAGCATCTCACCCGG
AAGCGCCCTGGGGCCGAGGCCACCGGGAAGGTGAATGTCAAGACGCTGCGTCTGGG
ACCGCTCAGCAAGGCTGGCTTCTACCTGGCCTTCCAGGACCAGGGTGCCTGCATGGC
CCTGCTATCCCTGCACCTCTTCTACAAAAAGTGCGCCCAGCTGACTGTGAACCTGAC
TCGATTCCCGGAGACTGTGCCTCGGGAGCTGGTTGTGCCCGTGGCCGGTAGCTGCGT
GGTGGATGCCGTCCCCGCCCCTGGCCCCAGCCCCAGCCTCTACTGCCGTGAGGATGG
CCAGTGGGCCGAACAGCCGGTCACGGGCTGCAGCTGTGCTCCGGGGTTCGAGGCAG
CTGAGGGGAACACCAAGTGCCGAGCCTGTGCCCAGGGCACCTTCAAGCCCCTGTCA
GGAGAAGGGTCCTGCCAGCCATGCCCAGCCAATAGCCACTCTAACACCATTGGATC
AGCCGTCTGCCAGTGCCGCGTCGGGTACTTCCGGGCACGCACAGACCCCCGGGGTG
CACCCTGCACCACCCCTCCTTCGGCTcatcatcaccatcaccattaagcggccgc

The sequence of the Globular domain+Cys-rich domain (B4EC-GC), precursor protein is:

MELRVLLCWASLAAALEETLLNTKLETADLKWVTFPQVDGQWEELSGLDEEQH
SVRTYEVCEVQRAPGQAHWLRTGWVPRRGAVHVYATLRFTMLECLSLPRAGRSCKET
FTVFYYESDADTATALTPAWMENPYIKVDTVAAEHLTRKRPGAEATGKVNVKTLRLGP
LSKAGFYLAFQDQGACMALLSLHLFYKKCAQLTVNLTRFPETVPRELVVPVAGSCVVD
AVPAPGPSPSLYCREDGQWAEQPVTGCSCAPGFEAAEGNTKCRACAQGTFKPLSGEGSC
QPCPANSHSNTIGSAVCQCRVGYFRARTDPRGAPCTTPPSAHHHHHH

For many uses, including therapeutic use, the leader sequence (first 15 amino acids, so that the processed form begins Leu-Glu-Glu . . . ) and the c-terminal hexahistidine tag may be removed or omitted.

The plasmid for the GC protein has the sequence:

AATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGT
GCCACCTGACGTCGACGGATCGGGAGATCTCCCGATCCCCTATGGTCGACTCTCAGT
ACAATCTGCTCTGATGCCGCATAGTTAAGCCAGTATCTGCTCCCTGCTTCTCTCTTGG
AGGTCGCTGAGTAGTGCGCGAGCAAAATTTAAGCTACAACAAGGCAAGGCTTGACC
GACAATTGCATGAAGAATCTGCTTAGGGTTAGGCGTTTTGCGCTGCTTCGCGATGTA
CGGGCCAGATATACGCGTTGACATTGATTATTGACTAGGCTTTTGCAAAAAGCTTTG
CAAAGATGGATAAAGTTTTAAACAGAGAGGAATCTTTGCAGCTAATGGACCTTCTA
GGTCTTGAAAGGAGTGCCTCGTGAGGCTCCGGTGCCCGTCAGTGGGCAGAGCGCAC
ATCGCCCACAGTCCCCGAGAAGTTGGGGGGAGGGGTCGGCAATTGAACCGGTGCCT
AGAGAAGGTGGCGCGGGGTAAACTGGGAAAGTGATGTCGTGTACTGGCTCCGCCTT
TTTCCCGAGGGTGGGGGAGAACCTATATAAGTGCAGTAGTCGCCGTGAACGTTCTT
TTTCGCAACGGGTTTGCCGCCAGAACACAGGTAAGTGCCGTGTGTGGTTCCCGCGGG
CCTGGCCTCTTTACGGGTTATGGGCCCTTGCGTGCCTTGAATTACTTCCACCTGGCTGC
AGTACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCCTGGGCGCT
GGGGCCGCCGCTGCGAATCTGGTGGCACCTTCGCGCCTGTCTCGCTGCTTTCGATA
AGTCTCTAGCCATTTAAAATTTTTGATGACCTGCTGCGACGCTTTTTTTCTGGCAAGA
TAGTCTTGTAAATGCGGGCCAAGATCTGCACACTGGTATTTCGGTTTTTGGGGCCGC
GGGCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTCTG
GTGCCTGCCTCGCGCCGCCGTGTATCGCCCCGCCCTGGGCGGCAAGGCTGGCCCGG
TCGGCACCAGTTGCGTGAGCGGAAAGATGGCCGCTTCCCGGCCCTGCTGCAGGGAG
CTCAAAATGGAGGACGCGGCGCTCGGGAGAGCGGGCGGGTGAGTCACCCACACAA
AGGAAAAGGGCCTTTCCGTCCTCAGCCGTCGCTTCATGTGACTCCACGGAGTACCGG
GCGCCGTCCAGGCACCTCGATTAGTTCTCGAGCTTTTGGAGTACGTCGTCTTTAGGTT
GGGGGGAGGGGTTTTATGCGATGGAGTTTCCCCACACTGAGTGGGTGGAGACTGAA
GTTAGGCCAGCTTGGCACTTGATGTAATTCTCCTTGGAATTTGCCCTTTTTGAGTTTG
GATCTTGGTTCATTCTCAAGCCTCAGACAGTGGTTCAAAGTTTTTTTCTTCCATTTCA
GGTGTCGTGAGGAATTAGCTTGGTACTAATACGACTCACTATAGGGAGACCCAAGCT
GGCTAGGTAAGCTTGGTACCGAGCTCGGATCCACTAGTCCAGTGTGGTGGAATTGCC
CTTtactagtccgccATGGAGCTCCGGGTGCTGCTCTGCTGGGCTTCGTTGGCCGCAGCTTT
GGAAGAGACCCTGCTGAACACAAAATTGGAAACTGCTGATCTGAAGTGGGTGACAT
TCCCTCAGGTGGACGGGCAGTGGGAGGAACTGAGCGGCCTGGATGAGGAACAGCAC
AGCGTGCGCACCTACGAAGTGTGTGACGTGCAGCGTGCCCCGGGCCAGGCCCACTG
GCTTCGCACAGGTTGGGTCCCACGGCGGGGCGCCGTCCACGTGTACGCCACGCTGC
GCTTCACCATGCTCGAGTGCCTGTCCCTGCCTCGGGCTGGGCGCTCCTGCAAGGAGA
CCTTCACCGTCTTCTACTATGAGAGCGATGCGGACACGGCACGGCCCTCACGCCAG
CCTGGATGGAGAACCCCTACATCAAGGTGGACACGGTGGCCGCGGAGCATCTCACC
CGGAAGCGCCCTGGGGCCGAGGCCACCGGGAAGGTGAATGTCAAGACGCTGCGTCT
GGGACCGCTCAGCAAGGCTGGCTTCTACCTGGCCTTCCAGGACCAGGGTGCCTGCAT
GGCCCTGCTATCCCTGCACCTCTTCTACAAAAAGTGCGCCCAGCTGACTGTGAACCT
GACTCGATTCCCGGAGACTGTGCCTCGGGAGCTGGTTGTGCCCGTGGCCGGTAGCTG
CGTGGTGGATGCCGTCCCCGCCCCTGGCCCCAGCCCCAGCCTCTACTGCCGTGAGGA
TGGCCAGTGGGCCGAACAGCCGGTCACGGGCTGCAGCTGTGCTCCGGGGTTCGAGG
CAGCTGAGGGGAACACCAAGTGCCGAGCCTGTGCCCAGGGCACCTTCAAGCCCCTG
TCAGGAGAAGGGTCCTGCCAGCCATGCCCAGCCAATAGCCACTCTAACACCATTGG
ATCAGCCGTCTGCCAGTGCCGCGTCGGGTACTTCCGGGCACGCACAGACCCCCGGG
GTGCACCTGCACCACCCCTCCTTCGGCTcatcatcaccatcaccattaagcggccgcAAGGGCAATT
CTGCAGATATCCAGCACAGTGGCGGCCGCTCGAGTCTAGAGGGCCCGCGGTTCGAA
GGTAAGCCTATCCCTAACCCTCTCCTCGGTCTCGATTCTACGCGTACCGGTCATCATC
ACCATCACCATTGAGTTTAAACCCGCTGATCAGCCTCGACTGTGCCTTCTAGTTGCC
AGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACA
ATAGCAGGCATGCTGGGGATGCGGTGGGCTCTATGGCTCTGAGGCGGAAAGAACC
AGCTGGGGCTCTAGGGGGTATCCCCACGCGCCCTGTAGCGGCGCATTAAGCGCGGC
GGGTGTGGTGGTTACGCGCAGCGTGACCGCTACACTTGCCAGCGCCCTAGCGCCCGC
TCCTTTCGCTTTCTTCCCTTCCTTTCTCGCCACGTTCGCCGGCTTTCCCCGTCAAGCTC
TAAATCGGGGCATCCCTTTAGGGTTCCGATTTAGTGCTTTACGGCACCTCGACCCCA
AAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCAAACTGG
AACAACACTCAACCCTATCTCGGTCTATTCTTTTGATTTATAAGGGATTTTGGGGATT
TCGGCCTATTGGTTAAAAAATGAGCTGATTTAACAAAAATTTAACGCGAATTAATTC
TGTGGAATGTGTGTCAGTTAGGGTGTGGAAAGTCCCCAGGCTCCCCAGGCAGGCAG
AAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCTC
CGCCCCATGGCTGACTAATTTTTTTTATTTATGCAGAGGCCGAGGCCGCCTCTGCCTC
TGAGCTATTCCAGAAGTAGTGAGGAGGCTTTTTTGGAGGCCTAGGCTTTTGCAAAAA
GCTCCCGGGAGCTTGTATATCCATTTTCGGATCTGATCAGCACGTGTTGACAATTAA
TCATCGGCATAGTATATCGGCATAGTATAATACGACAAGGTGAGGAACTAAACCAT
GGCCAAGCCTTTGTCTCAAGAAGAATCCACCCTCATTGAAAGAGCAACGGCTACAA
TCAACAGCATCCCCATCTCTGAAGACTACAGCGTCGCCAGCGCAGCTCTCTCTAGCG
ACGGCCGCATCTTCACTGGTGTCAATGTATATCATTTTACTGGGGGACCTTGTGCAG
AACTCGTGGTGCTGGGCACTGCTGCTGCTGCGGCAGCTGGCAACCTGACTTGTATCG
TCGCGATCGGAAATGAGAACAGGGGCATCTTGAGCCCCTGCGGACGGTGTCGACAG
GTGCTTCTCGATCTGCATCCTGGGATCAAAGCGCTAGTGAAGGACAGTGATGGACA
GCCGACGGCAGTTGGGATTCGTGAATTGCTGCCCTCTGGTTATGTGTGGGAGGGCTA
AGCACTTCGTGGCCGAGGAGCAGGACTGACACGTGCTACGAGATTTCGATTCCACC
GCCGCCTTCTATGAAAGGTTGGGCTTCGGAATCGTTTTCCGGGACGCCGGCTGGATG
ATCCTCCAGCGCGGGGATCTCATGCTGGAGTTCTTCGCCCACCCCAACTGTTTATTG
CAGCTTATAATGGTTACAAATAAAGCAATAGCATCACAAATTTCACAAATAAAGCA
TTTTTTTCACTGCATTCTAGTTGTGGTTTGTCCCAAACTCATCAATGTATCTTATCATGT
CTGTATACCGTCGACCTCTAGCTAGAGCTTGGCGTAATCATGGTCATAGCTGTTTCCT
GTGTGAAATTGTTATCCGCTCACAATTCCACACAACATACGAGCCGGAAGCATAAA
GTGTAAAGCCTGGGGTGCCTAATGAGTGAGCTAACTCACATTAATTGCGTTGCGCTC
ACTGCCCGCTTTCCAGTCGGGAAACCTGTCGTGCCAGCTGCATTAATGAATCGGCCA
ACGCGCGGGGAGAGGCGGTTGCGTATTGGGCGCTCTTCCGCTTCCTCGCTCACTGA
CTCGCTGCGCTCGGTCGTTCGGCTGCGGCGCGCGGTATCAGCTCACTCAAAGGCGGT
AATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAG
CGTGGCGCTTTCTCAATGCTCACGCTGTAGGTATCTCAGTTCGGTGTAGGTCGTTCGC
TCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTT
GAAGTGGTGGCCTAACTACGGCTACACTAGAAGGACAGTATTTGGTATCTGCGCTCT
GCTGAAGCCAGTTACCTTCGGAAAAAGAGTTGGTAGCTCTTGATCCGGCAAAGAAA
CCACCGCTGGTAGCGGTGGTTTTTTTGTTTGCAAGCAGCAGATTACGCGCAGAAAAA
AAGGATCTCAAGAAGATCCTTTGATCTTTTCTACGGGGTCTGACGCTCAGTGGAACG
AAAACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAACTT
GGTCTGACAGTTACCAATGCTTAATCAGTGAGGCACCTATCTCAGCGATCTGTCTAT
TTCGTCATCCATAGTTGCCTGACTCCCCGTCGTGTAGATAACTACGATACGGGAGG
GCTTACCATCTGGCCCCAGTGCTGCAATGATACCGCGAGACCCACGCTCACCGGCTC
CAGATTTATCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGTGGTG
TCACGCTCGTCGTTTGGTATGGCTTCATTCAGCTCCGGTTCCCAACGATCAAGGCGA
GTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCAT
AATTCTCTTACTGTCATGCCATCCGTAAGATGCTTTTCTGTGACTGGTGAGTACTCAA
CCAAGTCATTCTGAGAATAGTGTATGCGGCGACCGAGTTGCTCTTGCCCGGCGTCAA
TACGGGATAATACCGCGCCACATAGCAGAACTTTAAAAGTGCTCATCATTGGAAAA
CGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTG
GGTGAGCAAAAACAGGAAGGCAAAATGCCGCAAAAAAGGGAATAAGGGCGACACG
GAAATGTTGAATACTCATACTCTTCCTTTTTC

A nucleic acid encoding truncated human EphB4 protein comprising the globular domain, Cys-rich domain and the first FNIII domain (GCF) was prepared by PCR with oligonucleotides:

5SpeB4
TACTAGTCCGCCATGGAGCTCCGGGTGCTGCT
3NotB4GCF1
AGCGGCCGCTTAATGGTGATGGTGATGATGGACATTGACAGGCTCA
AATGGGA

TA cloned into pEF6. Sequence of the cloned fragment (SpeI-NotI fragment):

tactagtccgccATGGAGCTCCGGGTGCTGCTCTGCTGGGCTTCGTTGGCCGCAGCTT
TGGAAGAGACCCTGCTGAACACAAAATTGGAAACTGCTGATCTGAAGTGGGTGACA
TTCCCTCAGGTGGACGGGCAGTGGGAGGAACTGAGCGGCCTGGATGAGGAACAGCA
CAGCGTGCGCACCTACGAAGTGTGTGAAGTGCAGCGTGCCCCGGGCCAGGCCCACT
GGCTTCGCACAGGTTGGGTCCCACGGCGGGGCGCCGTCCACGTGTACGCCACGCTG
CGCTTCACCATGCTCGAGTGCCTGTCCCTGCCTCGGGCTGGGCGCTCCTGCAAGGAG
ACCTTCACCGTCTTCTACTATGAGAGCGATGCGGACACGGCCACGGCCCTCACGCCA
GCCTGGATGGAGAACCCCTACATCAAGGTGGACACGGTGGCCGCGGAGCATCTCAC
CCGGAAGCGCCCTGGGGCCGAGGCCACCGGGAAGGTGAATGTCAAGACGCTGCGTC
TGGGACCGCTCAGCAAGGCTGGCTTCTACCTGGCCTTCCAGGACCAGGGTGCCTGCA
TGGCCCTGCTATCCCTGCACCTCTTCTACAAAAAGTGCGCCCAGCTGACTGTGAACC
TGACTCGATTCCCGGAGACTGTGCCTCGGGAGCTGGTTGTGCCCGTGGCCGGTAGCT
GCGTGGTGGATGCCGTCCCCGCCCCTGGCCCCAGCCCCAGCCTCTACTGCCGTGAGG
ATGGCCAGTGGGCCGAACAGCCGGTCACGGGCTGCAGCTGTGCTCCGGGGTTCGAG
GCAGCTGAGGGGAACACCAAGTGCCGAGCCTGTGCCCAGGGCACCTTCAAGCCCCT
GTCAGGAGAAGGGTCCTGCCAGCCATGCCCAGCCAATAGCCACTCTAACACCATTG
GATCAGCCGTCTGCCAGTGCCGCGTCGGGTACTTCCGGGCACGCACAGACCCCCGG
GGTGCACCCTGCACCACCCCTCCTTCGGCTCCGCGGAGCGTGGTTTCCCGCCTGAAC
GGCTCCTCCCTGCACCTGGAATGGAGTGCCCCCCTGGAGTCTGGTGGCCGAGAGGA
CCTCACCTACGCCCTCCGCTGCCGGGAGTGCCGACCCGGAGGCTCCTGTGCGCCCTG
CGGGGGAGACCTGACTTTTGACCCCGGCCCCCGGGACCTGGTGGAGCCCTGGGTGG
TGGTTCGAGGGCTACGTCCGGACTTCACCTATACCTTGAGGTCACTGCATTGAACG
GGGTATCCTCCTTAGCCACGGGGCCCGTCCCATTTGAGCCTGTCAATGTCCATCATC
ACCATCACCATTAAgcggccgct

Sequence of the GCF precursor protein:

MELRVLLCWASLAAALEETLLNTKLETADLKWVTFPQVDGQWEELSGLDEEQH
SVRTYEVCEVQRAPGQAHWLRTGWVPRRGAVHVYATLRFTMLECLSLPRAGRSCKET
FTVFYYESDADTATALTPAWMENPYIKVDTVAAEHLTRKRPGAEATGKVNVKTLRLGP
LSKAGFYLAFQDQGACMALLSLHLFYKKCAQLTVNLTRFPETVPRELVVPVAGSCVVD
AVPAPGPSPSLYCREDGQWAEQPVTGCSCAPGFAEGNTKCRACAQGTFKPLSGEGSCQP
CPANSHSNTIGSAVCQCRVGYFRARTDPRGAPCTFTPPSAPRSVVSRLNGSSLHLEWSAPL
ESGGREDLTYALRCRECRPGGSCAPCGGDLTFDPGPRDLVEPWVVVRGLRPDFTYTFEV
TALNGVSSLATGPVPFEPVNVHHHHHH

For many uses, including therapeutic use, the leader sequence (first 15 amino acids, so that the processed form begins Leu-Glu-Glu . . . ) and the c-terminal hexahistidine tag may be removed or omitted.

Plasmid DNA sequence:

AATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCA
CCTGACGTCGACGGATCGGGAGATCTCCCGATCCCCTATGGTCGACTCTCAGTACAA
TCTGCTCTGATGCCGCATAGTTAAGCCAGTATCTGCTCCCTGCTTGTGTGTTGGAGGT
CGCTGAGTAGTGCGCGAGCAAAATTAAGCTACAACAAGGCAAGGCTTGACCGACA
ATTGCATGAAGAATCTGCTTAGGGTTAGGCGTTTTGCGCTGCTCGCGATGTACGGG
CCAGATATACGCGTTGACATGATTATTGACTAGGCTTTGCAAAAAGCTTTGCAAA
GATGGATAAAGTTTAAACAGAGAGGAATCTTTGCAGCTAATGGACCTTCTAGGTCT
TGAAAGGAGTGCCTCGTGAGGCTCCGGTGCCCGTCAGTGGGCAGAGCGCACATCGC
CCACAGTCCCCGAGAAGTTGGGGGGAGGGGTCGGCAATTGAACCGGTGCCTAGAGA
AGGTGGCGCGGGGTAAACTGGGAAAGTGATGTCGTGTACTGGCTCCGCCTTTCCC
GAGGGTGGGGGAGAACCGTATATAAGTGCAGTAGTCGCCGTGAACGTTCTTTTTCGC
AACGGGTTTGCCGCCAGAACACAGGTAAGTGCCGTGTGTGGTTCCCGCGGGCCTGG
CCTCTTACGGGTTATGGCCCTTGCGTGCCTTGAATTACTTCCACCTGGCTGCAGTAC
GTGATTCTTGATCCCGAGCTTCGGGTTGGAAGTGGGTGGGAGAGTTCGAGGCCTTGC
GCTTAAGGAGCCCCTTCGCCTCGTGCTTGAGTTGAGGCCTGGCCTGGGCGCTGGGGC
CGCCGCGTGCGAATCTGGTGGCACCTTCGCGCCTGTCTCGCTGCTTTCGATAAGTCT
CTAGCCATTTAAAATTTTTGATGACCTGCTGCGACGCTTTTTTTCTGGCAAGATAGTC
TTGTAAATGCGGGCCAAGATCTGCACACTGGTATTTCGGTTTTTGGGGCCGCGGGCG
GCGACGGGGCCCGTGCGTCCCAGCGCACATGTTCGGCGAGGCGGGGCCTGCGAGCG
CGGCCACCGAGAATCGGACGGGGGTAGTCTCAAGCTGGCCGGCCTGCTCTGGTGCC
TGGCCTCGCGCCGCCGTGTATCGCCCCGCCCTGGGCGGCAAGGCTGGCCCGGTCGGC
ACCAGTTGCGTGAGCGGAAAGATGGCCGCTTCCCGGCCCTGCTGCAGGGAGCTCAA
AATGGAGGACGCGGCGCTCGGGAGAGCGGGCGGGTGAGTCACCCACACAAAGGAA
AAGGGCCTTTCCGTCCTCAGCCGTCGCTTCATGTGACTCCACGGAGTACCGGGCGCC
GTCCAGGCACCTCGATTAGTTCTCGAGCTTTTGGAGTACGTCGTCTTTAGGTTGGGG
GGAGGGGTTTTATGCGATGGAGTTTCCCCACACTGAGTGGGTGGAGACTGAAGTTA
GGCCAGCTTGGCACTTGATGTAATTCTCCTTGGAATTTGCCCTTTTTGAGTTTGGATC
TTGGTTTCATTCTCAAGCCTCAGACAGTGGTTCAAAGTTTTTTTCTTCCATTCAGGTG
TCGTGAGGAATTAGCTTGGTACTAATACGACTCACTATAGGGAGACCCAAGCTGGCT
AGGTAAGCTTGGTACCGAGCTCGGATCCACTAGTCCAGTGTGGTGGAATTGCCCTta
ctagtccgccATGGAGCTCCGGGTGCTGCTCTGCTGGGCTTCGTTGGCCGCAGCTTTGGAA
GAGACCCTGCTGAACACAAAATTGGAAACTGCTGATCTGAAGTGGGTGACATTTCCCT
CAGGTGGACGGGCAGTGGGAGGAACTGAGCGGCCTGGATGAGGAACAGCACAGCG
TGCGCACCTACGAAGTGTGTGACGTGCAGCGTGCCCCGGGCCAGGCCCACTGGCTTC
GCACAGGTTGGGTCCCACGGCGGGGCGCCGTCCACGTGTACGCCACGCTGCGCTTC
ACCATGCTCGAGTGCCTGTCCCTGCCTCGGGCTGGGCGCTCCTGCAAGGAGACCTTC
ACCGTCTTCTACTATGAGAGCGATGCGGACACGGCCACGGCCCTCACGCCAGCCTG
GATGGAGAACCCCTACATCAAGGTGGACACGGTGGCCGCGGAGCATCTCACCCGGA
AGCGCCCTGGGGCCGAGGCCACCGGGAAGGTGAATGTCAAGACGCTGCGCCTGGGA
CCGCTCAGCAAGGCTGGCTTCTACCTGGCCTTCCAGGACCAGGGTGCCTGCATGGCC
CTGCTATCCCTGCACCTCTTCTACAAAAAGTGCGCCCAGCTGACTGTGAACCTGACT
CGATTCCCGGAGACTGTGCCTCGGGAGCTGGTTTGTGCCCGTGGCCGGTAGCTGCGTG
GTGGATGCCGTCCCCGCCCCTGGCCCCAGCCCCAGCCTCTACTGCCGTGAGGATGGC
CAGTGGGCCGAACAGCCGGTCACGGGCTGCAGCTGTGCTCCGGGGTTCGAGGCAGC
TGAGGGGAACACCAAGTGCCGAGCCTGTGCCCAGGGCACCTTCAAGCCCCTGTCAG
GAGAAGGGTCCTGCCAGCCATGCCCAGCCAATAGCCACTCTAACACCATTGGATCA
GCCGTCTGCCAGTGCCGCGTCGGGTACTTCCGGGCACGCACAGACCCCCGGGGTGC
ACCCTGCACCACCCCTCCTTCGGCTCCGCGGAGCGTGGTTTCCCGCCTGAACGGCTC
CTCCCTGCACCTGGAATGGAGTGCCCCCCTGGAGTCTGGTGGCCGAGAGGACCTCAC
CTACGCCCTCCGCTGCCGGGAGTGTCGACCCGGAGGCTCCTGTGCGCCCTGCGGGGG
AGACCTGACTTTTGACCCCGGCCCCCGGGACCTGGTGGAGCCCTGGGTGGTGGTTCG
AGGGCTACGTCCTGACTTCACCTATACCTTTGAGGTCACTGCATTGAACGGGGTATC
CTCCTTAGCCACGGGGCCCGTCCCATTTGAGCCTGTCAATGTCCATCATCACCATCA
CCATAAgcggccgctAAGGGCAATTCTGCAGATATCCAGCACAGTGGCGGCCGCTCGA
GTCTAGAGGGCCCGCGGTTCGAAGGTAAGCCTATCCCTAACCCTCTCCTCGGTCTCG
ATTCTACGCGTACCGGTCATCATCACCATCACCATTGAGTTTAAACCCGCTGATCAG
CCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGC
ATCGCATTGTCTGAGTAGGTGTCATTTCTATTCTGGGGGGTGGGGTGGGGCAGGACAG
CAAGGGGGAGGATTTGGGAAGACAATAGCAGGCATGCTGGGGATGCGGTGGGCTCTA
TGGCTTCTGAGGCGGAAAGAACCAGCTGGGGCTCTAGGGGGTATCCCCACGCGCCC
TGTAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCACG
TTCGCCGGCCTTTCCCCCGTCAAGCTCTAAATCGGGGCATCCCTTTAGGGTTCCGATTTA
GTGCTTTACGGCACCTCGACCCCAAAAAACTTGATTAGGGTGATGGTTCACGTAGTG
GGCCATCGCCCTGATAGACGGTTTTTCGCCCTTTGACGTGGAGTCCACGTTCTTTAA
TAGTGGACTCTTGTCCAACTGGAACAACACTCAACCCTATCTCGGTCTATTCTTTT
GATTTATAAGGGATTTTGGGGATTTCGGCCTATTGGTTAAAAAATGAGCTGATTTAA
CAAAAATTTAACGCGAATTAATTCTGTGGAATGTGTGTCAGTTAGGGTGTGGAAAGT
CCCCAGGCTCCCCAGGCAGGCAGAAGTATGCAAAGCATGCATCTCAATTAGTCAGC
AACCAGGTGTGGAAAGTCCCCAGGCTCCCCAGCAGGCAGAAGTATGCAAAGCATGC
ATCTCAATTAGTCAGCAACCATAGTCCCGCCCCTAACTCCGCCCATCCCGCCCCTAA
CTCCGCCCAGTTCCGCCCATTCTCCGCCCCATGGCTGACTAATTTTTTTTATTTATGC
AGAGGCCGAGGCCGCCTCTGCCTCTGAGCTATTCCAGAAGTAGTGAGGAGGCTTTTT
TGGAGGCCTAGGCTTTTGCAAAAAGCTCCCGGGAGCTTGTATATCCATTTTCGGATC
TGATCAGCACGTGTTGACAATTAATCATCGGCATAGTATATCGGCATAGTATAATAC
GACAAGGTGAGGAACTAAACCATGGCCAAGCCTTTGTCTCAAGAAGAATCCACCCT
CATTGAAAGAGCAACGGCTACAATCAACAGCATCCCCATCTCTGAAGACTACAGCG
TCGCCAGCGCAGCTCTCTCTAGCGACGGCCGCATCTTCACTGGTGTCAATGTATATC
ATTTTACTGGGGGACCTTGTGCAGAACTCGTGGTGCTGGGCACTGCTGCTGCTGCGG
CAGCTGGCACCTGACTTGTATCGTCGCGATCGGAAATGAGAACAGGGGCATCTTG
AGCCCCTGCGGACGGTGTCGACAGGTGCTTCTCGATCTGCATCCTGGGATCAAAGCG
ATAGTGAAGGACAGTGATGGACAGCCGACGGCAGTTGGGATTCGTGAATTGCTGCC
CTCTGGTTATGTGTGGGAGGGCTAAGCACTTCGTGGCCGAGGAGCAGGACTGACAC
GTGCTACGAGATTTCGATTCCACCGCCGCCTTCTATGAAAGGTTGGGCTTCGGAATC
GTTTTCCGGGACGCCGGCTGGATGATCCTCCAGCGCGGGGATCTCATGCTGGAGTTC
TTCGCCCACCCCAACTTGTTTATTGCAGCTTATAATGGTTACAAATAAAGCAATAGC
ATCACAAATTTCACAAATAAAGCATTTTTTTCACTGCATTCTAGTTGTGGTTTGTCCA
AACTCATCAATGTATCTTATCATGTCTGTATACCGTCGACCTCTAGCTAGAGCTTGGC
GTAATCAATGGTCATAGCTGTTTCCTGTGTGAAATTGTTATCCGCTCACAATTCCACAC
AACATACAGAGCCGGAAGCATAAAGTGTAAAGCCTGGGGTGCCTAATGAGTGAGCTA
ACTCACATTAATTGCGTTGCGCTCACTGCCCGCTTTCCAGTCGGGAAACCTGTCGTG
CCAGCTGCATTAATGAATCGGCCAACGCGCGGGGAGAGGCGGTTTGCGTATTGGGC
GCTCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTC
CCCCTGGAAGCTCCCTCGTGCGCTCTCCTGTTCCGACCCTGCCGCTTACCGGATACCT
GTCCGCCTTTCTCCCTTCGGGAAGCGTGGCGCTTTCTCAATGCTCACGCTGTAGGTAT
CTCAGTTCGGTGTAGGTCGTTCGCTCCAAGCTGGGCTGTGTGCACGAACCCCCCGTT
CAGCCCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAA
GCAGCAGATTACGCGCAGAAAAAAAGGATCTCAAGAAGATCCTTTGATCTTTTCTAC
GGGGTCTGACGCTCAGTGGAACGAAAACTCACGTTAAGGGATTTTGGTCATGAGATT
ATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGC
ACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCG
CGAGACCCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGGTT
AGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTT
CTGTGACTGGTGAGTACTCAACCAAGTCATTCTGAGAATAGTGTATGCGGCGACCGA
GTTGCTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATCTTT
TACTTTCACCAGCGTTTCTGGGTGAGCAAAAACAGGAAGGCAAAATGCCGCAAAAA
AGGAATAAGGCGACACGGAAATGTTGAATACTCATACTCTTCCTTTTTC

A vector encoding truncated human EphB4 protein having the Globular, Cys-rich and two FNIII domains with a c-terminal tag, GCF2 (v.3) was derived from pEF6-FL-hB4EC by digesting with EcoRV and BstBI, treating with Klenow and religating.

Amino acid sequence of encoded FL-hB4EC precursor (His-tagged):

MELRVLLCWASLAAALEETLLNTKLETADLKWVTFPQVDGQWEELSGLDEEQH
SVRTYEVCEVQRAPGQAHWLRTGWVPRRGAVHVYATLRFTMLECLSLPRAGRSCKET
FTVFYYESDADTATALTPAWMENPYIKVDTVAAEHLTRKRPGAEATGKVNVKTLRLGP
LSKAGFYLAFQDQGACMALLSLHLFYKKCAQLTVNLTRFPETVPRELVVPVAGSCVVD
AVPAPGPSPSLYCREDGQWAEQPVTGCSCAPGFEAAEGNTKCRACAQGTFKPLSGEGSC
QPCPANSHSNTIGSAVCQCRVGYFRARTDPRGAPCTTPPSAPRSVVSRLNGSSLHLEWSA
PLESGGREDLTYALRCRECRPGGSCAPCGGDLTFDPGPRDLVEPWVVVRGLRPDFTYTF
EVTALNGVSSLATGPVPFEPVNVTTDREVPPAVSDLRVTRSSPSSLSLAWAVPRAPSGAW
LDYEVKYHEKGAEGPSSVRFLKTSENRAELRGLKRGASYLVQVRARSEAGYGPFGQEH
HSQTQLDESEGWREQGSKRAILQWGKPIPNPLLGLDSTRTGHHHHHH

For many uses, including therapeutic use, the leader sequence (first 15 amino acids, so that the processed form begins Leu-Glu-Glu . . . ) and the c-terminal hexahistidine tag may be removed or omitted.

Plasmid DNA sequence:

aatattattgaagcatttatcagggttattgtctcatgagcggatacatatttgaatgtatttagaaaaataaacaaataggggttccgc
gcacatttccccgaaaagtgccacctgacgtcgacggatcgggagatctcccgatcccctatggtcgactctcagtacaatctgctctgatgc
cgcatagttaagccagtatctgctccctgcttgtgtgttggaggtcgctgagtagtgcgcgagcaaaatttaagctacaacaaggcaaggctt
gaccgacaattgcatgaagaatctgcttagggttaggcgttttgcgctgcttcgcgatgtacgggccagatatacgcgttgacattgattattg
actaggcttttgcaaaaagctttgcaaagatggataaagttttaaacagagaggaatctttgcagctaatggaccttctaggtcttgaaaggagt
gcctcgtgaggctccggtgcccgtcagtgggcagagcgcacatcgcccacagtccccgagaagttggggggaggggtcggcaattgaa
ccggtgcctagagaaggtggcgcggggtaaactgggaaagtgatgtcgtgtactggctccgcctttttcccgagggtgggggagaaccgt
atataagtgcagtagtcgccgtgaacgttctttttcgcaacgggtttgccgccagaacacaggtaagtgccgtgtgtggttcccgcgggcctg
gcctctttacgggttatggcccttgcgtgccttgaattacttccacctggctgcagtacgtgattcttgatcccgagcttcgggttggaagtggg
tgggagagttcgaggccttgcgcttaaggagccccttcgcctcgtgcttgagttgaggcctggcctgggcgctggggccgccgcgtgcga
atctggtggcaccttcgcgcctgtctcgctgctttcgataagtctctagccatttaaaatttttgatgacctgctgcgacgctttttttctggcaaga
tagtcttgtaaatgcgggccaagatctgcacactggtatttcggtttttggggccgcgggcggcgacggggcccgtgcgtcccagcgcaca
tgttcggcgaggcggggcctgcgagcgcggccaccgagaatcggacgggggtagtctcaagctggccggcctgctctggtgcctggcc
tcgcgccgccgtgtatcgccccgccctgggcggcaaggctggcccggtcggcaccagttgcgtgagcggaaagatggccgcttcccgg
ccctgctgcagggagctcaaaatggaggacgcggcgctcgggagagcgggcgggtgagtcacccacacaaaggaaaagggcctttcc
gtcctcagccgtcgcttcatgtgactccacggagtaccgggcgccgtccaggcacctcgattagttctcgagcttttggagtacgtcgtcttta
ggttggggggaggggttttatgcgatggagtttccccacactgagtgggtggagactgaagttaggccagcttggcacttgatgtaattctcc
gcttggtactaatacgactcactatagggagacccaagctggctaggtaagcttggtaccgagctcggatccactagtccagtgtggtggaa
ttgcccttATAAGCTTCCGCCATGGAGCTCCGGGTGCTGCTCTGCTGGGCTTCGTTGGCCG
CAGCTTTGGAAGAGACCCTGCTGAACACAAAATTGGAAACTGCTGATCTGAAGTGG
GTGACATTCCCTCAGGTGGACGGGCAGTGGGAGGAACTGAGCGGCCTGGATGAGGA
ACAGCACAGCGTGCGCACCTACGAAGTGTGTGAAGTGCAGCGTGCCCCGGGCCAGG
CCCACTGGCTTCGCACAGGTTGGGTCCCACGGCGGGGCGCCGTCCACGTGTACGCCA
CGCTGCGCTTCACCATGCTCGAGTGCCTGTCCCTGCCTCGGGCTGGGCGCTCCTGCA
AGGAGACCTTCACCGTCTTCTACTATGAGAGCGATGCGGACACGGCCACGGCCCTC
ACGCCAGCCTGGATGGAGAACCCCTACATCAAGGTGGACACGGTGGCCGCGGAGCA
TCTCACCCGGAAGCGCCCTGGGGCCGAGGCCACCGGGAAGGTGAATGTCAAGACGC
TGCGTCTGGGACCGCTCAGCAAGGCTGGCTTACCTGGCCTTCCAGGACCAGGGTG
CCTGCATGGCCCTGCTATCCCTGCACCTCTTCTACAAAAAGTGCGCCCAGCTGACTG
TGAACCTGACTCGATTCCCGGAGACTGTGCCTCGGGAGCTGGTTGTGCCCGTGGCCG
GTAGCTGCGTGGTGGATGCCGTCCCCGCCCCTGGCCCCAGCCCCAGCCTCTACTGCC
GTGAGGATGGCCAGTGGGCCGAACAGCCGGTCACGGGCTGCAGCTGTGCTCCGGGG
TTCGAGGCAGCTGAGGGGAACACCAAGTGCCGAGCCTGTGCCCAGGGCACCTTCAA
GCCCCTGTCAGGAGAAGGGTCCTGCCAGCCATGCCCAGCCAATAGCCACTCTAACA
CCATTGGATCAGCCGTCTGCCAGTGCCGCGTCGGGTACTTCCGGGCACGCACAGACC
CCCGGGGTGCACCCTGCACCACCCCTCCTTCGGCTCCGCGGAGCGTGGTTTCCCGCC
TGAACGGCTCCTCCCTGCACCTGGAATGGAGTGCCCCCCTGGAGTCTGGTGGCCGAG
AGGACCTCACCTACGCCCTCCGCTGCCGGGAGTGCCGACCCGGAGGCTCCTGTGCGC
CCTGCGGGGGAGACCTGACTTTGACCCCGGCCCCCGGGACCTGGTGGAGCCCTGG
GTGGTGGTTCGAGGGCTACGTCCGGACTTCACCTATACCTTTGAGGTCACTGCATG
AACGGGGTATCCTCCTTAGCCACGGGGCCCGTCCCATTTGAGCCTGTCAATGTCACC
ACTGACCGAGAGGTACCTCCTGCAGTGTCTGACATCCGGGTGACGCGGTCCTCACCC
AGCAGCTTGAGCCTGGCCTGGGCTGTTCCCCGGGCACCCAGTGGGGCGTGGCTGGA
CTACGAGGTCAAATACCATGAGAAGGGCGCCGAGGGTCCCAGCAGCGTGCGGTTCC
TGAAGACGTCAGAAAACCGGGCAGAGCTGCGGGGGCTGAAGCGGGGAGCCAGCTA
CCTGGTGCAGGTACGGGCGCGCTCTGAGGCCGGCTACGGGCCCTTCGGCCAGGAAC
ATCACAGCCAGACCCAACTGGATGAGAGCGAGGGCTGGCGGGAGCAGGGATCCAAa
agggcaattctgcagatcgaaggtaagcctatccctaaccctctcctcggtctcgattctacgcgtaccggtcatcatcaccatcaccattgag
tttaaacccgctgatcagcctcgactgtgccttctagttgccagccatctgttgtttgcccctcccccgtgccttccttgaccctggaaggtgcc
actcccactgtcctttcctaataaaatgaggaaattgcatcgcattgtctgagtaggtgtcattctattctggggggtggggtggggcaggaca
gcaagggggaggattgggaagacaatagcaggcatgctggggatgcggtgggctctatggcttctgaggcggaaagaaccagctgggg
ctctagggggtatccccacgcgccctgtagcggcgcattaagcgcggcgggtgtggtggttacgcgcagcgtgaccgctacacttgccag
cgccctagcgcccgctcctttcgctttcttcccttcctttctcgccacgttcgccggctttccccgtcaagctctaaatcggggcatccctttagg
gttccgatttagtgctttacggcacctcgaccccaaaaaacttgattagggtgatggttcacgtagtgggccatcgccctgatagacggtttttc
gccctttgacgttggagtccacgttctttaatagtggactcttgttccaaactggaacaacactcaaccctatctcggtctattcttttgatttataa
gggattttggggatttcggcctattggttaaaaaatgagctgatttaacaaaaatttaacgcgaattaattctgtggaatgtgtgtcagttagggt
gtggaaagtccccaggctccccaggcaggcagaagtatgcaaagcatgcatctcaattagtcagcaaccaggtgtggaaagtccccagg
ctccccagcaggcagaagtatgcaaagcatgcatctcaattagtcagcaaccatagtcccgcccctaactccgcccatcccgcccctaactc
cgcccagttccgcccattctccgccccatggctgactaattttttttatttatgcagaggccgaggccgcctctgcctctgagctattccagaag
tagtgaggaggcttttttggaggcctaggcttttgcaaaaagctcccgggagcttgtatatccattttcggatctgatcagcacgtgttgacaatt
aatcatcggcatagtatatcggcatagtataatacgacaaggtgaggaactaaaccatggccaagcctttgtctcaagaagaatccaccctca
ttgaaagagcaacggctacaatcaacagcatccccatctctgaagactacagcgtcgccagcgcagctctctctagcgacggccgcatctt
cactggtgtcaatgtatatcattttactgggggaccttgtgcagaactcgtggtgctgggcactgctgctgctgcggcagctggcaacctgac
ttgtatcgtcgcgatcggaaatgagaacaggggcatcttgagcccctgcggacggtgtcgacaggtgcttctcgatctgcatcctgggatca
aagcgatagtgaaggacagtgatggacagccgacggcagttgggattcgtgaattgctgccctctggttatgtgtgggagggctaagcact
tcgtggccgaggagcaggactgacacgtgctacgagatttcgattccaccgccgccttctatgaaaggttgggcttcggaatcgttttccgg
gacgccggctggatgatcctccagcgcggggatctcatgctggagttcttcgcccaccccaacttgtttattgcagcttataatggttacaaat
accgtcgacctctagctagagcttggcgtaatcatggtcatagctgtttcctgtgtgaaattgttatccgctcacaattccacacaacatacgag
ccggaagcataaagtgtaaagcctggggtgcctaatgagtgagctaactcacattaattgcgttgcgctcactgcccgctttccagtcggga
aacctgtcgtgccagctgcattaatgaatcggccaacgcgcggggagaggcggtttgcgtattgggcgctcttccgcttcctcgctcactga
ctcgctgcgctcggtcgttcggctgcggcgagcggtatcagctcactcaaaggcggtaatacggttatccacagaatcaggggataacgca
ggaaagaacatgtgagcaaaaggccagcaaaaggccaggaaccgtaaaaaggccgcgttgctggcgtttttccataggctccgcccccc
tgacgagcatcacaaaaatcgacgctcaagtcagaggtggcgaaacccgacaggactataaagataccaggcgtttccccctggaagctc
cctcgtgcgctctcctgttccgaccctgccgcttaccggatacctgtccgcctttctcccttcgggaagcgtggcgctttctcaatgctcacgct
gtaggtatctcagttcggtgtaggtcgttcgctccaagctgggctgtgtgcacgaaccccccgttcagcccgaccgctgcgccttatccggta
actatcgtcttgagtccaacccggtaagacacgacttatcgccactggcagcagccactggtaacaggattagcagagcgaggtatgtagg
cggtgctacagagttcttgaagtggtggcctaactacggctacactagaaggacagtatttggtatctgcgctctgctgaagccagttaccttc
ggaaaaagagttggtagctcttgatccggcaaacaaaccaccgctggtagcggtggtttttttgtttgcaagcagcagattacgcgcagaaa
aaaaggatctcaagaagatcctttgatcttttctacggggtctgacgctcagtggaacgaaaactcacgttaagggattttggtcatgagattat
caaaaaggatcttcacctagatccttttaaattaaaaatgaagttttaaatcaatctaaagtatatatgagtaaacttggtctgacagttaccaatg
cttaatcagtgaggcacctatctcagcgatctgtctatttcgttcatccatagttgcctgactccccgtcgtgtagataactacgatacgggagg
gcttaccatctggccccagtgctgcaatgataccgcgagacccacgctcaccggctccagatttatcagcaataaaccagccagccggaa
gggccgagcgcagaagtggtcctgcaactttatccgcctccatccagtctattaattgttgccgggaagctagagtaagtagttcgccagtta
atagtttgcgcaacgttgttgccattgctacaggcatcgtggtgtcacgctcgtcgtttggtatggcttcattcagctccggttcccaacgatcaa
ggcgagttacatgatcccccatgttgtgcaaaaaagcggttagctccttcggtcctccgatcgttgtcagaagtaagttggccgcagtgttatc
actcatggttatggcagcactgcataattctcttactgtcatgccatccgtaagatgcttttctgtgactggtgagtactcaaccaagtcattctga
gaatagtgtatgcggcgaccgagttgctcttgcccggcgtcaatacgggataataccgcgccacatagcagaactttaaaagtgctcatcatt
ggaaaacgttcttcggggcgaaaactctcaaggatcttaccgctgttgagatccagttcgatgtaacccactcgtgcacccaactgatcttca
gcatcttttactttcaccagcgtttctgggtgagcaaaaacaggaaggcaaaatgccgcaaaaaagggaataagggcgacacggaaatgtt
gaatactcatactcttcctttttc

A vector encoding a truncated human EphB4 protein having the normal leader sequence followed by the Cys-rich and two FNff domains (CF2) was prepared by deleting the globular domain. Overlap PCR was performed with oligonucleotides designed to delete G:

Fragment 1:
5′-primer - 5SpeB4 TACTAGTCCGCCATGGAGCTCCGGGTG
CTGCT
3′-primer - 3RevB4 CAGCTGagtttccaattttgtgttc
Fragment 2:
5overB4 - gaacacaaaattggaaactCAGCTGACTGTGAACCTGAC
3NotB4GCF2 - GCGGCCGCCCTGCTCCCGCCAGCCCTCGCT

(adds NotI site after the C-terminal B4EC FL sequence after 2nd fibronectin repeat to allow in-frame fusion to V5 and His-tag in pEF6). TA clone into pEF6, then cut with NotI, gel-purify and self ligate.

Sequence of the cloned fragment (SpeI-NotI fragment):

tactagtccgccATGGAGCTCCGGGTGCTGCTCTGCTGGGCTTCGTGGCCGCAGCTT
TGGAAGAGACCCTGCTGAACACAAAATTGGAAACTCAGCTGACTGTGAACCTGACT
CGATTCCCGGAGACTGTGCCTCGGGAGCTGGTTGTGCCCGTGGCCGGTAGCTGCGTG
GTGGATGCCGTCCCCGCCCCTGGCCCCAGCCCCAGCCTCTACTGCCGTGAGGATGGC
CAGTGGGCCGAACAGCCGGTCACGGGCTGCAGCTGTGCTCCGGGGTTCGAGGCAGC
TGAGGGGAACACCAAGTGCCGAGCCTGTGCCCAGGGCACCTTCAAGCCCCTGTCAG
GAGAAGGGTCCTGCCAGCCATGCCCAGCCAATAGCCACTCTAACACCATTGGATCA
GCCGTCTGCCAGTGCCGCGTCGGGTACTTCCGGGCACGCACAGACCCCCGGGGTGC
ACCCTGCACCACCCCTCCTTCGGCTCCGCGGAGCGTGGTTTCCCGCCTGAACGGCTC
CTCCCTGCACCTGGAATGGAGTGCCCCCCTGGAGTCTGGTGGCCGAGAGGACCTCAC
CTACGCCCTCCGCTGCCGGGAGTGCCGACCCGGAGGCTCCTGTGCGCCCTGCGGGG
GAGACCTGACTTTTGACCCCGGCCCCCGGGACCTGGTGGAGCCCTGGGTGGTGGTC
GAGGGCTACGTCCGGACTTCACCTATACCTTGAGGTCACTGCATTGAACGGGGTAT
CCTCCTTAGCCACGGGGCCCGTCCCATTTGAGCCTGTCAATGTCACCACTGACCGAG
AGGTACCTCCTGCAGTGTCTGACATCCGGGTGACGCGGTCCTCACCCAGCAGCTTGA
GCCTGGCCTGGGCTGTTCCCCGGGCACCCAGTGGGGCGTGGCTGGACTACGAGGTC
AAATACCATGAGAAGGGCGCCGAGGGTCCCAGCAGCGTGCGGTTCCTGAAGACGTC
AGAAAACCGGGCAGAGCTGCGGGGGCTGAAGCGGGGAGCCAGCTACCTGGTGCAG
GTACGGGCGCGCTCTGAGGCCGGCTACGGGCCCTTCGGCCAGGAACATCACAGCCA
GACCCAACTGGATGAGAGCGAGGGCTGGCGGGAGCAGGgcggccgc

CF2, precursor:

MELRVLLCWASLAAALEETLLNTKIETQLTVNLTRFPETVPRELVVPVAGSCVV
DAVPAPGPSPSLYCREDGQWAEQPVTGCSCAPGFEAAEGNTKCRACAQGTFKPLSGEG
SCQPCPANSHSNTIGSAVCQCRVGYFRARTDPRGAPCTTPPSAPRSVVSRLNGSSLHLEW
SAPLESGGREDLTYALRCRECRPGGSCAPCGGDLTFDPGPRDLVEPWVVVRGLRPDFTY
TFEVTALNGVSSLATGPVPFEPVNVTTDREVPPAVSDIRVTRSSPSSLSLAWAVPRAPSG
AWLDYEVKYHEKGAEGPSSVRELKTSENRAELRGLKRGASYLVQVRARSEAGYGPFGQ
EHHSQTQLDESEGWREQGGRSSLEGPRFEGKPIPNPLLGLDSTRTGHHHHHH

Plasmid DNA sequence:

AATTATTATTGAAGCATTTATCAGGGTTATTGTCTCATGAGCGGATACATATTT
GAATGTATTTAGAAAAATAAACAAATAGGGGTTCCGCGCACATTTCCCCGAAAAGT
GCCACCTGACGTCGACGGATCGGGAGATCTCCCGATCCCCTATGGTCGACTCTCAGT
ACAATCTGCTCTGATGCCGCATAGTTAAGCCAGTATCTGCTCCCTGCTTGTGTGTTGG
AGGTCGCTGAGTAGTGCGCGAGCAAAATTTAAGCTACAACAAGGCAAGGCTTGACC
GACAATTGCATGAAGAATCTGCTTAGGGTTAGGCGTTTTGCGCTGCTTCGCGATGTA
CGGGCCAGATATACGCGTTGACATTGATTATTGACTAGGCTTTTGCAAAAAGCTTTG
CAAAGATGGATAAAGTTTTAAACAGAGAGGAATCTTTGCAGCTAATGGACCTTCTA
GGTCTTGAAAGGAGTGCCTCGTGAGGCTCCGGTGCCCGTCAGTGGGCAGAGCGCAC
ATCGCCCACAGTCCCCGAGAAGTTGGGGGGAGGGGTCGGCAATTGAACCGGTGCCT
AGAGAAGGTGGCGCGGGGTAAACTGGGAAAGTGATGTCGTGTACTGGCTCCGCCTT
TTTCCCGAGGGTGGGGGAGAACCGTATATAAGTGCAGTAGTCGCGTGAACGTTCTT
TTTCGCAACGGGTTTGCCGCCAGAACACAGGTAAGTGCCGTGTGTGGTTCCCGCGGG
CCTGGCCTCTTTACGGGTTATGGCCCTTGCGTGCCTTGAATTACTTCCACCTGGCTGC
AGTACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCCTGGGCGCT
GGGGCCGCCGCGTGCGAATCTGGTGGCACCTTCGCGCCTGTCTCGCTGCTTTCGATA
AGTCTCTAGCCATTTAAAATTTTTGATGACCTGCTGCGACGCTTTTTTTCTGGCAAGA
TAGTCTTGTAAATGCGGGCCAAGATCTGCACACTGGTATTTCGGTTTTTGGGGCCGC
GGGCGGCGACGGGGCCCGTGCGTCCCAGCGCACATGTTCGGCGAGGCGGGGCCTGC
GAGCGCGGCCACCGAGAATCGGACGGGGTAGTCTCAAGCTGGCCGGCCTGCTCTG
GTGCCTGGCCTCGCGCCGCCGTGTATCGCCCCGCCCTGGGCGGCAAGGCTGGCCCGG
TCGGCACCAGTTGCGTGAGCGGAAAGATGGCCGCTTCCCGGCCCTGCTGCAGGGAG
CTCAAAATGGAGGACGCGGCGCTCGGGAGAGCGGGCGGGTGAGTCACCCACACAA
AGGAAAAGGGCCTTTCCGTCCTCAGCCGTCGCTTCATGTGACTGGACGGAGTACCGG
GCGCCGTCCAGGCACCTCGATTAGTTCTCGAGCTTTTGGAGTACGTCGTCTTTAGGTT
GGGGGGAGGGGTTTTATGCGATGGAGTTTCCCCACACTGAGTGGGTGGAGACTGAA
GTTAGGCCAGCTTGGCACTTGATGTAATTCTCCTTGGAATTTGCCCTTTTTGAGTTTG
GATCTTGGTTCATTCTCAAGCCTCAGACAGTGGTTCAAAGTTTTTTTCTTCCATTTCA
GGTGTCGTGAGGAATTAGCTTGGTACTAATACGACTCACTATAGGGAGACCCAAGCT
GGCTAGGTAAGCTTGGTACCGAGCTCGGATCCACTAGTCCAGTGTGGTGGAATTGCC
CTTtactagtccgccATGGAGCTCCGGGTGCTGCTCTGCTGGGCTTCGTTGGCCGCAGCTTT
GGAAGAGACCCTGCTGAACACAAAATTGGAAACTCAGCTGACTGTGAACCTGACTC
GATTCCCGGAGACTGTGCCTCGGGAGCTGGTTGTGCCCGTGGCCGGTAGCTGCGTGG
TGGATGCCGTCCCCGCCCCTGGCCCCAGCCCCAGCCTCTACTGCCGTGAGGATGGCC
AGTGGGCCGAACAGCCGGTCACGGGCTGCAGCTGTGCTCCGGGGTTCGAGGCAGCT
GAGGGGAACACCAAGTGCCGAGCCTGTGCCCAGGGCACCTTCAAGCCCCTGTCAGG
AGAAGGGTCCTGCCAGCCATGCCAGCCAATAGCCACTCTAACACCATTGGATCAG
CCGTCTGCCAGTGCCGCGTCGGGTACTTCCGGGCACGCACAGCCCCCGGGGTGCA
CCCTGCACCACCCCTCCTTCGGCTCCGCGGAGCGTGGTTTCCCGCCTGAACGGCTCC
TCCCTGCACCTGGAATGGAGTGCCCCCCTGGAGTCTGGTGGCCGAGAGGACCTCACC
TACGCCCTCCGCTGCCGGGAGTGTCGACCCGGAGGCTCCTGTGCGCCCTGCGGGGG
AGACCTGACTTTTGACCCCGGCCCCCGGGACCTGGTGGAGCCCTGGGTGGTGGTTCG
AGGGCTACGTCCTGACTTCACCTATACCTTTGAGGTCACTGCATTGAACGGGGTATC
CTCCTTAGCCACGGGGCCCGTCCCATTTGAGCCTGTCAATGTCACCACTGACCGAGA
GGTACCTCCTGCAGTGTCTGACATCCGGGTGACGCGGTCCTCACCCAGCAGCTTGAG
CCTGGCCTGGGCTGTTCCCCGGGCACCCAGTGGGGCTGTGCTGGACTACGAGGTCAA
ATACCATGCGAAGGGCGCCGAGGGTCCCAGCAGCGTGCGGTTCCTGAAGACGTCAG
AAAACCGGGCAGAGCTGCGGGGGCTGAAGCGGGGAGCCAGCTACCTGGTGCAGGT
ACGGGCGCGCTCTGAGGCCGGCTACGGGCCCTTCGGCCAGGAACATCACAGCCAGA
CCCAACTGGATGAGAGCGAGGGCTGGCGGGAGCAGGgcggccgcTCGAGTCTAGAGGG
CCCGCGGTTCGAAGGTAAGCCTATCCCTAACCCTCTCCTCGGTCTCGATTCTACGCG
TACCGGTCATCATCACCATCACCATTGAGTTTAAACCCGCTGATCAGCCTCGACTGT
GCCTTCTAGTTGCCAGCCATCTGTTGTTTGCCCCTCCCCCGTGCCTTCCTTGACCCTG
GAAGGTGCCACTCCCACTGTCCTTTCCTAATAAAATGAGGAAATTGCATCGCATTGT
CTGAGTAGGTGTCATTCTATTCTGGGGGGTGGGGTGGGGCAGGACAGCAAGGGGGA
GGATTGGGAAGACAATAGCAGGCATGCTGGGGATGCGGTGGGCTCTATGGCTTCTG
AGGCGGAAAGAACCAGCTGGGGCTCTAGGGGGTATCCCCACGCGCCCTGTAGCGGC
GCATTAAGCGCGGCGGGTGTGGTGGTTACGCGCAGCGTGACCGCTACACTTGCCAG
CGCCCTAGCGCCCGCTCCTTTCGCTTTCTTCCCTTCCTTTCTCGCCACGTTCGCCGGCT
TTCCCCGTCAAGCTCTAAATCGGGGCATCCCTTTAGGGTTCCGATTTAGTGCTTTACG
GCACCTCGACCCCAAAAAACTTGATTAGGGTGATGGTTCACGTAGTGGGCCATCGCC
CTGATAGACGGTTTTTCGCCCTTTGACGTTGGAGTCCACGTTCTTTCCTAGTGGACTC
TTGTTCCAAACTGGAACAACACTCAACCCTATCTCGGTCTATTCTTTTGATTTATAAG
GGATTTTGGGGATTTCGGCCTATTGGTTAAAAAATGAGCTGATTTAACAAAAATTTA
ACGCGAATTAATTCTGTGGAATGTGTGTCAGTTAGGGTGTGGAAAGTCCCCAGGCTC
CCCAGGCAGGCAGAAGTATGCAAAGCATGCATCTCAATTAGTCAGCAACCAGGTGT
GGAAGTCCCCAGGCTCCCCAGCAGGCAGAAGTATGCAAAGCATGCATCTCAATTA
GTCAGCAACCATAGTCCCGCCCCTAACTCCGCCCATCCCGCCCCTAACTCCGCCCAG
TTCCGCCCATTCTCCGCCCCATGGCTGACTAATTTTTTTTATTTATGCAGAGGCCGAG
GCCGCCTCTGCCTCTGAGCTATTCCAGAAGTAGTGAGGAGGCTTTTTTGGAGGCCTA
GGCTTTTGCAAAAAGCTCCCGGGAGCTTGTATATCCATTTTCGGATCTGATCAGCAC
GTGTTGACAATTAATCATCGGCATAGTATATCGGCATAGTATAATACGACAAGGTGA
GGAACTAAACCATGGCCAAGCCTTTGTCTCAAGAAGAATCCACCCTCATTGAAAGA
GCAACGGCTACAATCAACAGCATCCCCATCTCTGAAGACTACAGCGTCGCCAGCGC
AGCTCTCTCTAGCGACGGCCGCATCTTCACTGGTGTCAATGTATATCATTTTACTGGG
GGACCTTGTGCAGAACTCGTGGTGCTGGGCACTGCTGCTGCTGCGGCAGCTGGCAAC
CTGACTTGTATCGTCGCGATCGGAAATGAGAACAGGGGCATCTTGAGCCCCTGCGG
ACGGTGTCGACAGGTGCTTCTCGATCTGCATCCTGGGATCAAAGCGATAGTGAAGG
ACAGTGATGGACAGCCGACGGCAGTTGGGATTCGTGAATTGCTGCCCTCTGGTTATG
TGTGGGAGGGCTAAGCACTTCGTGGCCGAGGAGCAGGACTGACACGTGCTACGAGA
TTTCGATTCCACCGCCGCCTTCTATGAAAGGTTGGGCTTCGGAATCGTTTTCCGGGAC
GCCGGCTGGATGATCCTCCAGCGCGGGGATCTCATGCTGGAGTTCTTCGCCCACCCC
AACTTGTTTATTGCAGCTTATAATGGTTACAAATAAAGCAATAGCATCACAAATTTC
ACAAATAAAGCATTTTTTTCACTGCATTCTAGTTGTGGTTTGTCCAAACTCATCAATG
TATCTTATCATGTCTGTATACCGTCGACCTCTAGCTAGAGCTTGGCGTAATCATGGTC
ATAGCTGTTTCCTGTGTGAAATTGTTATCCGCTCACAATTCCACACAACATACGAGC
CGGAAGCATAAAGTGTAAAGCCTGGGGTGCCTAATGAGTGAGCTAACTCACATTAA
TTGCGTTGCGCTCACTGCCCGCTTTCCAGTCGGGAAACCTGTCGTGCCAGCTGCATT
AATGAATCGGCCAACGCGCGGGGAGAGGCGGTTTGCGTATTGGGCGCTCTTCCGCTT
CCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTTGCTGG
CGTTTTTCCATAGGCTCCGCCCCCCTGACGAGCATCACAAAAATCGACGCTCAAGTC
AGAGGTGGCGAAACCCGACAGGACTATAAAGATACCAGGCGTTTCCCCCTGGAAGC
TCCCTCGTGCGCTCTCCTGTTCCGACCCTGCCGCTTACCGGATACCTGTCCGCCTTTC
TCCCTTCGGGAAGCGTGGCGCTTTCTCAATGCTCACGCTGTAGGTATCTCAGTTCGGT
GTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATC
GCCACTGGCAGCAGCCACTGGTAACAGGATTAGCAGAGCGCGGTATGTAGGCGGTG
CTACAGAGTTCTTGAAGTGGTGGCCTAACTACGGCTACACTAGAAGGACAGTATTTG
GTATCTGCGCTCTGCTGAAGCCAGTTACCTTCGGAAAAAGAGTTGGTAGCTCTTGAT
CCGGCAAACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGGTCTGACG
CTCAGTGGAACGAAAACTCACGTTAAGGGATTTTGGTCATGAGATTATCAAAAAGG
ATCTTCACCTAGATCCTTTTAAATTAAAAATGAAGTTTTAAATCAATCTAAAGTATAT
ATGAGTAAACTTGGTCTGACAGTTACCAATGCTTAATCAGTGAGGCACCTATCTCAG
CGATCTGTCTATTTCGTTCATCCATAGTTGCCTGACTCCCCGTCGTGTAGATAACTAC
GATACGGGAGGGCTTACCATCTGGCCCCAGTGCTGCAATGATACCGCGAGACCCAC
GCTCACCGGCTCCAGATTTATCAGCAATAAACCAGCCAGCCGGAAGGGCCGAGCGC
AGAAGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCTACA
GGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGAT
CCAGTTCGATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGGGAATA
AGGGCGACACGGAAATGTTGAATACTCATACTCTTCCTTTTTC

A vector encoding a preferred GCF2 truncated protein, lacking any c-terminal tags, such as a hexahistidine tag was derived from pEF6-B4ECv3-V5-His by re-amplifying the 3′ (C-terminal) part of B4ECv3 to eliminate V5 and His tags ands subcloning back into pEF6-B4ECv3-V5-His.

PCR primers used:
IntB4-3: CATTGGATCAGCCGTCTGCC
and
B4ECv3FIN
(tgtttaaacTTACTGCTCCCGCCAGCCCTCGCTCTCATCCAGTT).

The fragment with the correct N-terminal part of B4ECv3 was cut out from pEF6-B4ECv3-V5-His and subcloned into Kpn I-cut pEF6-Int3-B4ECv3FIN intermediate construct.

Sequence of the whole HindIII-PmeI fragment is:

AAGCTTCCGCCATGGAGCTCCGGGTGCTGCTCTGCTGGGCTTCGTTGGCCGCA
GCTTTGGAAGAGACCCTGCTGAACACAAAATTGGAAACTGCTGATCTGAAGTGGGT
GACATTCCCTCAGGTGGACGGGCAGTGGGAGGAACTGAGCGGCCTGGATGAGGAAC
AGCACAGCGTGCGCACCTACGAAGTGTGTGAAGTGCAGCGTGCCCCGGGCCAGGCC
CACTGGCTTCGCACAGGTTGGTCCCACGGCGGGGCGCCGTCCACGTGTACGCCAC
GCTGCGCTTCACCATGCTCGAGTGCCTGTCCCTGCCTCGGGCTGGGCGCTCCTGCAA
GGAGACCTTCACCGTCTTCTACTATGAGAGCGATGCGGACACGGCCACGGCCCTCAC
GCCAGCCTGGATGGAGAACCCTACATCAAGGTGGACACGGTGGCCGCGGAGCATC
TCACCCGGAAGCGCCCTGGGGCCGAGGCCACCGGGAAGGTGAATGTCAAGACGCTG
CGTCTGGACCGCTCAGCAAGGCTGGCTTCTACCTGGCCTTCCAGGACCAGGGTGCC
TGCATGGCCCTGCTATCCCTGCACCTCTTCTACAAAAAGTGCGCCCAGCTGACTGTG
AACTGACTCGATTCCCGGAGACTGTGCCTCGGGAGCTGGTTGTGCCCGTGGCCGGT
AGCTGCGTGGTGGATGCCGTCCCCGCCCCTGGCCCCAGCCCCAGCCTCTACTGCCGT
GAGGATGGCCAGTGGGCCGAACAGCCGGTCACGGGCTGCAGCTGTGCTCCGGGGTT
CGAGGCAGCTGAGGGGAACACCAAGTGCCGAGCCTGTGCCCAGGGCACCTTCAAGC
CCCTGTCAGGAGAAGGGTCCTGCCAGCCATGCCCAGCCAATAGCCACTCTAACACC
ATTGGATCAGCCGTCTGCCAGTGCCGCGTCGGGTACTTCCGGGCACGCACAGACCCC
CGGGGTGCACCCTGCACCACCCCTCCTTCGGCTCCGCGGAGCGTGGTTCCCGCCTG
AACGGCTCCTCCCTGCACCTGGAATGGAGTGCCCCCCTGGAGTCTGGTGGCCGAGA
GGACCTCACCTACGCCCTCCGCTGCCGGGAGTGCCGACCCGGAGGCTCCTGTGCGCC
CTGCGGGGGAGACCTGACTTTTGACCCCGGCCCCCGGGACCTGGTGGAGCCCTGGG
TGGTGGTTCGAGGGCTACGTCCGGACTTCACCTATACCTTTGAGGTCACTGCATTGA
ACGGGGTATCCTCCTTAGCCACGGGGCCCGTCCCATTTGAGCCTGTCAATGTCACCA
CTGACCGAGAGGTACCTCCTGCAGTGTCTGACATCCGGGTGACGCGGTCCTCACCCA
GCAGCTTGAGCCTGGCCTGGGCTGTTCCCCGGGCACCCAGTGGGGCGTGGCTGGACT
ACGAGGTCAAATACCATGAGAAGGGCGCCGAGGGTCCCAGCAGCGTGCGGTTCCTG
AAGACGTCAGAAAACCGGGCAGAGCTGCGGGGGCTGAAGCGGGGAGCCAGCTACC
TGGTGCAGGTACGGGCGCGCTCTGAGGCCGGCTACGGGCCCTTCGGCCAGGAACAT
CACAGCCAGACCCAACTGGATGAGAGCGAGGGCTGGCGGGAGCAGTAAgtttaaac

The precursor sequence of the preferred GCF2 protein (also referred to herein as GCF2F) is:

MELRVLLCWASLAAALEETLLNTKLETADLKWVTFPQVDGQWEELSGLDEEQHSVRT
YEVCEVQRAPGQAHWLRTGWVPRRGAVHVYATLRFTMLECLSLPRAGRSCKETFTVF
YYESDADTATALTPAWMENPYIKVDTVAAEHLTRKRPGAEATGKVNVKTLRLGPLSKA
GFYLAFQDQGACMALLSLHLFYKKCAQLTVNLTRFPETVPRELVVPVAGSCVVDAVPA
PGPSPSLYCREDGQWAEQPVTGCSCAPGFEAAEGNTKCRACAQGTFKYLSGEGSCQPCP
ANSHSNTIGSAVCQCRVGYFRARTDPRGAPCTTPPSAPRSVVSRLNGSSLHLEWSAPLES
GGREDLTYALRCRECRPGGSCAPCGGDLTFDPGPRDLVEPWVVVRGLRPDFTYTFEVT
ALNGVSSLATGPVPFEPVNVFFDREVPPAVSDIRVTRSSPSSLSLAWAVPRAPSGAWLDY
EVKYHEKGAEGPSSVRFLKTSENRAELRGLKRGASYLVQVRARSEAGYGPFGQEHHSQ
TQLDESEGWREQ

The processed sequence is:

LEETLLNTKLETADLKWVTFPQVDGQWEELSGLDEEQHSVRTYEVCEVQRAPG
QAHWLRTGWVPRRGAVHVYATLRFTMLECLSLPRAGRSCKETFTVFYYESDADTATAL
TPAWMENPYIKVDTVAAEHLTRKRPGAEATGKVNVKTLRLGPLSKAGFYLAFQDQGA
CMALLSLHLFYKKCAQLTVNLTRFPETVPRELVVPVAGSCVVDAVPAPGPSPSLYCRED
GQWAEQPVTGCSCAPGFEAAEGNTKCRACAQGTFKPLSGEGSCQPCPANSHSNTIGSAV
CQCRVGYFRARTDPRGAPCTTPPSAPRSVVSRLNGSSLHLEWSAPLESGGREDLTYALR
CRECRPGGSCAPCGGDLTFDPGPRDLVEPWVVVRGLRPDFTYTFEVTALNGVSSLATGP
VPFEPVNVTTDREVPPAVSDIRVTRSSPSSLSLAWAVPRAPSGAWLDYEVKYHEKGAEG
PSSVRFLKTSENRAELRGLKRGASYLVQVRARSEAGYGPFGQEHHSQTQLDESEGWREQ

2) Mammalian Cell Culture and Transfections

HEK293T (human embryonic kidney line) cells were maintained in DMEM with 10% dialyzed fetal calf serum and 1% penicillin/streptomycin/neomycin antibiotics. Cells were maintained at 37° C. in a humidified atmosphere of 5% CO₂/95% air. Transfections were performed using Lipofectamine 2000 reagent (Invitrogen) according to the manufacturer's protocol. One day before transfections, 293T cells were seeded at a high density to reach 80% confluence at the time of transfection. Plasmid DNA and Lipofectamine reagent at 1:3 ratio were diluted in Opti-MEM I reduced serum medium (Invitrogen) for 5 min and mixed together to form DNA:Lipofectamine complex. For each 10 cm culture dish, 10 μg of plasmid DNA was used. After 20 min, above complex was added directly to cells in culture medium. After 16 hours of transfection, medium was aspirated, washed once with serum free DMEM and replaced with serum free DMEM. Secreted proteins were harvested after 48 hours by collecting conditional medium. Conditional medium was clarified by centrifugation at 10,000 g for 20 min, filtered through 0.2 μm filter and used for purification.

3) Generating Stable Cell Lines

To create stable cell lines producing EphB4 ECv3 and EphB4ECnt HEK293 or HEK293T cells were transfected with either pEF6-B4ECv3 or pEF6-B4EC-NT plasmid constructs as described above and selected using antibiotic Blasticidin. After 24 hours of transfection, cells were seeded at low density. Next day, cells were treated with 10 μg/ml of Blasticidin. After two weeks of drug selection, surviving cells were pooled and selected further for single cell clone expansion. After establishing stable cells, they were maintained at 4 μg/ml Blasticidin. Conditioned media were tested to confirm expression and secretion of the respective recombinant proteins. Specificity of expression was confirmed by Western blot with anti-B4 monoclonal or polyclonal antibodies and B2EC-AP reagent binding and competition assays.

4) Protein Purification

HEK293 cells were transiently transfected with a plasmid encoding secreted form of EphB4ectodomain (B4ECv3). Conditional media was harvested and supplemented with 10 mM imidazole, 0.3 M NaCl and centrifuged at 20,000 g for 30 min to remove cell debris and insoluble particles. 80 ml of obtained supernatant were applied onto the pre-equilibrated column with 1 ml of Ni-NTA-agarose (Qiagen) at the flow rate of 10 ml/h. After washing the column with 10 ml of 50 mM Tris-HCl, 0.3 M NaCl and 10 mM imidazole, pH 8, remaining proteins were eluted with 3 ml of 0.25 M imidazole. Eluted proteins were dialyzed against 20 mM Tris-HCl, 0.15 M NaCl, pH 8 overnight. Purity and identity of B4ECv3 was verified by PAGE/Coomassie G-250 and Western blot with anti-Eph.B4 antibody. Finally, the concentration of B4ECv3 was measured, and the protein was aliquoted and stored at −70° C.

B4EC-FC protein and B2EC-FC protein were similarly purified.

Generation of an EphB4-Serum Albumin fusion protein:

Human serum albumin fragment in XbaI-NotI form was PCRed out for creating fusion with GCF2 to extend GCF2 half-life and TA-cloned in pEF6. In the next step, the resulting vector was cut with Xba I (partial digestion) and the HSA fragment (1.8 kb) was cloned into Xba I site of pEF6-GCF2-Xba to create fusion expression vector. The resulting vector had a point mutation C to T leading to Thr to Ile substitution in position 4 of the mature protein. It was called pEF6-GCF2-HSAmut. In the next cloning step, the mutation was removed by substituting wild type KpnI fragment from pEF6-GCF2-IF (containing piece of the vector and N-terminal part of GCF2) for the mutated one, this final vector was called pEF6-GCF2. Note that N-terminal junction site has changed as a result because the new Kpn fragment came from a different GCF2 expression vector. The DNA sequence of pEF6-GCF2 was confirmed.

The amino acid of the HSA-EphB4 precursor protein is as follows:

MELRVLLCWASLAAALEETLLNTKLETADLKWVTFPQVDGQWEELSGLDEEQHSVRT
YEVCDVQRAPGQAHWLRTGWVPRRGAVHVYATLRFTMILECLSLPRAGRSCKETFTVF
YYESDADTATALTPAWMIENPYLKVDTVAAEHLTRKRPGAEATGKVNVKTLRLGPLSKA
GFYLAFQDQGACMALLSLHLFYKKCAQLTVNLTRFPETVPRELVVPVAGSCVVDAVPA
PGPSPSLYCREDGQWAEQPVTGCSCAPGFEAAEGNTKCRACAQGTFKPLSGEGSCQPCP
ANSHSNTIGSAVCQCRVGYFRARTDPRGAPCTTPPSAPRSVVSRLNGSSLHLEWSAPLES
GGREDLTYALRCRECRPGGSCAPCGGDLTFDPGPRDLVEPWVVVRGLRPDFTYTFEVT
ALNGVSSLATGPVPFEPVNVTTDREVPPAVSDTRVTRSSPSSLSLAWAVPRAPSGAVLDY
EVKYHEKGAEGPSSVRFLKTSENRAELRGLKRGASYLVQVRARSEAGYGPFGQEHHSQ
TQLDESEGWREQSRDAHKSEVAHRFKDLGEENFKALVLIAFAQYLQQCPFEDHVKLVN
EVTEFAKTCVADESAENCDKSLHTLFGDKLCTVATLRETYGEMADCCAKQEPERNECF
LQHKDDNPNLPRLVRPEVDVMCTAFHDNEETFLKKYLYEIARRHPYFYAPELLFFAKRY
KAAFTECCQAADKAACLLPKLDELRDEGKASSAKQRLKCASLQKFGERAFKAWAVAR
LSQRFPKAEFAEVSKLVTDLTKVHTECCHGDLLECADDRADLAKYICENQDSISSKLKE
CCEKPLLEKSHCIAEVENDEMPADLPSLAADFVESKDVCKNYAEAKDVFLGMFLYEYA
RRHPDYSVVLLLRLAKTYETTLEKCCAAADPHECYAKVFDEFKPLVEEPQNLIKQNCEL
FKQLGEYKFQNALLVRYTKKVPQVSTPTLVEVSRNLGKVGSKCCKHPEAKRMPCAEDY
LSVVLNQLCVLHEKTPVSDRVTKCCTESLVNRRPCFSALEVDETYVPKEFNAETFTFHA
DICTLSEKERQIKKQTALVELVKHKPKATKEQLKAVMDDFAAFVEKCCKADDKETCFA
EEGKKLVAASQAALGL

The mature form of the HSA-EphB4 protein is as follows

LEETLLNTKLETADLKWVTFPQVDGQWEELSGLDEEQHSVRTYEVCDVQRAPGQAHW
LRTGWVPRRGAVHVYATLRFTMLECLSLPRAGRSCKETFTVFYYESDADTATALTPAW
MENPYIKVDTVAAEHLTRKRPGAEATGKVNVKTLRLGPLSKAGFYLAFQDQGACMAL
LSLHLFYKKCAQLTVNLTRFPETVPRELVVPVAGSCVVDAVPAPGPSPSLYCREDGQWA
EQPVTGCSCAPGFEAAEGNTKCRACAQGTFKPLSGEGSCQPCPANSHSNTIGSAVCQCR
VGYFRARTDPRGAPCTTPPSAPRSVVSRLNGSSLHLEWSAPLESGGREDLTYALRCREC
RPGGSCAPCGGDLTFDPGPRDLVEPWVVVRGLRPDFTYTFEVTALNGVSSLATGPVPFE
PVNVTTDREVPPAVSDTRVTRSSPSSLSLAWAVPRAPSGAVLDYEVKYHEKGAEGPSSV
RFLKTSENRAELRGLKRGASYLVQVRARSEAGYGPFGQEHHSQTQLDESEGWREQSRD
AHKSEVAHRFKDLGEENFKALVLIAFAQYLQQCPFEDHVKLVNEVTEFAKTCVADESA
ENCDKSLHTLFGDKLCTVATLRETYGEMADCCAKQEPERNECFLQHKDDNPNLPRLVR
PEVDVMCTAFHDNEETFLKKYLYEIARRHPYFYAPELLFFAKRYKAAFTECCQAADKA
ACLLPKLDELRDEGKASSAKQRLKCASLQKEGERAFKAWAVARLSQRFPKAEFAEVSK
LVTDLTKVHTECCHGDLLECADDRADLAKYICENQDSISSKLKECCEKPLLEKSHCIAEV
ENDEMPADLPSLAADFVESKDVCKNYAEAKDVFLGMFLYEYARRHPDYSVVLLLRLA
KTYETTLEKCCAAADPHECYAKVFDEFKPLVEEPQNLLKQNCELFKQLGEYKFQNALLV
RYTKKVPQVSTPTLVEVSRNLGKVGSKCCKHPEAKRMPCAEDYLSVVLNQLCVLHEKT
PVSDRVTKCCTESLVNRRPCFSALEVDETYVPKEFNAETFTFHADICTLSEKERQIKKQT
ALVELVKHKPKATKEQLKAVMDDFAAFVEKCCKADDKETCFAEEGKKLVAASQAALGL

The DNA sequence of the pEF6-GCF2 is as follows:

aatattattgaagcatttatcagggttattgtctcatgagcggatacatatttgaatgtatttagaaaaataaacaaataggggttccgcgcacatt
tccccgaaaagtgccacctgacgtcgacggatcgggagatctcccgatcccctatggtcgactctcagtacaatctgctctgatgccgcata
gttaagccagtatctgctccctgcttgtgtgttggaggtcgctgagtagtgcgcgagcaaaatttaagctacaacaaggcaaggcttgaccga
caattgcatgaagaatctgcttagggttaggcgttttgcgctgcttcgcgatgtacgggccagatatacgcgttgacattgattattgactaggc
ttttgcaaaaagctttgcaaagatggataaagttttaaacagagaggaatctttgcagctaatggaccttctaggtcttgaaaggagtgcctcgt
gaggctccggtgcccgtcagtgggcagagcgcacatcgcccacagtccccgagaagttggggggaggggtcggcaattgaaccggtg
cctagagaaggtggcgcggggtaaactgggaaagtgatgtcgtgtactggctccgcctttttcccgagggtgggggagaaccgtatataag
tgcagtagtcgccgtgaacgttctttttcgcaacgggtttgccgccagaacacaggtaagtgccgtgtgtggttcccgcgggcctggcctctt
tacgggttatggcccttgcgtgccttgaattacttccacctggctgcagtacgtgattcttgatcccgagcttcgggttggaagtgggtgggag
agttcgaggccttgcgcttaaggagccccttcgcctcgtgcttgagttgaggcctggcctgggcgctggggccgccgcgtgcgaatctggt
ggcaccttcgcgcctgtctcgctgctttcgataagtctctagccatttaaaatttttgatgacctgctgcgacgctttttttctggcaagatagtctt
gtaaatgcgggccaagatctgcacactggtatttcggtttttggggccgcgggcggcgacggggcccgtgcgtcccagcgcacatgttcg
gcgaggcggggcctgcgagcgcggccaccgagaatcggacgggggtagtctcaagctggccggcctgctctggtgcctggcctcgcg
ccgccgtgtatcgccccgccctgggcggcaaggctggcccggtcggcaccagttgcgtgagcggaaagatggccgcttcccggccctg
ctgcagggagctcaaaatggaggacgcggcgctcgggagagcgggcgggtgagtcacccacacaaaggaaaagggcctttccgtcct
cagccgtcgcttcatgtgactccacggagtaccgggcgccgtccaggcacctcgattagttctcgagcttttggagtacgtcgtctttaggttg
gggggaggggttttatgcgatggagtttccccacactgagtgggtggagactgaagttaggccagcttggcacttgatgtaattctccttgga
gtactaatacgactcactatagggagacccaagctggctaggtaagcttggtaccgagctcggatccactagtccagtgtggtggaattgcc
cttCAAGCTTGCCGCCACCATGGAGCTCCGGGTGCTGCTCTGCTGGGCTTCGTTGGCCG
CAGCTTTGGAAGAGACCCTGCTGAACACAAAATTGGAACTGCTGATCTGAAGTGG
GTGACATTCCCTCAGGTGGACGGGCAGTGGGAGGAACTGAGCGGCCTGGATGAGGA
ACAGCACAGCGTGCGCACCTACGAAGTGTGTGACGGCAGCGTGCCCCGGGCCAGG
CCCACTGGCTTCGCACAGGTTGGGTCCCACGGCGGGGCGCCGCTCCACGTGTACGCCA
CGCTGCGCTTCACCATGCTCGAGTGCCTGTCCCTGCCTCGGGCTGGGCGCTCCTGCA
AGGAGACCTTCACCGTCTTCTACTATGAGAGCGATGCGGACACGGCCACGGCCCTC
ACGCCAGCCTGGATGGAGAACCCCTACATCAAGGTGGACACGGTGGCCGCGGAGCA
TCTCACCCGGAAGCGCCCTGGGGCCGAGGCCACCGGGAAGGTGAATGTCAAGACGC
TGCGCCTGGGACCGCTCAGCAAGGCTGGCTTCTACCTGGCCTTCCAGGACCAGGGTG
CCTGCATGGCCCTGCTATCCCTGCACCTCTTCTACAAAAAGTGCGCCCAGCTGACTG
TGAACCTGACTCGATTCCCGGAGACTGTGCCTCGGGAGCTGGTTGTGCCCGTGGCCG
GTAGCTGCGTGGTGGATGCCGTCCCCGCCCCTGGCCCCAGCCCCAGCCTCTACTGCC
GTGAGGATGGCCAGTGGGCCGAACAGCCGGTCACGGGCTGCAGCTGTGCTCCGGGG
TTCGAGGCAGCTGAGGGGAACACCAAGTGCCGAGCCTGTGCCCAGGGCACCTTCAA
GCCCCTGTCAGGAGAAGGGTCCTGCCAGCCATGCCCAGCCAATAGCCACTCTAACA
CCATTGGATCAGCCGTCTGCCAGTGCCGCGTCGGGTACTTCCGGGCACGCACAGACC
CCCGGGGTGCACCTGCACCACCCCTCCTTCGGCTCCGCGGAGCGTGGTTTCCCGCC
TGAACGGCTCCTCCCTGCACCTGGAATGGAGTGCCCCCCTGGAGTCTGGTGGCCGAG
AGGACCTCACCTACGCCCTCCGCTGCCGGGAGTGTCGACCCGGAGGCTCCTGTGCGC
CCTGCGGGGGAGACCTGACTTTTGACCCCGCCCCCGGGACCTGGTGGAGCCCTGG
GTGGTGGTTCGAGGGCTACGTCCTGACTTCACCTATACCTTTGAGGTCACTGCATTG
AACGGGGTATCCTCCTTAGCCACGGGGCCCGTCCATTTGAGCCTGTCAATGTCACC
ACTGACCGAGAGGTACCTCCTGCAGTGTCTGACATCCGGGTGACGCGGTCCTCACCC
AGCAGCTTGAGCCTGGCCTGGGCTGTTCCCCGGGCACCCAGTGGGGCTGTGCTGGAC
TACGAGGTCAAATACCATGAGAAGGGCGCCGAGGGTCCCAGCAGCGTGCGGTTCCT
GAAGACGTCAGAAAACCGGGCAGAGCTGCGGGGGCTGAGCGGGGAGCCAGCTAC
CTGGTGCAGGTACGGGCGCGCTCTGAGGCCGGCTACGGGCCCTTCGGCCAGGAACA
TCACAGCCAGACCCAACTGGATGAGAGCGAGGGCTGGCGGGAGCAGtctagaGATGCA
CACAAGAGTGAGGTTGCTCATCGGTTAAAGATTTGGGAGAAGAAAATTTCAAAGC
CTTGGTGTTGATTGCCTTTGCTCAGTATCTTCAGCAGTGTCCATTTGAAGATCATGTA
AAATTAGTGAATGAAGTAACTGAATTTGCAAAAACATGTGTAGCTGATGAGTCAGC
TGAAAATTGTGACAAATCACTTCATACCCTTTTTGGAGACAAATTATGCACAGTTGC
AACTCTTCGTGAAACCTATGGTGAAATGGCTGACTGCTGTGCAAAACAAGAACCTG
AGAGAAATGAATGCTTCTTGCAACACAAAGATGACAACCCAAACCTCCCCCGATTG
GTGAGACCAGAGGTTGATGTGATGTGCACTGCTTTTCATGACAATGAAGAGACATTT
TTGAAAAAATACTTATATGAAATTGCCAGAAGACATCCTTACTTTTATGCCCCGGAA
CTCCTTTTCTTTGCTAAAAGGTATAAAGCTGCTTTTACGAATGTTGCCAAGCTGCTG
ATAAAGCTGCCTGCCTGTTGCCAAAGCTCGATGAACTTCGGGATGAAGGGAAGGCT
TCGTCTGCCAAACAGAGACTCAAATGTGCCAGTCTCCAAAAATTTGGAGAAGAGC
TTTCAAAGCATGGGCAGTGGCTCGCCTGAGCCAGAGATTTCCCAAAGCTGAGTTTGC
AGAAGTTTCCAAGTTAGTGACAGATCTTACCAAAGTCCACACGGAATGCTGCCATGG
AGATCTGCTTGAATGTGCTGATGACGGGCGGACCTTGCCAAGTATATCTGTGAAAA
TCAGGATTCGATCTCCAGTAAACTGAAGGAATGCTGTGAAAAACTCTGTTGGAAA
AATCCCACTGCATTGCCGAAGTGGAAAATGATGAGATGCCTGCTGACTTGCCTTCAT
TAGCTGCTGATTTTGTTGAAAGTAAGGATGTTTGCAAAAACTATGCTGAGGCAAAGG
ATGTCTTCCTGGGCATGTTTTTGTATGAATATGCAAGAAGGCATCCTGATTACTCTGT
CGTGCTGCTGCTGAGACTTGCCAAGACATAATGAAACCACTCTAGAGAAGTGCTGTGC
CGCTGCAGATCCTCATGAATGCTATGCCAAAGTGTTCGATGAATTTAAACCTCTTGT
GGAAGAGCCTCAGAATTTAATCAAACAAAACTGTGAGCTTTTTAAGCAGCTTGGAG
AGTACAATTCCAGAATGCGCTATTAGTTCGTTACACCAAGAAAGTACCCCAAGTGT
CAACTCCAACTCTTGTAGAGGTCTCAAGAAACCTAGGAAAAGTGGGCAGCAAATGT
TGTAAACATCCTGAAGCAAAAAGAATGCCCTGTGCAGAAGACTATCTATCCGTGGTC
CTGAACCAGTTATGTGTGTTGCATGAGAAAACGCCAGTAAGTGACAGAGTCACAAA
ATGCTGCACAGAGTCCTTGGTGAACAGGCGACCATGCTTTTCAGCTCTGGAAGTCGA
TGAAACATACGTTCCCAAAGAGTTTAATGCTGAAACATTCACCTTCCATGCAGATAT
ATGCACACTTTCTGAGAAGGAGAGACAAATCAAGAAACAAACTGCACTTGTTGAGC
TTGTGAAACACAAGCCCAAGGCAACAAAAGAGCAACTGAAAGCTGTTATGGATGAT
TTCGCAGCTTTTGTAGAGAAGTGCTGCAAGGCTGACGATAAGGAGACCTGCTTTGCC
GAGGAGGGTAAAAAACTTGTTGCTGCAAGTCAAGCTGCCTTAGGCTTATAAtagcggccg
cttaagggcaattctgcagatatccagcacagtggcggccgctcgagtctagagggcccgcggttcgaaggtaagcctatccctaaccctc
tcctcggtctcgattctacgcgtaccggtcatcatcaccatcaccartgagtttaaacccgctgatcagcctcgactgtgccttctagttgccag
ccatctgttgtttgcccctcccccgtgccttccttgaccctggaaggtgccactcccactgtcctttcctaataaaatgaggaaattgcatcgcat
tgtctgagtaggtgtcattctattctggggggtggggtggggcaggacagcaagggggaggattgggaagacaatagcaggcatgctgg
ggatgcggtgggctctatggcttctgaggcggaaagaaccagctggggctctagggggtatccccacgcgccctgtagcggcgcattaa
gcgcggcgggtgtggtggttacgcgcagcgtgaccgctacacttgccagcgccctagcgcccgctcctttcgctttcttcccttcctttctcg
ccacgttcgccggctttccccgtcaagctctaaatcggggcatccctttagggttccgatttagtgctttacggcacctcgaccccaaaaaact
tgattagggtgatggttcacgtagtgggccatcgccctgatagacggtttttcgccctttgacgttggagtccacgttctttaatagtggactctt
gttccaaactggaacaacactcaaccctatctcggtctattcttttgatttataagggattttggggatttcggcctattggttaaaaaatgagctg
atttaacaaaaatttaacgcgaattaattctgtggaatgtgtgtcagttagggtgtggaaagtccccaggctccccaggcaggcagaagtatg
caaagcatgcatctcaattagtcagcaaccaggtgtggaaagtccccaggctccccagcaggcagaagtatgcaaagcatgcatctcaatt
agtcagcaaccatagtcccgcccctaactccgcccatcccgcccctaactccgcccagttccgcccattctccgccccatggctgactaattt
tttttatttatgcagaggccgaggccgcctctgcctctgagctattccagaagtagtgaggaggcttttttggaggcctaggcttttgcaaaaag
ctcccgggagcttgtatatccattttcggatctgatcagcacgtgttgacaattaatcatcggcatagtatatcggcatagtataatacgacaag
gtgaggaactaaaccatggccaagcctttgtctcaagaagaatccaccctcattgaaagagcaacggctacaatcaacagcatccccatctc
tgaagactacagcgtcgccagcgcagctctctctagcgacggccgcatcttcactggtgtcaatgtatatcattttactgggggaccttgtgca
gaactcgtggtgctgggcactgctgctgctgcggcagctggcaacctgacttgtatcgtcgcgatcggaaatgagaacaggggcatcttga
gcccctgcggacggtgtcgacaggtgcttctcgatctgcatcctgggatcaaagcgatagtgaaggacagtgatggacagccgacggca
gttgggattcgtgaattgctgccctctggttatgtgtgggagggctaagcacttcgtggccgaggagcaggactgacacgtgctacgagattt
cgattccaccgccgccttctatgaaaggttgggcttcggaatcgttttccgggacgccggctggatgatcctccagcgcggggatctcatgc
tggagttcttcgcccaccccaacttgtttattgcagcttataatggttacaaataaagcaatagcatcacaaatttcacaaataaagcatttttttca
ctgcattctagttgtggtttgtccaaactcatcaatgtatcttatcatgtctgtataccgtcgacctctagctagagcttggcgtaatcatggtcata
gctgtttcctgtgtgaaattgttatccgctcacaattccacacaacatacgagccggaagcataaagtgtaaagcctggggtgcctaatgagt
gagctaactcacattaattgcgttgcgctcactgcccgctttccagtcgggaaacctgtcgtgccagctgcattaatgaatcggccaacgcgc
ggggagaggcggtttgcgtattgggcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagcaaaaggccagcaaaaggccag
gaaccgtaaaaaggccgcgttgctggcgtttttccataggctccgcccccctgacgagcatcacaaaaatcgacgctcaagtcagaggtgg
cgaaacccgacaggactataaagataccaggcgtttccccctggaagctccctcgtgcgctctcctgttccgaccctgccgcttaccggata
cctgtccgcctttctcccttcgggaagcgtggcgctttctcaatgctcacgctgtaggtatctcagttcggtgtaggtcgttcgctccaagctgg
gctgtgtgcacgaaccccccgttcagcccgaccgctgcgccttatccggtaactatcgtcttgagtccaacccggtaagacacgacttatcg
ccactggcagcagccactggtaacaggattagcagagcgaggtatgtaggcggtgctacagagttcttgaagtggtggcctaactacggct
acactagaaggacagtatttggtatctgcgctctgctgaagccagttaccttcggaaaaagagttggtagctcttgatccggcaaacaaacca
ccgctggtagcggtggtttttttgtttgcaagcagcagattacgcgcagaaaaaaaggatctcaagaagatcctttgatcttttctacggggtct
gacgctcagtggaacgaaaactcacgttaagggattttggtcatgagattatcaaaaaggatcttcacctagatccttttaaattaaaaatgaag
ttttaaatcaatctaaagtatatatgagtaaacttggtctgacagttaccaatgcttaatcagtgaggcacctatctcagcgatctgtctatttcgtt
catccatagttgcctgactccccgtcgtgtagataactacgatacgggagggcttaccatctggccccagtgctgcaatgataccgcgagac
ccacgctcaccggctccagatttatcagcaataaaccagccagccggaagggccgagcgcagaagtggtcctgcaactttatccgcctcc
atccagtctattaattgttgccgggaagctagagtaagtagttcgccagttaatagtttgcgcaacgttgttgccattgctacaggcatcgtggt
gtcacgctcgtcgtttggtatggcttcattcagctccggttcccaacgatcaaggcgagttacatgatcccccatgttgtgcaaaaaagcggtt
agctccttcggtcctccgatcgttgtcagaagtaagttggccgcagtgttatcactcatggttatggcagcactgcataattctcttactgtcatg
ccatccgtaagatgcttttctgtgactggtgagtactcaaccaagtcattctgagaatagtgtatgeggcgaccgagttgctcttgcccggcgt
caatacgggataataccgcgccacatagcagaactttaaaagtgctcatcattggaaaacgttcttcggggcgaaaactctcaaggatcttac
cgctgttgagatccagttcgatgtaacccactcgtgcacccaactgatcttcagcatcttttactttcaccagcgtttctgggtgagcaaaaaca
ggaaggcaaaatgccgcaaaaaagggaataagggcgacacggaaatgttgaatactcatactcttcctttttc

A. Cell Culture and Transfections:

The human embryonic kidney cell line, 293T cells, were maintained in DMEM with 10% dialyzed fetal calf serum and 1% penicillin/streptomycin/neomycin antibiotics. Cells were maintained at 37° C. in a humidified atmosphere of 5% CO2/95% air.

Transfections of plasmids encoding EphB4 ectodomain, fragments thereof, and EphB4-HSA fusions were performed using Lipofectamine 2000 reagent (Invitrogen) according to suggested protocol. One day before transfections, 293T cells were seeded at a high density to reach 80% confluence at the time of transfection. Plasmid DNA and Lipofectamine reagent at 1:3 ratio were diluted in Opti-MEM I reduced serum medium (Invitrogen) for 5 min and mixed together to form DNA-Lipofectamine complex. For each 10 cm culture dish, 10 μg of plasmid DNA was used. After 20 min, the above complex was added directly to cells in culture medium. After 16 hours of transfection, medium was aspirated, washed once with serum free DMEM and replaced with serum free DMEM. Secreted proteins were harvested after 48 hours by collecting conditional medium. Conditional medium was clarified by centrifugation at 10,000 g for 20 min and filtered through 0.2μ filter and used for purification.

B. Chromatographic Separation of EphB4 Ectodomain and EphB4 Ectodomain-HSA Fusion Protein

The EphB4 ectodomain fused to HSA was purified as follows: 700 ml of media was harvested from transiently transfected 293 cells grown in serum free media and concentrated up to final volume of 120 ml. Membrane: (Omega, 76 mm), 50 kDa C/O. After concentration, pH of the sample was adjusted by adding 6 ml of 1M NaAc, pH 5.5. Then sample was dialyzed against starting buffer (SB): 20 mM NaAc, 20 mM NaCl, pH 5.5 for O/N. 5 ml of SP-Sepharose was equilibrated with SB and sample was loaded. Washing: 100 ml of SB. Elution by NaCl: 12 ml/fraction and increment of 20 mM. Most of the EphrinB2 binding activity eluted in the 100 mM and 120 mM fractions.

Fractions, active in EphrinB2 binding assay (See SP chromatography, fractions # 100-120 mM) were used in second step of purification on Q-column. Pulled fractions were dialyzed against starting buffer#2 (SB2): 20 mM Tris-HCl, 20 mM NaCl, pH 8 for O/N and loaded onto 2 ml of Q-Sepharose. After washing with 20 ml of SB2, absorbed protein was eluted by NaCl: 3 ml/fraction with concentrational increment of 25 mM. Obtained fractions were analyzed by PAGE and in Ephrin-B2 binding assay. The 200 mM and 225 mM fractions were found to contain the most protein and the most B2 binding activity.

Soluble EphB4 ectodomain protein was purified as follows: 300 ml of conditional medium (see: Cell culture and transfections) were concentrated up to final volume of 100 ml, using ultrafiltration membrane with 30 kDa C/O. After concentration, pH of the sample was adjusted by adding 5 ml of 1 M Na-Acetate, pH 5.5. Then sample was dialyzed against starting buffer (StB): 20 mM Na-Acetate, 20 mM NaCl, pH 5.5 for O/N. 5 ml of SP-Sepharose was equilibrated with StB and sample was loaded. After washing the column with 20 ml of StB, absorbed proteins were eluted by linear gradient of concentration of NaCl (20-250 mM and total elution volume of 20 column's volumes). Purity of the proteins was analyzed by PAGE.

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

We claim:

1. A method for identifying a tumor that is suitable for treatment with an inhibitor of EphB4 expression or function, the method comprising detecting in a tumor a cell having one or more of the following characteristics:
(a) abnormally high expression of EphB4 protein;
(b) abnormally high expression of EphB4 mRNA; and
(c) gene amplification of the EphB4 gene;
wherein a cell having one or more of characteristics (a), (b) and/or (c) is suitable for treatment with an inhibitor of EphB4 expression or function.

2. The method of claim 1, wherein the tumor is selected from the group consisting of a squamous cell carcinoma of the head and neck (HNSCC), a prostate tumor cell, a colorectal carcinoma cell, a lung tumor cell, a bladder tumor cell, and a brain tumor cell.

3. A method for evaluating gene amplification of the EphB4 gene in a test cell, comprising detecting the EphB4 gene copy number in a test cell, wherein an increase in the EphB4 gene copy number in the test cell relative to that in a control cell is indicative of gene amplification of the EphB4 gene in the test cell.

4. The method of claim 3, wherein the EphB4 gene copy number is detected by a hybridization-based assay.

5. The method of claim 4, wherein the hybridization-based assay is selected from the group consisting of Southern blot, in situ hybridization (ISH), and comparative genomic hybridization (CGH).

6. The method of claim 3, wherein the EphB4 gene copy number is detected by an amplification-based assay.

7. The method of claim 6, wherein the amplification-based assay is a quantative PCR.

8. The method of claim 3, wherein the EphB4 gene copy number is detected by using a microarray-based platform.

9. The method of claim 3, wherein the test cell is a mammalian cell.

10. The method of claim 9, wherein the test cell is a human cell.

11. The method of claim 3, wherein the test cell is a tumor cell.

12. The method of claim 11, wherein the tumor cell is selected from the group consisting of a squamous cell carcinoma of the head and neck (HNSCC), a prostate tumor cell, a breast tumor cell, a colorectal carcinoma cell, a lung tumor cell, a bladder tumor cell, and a brain tumor cell.

13. The method of claim 3, wherein the test cell is obtained from: (a) a subject suspected of having a tumor; (b) a subject that is known to have a tumor; (c) a tumor tissue; (d) a primary tumor; (e) a tissue that is suspected of harboring metastatic cells derived from the primary tumor; and (f) a lymph node or bone marrow.

14. The method of claim 3, wherein the test cell is present in a bodily fluid selected from the group consisting of blood, serum, plasma, a blood-derived fraction, lymph fluid, pleural fluid, stool, urine, and a colonic effluent.

15. The method of claim 3, wherein the control cell has an EphB4 gene copy number of two copies per cell.

16. The method of claim 3, wherein the test cell is present in a pool of test cells.

17. A method for evaluating the cancer status of a cell in a subject, comprising:
a) obtaining a test cell from a subject suspected of having or known to have a tumor;
b) detecting the EphB4 gene copy number in the test cell,
wherein an increase in the EphB4 gene copy number in the test cell relative to that in a control cell indicates that the test cell is a tumor cell.

18. A method for evaluating the prognosis of a subject, comprising:
a) obtaining a test cell from a subject suspected of having or known to have a tumor;
b) detecting an indicator of elevated EphB4 activity in the test cell,
wherein an increase in the indicator of EphB4 activity in the test cell relative to that in a control cell indicates that the subject is at increased risk for having or developing a metastatic cancer.

19. A kit for detecting gene amplification of the EphB4 gene in a test cell, comprising:
a) one or more nucleic acid capable of hybridizing to the EphB4 gene under high stringency conditions; and
b) a control nucleic acid comprising human genomic DNA having one copy of EphB4 at the normal position.

20. A method for treating a patient suffering from a cancer, comprising:
(a) identifying in the patient a tumor having a plurality of tumor cells having a gene amplification of the EphB4 gene; and
(b) administering to the patient an EphB4-selective therapeutic compound selected from the group consisting of:
(i) a nucleic acid compound that hybridizes to an EphB4 transcript under physiological conditions and decreases the expression of EphB4 in a cell; and
(ii) a polypeptide that inhibits a cellular function of EphB4.


SEQUENCE LISTING (368 sequences)

SEQ ID NO: 1 - DNA, 29 bp, Unknown
  Oligonucleotide
ggatccgccatggagctccgggtgctgct

SEQ ID NO: 2 - DNA, 36 bp, Unknown
  Oligonucleotide
tggatccctgctcccgccagccctcgctctcatcca

SEQ ID NO: 3 - DNA, 28 bp, Unknown
  Oligonucleotide
tggatccaccatggctgtgagaagggac

SEQ ID NO: 4 - DNA, 53 bp, Unknown
  Oligonucleotide
attaatggtgatggtgatgatgactacccacttcggaaccgaggatgttgttc

SEQ ID NO: 5 - DNA, 31 bp, Unknown
  Oligonucleotide
taaagcttccgccatggctgtgagaagggac

SEQ ID NO: 6 - DNA, 34 bp, Unknown
  Oligonucleotide
taggatccacttcggaaccgaggatgttgttccc

SEQ ID NO: 7 - DNA, 31 bp, Unknown
  Oligonucleotide
ataagcttccgccatggagctccgggtgctg

SEQ ID NO: 8 - DNA, 34 bp, Unknown
  Oligonucleotide
ttggatcctgctcccgccagccctcgctctcatc

SEQ ID NO: 9 - DNA, 21 bp, Unknown
  Oligonucleotide
tcagtactgcggggccggtcc

SEQ ID NO: 10 - DNA, 20 bp, Unknown
  Oligonucleotide
tcctgtcccacccggggttc

SEQ ID NO: 11 - DNA, 20 bp, Unknown
  Oligonucleotide
ccggcttggcctgggacttc

SEQ ID NO: 12 - DNA, 20 bp, Unknown
  Oligonucleotide
atgtgctggacactggccaa

SEQ ID NO: 13 - DNA, 20 bp, Unknown
  Oligonucleotide
gattttcttctggtgtcccg

SEQ ID NO: 14 - DNA, 20 bp, Unknown
  Oligonucleotide
ccagagtgactccgattcgg

SEQ ID NO: 15 - DNA, 20 bp, Unknown
  Oligonucleotide
agcaggtcctcagcagagat

SEQ ID NO: 16 - DNA, 20 bp, Unknown
  Oligonucleotide
ctggctgaccagctcgaagg

SEQ ID NO: 17 - DNA, 20 bp, Unknown
  Oligonucleotide
agccaaagccagcggctgcg

SEQ ID NO: 18 - DNA, 20 bp, Unknown
  Oligonucleotide
aaactttcttcgtatcttcc

SEQ ID NO: 19 - DNA, 20 bp, Unknown
  Oligonucleotide
cattttgatggcccgaagcc

SEQ ID NO: 20 - DNA, 20 bp, Unknown
  Oligonucleotide
actcgcccacagagccaaaa

SEQ ID NO: 21 - DNA, 20 bp, Unknown
  Oligonucleotide
gctgagtagtgaggctgccg

SEQ ID NO: 22 - DNA, 20 bp, Unknown
  Oligonucleotide
ctggtccaggagagggtgtg

SEQ ID NO: 23 - DNA, 20 bp, Unknown
  Oligonucleotide
aggccccgccattctcccgg

SEQ ID NO: 24 - DNA, 20 bp, Unknown
  Oligonucleotide
gccacgattttgaggctggc

SEQ ID NO: 25 - DNA, 20 bp, Unknown
  Oligonucleotide
ggggttccggatcatcttgt

SEQ ID NO: 26 - DNA, 20 bp, Unknown
  Oligonucleotide
ccagggcgctgaccacctgg

SEQ ID NO: 27 - DNA, 20 bp, Unknown
  Oligonucleotide
gggaagcggggccgggcatt

SEQ ID NO: 28 - DNA, 20 bp, Unknown
  Oligonucleotide
ccggtctttctgccaacagt

SEQ ID NO: 29 - DNA, 20 bp, Unknown
  Oligonucleotide
ccagcatgagctggtggagg

SEQ ID NO: 30 - DNA, 20 bp, Unknown
  Oligonucleotide
gaggtgggacagtctggggg

SEQ ID NO: 31 - DNA, 20 bp, Unknown
  Oligonucleotide
cgggggcagccggtagtcct

SEQ ID NO: 32 - DNA, 20 bp, Unknown
  Oligonucleotide
gttcaatggcattgatcacg

SEQ ID NO: 33 - DNA, 20 bp, Unknown
  Oligonucleotide
tcctgattgctcatgtccca

SEQ ID NO: 34 - DNA, 20 bp, Unknown
  Oligonucleotide
gtacggcctctccccaaatg

SEQ ID NO: 35 - DNA, 20 bp, Unknown
  Oligonucleotide
acatcacctcccacatcaca

SEQ ID NO: 36 - DNA, 20 bp, Unknown
  Oligonucleotide
atcccgtaactccaggcatc

SEQ ID NO: 37 - DNA, 20 bp, Unknown
  Oligonucleotide
actggcggaagtgaacttcc

SEQ ID NO: 38 - DNA, 20 bp, Unknown
  Oligonucleotide
ggaaggcaatggcctccggg

SEQ ID NO: 39 - DNA, 20 bp, Unknown
  Oligonucleotide
gcagtccatcggatgggaat

SEQ ID NO: 40 - DNA, 20 bp, Unknown
  Oligonucleotide
ctttcctcccagggagctcg

SEQ ID NO: 41 - DNA, 20 bp, Unknown
  Oligonucleotide
tgtaggtgggatcggaagag

SEQ ID NO: 42 - DNA, 20 bp, Unknown
  Oligonucleotide
ttctcctccaggaatcggga

SEQ ID NO: 43 - DNA, 20 bp, Unknown
  Oligonucleotide
aaggccaaagtcagacactt

SEQ ID NO: 44 - DNA, 20 bp, Unknown
  Oligonucleotide
gcagacgaggttgctgttga

SEQ ID NO: 45 - DNA, 20 bp, Unknown
  Oligonucleotide
ctaggatgttgcgagcagcc

SEQ ID NO: 46 - DNA, 20 bp, Unknown
  Oligonucleotide
aggtctcggtggacgtagct

SEQ ID NO: 47 - DNA, 20 bp, Unknown
  Oligonucleotide
catctcggcaaggtaccgca

SEQ ID NO: 48 - DNA, 20 bp, Unknown
  Oligonucleotide
tgcccgaggcgatgccccgc

SEQ ID NO: 49 - DNA, 20 bp, Unknown
  Oligonucleotide
agcatgcccacgagctggat

SEQ ID NO: 50 - DNA, 20 bp, Unknown
  Oligonucleotide
gactgtgaactgtccgtcgt

SEQ ID NO: 51 - DNA, 20 bp, Unknown
  Oligonucleotide
ttagccgcaggaaggagtcc

SEQ ID NO: 52 - DNA, 20 bp, Unknown
  Oligonucleotide
agggcgccgttctccatgaa

SEQ ID NO: 53 - DNA, 20 bp, Unknown
  Oligonucleotide
ctctgtgagaatcatgacgg

SEQ ID NO: 54 - DNA, 20 bp, Unknown
  Oligonucleotide
gcatgctgttggtgaccacg

SEQ ID NO: 55 - DNA, 20 bp, Unknown
  Oligonucleotide
ccctccaggcggatgatatt

SEQ ID NO: 56 - DNA, 20 bp, Unknown
  Oligonucleotide
ggggtgctcgaactggccca

SEQ ID NO: 57 - DNA, 20 bp, Unknown
  Oligonucleotide
tgatggaggcctcgctcaga

SEQ ID NO: 58 - DNA, 20 bp, Unknown
  Oligonucleotide
aactcacgccgctgccgctc

SEQ ID NO: 59 - DNA, 20 bp, Unknown
  Oligonucleotide
cgtgtagccacccttcaggg

SEQ ID NO: 60 - DNA, 20 bp, Unknown
  Oligonucleotide
tcttgattgccacacagctc

SEQ ID NO: 61 - DNA, 20 bp, Unknown
  Oligonucleotide
tccttcttccctggggcctt

SEQ ID NO: 62 - DNA, 20 bp, Unknown
  Oligonucleotide
gagccgcccccggcacacct

SEQ ID NO: 63 - DNA, 20 bp, Unknown
  Oligonucleotide
cgccaaactcacctgcacca

SEQ ID NO: 64 - DNA, 20 bp, Unknown
  Oligonucleotide
atcacctcttcaatcttgac

SEQ ID NO: 65 - DNA, 20 bp, Unknown
  Oligonucleotide
gtaggagacatcgatctctt

SEQ ID NO: 66 - DNA, 20 bp, Unknown
  Oligonucleotide
ttgcaaattccctcacagcc

SEQ ID NO: 67 - DNA, 20 bp, Unknown
  Oligonucleotide
tcattagggtcttcataagt

SEQ ID NO: 68 - DNA, 20 bp, Unknown
  Oligonucleotide
gaaggggtcgatgtagacct

SEQ ID NO: 69 - DNA, 20 bp, Unknown
  Oligonucleotide
tagtaccatgtccgatgaga

SEQ ID NO: 70 - DNA, 20 bp, Unknown
  Oligonucleotide
tactgtccgtgtttgtccga

SEQ ID NO: 71 - DNA, 20 bp, Unknown
  Oligonucleotide
atattctgcttctctcccat

SEQ ID NO: 72 - DNA, 20 bp, Unknown
  Oligonucleotide
tgctctgcttcctgaggcag

SEQ ID NO: 73 - DNA, 20 bp, Unknown
  Oligonucleotide
agaactgcgaccacaatgac

SEQ ID NO: 74 - DNA, 20 bp, Unknown
  Oligonucleotide
caccaggaccaggaccacac

SEQ ID NO: 75 - DNA, 20 bp, Unknown
  Oligonucleotide
ccacgactgccgtgcccgca

SEQ ID NO: 76 - DNA, 20 bp, Unknown
  Oligonucleotide
atcagggccagctgctcccg

SEQ ID NO: 77 - DNA, 20 bp, Unknown
  Oligonucleotide
ccagccctcgctctcatcca

SEQ ID NO: 78 - DNA, 20 bp, Unknown
  Oligonucleotide
gttgggtctggctgtgatgt

SEQ ID NO: 79 - DNA, 20 bp, Unknown
  Oligonucleotide
tcctggccgaagggcccgta

SEQ ID NO: 80 - DNA, 20 bp, Unknown
  Oligonucleotide
gccggcctcagagcgcgccc

SEQ ID NO: 81 - DNA, 20 bp, Unknown
  Oligonucleotide
gtacctgcaccaggtagctg

SEQ ID NO: 82 - DNA, 20 bp, Unknown
  Oligonucleotide
gctccccgcttcagcccccg

SEQ ID NO: 83 - DNA, 20 bp, Unknown
  Oligonucleotide
cagctctgcccggttttctg

SEQ ID NO: 84 - DNA, 20 bp, Unknown
  Oligonucleotide
acgtcttcaggaaccgcacg

SEQ ID NO: 85 - DNA, 20 bp, Unknown
  Oligonucleotide
ctgctgggaccctcggcgcc

SEQ ID NO: 86 - DNA, 20 bp, Unknown
  Oligonucleotide
cttctcatggtatttgacct

SEQ ID NO: 87 - DNA, 20 bp, Unknown
  Oligonucleotide
cgtagtccagcacagcccca

SEQ ID NO: 88 - DNA, 20 bp, Unknown
  Oligonucleotide
ctgggtgcccggggaacagc

SEQ ID NO: 89 - DNA, 20 bp, Unknown
  Oligonucleotide
ccaggccaggctcaagctgc

SEQ ID NO: 90 - DNA, 20 bp, Unknown
  Oligonucleotide
tgggtgaggaccgcgtcacc

SEQ ID NO: 91 - DNA, 20 bp, Unknown
  Oligonucleotide
cggatgtcagacactgcagg

SEQ ID NO: 92 - DNA, 20 bp, Unknown
  Oligonucleotide
aggtacctctcggtcagtgg

SEQ ID NO: 93 - DNA, 20 bp, Unknown
  Oligonucleotide
tgacattgacaggctcaaat

SEQ ID NO: 94 - DNA, 20 bp, Unknown
  Oligonucleotide
gggacgggccccgtggctaa

SEQ ID NO: 95 - DNA, 20 bp, Unknown
  Oligonucleotide
ggaggataccccgttcaatg

SEQ ID NO: 96 - DNA, 20 bp, Unknown
  Oligonucleotide
cagtgacctcaaaggtatag

SEQ ID NO: 97 - DNA, 20 bp, Unknown
  Oligonucleotide
gtgaagtcaggacgtagccc

SEQ ID NO: 98 - DNA, 20 bp, Unknown
  Oligonucleotide
tcgaaccaccacccagggct

SEQ ID NO: 99 - DNA, 20 bp, Unknown
  Oligonucleotide
ccaccaggtcccgggggccg

SEQ ID NO: 100 - DNA, 20 bp, Unknown
  Oligonucleotide
gggtcaaaagtcaggtctcc

SEQ ID NO: 101 - DNA, 20 bp, Unknown
  Oligonucleotide
cccgcagggcgcacaggagc

SEQ ID NO: 102 - DNA, 20 bp, Unknown
  Oligonucleotide
ctccgggtcggcactcccgg

SEQ ID NO: 103 - DNA, 20 bp, Unknown
  Oligonucleotide
cagcggagggcgtaggtgag

SEQ ID NO: 104 - DNA, 20 bp, Unknown
  Oligonucleotide
gtcctctcggccaccagact

SEQ ID NO: 105 - DNA, 20 bp, Unknown
  Oligonucleotide
ccaggggggcactccattcc

SEQ ID NO: 106 - DNA, 20 bp, Unknown
  Oligonucleotide
aggtgcagggaggagccgtt

SEQ ID NO: 107 - DNA, 20 bp, Unknown
  Oligonucleotide
caggcgggaaaccacgctcc

SEQ ID NO: 108 - DNA, 20 bp, Unknown
  Oligonucleotide
gcggagccgaaggaggggtg

SEQ ID NO: 109 - DNA, 20 bp, Unknown
  Oligonucleotide
gtgcagggtgcaccccgggg

SEQ ID NO: 110 - DNA, 20 bp, Unknown
  Oligonucleotide
gtctgtgcgtgcccggaagt

SEQ ID NO: 111 - DNA, 20 bp, Unknown
  Oligonucleotide
acccgacgcggcactggcag

SEQ ID NO: 112 - DNA, 20 bp, Unknown
  Oligonucleotide
acggctgatccaatggtgtt

SEQ ID NO: 113 - DNA, 20 bp, Unknown
  Oligonucleotide
agagtggctattggctgggc

SEQ ID NO: 114 - DNA, 20 bp, Unknown
  Oligonucleotide
atggctggcaggacccttct

SEQ ID NO: 115 - DNA, 20 bp, Unknown
  Oligonucleotide
cctgacaggggcttgaaggt

SEQ ID NO: 116 - DNA, 20 bp, Unknown
  Oligonucleotide
gccctgggcacaggctcggc

SEQ ID NO: 117 - DNA, 20 bp, Unknown
  Oligonucleotide
acttggtgttcccctcagct

SEQ ID NO: 118 - DNA, 20 bp, Unknown
  Oligonucleotide
gcctcgaaccccggagcaca

SEQ ID NO: 119 - DNA, 20 bp, Unknown
  Oligonucleotide
gctgcagcccgtgaccggct

SEQ ID NO: 120 - DNA, 20 bp, Unknown
  Oligonucleotide
gttcggcccactggccatcc

SEQ ID NO: 121 - DNA, 20 bp, Unknown
  Oligonucleotide
tcacggcagtagaggctggg

SEQ ID NO: 122 - DNA, 20 bp, Unknown
  Oligonucleotide
gctggggccaggggcgggga

SEQ ID NO: 123 - DNA, 20 bp, Unknown
  Oligonucleotide
cggcatccaccacgcagcta

SEQ ID NO: 124 - DNA, 20 bp, Unknown
  Oligonucleotide
ccggccacgggcacaaccag

SEQ ID NO: 125 - DNA, 20 bp, Unknown
  Oligonucleotide
ctcccgaggcacagtctccg

SEQ ID NO: 126 - DNA, 20 bp, Unknown
  Oligonucleotide
ggaatcgagtcaggttcaca

SEQ ID NO: 127 - DNA, 20 bp, Unknown
  Oligonucleotide
gtcagctgggcgcacttttt

SEQ ID NO: 128 - DNA, 20 bp, Unknown
  Oligonucleotide
gtagaagaggtgcagggata

SEQ ID NO: 129 - DNA, 20 bp, Unknown
  Oligonucleotide
gcagggccatgcaggcaccc

SEQ ID NO: 130 - DNA, 20 bp, Unknown
  Oligonucleotide
tggtcctggaaggccaggta

SEQ ID NO: 131 - DNA, 20 bp, Unknown
  Oligonucleotide
gaagccagccttgctgagcg

SEQ ID NO: 132 - DNA, 20 bp, Unknown
  Oligonucleotide
gtcccagacgcagcgtcttg

SEQ ID NO: 133 - DNA, 20 bp, Unknown
  Oligonucleotide
acattcaccttcccggtggc

SEQ ID NO: 134 - DNA, 20 bp, Unknown
  Oligonucleotide
ctcggccccagggcgcttcc

SEQ ID NO: 135 - DNA, 20 bp, Unknown
  Oligonucleotide
gggtgagatgctccgcggcc

SEQ ID NO: 136 - DNA, 20 bp, Unknown
  Oligonucleotide
accgtgtccaccttgatgta

SEQ ID NO: 137 - DNA, 20 bp, Unknown
  Oligonucleotide
ggggttctccatccaggctg

SEQ ID NO: 138 - DNA, 20 bp, Unknown
  Oligonucleotide
gcgtgagggccgtggccgtg

SEQ ID NO: 139 - DNA, 20 bp, Unknown
  Oligonucleotide
tccgcatcgctctcatagta

SEQ ID NO: 140 - DNA, 20 bp, Unknown
  Oligonucleotide
gaagacggtgaaggtctcct

SEQ ID NO: 141 - DNA, 20 bp, Unknown
  Oligonucleotide
tgcaggagcgcccagcccga

SEQ ID NO: 142 - DNA, 20 bp, Unknown
  Oligonucleotide
ggcagggacaggcactcgag

SEQ ID NO: 143 - DNA, 20 bp, Unknown
  Oligonucleotide
catggtgaagcgcagcgtgg

SEQ ID NO: 144 - DNA, 20 bp, Unknown
  Oligonucleotide
cgtacacgtggacggcgccc

SEQ ID NO: 145 - DNA, 20 bp, Unknown
  Oligonucleotide
cgccgtgggacccaacctgt

SEQ ID NO: 146 - DNA, 20 bp, Unknown
  Oligonucleotide
gcgaagccagtgggcctggc

SEQ ID NO: 147 - DNA, 20 bp, Unknown
  Oligonucleotide
ccggggcacgctgcacgtca

SEQ ID NO: 148 - DNA, 20 bp, Unknown
  Oligonucleotide
cacacttcgtaggtgcgcac

SEQ ID NO: 149 - DNA, 20 bp, Unknown
  Oligonucleotide
gctgtgctgttcctcatcca

SEQ ID NO: 150 - DNA, 20 bp, Unknown
  Oligonucleotide
ggccgctcagttcctcccac

SEQ ID NO: 151 - DNA, 20 bp, Unknown
  Oligonucleotide
tgcccgtccacctgagggaa

SEQ ID NO: 152 - DNA, 20 bp, Unknown
  Oligonucleotide
tgtcacccacttcagatcag

SEQ ID NO: 153 - DNA, 20 bp, Unknown
  Oligonucleotide
cagtttccaattttgtgttc

SEQ ID NO: 154 - DNA, 20 bp, Unknown
  Oligonucleotide
agcagggtctcttccaaagc

SEQ ID NO: 155 - DNA, 20 bp, Unknown
  Oligonucleotide
tgcggccaacgaagcccagc

SEQ ID NO: 156 - DNA, 20 bp, Unknown
  Oligonucleotide
agagcagcacccggagctcc

SEQ ID NO: 157 - DNA, 20 bp, Unknown
  Oligonucleotide
agcagcacccggagctccat

SEQ ID NO: 158 - DNA, 21 bp, Unknown
  Oligonucleotide
gtgcagggatagcagggccat

SEQ ID NO: 159 - DNA, 21 bp, Unknown
  Oligonucleotide
aaggaggggtggtgcacggtg

SEQ ID NO: 160 - DNA, 21 bp, Unknown
  Oligonucleotide
ttccaggtgcagggaggagcc

SEQ ID NO: 161 - DNA, 21 bp, Unknown
  Oligonucleotide
gtggtgacattgacaggctca

SEQ ID NO: 162 - DNA, 21 bp, Unknown
  Oligonucleotide
tctggctgtgatgttcctggc

SEQ ID NO: 163 - DNA, 18 bp, Unknown
  Oligonucleotide
gccgctcagttcctccca

SEQ ID NO: 164 - DNA, 18 bp, Unknown
  Oligonucleotide
tgaaggtctccttgcagg

SEQ ID NO: 165 - DNA, 21 bp, Unknown
  Oligonucleotide
cgcggccaccgtgtccacctt

SEQ ID NO: 166 - DNA, 21 bp, Unknown
  Oligonucleotide
cttcagggtcttgattgccac

SEQ ID NO: 167 - DNA, 20 bp, Unknown
  Oligonucleotide
atggaggcctcgctcagaaa

SEQ ID NO: 168 - DNA, 21 bp, Unknown
  Oligonucleotide
catgcccacgagctggatgac

SEQ ID NO: 169 - DNA, 21 bp, Unknown
  Oligonucleotide
aaattggaaactgctgatctg

SEQ ID NO: 170 - DNA, 21 bp, Unknown
  Oligonucleotide
aattggaaactgctgatctga

SEQ ID NO: 171 - DNA, 21 bp, Unknown
  Oligonucleotide
aaactgctgatctgaagtggg

SEQ ID NO: 172 - DNA, 21 bp, Unknown
  Oligonucleotide
aactgctgatctgaagtgggt

SEQ ID NO: 173 - DNA, 21 bp, Unknown
  Oligonucleotide
aatgtcaagacgctgcgtctg

SEQ ID NO: 174 - DNA, 21 bp, Unknown
  Oligonucleotide
aagtgggtgacattccctcag

SEQ ID NO: 175 - DNA, 21 bp, Unknown
  Oligonucleotide
aaggtgaatgtcaagacgctg

SEQ ID NO: 176 - DNA, 21 bp, Unknown
  Oligonucleotide
aaggagaccttcaccgtcttc

SEQ ID NO: 177 - DNA, 21 bp, Unknown
  Oligonucleotide
aaaaagtgcgcccagctgact

SEQ ID NO: 178 - DNA, 21 bp, Unknown
  Oligonucleotide
aatagccactctaacaccatt

SEQ ID NO: 179 - DNA, 21 bp, Unknown
  Oligonucleotide
aacaccattggatcagccgtc

SEQ ID NO: 180 - DNA, 21 bp, Unknown
  Oligonucleotide
aatgtcaccactgaccgagag

SEQ ID NO: 181 - DNA, 21 bp, Unknown
  Oligonucleotide
aaataccatgagaagggcgcc

SEQ ID NO: 182 - DNA, 21 bp, Unknown
  Oligonucleotide
aagacgtcagaaaaccgggca

SEQ ID NO: 183 - DNA, 21 bp, Unknown
  Oligonucleotide
aacatcacagccagacccaac

SEQ ID NO: 184 - DNA, 21 bp, Unknown
  Oligonucleotide
aagcagagcaatgggagagaa

SEQ ID NO: 185 - DNA, 21 bp, Unknown
  Oligonucleotide
aatgggagagaagcagaatat

SEQ ID NO: 186 - DNA, 21 bp, Unknown
  Oligonucleotide
aagcagaatattcggacaaac

SEQ ID NO: 187 - DNA, 21 bp, Unknown
  Oligonucleotide
aatattcggacaaacacggac

SEQ ID NO: 188 - DNA, 21 bp, Unknown
  Oligonucleotide
aaacacggacagtatctcatc

SEQ ID NO: 189 - DNA, 21 bp, Unknown
  Oligonucleotide
aacacggacagtatctcatcg

SEQ ID NO: 190 - DNA, 21 bp, Unknown
  Oligonucleotide
aaaagagatcgatgtctccta

SEQ ID NO: 191 - DNA, 21 bp, Unknown
  Oligonucleotide
aatgaggctgtgagggaattt

SEQ ID NO: 192 - DNA, 21 bp, Unknown
  Oligonucleotide
aagaccctaatgaggctgtga

SEQ ID NO: 193 - DNA, 21 bp, Unknown
  Oligonucleotide
aaagagatcgatgtctcctac

SEQ ID NO: 194 - DNA, 21 bp, Unknown
  Oligonucleotide
aagagatcgatgtctcctacg

SEQ ID NO: 195 - DNA, 21 bp, Unknown
  Oligonucleotide
aagaggtgattggtgcaggtg

SEQ ID NO: 196 - DNA, 21 bp, Unknown
  Oligonucleotide
aagattgaagaggtgattggt

SEQ ID NO: 197 - DNA, 21 bp, Unknown
  Oligonucleotide
aacagcatgcccgtcatgatt

SEQ ID NO: 198 - DNA, 21 bp, Unknown
  Oligonucleotide
aagaaggagagctgtgtggca

SEQ ID NO: 199 - DNA, 21 bp, Unknown
  Oligonucleotide
aaggagagctgtgtggcaatc

SEQ ID NO: 200 - DNA, 21 bp, Unknown
  Oligonucleotide
aatcaagaccctgaagggtgg

SEQ ID NO: 201 - DNA, 21 bp, Unknown
  Oligonucleotide
aaacgacggacagttcacagt

SEQ ID NO: 202 - DNA, 21 bp, Unknown
  Oligonucleotide
aacgacggacagttcacagtc

SEQ ID NO: 203 - DNA, 21 bp, Unknown
  Oligonucleotide
aacatcctagtcaacagcaac

SEQ ID NO: 204 - DNA, 21 bp, Unknown
  Oligonucleotide
aacagcaacctcgtctgcaaa

SEQ ID NO: 205 - DNA, 21 bp, Unknown
  Oligonucleotide
aactcttccgatcccacctac

SEQ ID NO: 206 - DNA, 21 bp, Unknown
  Oligonucleotide
aagtgtctgactttggccttt

SEQ ID NO: 207 - DNA, 21 bp, Unknown
  Oligonucleotide
aacctcgtctgcaaagtgtct

SEQ ID NO: 208 - DNA, 21 bp, Unknown
  Oligonucleotide
aaagtgtctgactttggcctt

SEQ ID NO: 209 - DNA, 21 bp, Unknown
  Oligonucleotide
aatcaggacgtgatcaatgcc

SEQ ID NO: 210 - DNA, 21 bp, Unknown
  Oligonucleotide
aaagattcccatccgatggac

SEQ ID NO: 211 - DNA, 21 bp, Unknown
  Oligonucleotide
aagattcccatccgatggact

SEQ ID NO: 212 - DNA, 21 bp, Unknown
  Oligonucleotide
aagttcacttccgccagtgat

SEQ ID NO: 213 - DNA, 21 bp, Unknown
  Oligonucleotide
aagatacgaagaaagtttcgc

SEQ ID NO: 214 - DNA, 21 bp, Unknown
  Oligonucleotide
aatgggaagatacgaagaaag

SEQ ID NO: 215 - DNA, 21 bp, Unknown
  Oligonucleotide
aatgccattgaacaggactac

SEQ ID NO: 216 - DNA, 21 bp, Unknown
  Oligonucleotide
aaaatcgtggcccgggagaat

SEQ ID NO: 217 - DNA, 21 bp, Unknown
  Oligonucleotide
aaaatcttggccagtgtccag

SEQ ID NO: 218 - DNA, 21 bp, Unknown
  Oligonucleotide
aaatcttggccagtgtccagc

SEQ ID NO: 219 - DNA, 21 bp, Unknown
  Oligonucleotide
aagaaagtttcgcagccgctg

SEQ ID NO: 220 - DNA, 21 bp, Unknown
  Oligonucleotide
aagaaaatcttggccagtgtc

SEQ ID NO: 221 - DNA, 21 bp, Unknown
  Oligonucleotide
aatcttggccagtgtccagca

SEQ ID NO: 222 - RNA, 21 nt, Unknown
  Oligonucleotide
gagacccugcugaacacaauu

SEQ ID NO: 223 - RNA, 21 nt, Unknown
  Oligonucleotide
ggugaaugucaagacgcuguu

SEQ ID NO: 224 - RNA, 21 nt, Unknown
  Oligonucleotide
caucacagccagacccaacuu

SEQ ID NO: 225 - RNA, 21 nt, Unknown
  Oligonucleotide
cucuuccgaucccaccuacuu

SEQ ID NO: 226 - RNA, 21 nt, Unknown
  Oligonucleotide
cucuuccgaucccaccuacuu

SEQ ID NO: 227 - DNA, 20 bp, Unknown
  Oligonucleotide
tcagaccttgtagtaaatgt

SEQ ID NO: 228 - DNA, 20 bp, Unknown
  Oligonucleotide
tcgccgggctctgcgggggc

SEQ ID NO: 229 - DNA, 20 bp, Unknown
  Oligonucleotide
atctcctggacgatgtacac

SEQ ID NO: 230 - DNA, 20 bp, Unknown
  Oligonucleotide
cgggtgcccgtagtccccgc

SEQ ID NO: 231 - DNA, 20 bp, Unknown
  Oligonucleotide
tgaccttctcgtagtgaggg

SEQ ID NO: 232 - DNA, 20 bp, Unknown
  Oligonucleotide
cagaagacgctgtccgcagt

SEQ ID NO: 233 - DNA, 20 bp, Unknown
  Oligonucleotide
ccttagcgggatgataatgt

SEQ ID NO: 234 - DNA, 20 bp, Unknown
  Oligonucleotide
cactgggctctgagccgttg

SEQ ID NO: 235 - DNA, 20 bp, Unknown
  Oligonucleotide
ttgttgccgctgcgcttggg

SEQ ID NO: 236 - DNA, 20 bp, Unknown
  Oligonucleotide
tgtggccagtgtgctgagcg

SEQ ID NO: 237 - DNA, 20 bp, Unknown
  Oligonucleotide
acagcgtggtcgtgtgctgc

SEQ ID NO: 238 - DNA, 20 bp, Unknown
  Oligonucleotide
ggcgagtgcttcctgtgtct

SEQ ID NO: 239 - DNA, 20 bp, Unknown
  Oligonucleotide
cctccggtacttcagcaaga

SEQ ID NO: 240 - DNA, 20 bp, Unknown
  Oligonucleotide
ggaccaccagcgtgatgatg

SEQ ID NO: 241 - DNA, 20 bp, Unknown
  Oligonucleotide
atgacgatgaagatgatgca

SEQ ID NO: 242 - DNA, 20 bp, Unknown
  Oligonucleotide
tcctgaagcaatccctgcaa

SEQ ID NO: 243 - DNA, 20 bp, Unknown
  Oligonucleotide
ataaggccacttcggaaccg

SEQ ID NO: 244 - DNA, 20 bp, Unknown
  Oligonucleotide
aggatgttgttccccgaatg

SEQ ID NO: 245 - DNA, 20 bp, Unknown
  Oligonucleotide
tccggcgctgttgccgtctg

SEQ ID NO: 246 - DNA, 20 bp, Unknown
  Oligonucleotide
tgctagaacctggatttggt

SEQ ID NO: 247 - DNA, 20 bp, Unknown
  Oligonucleotide
tttacaaagggacttgttgt

SEQ ID NO: 248 - DNA, 20 bp, Unknown
  Oligonucleotide
cgaacttcttccatttgtac

SEQ ID NO: 249 - DNA, 20 bp, Unknown
  Oligonucleotide
cagcttctagttctggacgt

SEQ ID NO: 250 - DNA, 20 bp, Unknown
  Oligonucleotide
cttgttggatctttattcct

SEQ ID NO: 251 - DNA, 20 bp, Unknown
  Oligonucleotide
ggttgatccagcagaacttg

SEQ ID NO: 252 - DNA, 20 bp, Unknown
  Oligonucleotide
catcttgtccaactttcatg

SEQ ID NO: 253 - DNA, 20 bp, Unknown
  Oligonucleotide
aggatcttcatggctcttgt

SEQ ID NO: 254 - DNA, 20 bp, Unknown
  Oligonucleotide
ctggcacacccctccctcct

SEQ ID NO: 255 - DNA, 20 bp, Unknown
  Oligonucleotide
ggttatccaggccctccaaa

SEQ ID NO: 256 - DNA, 20 bp, Unknown
  Oligonucleotide
gacccatttgatgtagatat

SEQ ID NO: 257 - DNA, 20 bp, Unknown
  Oligonucleotide
aatgtaataatctttgttct

SEQ ID NO: 258 - DNA, 20 bp, Unknown
  Oligonucleotide
tctgaaattctagaccccag

SEQ ID NO: 259 - DNA, 20 bp, Unknown
  Oligonucleotide
aggttagggctgaattcttg

SEQ ID NO: 260 - DNA, 20 bp, Unknown
  Oligonucleotide
aaacttgatggtgaatttga

SEQ ID NO: 261 - DNA, 20 bp, Unknown
  Oligonucleotide
tatcttggtctggtttggca

SEQ ID NO: 262 - DNA, 20 bp, Unknown
  Oligonucleotide
cagttgaggagaggggtatt

SEQ ID NO: 263 - DNA, 20 bp, Unknown
  Oligonucleotide
ttccttcttaatagtgcatc

SEQ ID NO: 264 - DNA, 20 bp, Unknown
  Oligonucleotide
tgtctgcttggtctttatca

SEQ ID NO: 265 - DNA, 20 bp, Unknown
  Oligonucleotide
accatataaactttataata

SEQ ID NO: 266 - DNA, 20 bp, Unknown
  Oligonucleotide
ttcatactggccaacagttt

SEQ ID NO: 267 - DNA, 20 bp, Unknown
  Oligonucleotide
tagagtccactttggggcaa

SEQ ID NO: 268 - DNA, 20 bp, Unknown
  Oligonucleotide
ataatatccaatttgtctcc

SEQ ID NO: 269 - DNA, 20 bp, Unknown
  Oligonucleotide
tatctgtgggtatagtacca

SEQ ID NO: 270 - DNA, 20 bp, Unknown
  Oligonucleotide
gtccttgtccaggtagaaat

SEQ ID NO: 271 - DNA, 20 bp, Unknown
  Oligonucleotide
ttggagttcgaggaattcca

SEQ ID NO: 272 - DNA, 20 bp, Unknown
  Oligonucleotide
atagataggctctaaaacta

SEQ ID NO: 273 - DNA, 20 bp, Unknown
  Oligonucleotide
tcgatttggaaatcgcagtt

SEQ ID NO: 274 - DNA, 20 bp, Unknown
  Oligonucleotide
ctgcataaaaccatcaaaac

SEQ ID NO: 275 - DNA, 20 bp, Unknown
  Oligonucleotide
accccagcagtacttccaca

SEQ ID NO: 276 - DNA, 20 bp, Unknown
  Oligonucleotide
cggagtcccttctcacagcc

SEQ ID NO: 277 - DNA, 20 bp, Unknown
  Oligonucleotide
gagtcccttctcacagccat

SEQ ID NO: 278 - DNA, 21 bp, Unknown
  Oligonucleotide
aactgcgatttccaaatcgat

SEQ ID NO: 279 - DNA, 21 bp, Unknown
  Oligonucleotide
aactccaaatttctacctgga

SEQ ID NO: 280 - DNA, 21 bp, Unknown
  Oligonucleotide
aatttctacctggacaaggac

SEQ ID NO: 281 - DNA, 21 bp, Unknown
  Oligonucleotide
aaatttctacctggacaagga

SEQ ID NO: 282 - DNA, 21 bp, Unknown
  Oligonucleotide
aaggactggtactatacccac

SEQ ID NO: 283 - DNA, 21 bp, Unknown
  Oligonucleotide
aagtggactctaaaactgttg

SEQ ID NO: 284 - DNA, 21 bp, Unknown
  Oligonucleotide
aaactgttggccagtatgaat

SEQ ID NO: 285 - DNA, 21 bp, Unknown
  Oligonucleotide
aaaactgttggccagtatgaa

SEQ ID NO: 286 - DNA, 21 bp, Unknown
  Oligonucleotide
aagaccaagcagacagatgca

SEQ ID NO: 287 - DNA, 21 bp, Unknown
  Oligonucleotide
aaagaccaagcagacagatgc

SEQ ID NO: 288 - DNA, 21 bp, Unknown
  Oligonucleotide
aagtttcaagaattcagccct

SEQ ID NO: 289 - DNA, 21 bp, Unknown
  Oligonucleotide
aagaattcagccctaacctct

SEQ ID NO: 290 - DNA, 21 bp, Unknown
  Oligonucleotide
aattcagccctaacctctggg

SEQ ID NO: 291 - DNA, 21 bp, Unknown
  Oligonucleotide
aactgtgccaaaccagaccaa

SEQ ID NO: 292 - DNA, 21 bp, Unknown
  Oligonucleotide
aaatgggtctttggagggcct

SEQ ID NO: 293 - DNA, 21 bp, Unknown
  Oligonucleotide
aagatcctcatgaaagttgga

SEQ ID NO: 294 - DNA, 21 bp, Unknown
  Oligonucleotide
aaagttggacaagatgcaagt

SEQ ID NO: 295 - DNA, 21 bp, Unknown
  Oligonucleotide
aagagccatgaagatcctcat

SEQ ID NO: 296 - DNA, 21 bp, Unknown
  Oligonucleotide
aagttggacaagatgcaagtt

SEQ ID NO: 297 - DNA, 21 bp, Unknown
  Oligonucleotide
aagatgcaagttctgctggat

SEQ ID NO: 298 - DNA, 21 bp, Unknown
  Oligonucleotide
aagttctgctggatcaaccag

SEQ ID NO: 299 - DNA, 21 bp, Unknown
  Oligonucleotide
aaccaggaataaagatccaac

SEQ ID NO: 300 - DNA, 21 bp, Unknown
  Oligonucleotide
aaagatccaacaagacgtcca

SEQ ID NO: 301 - DNA, 21 bp, Unknown
  Oligonucleotide
aagatccaacaagacgtccag

SEQ ID NO: 302 - DNA, 21 bp, Unknown
  Oligonucleotide
aacaagacgtccagaactaga

SEQ ID NO: 303 - DNA, 21 bp, Unknown
  Oligonucleotide
aagacgtccagaactagaagc

SEQ ID NO: 304 - DNA, 21 bp, Unknown
  Oligonucleotide
aaatggaagaagttcgacaac

SEQ ID NO: 305 - DNA, 21 bp, Unknown
  Oligonucleotide
aactagaagctggtacaaatg

SEQ ID NO: 306 - DNA, 21 bp, Unknown
  Oligonucleotide
aatggaagaagttcgacaaca

SEQ ID NO: 307 - DNA, 21 bp, Unknown
  Oligonucleotide
aagctggtacaaatggaagaa

SEQ ID NO: 308 - DNA, 21 bp, Unknown
  Oligonucleotide
aacaagtccctttgtaaaacc

SEQ ID NO: 309 - DNA, 21 bp, Unknown
  Oligonucleotide
aagaagttcgacaacaagtcc

SEQ ID NO: 310 - DNA, 21 bp, Unknown
  Oligonucleotide
aagttcgacaacaagtccctt

SEQ ID NO: 311 - DNA, 21 bp, Unknown
  Oligonucleotide
aaaaccaaatccaggttctag

SEQ ID NO: 312 - DNA, 21 bp, Unknown
  Oligonucleotide
aaaccaaatccaggttctagc

SEQ ID NO: 313 - DNA, 21 bp, Unknown
  Oligonucleotide
aaccaaatccaggttctagca

SEQ ID NO: 314 - DNA, 21 bp, Unknown
  Oligonucleotide
aaatccaggttctagcacaga

SEQ ID NO: 315 - DNA, 21 bp, Unknown
  Oligonucleotide
aatccaggttctagcacagac

SEQ ID NO: 316 - DNA, 21 bp, Unknown
  Oligonucleotide
aacaacatcctcggttccgaa

SEQ ID NO: 317 - DNA, 21 bp, Unknown
  Oligonucleotide
aacatcctcggttccgaagtg

SEQ ID NO: 318 - DNA, 21 bp, Unknown
  Oligonucleotide
aagtggccttatttgcaggga

SEQ ID NO: 319 - RNA, 21 nt, Unknown
  Oligonucleotide
gcagacagaugcacuauuauu

SEQ ID NO: 320 - RNA, 21 nt, Unknown
  Oligonucleotide
cugcgauuuccaaaucgauuu

SEQ ID NO: 321 - RNA, 21 nt, Unknown
  Oligonucleotide
ggacugguacuauacccacuu

SEQ ID NO: 322 - protein, 570 aa, Unknown
  Recombinant B4ECv3 protein
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQGSKRAILQIEGKPIPNPLLGLDSTRTGHHHHHH

SEQ ID NO: 323 - protein, 555 aa, Unknown
  Recombinant B4ECv3NT protein
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQGSKRAILQISSTVAAARV

SEQ ID NO: 324 - protein, 233 aa, Unknown
  Recombinant B2EC protein
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVGSHHHHHH

SEQ ID NO: 325 - protein, 771 aa, Unknown
  Recombinant B4ECv3-FC protein
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQDPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 459 aa, Unknown
  Recombinant B2EC-FC protein
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVDPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - DNA, 26000 bp, Homo sapiens
ggggtttcatcatgttggccaggctggtcttgaactcctgacctcaaatgatccgcctgcctctgcctcccaaaatgctgggactacaggcgtgagccaccgcgcccgccacacccaccttttctttaccgttgtttcctcgatttttctctactccctagcgcagcttagtgcgcgcctcctctggacatttttcagggcttggttgcgcgcacagtaggtccccaacactgaatgtttatggggtgactgtgtgaacgttcgctgcaaggctatccaaactgggattgctccttgaggccccctgggcggccgtcaattctccaaagcttctactcccttttccttccttttcccccaaaacgcagtccctgcgcccactagagggtggtgggcgcatccaagagcggcatctagagtccgcagcaaggtcagagcgggctttgtgtgcgcggtgaacatttacgtgcacgcctgggcggccctccgtgttgctgctgggtgtgtgttttctctgctccctggtgccagccgggttcgggcctgtcccgggggtccctgggccccagccccgacatgctcggtcctggacagcgcgcaccgccacggcgcacatctgggcggtcccggggttcctcacccgccgcccctcccccttctccaaactttctctcaacttcccgacctgctccactcggtgcccctctccgcttccctcatgaattattcagtagcgtgagctccaatcagcgcgcccggggctcactcgcggagcccccgcgttgggagagctgcccccgccccccgcgcgcccctccctcccgggcccggcgccgcccggcccagttccagcgcagctcagcccctgcccggcccggcccgcccggctccgcgccgcagtctccctccctcccgctccgtccccgctcgggctcccaccatccccgcccgcgaggagagcactcggcccggcggcgcgagcagagccactccagggagggggggagaccgcgagcggccggctcagcccccgccacccggggcgggaccccgaggccccggagggaccccaactccagccacgtcttgctgcgcgcccgcccggcgcggccactgccagcacgctccgggcccgccgcccgcgcgcgcggcacagacgcggggccacacttggcgccgccgcccggtgccccgcacgctcgcatgggcccgcgctgagggccccgacgaggagtcccgcgcggagtatcggcgtccacccgcccagggagagtcagacctgggggggcgagggccccccaaactcagttcggatcctacccgagtgaggcggcgccatggagctccgggtgctgctctgctgggcttcgttggccgcagctttggaaggtgagtttccttgcgggggggggcgcaccccgtcactcctgggacctcccccccaacatctgggcctcggagtggaggggccggcctctgactacccctacccgggcactgcagtcccaaacacttcggaccgatagtgctggaacgggaggggggcggggaagaggcgcccgacgggtagtggagttttcttttgtttgggaaagagatggagtctggctacgacccgggacattcccctgcccgggctccccgaactctcactgctgattacatacgcccctggctgcctttcctttcctccctaccccactattcaaaactatctgcaaagtttctgtcccagtcccacctcccgccgtacatgagggaaggtttctggagaagcaacagcagacaaggcacaacttttcgtgctaggccctaaaacgacccccagcgccaattccttagcgatcacaccttgatcctccagttccacactcctgcaacaggatggcctcctttgcattcacacagcaaacccccaaaccgctctcccgcccactgctcctgcccctggtatagggtggctccttggtttctacaggctgcaccccatccctttaaatgcggtctagaccccggccccaggtgagtcccgggcttcccttgagacctaggagcgggtagaaactgacctacacagcccccaggtagaaactgacctacacagcccccacatcgccctaactaacccagtctatctcccacctcctggtctctccaagcatttctttggccatggatcgctgtccctcctggtcccctaaagggggagccaagagccctagaaactctcctgtgtccctaatgtcctttcagtgagctgccaacacccccctttctctgtctggtatgaaagtggttatggggcggtaggctatgagggactcccaaagggaaggattcagcggcgttagaaaaaccctctccccctggctgggcaggactgccctgggctggggatcaaaggctaggtgtggggttgggagtgaggggaggcttgcccagctcagagaacggagaagggggaacaaaaaccatgaacgaggggaagaggaaggccaaaggggtggaaaaaccacgaggacgaggtgtggtgagaaggaaagacgcaaagaggaaatggtgattgtgacacctattacctgagtgtttccaagcaccaggcctgtgctgagcgccttacaaatattaatttcacccatccagcaacgctaagggtggtgctattattgcccccatttttcagatgaggaggctggggcttagttaaggttaagtagtttatccaaggccctgtgccgcgaggaacagcgagaagtggaggccgaaagcgaaggagagatagtgactgtcagaaagagaaacggaggtggacagagagtggaggagagataggtgagagacatgcgaactgacagatcaaagcgtggctgcagctgagctgggacgcagaaagggagcctgcgcttgctctgggctgcggacagcccgaggcagagacagtgtgtaaattggagacaggaaaacactatcccggctggaacaatggagggtggagacggcagcctctatccacccccttcccagaacccgggcatcctgtccccagtgagcagggctgtctcttgccacccatggggaccttgcgcctctcacctcaggctggctggcttcccatctgacccctagctggaggacatcatttggtccccaggaagaggctgcctcacccaccctctttctcttctctcctgcagctcccatggggtgggagccaggtgttctggctcccctctccacccttcccagcgcccaatgccccccacattgccggcccccgaggggattcctgtaccctccctcctccactctccactgccaggggctgtgcagtttttcctaatccccccccttcctccagtgcctgtcccctcccccgatgatccgagccaagccaggtgtgttcacccctcccattcataccgccccccagaatctcctcccctctgccttcccataaccaaatccagatgtgaggcctcggcgggagcctgggaaccctagcatcccgacctccagtgcttcctgatcagggcactcgtggggagggaggtactgggatgggggccagggctatgccccaggcacggagcgctcccttcaaggagggaaggacggggtgtttggtctgaaagcagagaggggtcttggacagggaatgaaattgtggggtagagaggctgattctgggacttaggggaggaaacgtggaggctgagacaagaggttcccctcccacaccagcagcctctgctcgtgggggtcaggaccagggcgcagctctcattttaaccctttctgagctgccgccccttctccccgtacattttgatctccctccctcctccagggaggcctagatctggggtatcccaagggagccccatgcctaccagatgttgggggtggggttggcacttagcagaagaggccagaaatcaggcgggtgcagagggcagggcttgctcccctcttggccccccaactcctctagctcagagctaagaggatccacctgcctcggttcccagggatctggtcttcctgacctccctcccccaccccaggcactgactctgtctctctgtctgtctcagagaccctgctgaacacaaaattggaaactgctgatctgaagtgggtgacattccctcaggtggacgggcaggtgagagctgcacccaggagctggagctctggagggaaactgagggaggagagggcgcctgtgccgcctgctttctgtgtgccactcctctcccctgtccccccagatgacagcagccccagcagtgtcgtctgagcccttctcagaggcgccctcctcgcagtaccagcagcccccctttctcagtccctctcactttataggattcaccccatgcagccctctccctggcggctccccagcccccttgctgacctccttctctgcacagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtacctgggtgcccccagggctcagccacagccaaggtgggattccagccagcaggcccgtggcctggagggcagccgatgtagttgcgaggcctctggcccgcgcgctgggggctggaagcaggaggcttaggtctggggagggaagggggtgatcttctgggcggaggagcagaatatacgggggctgcctggcccggcccccagggaggcccaagggtcaggcttctcctccagtcacctcaaccaccctaccccactgtgctccagccacactgagtttctcccattccctgactgcacctggctggtttccagctcaagactttgcagcggtgatgtctccacctgggggcctctctgcctctcacacccctacttgtcttcggagttccagctcccgagatcttgcctgtgccaccttggctgactctctcctccctacaatcctgcatacctctgtccacctgcctgtctcggcactcattttactttatttatttttcttttatatctatatttttaaagcggggtcttctacgttacccaggctggtctctaactcctgggctcaagagatttctcccacctcggcctcctaaagtgctgggattataggcatgaggcactacgcccggcctcatggtactttataacttccccaggattcattcatcgctgtctccttgactctgaggtcaaggcctggcatggcgtcagtgtcagtaaatgtttgtagaacgagtgaataaaaagggggagaggtgcaggccagaggccgggcatatcgcaggagctttgcaaggctgaatggacagtgtgggggcctgcagaaagtgtgccctggggaaggtggagggaagattctggaacgggaaccaaggaggtccgggagggtgagctgggaagaacacaacagtccgctgggtcctcagggagtggggacagcagcggtgtgcctcccccccgccggcaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgaggtgagagctggagcttcccctgcgactgctgctcatccgggggagagtcctgaactccactcaggacccacttcttaagtttccattttgtatagttagatgttgaaatggaggcttgctctgtcacccaggctggagtgcagtggcacaatctctgctcaactgcaacctttgcctcccgggtccctgttcaagcagttctcctgcctcagcctcgtgagtagctgggactacaggcacacgccaccacgcccggctaatttttgtattttagtagagacggggtttcgccatgttggccaggctggtctcgaactcctgacctgaagtgatttgcccgcctcggcctcccaaagtgctgggattacaggcgtgcgtcaccacacccagctggaaaaaaaaaagactttattttcacctgaaattcattaatttccacttgaaattccacctgcagttgtagcaggacctgacacttgggccccatggaaatcacaggtattgcctgacacagtggttcatgcccatagtgccagcactttgagatgccaaggtgggaggatcacttgagcccaggagttcgagatcagcctgggtgacagagcaagaccccgtctctaaaaaaaatttttttttttttttcaagacagagtcttgctctgtcgcccaggctggagtgcagtggtgcgatctcggctcactgcaagctccgcctcccaagttaacaccattctcctgcctcagcctcccgagtagctgggactacaggccccgccaccacgcccggctaatttcttgtatttttagtagagatggagtttcaccgtgttagccaggatggtctcgatctcctgacctcatgatctgcccgccttggcctcccaaagtgctgggattacaggtgtgagccaccacacccggattacaaaaactttttagataattatctgggcgacctgcctgaccaacatggagaaaccctgtctctactaaaaatacaaaattagccggacatggtggcgcatgcctgtaatcccagctacttgggaggctgaggcaggagaatcatttgaacccaggaagcagaggttgcggtaagccgagatcatgccactgcactccggtctgggagtgcactccaacaagaaggagtttcgctctttttgcccaggctggagtgcagtggtgggatctcagctcaccgcaacctccacctcccgggttcaggcgattctcctgcctcagcctcccaaggagtagctgggattataggtatgcatcgtcacacccggctacttttgtatttttagtagaggcaggtttccaccatgttggccaggctggtcttgaactcaagtgatctgccctctttggcctccttctcaggaaaaaaaaaaaatcacaggtatttacaggccattccaagtgccaaaagattgtttttgctcatggtgacttcagtatcacagatgttaggagacttgctgctatatgttaagaaagaagcacaaatgttgctgtagcccaaacttttttcctcatgtttcattgcatttcagcttaattggtttccctggtattcctatgtattttgtggagtgcttttaaaatcataagttggagtagaggtctttctgtgggcttcaccagactgccgagatcagggtcgaaacaggtgaggaccccttctctggagagagtctcctttctcctctaagaggaaaggttttgagatcttttgtccattttcccaccttagcacttcatcagccttaaaagaagctggaatttttttttttttttttggagatgggatctcgatatgttgcccaggctggtcttgaaccccttggctcaagcgatcctccagcctcagcctcccaaagtgctgggattcgaggcatgagccaccgagcccaccgtgcagatggatgtttttgtgcatgcttttgatgaatgctttctctctctcagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccagtaagtgaccagcacccaggtgcagttcactggggaggggtcacagacctctgaggtggaccctcacatggcccccatcctccctgggcttcttccctttgtccctggcatgcttgtccctagcccggaggaacatgtggagcccactgtctccaaggcaagagtccagcatggctgctggtgcctccattgccctctccccaccaccgcagagcaggtcggcctctgcctgactccctggtctcctgcagcccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagagggtgagacttgggggctggggcggctggtggtctggcgggagagatgtcactgagggcctgaaggggagaggcaggggctgtgaagttgggtaccccggaagtgtgaggggctaaggctttgggggcaagaggcagaaagagggcaatggctgggcgcagtggctcacgcctgtaatcccagcactttcagaggctgagacaggcggatcacttgagccctggagttcaagaccagcctgggtaacataggaagatctctctacaaaaaataaaaatattagccaggcgaggtggtgcatgcctgtggtcccagctactcaagaggctgaggcaggaggattgcttgagcccaggagtcggaggctgcagtgagctatgatcgcaccgctgcatgccagcctgggtgacagagcagtgtgagatcctctctcaaaataaatgaataagaaagagagggtgaggagctcgtaaagctgggctggagagttaagtacaggaaggcccccagtgggactggggccagagagaatcagaaggaattctcgaaacagccagggggaaattgagacaagtgtagccagcagaggaagtgttggaaaagataagggacatggccaggctgatcacaaggtcaggagttcaagactagcctggccaacgtggtgaaaccccatgtctactaaaaataaaaaaattagccaggcatggtggtgggcacctgtaatccacttgggaagcaaccagaagaattgcttgaacccaggaggcggaggttgcagtaagctgagactgcgccactgcactccagcctgggtgatagagcacgactccgtctcgaaaaaaaaaattttttttaagttaagggacagagctaccatgcacaagggttccctgtgtctctgcctctcacagtacctcctgcagtgtctgacatccgggtgacgcggtcctcacccagcagcttgagcctggcctgggctgttccccgggcacccagtggggctgtgctggactacgaggtcaaataccatgagaaggtaaggccatcccccagccctggggtgggtgggcaatgggttgtgctctcctggctgggacacctgggttgcaggcacctggcaggcatttgaattccagctctgccatggattccctgggcagccttgggtaagccccttggcctgtctgagcctcagactcttcatctataaaatagttactgtaatagttaccagcagctggacacagtggctgaggttgggtgcggtggctcacgcctgtaataccaagcactttgggaggctgaggcgggcagaatgcttgagcctaggagtttgagaccagcctgggcaacatggtgaaacttcatctctataaaaaacttaaaatgggccgggcgcggtagcttacgcctgtaatcccagcactttgggaggccgaggtgggcggatcacaaggtcaggagtatcgagaccatcctggctaacacggtgaaaccccatctctactaaaaatacaaaaaattagccaggcgcggtggcaggcgcctgtagtcccagctactcgggaggctgaggcaggagaatggcgtgaacccaggaggcggagcttgcagtgagccgagatagcgccactgcagtccggcctgggcgaaagaacaagactctgtctccaaaaaaaaaaaaaaaaaaaaaaaacgcaaaaaatacttaaaatgaaaaaaattagactgggcacagtggctcatgcctgtaatcccggcactttgggaggccgaggtgggtagaacacctggggtgaagagttcgagaccagcctggccaacaaggtgaaatccccgtctctactacaaatagcaaaatcagctgagtgtgttggcgggcccctgtaatcccagctactcaggaggctgagacaggagaatcactggaacccaagtgattctcgacttgaggtcgaggctgcagtgagtcgtgtttgcaccattgcattccagcctgagaaagtgagaccttgtcttaaaaaaaaggaatgatattatgaatacagcacatggcttgcatgcgtaagttctcccaaaggcctcaccagttgcaaggcaggctagtgatgggagtggagggcgagggaaggaggcaggaagagcaacaggaacttgggttcccgggtgacggccaccccactacctctcccggacagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatggtgagcctggggaagggggtgagggtgggggttggaaagacccccaaagttcctgggaagaccccaggtctccaaagtcccatcatcttttttttttttttttttttttgagatggagtcttgctctgtccctcaggctggagtgcagtggcaccatctccgctcactgcaacctccgcctcccggattcaagccattctcctgcctcagcctcccgagtagctgggattacaggcgcctgccaccgcgcctggccgattttttgtatttttagtagagacggggcttcaccgcgttggccaggctggtctcgaactcctgaccttgtgattcgcccgcctcggcctcccgaagtgctgggattacaggcatgagccactgcacccggtcaaagtcctatcttcatgtccttcttcctgtggatcacatggcatgccctagagaggagagaacgtaagatgtcgaaaccaaaaccaacagctgagttttgtgaagtctggcctgcttcactctgtacccccaggctggagcgcagttgctcgatcaaagctcactgcacagccaggcacagtggctcaccctgtaaccccagcactttgggaggctgaagcaggaggatcacttgaggtcaggagttcgagaccagtctgaccagcatggtgaaaccgcgtctctactaaaaatatagaagttagctgagcgtggtggtgcacacctgtaatcccagctactcgggaggctgaggcaggagaatcgcttgaacctgggaggtggaggttgcagtgagctgagattgtgccagtgcactccagcctgggcaacagagcaagactctgtctcaaaaaaaaaaaagctcaccgcaggcttgacttttagcaacaacctgacccctgagctccccattccccatccaacaaaatgggaatatcatgaagcttcctgcagggctttgaggattggaggtaacaggttatttttaatatgctaggccagtggctttcttttttctttcacatttttttttttgagacggagtctcactctgttgcccaggctggagtgcggtggcgcgatctcagctcaccgcaagctccacctcctggtctcgatctgctgacctcctgatccacccgcctcggcttcccgaaatgctgggactgctggcgtgagccaccacgcccggcctaactttttcttttttttaagagacacggtcttttttatcacccaggctggagtgcggtggcaccatcatagctcattgcagcctacaactcccgagctcaaccaatccttccaccttagcctcccaagtagctggggctataggcatgtgctaccgtgctcaactaaatttttttttatgttttgttgagacagtttccctatgttgcccaggctggtctcaaattcctgacctcgagcaatcctcccgcatcggcctcccaaagtgctgggattacaggcatgagccgccacacccagcattggaccagtggctttctaaaccttgtaattttctgtaatagctttactgaaatacagttcccctgccatacaatttgcctgttcaaagtgtacaatcgatgacttttgatacattcacagaattgtgcagtcaccaccacaagtaattttgggacattttcagcaccctcaaaagagaccctatagcccttagccatcaccccccacccagatctttctgttgccttagtccctggcaagcactaacccactttctgtcttgaaatcttccagtgtggtcttttgtgactgttcaccgagcagaatgttttcaaggtttatgtatgttgtagtatatatccgtgggtttttttggttgtggtttgttttttgtttgttttggaaacagggtctcgctctgtcacccaggctggagtgcagtggttcaattacagctcactgcagcctcaacctcccaggctcaagtgatcctcccacctcagcctcccaagcagctgggactgtaggcatgagccaccatgcccagctaattttttttggtattttttgtaaagacagggtttcaccatgtttcccaggctggtctcgaactcctgagctcaggcaatccacccacctcagcctcccaaagtgctgtgattacaggcatgagccactggacctggcctgttttttgtttttgttttgaacacacgattttgctttgtcacccaggctggaatgtaatggtctgatcatagtgcattgcagcctcaaactcctgggctcaagcgatcctcctacctcagcctcctgagtatctgggaccacacgtgctcaccaccatgcttggctaattattattattttttgatagagacggggtcttgctatgtttcccaggctggtcttgaacacctggcctcacacaatcctcccacctcagtatctcagagtgctgggattacaggcatgagccactgctcctggccaatatttcatttctttttatggagacgtaataatcagttgtatggaaatagctgattttgttttttattgtatcttttggtgaacatttcaattgtatcgactttttggataaaaacctgaaaatgtttcacctttagaacgtttcattgaatggagatttttttgtggactctggtatttatactagaaccaaatcaaaaccactctggcggctgggcatgcctaggctggtttgagactagcctgtccaacctggtgaaagcccatctctactaaaaatacacaaattagccgagcatggtggtacacacctgtaatcccagctactcaggaggctgaggcaggagaatcgcagaacccgggaggcggagattgcagtgagctgagattgcgccactgcactccagcctgggcgacagagtgagactgcgtctcaaaaaaacaaacaaaaaattactctggcagtaagaaaagatttcgaaacttcctcccttgccctgaggtacttcagaggagcctgctggcccctgggggagagtttgaaacccactgtttgttccctgaccttgcctgcttgtgtcctctccctccacctgtcccctgtactggggacctgttctcaggagatcacagttcattgctcaaagccggggctggggcctcctacaggaccatcagtttctcctgatcagcagcctttccttccgcagagagcgagggctggcgggagcagctggccctgattgcgggcacggcagtcgtgggtgtggtcctggtcctggtggtcattgtggtcgcagttctctgcctcaggtaagggctctgacacccagaggcccctggaagccctcagttgatggccacctgcctgggtgctacaggacaagcctttctggctgtccccagcctctttttacttgaaatcttctccaatccctgctccttcctttggtgtgtgtgcctcataaagatgtgtgactcagtttaccttttgttcctttcccatcggctacaggaagcagagcaatgggagagaagcagaatattcggacaaacacggacagtatctcatcggacatggtgggttgccctaatttgatgggaataggggcttggggccgggtgtggtggctcctatctataatcccagcactttgggaggcagaggtgggcagatcacttgaggtcaggagttcgagaccagcctggccaacatgttgaaactccatctctataaaaaatacatcagtcagccaggcatggtggtgggcacctgtaatcccagctactcaggaggctgaggcagaagaatcattttaacccgggaggcggagattgcagtgagccaagatcgcgccactgcgctccaggcctgggtgacagagcgagactccatctcaggaaaaaaaaaaaaaaaaaaaaaaaccacggagacaggggtttggggctaaaagctatgagccgagcctccgagtccagtgggagttaattcccagctgacggggccctgcctgatttctcaggtactaaggtctacatcgaccccttcacttatgaagaccctaatgaggctgtgagggaatttgcaaaagagatcgatgtctcctacgtcaagattgaagaggtgattggtgcaggtgagagccgaaggctgcccgggcacctgggaacgaagcgggggtgggcagggccacactggagcgggagagctgatgacctctgcgtccttgtttgaaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcgggtgagcaccctccctggcttctgcggccacccggagttcccacttacacccagaggccacttgggttaagaagccaggacagacagtgggtcccaggtcacctcctccagccttttcctcttgggctaagccctggtcctctgccttttcttttttttaagacagagcctcgctctgtcgcccaggctggagtgcagtggcgcgatctcggctcattgctgtctccacctccagggttcaagcgattctcctgcctcagtctcccaagtagctggtactataggcatgcaccaccatgctgactaatttttgtatttttagtagacacagggtttcaccatgtaggccaggctggtatcaaactcctgacctcaagtgatctccccacctcagcctcccaaagtgctggtattacaggtgtgaggcaccacgcctggccagccctctgcctttaattttccctctgggaaaggctgggctcctgggaccttcctttcccactgccccatacagctgaaggttgtcattccttctttttttttttaattttgttttaattgaattttttttttttgagatggagtttcactcttgttgcccaggccggagtgcaatggcaagatcttggctcaccgcaacctccgcctcccaggttcaagcgattctcctgccttagcctccccagtagctgggattataggcatgtgccaccacgcttgactaattttgtatttttagtagagacgggggtttctctgtgttggtcaggctggtctcgaactcccgacctcaggtgatccgcctgcctcggcctcccaaagtgctgggattacagacgtgagccaccgcgcccggccaattttttttttttttttttaagacagagtctcactctgtcctctaggctggagtgcagtggtgcattcatagctcactgtagccttgacctcctgggctcaagtgatcctcccgcctcagcctcctgagtagctggaactacactcatgtaccaccatgctcagcaaatttttaaaattttttgtagagacaggatctcgataggttgcccaggctggtctgaactcctggcctcaagcgagcctccctcctcagcctcccacagcactgggattgcaggcatgagccactgtgcctggcctgtcattccttcttttgacaaatatttactgagtgctttctacgcaccggtcatcctcccagtccccaggaataaagctatacacacggcaaactggatttctcctcttggggagcagagggtctaatggggcagggggactgaaaattagcaagtaaatagacaggctttttaaaaaagtaaacaaatcatttcaaatgtgaaaaaaagcaaacggggtccttcatgcagatgtggctagagaggaaagagaactgcttaatttatttggtcactttaccagattttactgactttttttttttttttaactttattaagcttttcttttttcttgagatggagtttccatctgtcacccaggctggagtgcagtggtgcgttcttggctcaccgcaacgtccacctcctgggttcaagtgattctcctgcctcagcctcctgagtagcttggaattgcatggcatgcaccaccatacccagctgatgtttgtatttttagtagagacagggtttcatcatgttgcccaggctggtcttgaactcctgggctcaagtgatccacccatctcggcccctcaaagtgctgggattacaggcatgagccaccatgcctggcctaggcatctttttaaaaaaatcaaaacatttttctatgtagcaaaataacattgcattgaacagagttatagcgattccctagcgtcattgaatacccagttgattttcacgtttctctagttgttctaaagatgtccttcactgctgctttattccaaccaggatccagttcaagaccgggctttgtacctggttattatatatattttatttatttattttagaaacaaggtcttgccctttcgcccagtttagagtgcagtggtgcaatcatagctcactgcagcctccaaactccttggctcaggtgatcctcctgcctcagcctcctgggtagctggaactacaggtgcacaccaccacacctggctaatttttaaattttttacggagatgggggtctcgctatgttgcccaggctggtctcaaactcctggactcaagcgatcctccctccttaacctctcaaagtgctgggattacaggcgtgagccaccacgcctgctgattattatattttcgagcctctctaaatcttgagcagttcctcatgatgacactgacacactgaagggttaggtcccttgtccgcctgaatgtcttgatttctggatttatgaaattcttcttatgggatcatttagcttgtctctctgtatttcctgtaagagaagctctatctgatgtggggtttttttggttttgtttgtttgttttttgagatggagtcctgctgtcgcccaggctggagtgcagtggcacaatctcggctcactgcaacctccgcctcctgggttcaagagattcttctgcctcagcctcctgagtagctgggactacaggcgagtgccaccatgcccagctaatttttgtatttttagtagagacagggtttcaccatattggccaggatggtctcgaacttctgacctcgtgatctgcccaccacctcagcctcccacagtgctgggattacaggcatgagccactatgcccggctaatttttgtatttttagtagagacagggcttcgccatgttggccaggctgatctgaaacccctggcctcaagccatccaccctccttggcctcccaaagtgctgggattaaacgcgtgagccaccgtgcctggtcgaagagacagaaagggtcttaaaggttcagtgacacacacctgtaatcccagcactttgggaagctgaggctggtggatcactcgaggccaggagttagagatcaccctgggcaacatggtgaaaccccgtctctacacaaaatacaaaaatgggcagagcatgatggtgcatatctgtagtcccagctactcgggaggctgaggcgggaggatcacttaagcctgggagatcgaggctgtagtgagccatcattgcactactgcattccagcctgggcgatcccatctcttaaaaagagagagagatgggaagaccagcacaggtgaaactggtgaacagaggagagatggtagatgctgcattgggcagtgtgacgggaacccgctggagggctttggcaggagagtagtttaagaggatcccagctgggcacagtggctcacacttgtgatcccagcacttggggaggccggggcaggtggatcacttgaggtcaggagttcgagaccagcctggccaacatggtgaaaccctgtctgtactaaaaatacaaaaaccagccaggcatggtggtgcacccctgtaatcccagctactcaggagactaagacaggagaatcgcttgaactcaggaggcagaggttgcagtgagccaagatcacgccactttactccagcctgggcagtagagcgagactccatctcaaaaaaataaataaataaaaagacctctttgctgggtgctagggagcaagagcaggagctgggagaggcctgcagcagaagcctgttgccagcatccaggccgtggggtgaagggaagggtttggatttgggacatgtcttggaagcatcaccagcagaacttgctgatggattggaagtggctggtgagggagaaaagggggtcaaaggaaactctgaggtctataccctgaccatctggcaagtggtggtgttgccacaaactgagcggggagtagggcaggtgcaggtctggaggatggattcaaaattcagtttttggagtctatgtccctggttctgtagggctgcagatggtctgccaaatcttagcggaacccagaatacgggatttgtttactgtctgtgacttgttggtttccctggtgagagcaaactctttaaaggtcaaggttgggcttcagaccttggtttttgcaccgatcattggtcatactgcagttcctcactcttctcttgcaaatccatacacagctagtccaagagagctgaacagctttgtggttggatcagcaccaatgtatctccacctgtagacgggttgctcaggtgactcatgcctgtaatcccagcaccttgggaggccaaggtgggaagattgcttgaggccaggagttggagacaagcctgggaaacacagtgagaccccatatctaccaaaaaaacccctttgttttaattagccaggtgcagtggtgtgcacctatagtcccagctactaaggaggctgaggcagaaggatcatttgagcccaggagtttaaggctgcggtgaaccatgatcgtgccactgcactccaacctgggggaaagaaagagaccttgtctctaaaaaaactaaaaaacagaaaagcatttgttgagtatttcctgggtataaagcagtgtaccaggttaaatggaaggaaaagttgaaataatttttcaactcataatccgattgggagagactgaatgcttaccattgaagcaggaaccattgtaagcaatgtgttgtgatactgtagcaagagctgagaaaacttgggaaaagagaaaggaggaaggctcacctgagggagttggggggcttgccctacaggtgagttgtgaggtgggtctggaagtgacagatgcagtttaggaagtggacgggaggctgggtacggtgactcaacatctgtaatcccagtgctttgggagacccaggcggaaggatcgcttcaggccaggagttaaagaccagcctgggcaacatagtgggaacctatctctactaaaaattaaaaaattatccaggcataatggcacatgcctattgttccagctactcaggaggcttgcctgagcccaggaggttgaggctgcagtgagctatgatggcaccactgcactccagcctgggcgacagaacaagaccctgtctctaaaaaaaaaagatgtggatgggagggggaacggtgggtgggctgtcctcaccaagcccccaccctatctgctctccagctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctggtaatgctgggggtaatactgggtgtgagcttcttagggccaggtgggcagggcaggttggaaaggtgggaggctgagggtttggcagccctgctccagggagaggatacaggagcaggctgtgggtggggggacagtcagctccaggaagccgacttccagatgtctaggaaaataacagttggataacctgggcaacatagcaagaccccatctctacaaaaaaattaaaagattagccaggcgcagtggcatgcacctgtagtcccagctacttgggaggttgaggcaggaggattgcttaagcccaggagttggaggctgcagtgagctatgaatgtgccactgtactgcagactgggcgacagagcaagaccctgtctcaaaagaacagtggccaggtgtggtggctcacgcctgtaaatccagcactttgggaggctgaggcaggaggatcgcctgaggtcaggagttcgagaccagcctggccaacatgggaaaaccctgtcgctactaaaaatacaaaattagctgagggtggtggtacacgcctgtaatccgagctactcaggaggctgaggtaggagaaccagttgaacccgggaggcggagtttcagtgagccaagatcgcaccactgcactccaacctgggcaaacagagttggagagtaggaggcttggggcctgagctagggggaaaaagcagaggcaggtgggggactggggggcagtgtgctgggtctggtgagtccctcagtgagtcccccagctcaccttttctcctttttctgcagggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtaagtgtcccgtggtcctaccaagctttcctcgagtgttctctcacctgggatttggggtgaagggtgggttcccagagagtcatcactgctgggttcttgagaccatggagatgacaaaaaggagaattgatctttgtatcaaagagttgagatacagggccaggcctagtggctcaagcctgtaatcccagcactttgggaggccaaggtgggcagatcacctaaggttaggagttcaagaccagcctggccaacatggtgaaaccccgtctctaaaaaaatacaaaaaattagcccagcatgatgggcgggtgcctgtaatcccagctactcaggaggctgagacaggataatcgcttgaacccaggaacagaggttgcagtgagctgagatcacgccattgctttccagcctgggcaactgagcgagactctgtcttaataaataaataaaagagttgggtacagcatatttgggtcgcagaaggatgcagagatggagggcagggttgagaggtaacatgtctgtatcatagcccaagagctgctggggccttcagccacagagagcttcaactccggctaggaggattcctggatctgttattttttggggggctgtggctcctatcctaccatcttccaagtcaccatttcctgggcctgttagcatctttgcttttcctggacagcctcacccagagcttcttcccctctttccaggtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcgggtgaggactgcagagaatgggccctccttcccgctctctgcccccactccttgcccagaagtgtccgttcattggtgttgggtgggagggcctctgtccgcctctgcaaggctgggttccacctcctcccccggacctgggcctggtactcagcattcctccccatccttgccccctagggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgagtaagcagtggcaggagctggagtggggctgggagagcggggcagctggagtcaggcccacggggtctccaggggcttttggggtcagcttcgggtgccaatgctgtcttcttgcactgcgctcatgccatgcctagaagggccccagaggagcagtcacagccccatggagctgaggacccaaggactctttggggccagcctgcccgcctcacctcctcctgccatcacagccctgggccatcgcgcttccgcctctcacttctagctatctttgtgcatctatctgcattccaggcccggctctcacggtaacaatgtgtcaactcgggttctctttttccaaccataaaaggagaagattgggctaggttttggagatcctcttcagcttttatgtgaaatggttttatgattccttgcctcccaaaggctgcgtatccccacttggcctttgtctgctactccccctttctgccttcccgttcctctcccaagatctcctctcaccccaggttgaataacagaaatagaaggaatagaaatctgaaggccgggcatggtggctcatgcctgtaatgccagcactttgggaggccgaggtgggcagatcacttgaggttaggagttcgagaccattgtggacaacttggtgaaaccttatgtctactaaaaatacaaaaattagctgggcatggtggtgcgtgcctgtaataccagctactgaggaggctgaggcaggagaatcgcttgaacccgggaggtggaggttgcagtgagccgagatcgcaccactgcactccagcctggatgacagagtgaaattccatctcaaaaaaaaaaaaaaaaaaaaaaaagaaatgtgaaggccaggtggtggctcacgcctgtaatctcagcactttgggaggctcaggtggaccgattgcttgagcccaggagtttgagagcagcctggccaaaatagcaaaaccccatctctacaaaacaaaaacaaaaaaattagctgggcatggtggtgcgtgcctgtggtcccagctactcaggaggctagagccagagggtctcaggccagtctgcccctgccccacggggcctgggcacatccctccctaattcttcccagcctctctctgacccagggggcctcctctcccttttttccccttatctcagcctccagccatcagcaacctcctcttcctctccacccagctcttcctctcccacttcggccttttctttctcacactccatttccctctacggcaatctgtgcagcctcttcccccagtctcattttgcgggcttttctctcttttctttccttccctggcacccaagccaaaggccctgcctctggcctccagccctacccccttctgcggttgcacagaaggatggctgcccagctcttaaaaaaactgcccgggaactgttgacatctgttctccctcccccgctggcttttctgattggcttacaatcctgaggctaggaccgtctcaggagccaagagaggagagcggccacagggaacctagggtctcaccaagctctcctttccttctgcagggacctgctccgaatcggagtcactctggcgggacaccagaagaaaatcttggccagtgtccagcacatgaagtcccaggccaagccgggaaccccgggtgggacaggaggaccggccccgcagtactgacctgcaggaactccccaccccagggacaccgcctccccattttccggggcagagtggggactcacagaggcccccagccctgtgccccgctggattgcactttgagcccgtggggtgaggagttggcaatttggagagacaggatttgggggttctgccataataggaggggaaaatcaccccccagccacctcggggaactccagaccaagggtgagggcgcctttccctcaggactgggtgtgaccagaggaaaaggaagtgcccaacatctcccagcctccccaggtgcccccctcaccttgatgggtgcgttcccgcagaccaaagagagtgtgactcccttgccagctccagagtgggggggctgtcccagggggcaagaaggggtgtcagggcccagtgacaaaatcattggggtttgtagtcccaacttgctgctgtcaccaccaaactcaatcatttttttcccttgtaaatgcccctcccccagctgctgccttcatattgaaggtttttgagttttgtttttggtcttaatttttctccccgttccctttttgtttcttcgttttgtttttctaccgtccttgtcataactttgtgttggagggaacctgtttcactatggcctcctttgcccaagttgaaacaggggcccatcatcatgtctgtttccagaacagtgccttggtcatcccacatccccggaccccgcctgggacccccaagctgtgtcctatgaaggggtgtggggtgaggtagtgaaaagggcggtagttggtggtggaacccagaaacggacgccggtgcttggaggggttcttaaattatatttaaaaaagtaactttttgtataaataaaagaaaatgggacgtgtcccagctccaggggtgatgggggtgatggactagatttctaaggagagtggggctgggtagggagggctttgtggctgaccgagaggtgtcagaggtctggaggctgcagggctgtaggggctggaacttggttatcagccccagggtatgtttgaggtggtggggtgggggccgagcgagatgaatcattcgcagctgcttctaacgtctc

SEQ ID NO: 328 - DNA, 4235 bp, Homo sapiens
ctcggcccggcggcgcgagcagagccactccagggagggggggagaccgcgagcggccggctcagcccccgccacccggggcgggaccccgaggccccggagggaccccaactccagccacgtcttgctgcgcgcccgcccggcgcggccactgccagcacgctccgggcccgccgcccgcgcgcgcggcacagacgcggggccacacttggcgccgccgcccggtgccccgcacgctcgcatgggcccgcgctgagggccccgacgaggagtcccgcgcggagtatcggcgtccacccgcccagggagagtcagacctgggggggcgagggccccccaaactcagttcggatcctacccgagtgaggcggcgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctgattgcgggcacggcagtcgtgggtgtggtcctggtcctggtggtcattgtggtcgcagttctctgcctcaggaagcagagcaatgggagagaagcagaatattcggacaaacacggacagtatctcatcggacatggtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcggctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctgggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcggggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgaggacctgctccgaatcggagtcactctggcgggacaccagaagaaaatcttggccagtgtccagcacatgaagtcccaggccaagccgggaaccccgggtgggacaggaggaccggccccgcagtactgacctgcaggaactccccaccccagggacaccgcctccccattttccggggcagagtggggactcacagaggcccccagccctgtgccccgctggattgcactttgagcccgtggggtgaggagttggcaatttggagagacaggatttgggggttctgccataataggaggggaaaatcaccccccagccacctcggggaactccagaccaagggtgagggcgcctttccctcaggactgggtgtgaccagaggaaaaggaagtgcccaacatctcccagcctccccaggtgcccccctcaccttgatgggtgcgttcccgcagaccaaagagagtgtgactcccttgccagctccagagtgggggggctgtcccagggggcaagaaggggtgtcagggcccagtgacaaaatcattggggtttgtagtcccaacttgctgctgtcaccaccaaactcaatcatttttttcccttgtaaatgcccctcccccagctgctgccttcatattgaaggtttttgagttttgtttttggtcttaatttttctccccgttccctttttgtttcttcgttttgtttttctaccgtccttgtcataactttgtgttggagggaacctgtttcactatggcctcctttgcccaagttgaaacaggggcccatcatcatgtctgtttccagaacagtgccttggtcatcccacatccccggaccccgcctgggacccccaagctgtgtcctatgaaggggtgtggggtgaggtagtgaaaagggcggtagttggtggtggaacccagaaacggacgccggtgcttggaggggttcttaaattatatttaaaaaagtaactttttgtataaataaaagaaaatgggacgtgtcccagctccaggggt

SEQ ID NO: 329 - DNA, 43948 bp, Homo sapiens
gcgcctcggagctgcctgcgggcgcacgccgtcttccccgccagtctgccccggaggattgggggtcccagcctgcgtcccgtcagtcccttcttggcccggagtgcgcggagctgggagtggcttcgccatggctgtgagaagggactccgtgtggaagtactgctggggtgttttgatggttttatgcagaactgcgatttccaaatcgatagttttagagcctatctattggaattcctcgaactccaagtaagtggcgtccgcgatccccctatgtccccgccccggggtccgccgcgccgtccgggcgggaggaggggtcagtccgcggggcctcggagcctgtttctggaacctcggttccccgtcccccacccccaacccccgccccatttcactaggtggagactcctcgctcggctttccaacccgagccccgctggaacggacggtctctccgcctttcctcccccgaacgctcccaggcgctaaaagctactatcggctcgggtgtcaagtccgggaaggtgtccgatggcgatacctgaccctctcctgttttcgaggacgaaggacatggccacaatctaggctggccggcacgcggggactggtgggctctggagagaggcggagatgctgcattcgcggggagcgcgggcggcgtggtccggggcccgcgggcgggcgaccggggtggcaggacgctggcagcgaagcgcgttctggagaggggagcctggagtcgctacgctgcccgcagagccctggagccggggcgccttggcaccgcgccgccagcccgagggtgcgcggggagctcgcctgcttcgcaggagaactcgggcgtcgagccctttcctccgcgccggggagacgggccttaggcttctccctgagggcccgccgcacctcggcctcccgcttcgttcataagccggtagccccggagtatgcggtctcgatggccgacctgattgtaatgcacttcctataaaagcttagggccctgcccagtcgacactgctcctgaagccttctccctcgggaccctggtaggaatgggatccttaggatcagatttgctcttaccggactctacagccgggagcgagccaggccttgtggagagtaactttcagtttgggccaccagagtgcattcagaatttagaaaatcccatccatccctaaatctgtgtggtcataactcgtagtcatctgggtattcagtactgtgtatccccttatttcgaatcacagccaaaacatattttacagaatcttggaattgtagtctcgggaaacttggagaagaagtatgcagacattagctggtttctggagaaaacgtttgagatcagaagcaaaatcaatggcctaattgaagttgagcaagttgggcctggttttaggagaaaagaaatgggggattgatttagaaatcacgtcttaaaggagtgtgtccattctcttaaaagtgtcaaatttcaaattcactaacatgttaaccaagaatcccttcatgaaaagggcgaaaacgtcggttacaaatcggtttaaacaaatgtttgtatgatgctagaaggcactttcaacaccgctcatacggagaagttacttagctctgcctccttccatgtagtctgctcttgcatggattatatttttaatgtaaattgttgtatttgctgatgaagtactggcggcggcatctttgcatcgatgccggctcgggaggcgccaggtggtgccggaaggagccgggctaggacctcgcgcagcagcgggtcccggagtccgggagaggcgggcgggcgggcgaggcggtcgcggggagcccgcggcgccgctgcccgcccggtgcctccagaggtcactcttccatgcggaatcgcgcagcgccaggcctcgcccctcccccaggccgcctgctccagccactctgcactttcactgaccggttctctttgaggctgtttttttttttcttatgaggatttaatatttctgtttaaatctagttgaaagcaattccgttagcctcttcagcgtttagttcggtgtgtgtatctttatctttgcgctatattaactattagtttgtgtgtatccggtaggagaattagaaatacctagttgggagaaaaagaaaagtagaacaatagttatttcaacctaaggtttagacgttaataacttctttttgtaatgtgtcgagatggggggtcctggggggaggtgacaggtactcaccactccccccccccattctgatgatgaagatgagtctgtctttccagctatgtccagacctgcgagggccctgcgtttctggaagcctgccgtttgcgcggttgaggttgctgctgctgtcttgtcctccacagcagcatttcttttaaaattctcctgataacggcctgcctggatgactggataatgtgtgcctggaaaaggtctcccttgcagctgaatgctagctccagagatcagaaagatttcttcctgtaggagccataggaaagagtcctctctaagtttttgagaatgcatacaaccccctgatgacagggggtcgctttccttggggaagttttatatttatttccagaggaaagtttgaatcggtaaatatgatgtggcaggaaggtaatcaaatgcattgaagtttcacatcagttcctatgaactgtggaacaattcatttgtaatgaagccgccatcagtaattagatttgtttcattcagaggtcagcttttttagcaggtggtcgacacagggagcatgcagcagctgtttggatacagggtccagaaaaccctttgtaaattcagcgtctccgtaactactttaatcacattgtcggctctcccgtccctgactgtatgtaataatggaaagatgtcctgcgtgctgaaacagtagctgccctgttaggttattcacattgctttgatacgttctggtagagttgggtccgttgtagccattttggttgtttaaagttttggttttttttttgttttttttttaattcagcagagaacagtaatgcctagcttccgtttttaacttaacacttcagtagaacattttcttccaagagggagattttggcctaagtaaagtagtgggctcttttttaaaaaaaaattaattttactttaatgtgagcaaatctgtattggtatggtgttctgcaatgcattacactgactttgaaaatttcgagtactaatgccttatgtctggggttaccattccctgtgcatcacatactagttagttaacatagcattttgcttttcccatgtaattttttccctatataatactggattcctgatactaattgacttgatacaaaagaatggctggatgatatccagataacgtataatacatgggcttcaccacaatcaggctctgaataaatacagacctgtcagagattgataaaataaactacaatggatagtgctgtttaaacagtccattcaataacatatataagccagcctgccttccattgtgtctgaaattcttatttttgtaggtaaacaaatgcacattcagcactgattgaatagccccttgaactatgctccacagtttgcgtttgggttaatcttgtcggttttaatatagagagaaaaaagctcaaagcaccaggggtggaattgttagtgctttcacatccacattcctcacattttgtcaggatgataaactgtaggtaatggactgtcgttgttctgcaggacaactgagccaggcagagcacaaagactaagctaaagcgatacctcacaacatgcttggtagccttcttttcagatgagaatttatttgagaatcatgtgtctagggactgcacatcttaacctcaacagttacagcttcaagccccagaaacaggagctggaggttaagatgatttgctaagcacctggttctaaatcttttacaaagcataagctgttgacgctggttctgccgacgcaaagacatgcagatgactccaacatttccagaggcttctgacttaagctaaagtgtgtggacaggtgaattcgccatgggcctggagaccagcttgctaaaaactatgtgtttgaatggttcctccagacagagtcagctgaagaacaattggtggatttatattaaaacctcttgtctgtaaacttactgaggtgcatccttcggttggtggatcagtgagataattgccttcagatggacattgcaactggagcaactaaatccttgctgtctttccttcctctgaaatcttccaggtagctcccgagagcttcagtatgacaccaaacttcgggcgacgttttagagtgcgttcacctaatgggaaactattcgagatcccagcgtgactgcagtaatgcgtcataggaatgggagtggcaggggaaaaggaaatacagattgtagaccctaataaaaaaatttttaggaaagatatttctttaacgttttatgagaacttcattcttaaaatacttaattgcaaattagacaaatagaagtgctcttctaaggaaggtgattaaactggtcctcctatcagcctaatctctgcctgcctttgctgctgacataaagaacctgtttttcaggtcacttaatatacatctacatagatttgcttatgagctcaccctttgtgtagcggagtagagccttaaagaggagtgctcaactgtttaaaatattttgattaaaatatgcagaacccatagaactataagcttctagtcaggaattagctctttcagggaacagctccccccttctttttaaggggggaattagaaggaggctgggggaggaatataagaacagcaaagaaggaaggatagcaaatgggacatgttccgaacagcttggaaaaactcctgtggcttcattgtctctataaagccaaagaatacaaagacataagcaattcagcccttctcccatgatggaagatgtaaaccgttgacatgcctcccctgtttaacttgtttaattctcattttaaattcagcacgatactagccgtgtgaactctgaagatttctttagtaatccattttgtagttccgaatcaaaaacaaagtgaaagggtctgacacaatttgcttttatttttaggcaaatcaaccctggtcatagttaataaggggattacaactcagactaggtctttacagatgtgatgtaaatcaagggcagagtataaagaaactgatcccttttgattgaagtatagtaaaaaggcatagagaaactagcagcagtaatctgattgtatggcaataaaaccaccattttctgtctttcagataaaaataatgtggtaaatccatgcagttcataagatgtaaaggcagataaagggtgaagccatggcaacatatagattagcttgatgttagaaatgacacgtctctgaaaagggcgcgggacgaaggcccttgcctccaggctgttgggcattatgtgagaaccacacagacttggaaactgggattaggaagtatgaaagctctacttgtggtctgggatggctgaggcagtaaagaaaagctgctcagttcttgctcattggtggtggataatatggcaaaggtagatttcattgactgccttttttatagattgagattggggctgattaaaacttcagatcactgcagttgttagggcctgggagattttcctttttaactcctggcctaacagcagcagccgttctgtaggattaactgcacttcgcggtcgttgccttaatctatttgggcttcaggcagggacatgctgggaaggaacagagaccagaggggataggtagggctggggttatctgaaaagaaaacagagaccttttgatttcagccatcttttcagacccagctccctctcccgctgcatgggagaagcaaaggtaaacaggacacattgtccctctccctcagccacagagctcttctgtgagttttgtctttcccaccctggaaaaaaagataaaatacaatttttaaaaggggagggaggaatttagttttaattcaaatgagtagtaatccaatatgccaaaagcagtgggctctacctagatgtaattttactcgtaaatgtgagtcttaaactttgagttgaatggggcaggctgttagaggtggtgtaaattacaggattataaaaatgttagtgctgcccagccttaaagtcaaaaacagaaaaatctctgtgctgttgagtcttcccgccctctctcctgaacaaccttgtaagtaagctagacttttgtttttgccttccatactttccatttcagccattaaacaaaataagccattgaaaccacgattgggttccatgcagagtgacatccgcaatcgggtcaagccagaaggaaatacttgctcgattgccccctatttggcattacaggaaagtctccacactttggaagagtctgaactctcaagacattgaaaatgccaaaggctgcaaacaccctgtgtctttcttgatggagtgcatcttggtgtgttttacaaaggggaattcagtgctgtttttttgttgttgttgttgttttttttttttaaagagcagcatagggcccttctagactcttggattctgtgtctgacaaaaatggtcattaaatgagcaatattataatttagacccatttcactgattttgttccaaattctcaactgacttgagcatctgtttggggctgtagatacattgcccttgttgactgtttttctcgtttctatgggaattactgtagccattactatgtagctttcatagactcaaaacatttttaaagtattgcatataggctggccatatccagtgcctgttactttaccttctttttctaacttaatgcagcagtctgtattaacagatccatttcatttgtctagcttcatcagagagaggctaccccctgatttacaggctgctcacatccaagcaccttgcattctacacttgacagtgattgctaatggcccattcaactaaagtatttgcttgttaacagggaacagaacatgataaatgtccagcaagcttgctgcctccttcagcttttcaaacgcagactggtgcatatttatggcaggcaaatgacaaaagaaaaagctgaattgccctggcctccagctttctatcagaaacagggttaaagtgattaaagcaatcattcaagaaagccctgccgtttgtttactaaccttcatccaacatttagctttgtagtctacctgtgagaagatatttcagaagtattagagataaggaaggaggatctagcaaaccagtgaaaagagtaggtgaccagttataaaatgctttccatgcacattgaatgccaggcgaacctatttctgttattccagcagacaatcagcagtggctctagattattaacatattttcctttcatgtataaattcaaatatgtaattctagtccaaagcattctgtggctggtaagcacatacttgctgatttcaaataagaaaacatagcaagggaaagctccattaaacaagttgtttctgcccttagtaattctctaaacaagataggaagaaaaagtggacagtagtggagtattaatagtgtgctcttttcattctctaaagcacgagtaagtaagcgttcaaactactctgtggtgggcatacatttagagcgctgtgaatgaaccactgctgttctgccatacttaatttatttatattattatttttattttattgttgtttttatgtattattataattatttatttatattactaatttattttctcaatttaaatcctgttgcatccaattttaattacagtttttgtatctgccttcccatacttgctacccacgtccccattgccactgcggccttatccatgttttctgtgtacaccactctcgtatcaccccagaataattatgagtgctacccagacttttgaaaccactagagtcaacatgtttgtctttgaggaaagccaatgatgctttagcatttttggcaggggtggatgtgtgtttaagtggggtgggtgcagctccttattgtctgcctattctactgttgttcccaatccacattccctgcggggcacctaacctgtgtgcatagcaaagaatttccgaccttcagagccagaagtgtttctcaattgatctcttccagcctagggttatagctgatgaattataatccttgctctttccacacctttacctgggcttaccatggccctaaaacatttgcccagaatcagaattgtctcatgagtgagtggggcaaggcaaatcctgttccagaccagctgagaatgtacctagctgcagaagaagttagaaagtgtcatcttttacttatctaccagaactatattcgaggtacattttagatttaaaaaaaaagcaagttctcgtaggccttgaatcccccccttgctatgggaaaatggatcattattataatggactgtccagtaaagttcatgatttctcctagacatgttctctctctttatgacctagatcaagagtgatctctttaagtcttttcttcataatcccacagcactttgtacttagatgtacttagaaagaaccatatacacggtacgtcatgattgatatgcaagccttcaccactctacctgtcctaaaagtcagggacacaccttcttcatttcatcagtccctacttctatccagcattggcatccagtaagtattagtggaatggacagacaacccgaatttgtgctgatggcagtttaccctgttttaactgtcatccttctgctactagacatggatgagacctgagacgatgggactgctcagaggtccctggctcttgaactttagggcaccagaatcccctgcagggcttgagaaaacaggggtttctgggccccacccccagagttcctgattcctgaggtctggggtggggcttgaagatggacatgtttaacaagctcccaggtgacgctggcaactgctgcctcagggccatgctgagaaccctcgccctacacaaacctttctgggaaaacaactcaacattaaagctgtttggggatctctgaagaaatctgtagtccttgccttgttgggggagcatcagggatctaaccattgatggtggagtatttgttgttaattcagcaagcaactattaagtgttaggcctgttactcggctctaacaatacaaggcagagtgacctgtaccctcgagatttaaagtctaagtcctgtagagagaagcccaggtgggagcaagcacatttagagttaggtgcttggtgcaaggtggggacacagaagaagggaatggcatttgcctctggaggggtccggaaacagcctagggaggaggagcttgagtcttgaaatactgtgggcatctctaagcaaagtcacagtagacagctgaaataaagaaaatagtaagcaagccaaagaaacagtatttcagccaagggcagcgtgtgtctatcacgtccacctgtgaacacgtcccaggattctctgcatccggccattgctcaagacagatccctcacaggaacagctaagccactgatttcagctacctgttcacgtgagaattatcagtacctactgcttttcaaaatgagtatgatcatggataggtgaggcaattcagtttcgcagagacagtagggcaagtgccactgtagtttagttaagggcacatgctttagagtttggctatgtgagtccaatcccagtttagccatttattagctgggtagctttaggagcagtagccttagtgtctctcagttgtcccatctctataatagggacaataacataatagtgctgaataaaagagtaacaaaattttggtcaacatttaatgtatttaaagagctaagctccgtgattggcacaatgaaccaatcaatcaaacaccagttgttattaataaaagtcagttgaatatgtactgtgtgcctggccgtggttcaatttgcctttgcatacaaggaaaaaattaaaatactctgttaataaagactatagcataatactttcaccttaaacttcttgatgttaatttattttgtttacctgccaaacttctactcattccttatgactttctgctacatgaaacaccctttgtaattcttttgtcctattaaattaagttctctctcctctgctttcctgcttttggtgctttctaataacacttttaaccctggactttctcattcagctgtgcaactgtggactgagaggaggctctttgaattcattttgtatattctagtagagagtactgtgagcagttgggttgttgaatgaatacattaattcaacctggagggatgggcagtattgcattttttacattgatattacatgatatttagaaaactgcttaactggtggacgttgttttattaacagcattttgtgtatagcactcactatgtgccagctgctattctaactgcctgacaaatactcctgaaaccttcatggtaaccatatgagggaagcacttttaatatatccataataccaacggggagactgtggccaaattggttaattaacttagccaaagtcatattgaactaataagtggatttaaacccagctagtctggggccagggtccctcttttaatcttctgcctcctgcttatgctgttgcatggagtagtctttatcatataactaaattaagcatgcatttgcttaaagcagtgcatacatgatggatcaaaaagtttgtggtataattggtttaattctgtcattatccattttgatttatagtcactttcttatgatggtcgtgtagttttaaatggaacctttgaatctttgatataataaggttatgtcaaatcttgggtataataaggttatacccaatggaaacagaataatgatcagcccatttaaaggatgactggagagttattacaatacataatagtcatgcatatattgagtagtattcctttggtaacattttccttttaaaaattgtaacatttgattgttccttgttgggagaaaaggaggtcagatttttgaggggagatccatttggtgagatgctgagtgtgtgtcaagctaaggagatagtatgacatcttttttagagtctagtcacaattaaatgccattttattttggattttgggatccgtgccagcttccagcttgtcagagctgagaagactcaaatcaagtccaggcttatttctacagcaaactgggattctggcttcttgccggtggattcattcagtacagcccatctggcttttgatgttctgcaagtttggagccatttgttgaaggaagccaggcggtgaatattggtggtcctggggttctcttgactccaagtggtgccccttggtttgcattttcaccatgcttagcatctgcttacctggagaccatgcagccgccggccagaggtctccaacaaccaaatcttcatgccttttagaactcagagtccccagcacatcctccttcctcctccttgtccaattactttcatgcagttctcagtagctgcttgtttgaatcacttatagtatttaacttctagggtgtttttgggttttggtcaaggtaattccaggctgaatgtggtgactaagcaggaaataaatgggtcgtcctcaaagttacagtggagcgctgtttctattttcctaaggtacacagttgtgggggcgatccgtatggaagtcaggaacccagtctgattttgcttccttttgatggtagcagtacagacctggctgttttgtagcctgctttgtttttcttccttttcttccctaacttcacgggctgtggcaaagccctgagacgtgcaggaaaatgtctcctgtcatacgcccacagcagacctagccctgaccctcctctgaagcccaggaaggaggtatctgtgaagcagcctgcttgtaaagcaattgcacacagccttgtaaactgtgttactgggctgattatacttgattggcaaggtgaatctcttatagcaaaagagaacttggagagttttatctcatcttatgccttattaatttgttcattctttaattacacagccacctattgagcaccctatttatgcaaggtacctggtcgggggtcagagggagggtcccatggtaaacgagacagactcaatcctggaggagcaggaatggcagcccctcgctgggctgttggccccaccaaaagggaaaggtttcattttaataatacatgggtgaatcatttttgtcaataggcaaaattctttgtagttaaaaaaaaatatgatggtaggaaggaaagggatgggcagagggttaaaacaaaagatatgctctccctaactctagattgtagtattgttatgcttgtcactgtagctgaattccatttctttgagttttttcaatgccaaggcattccctgtatgacttacgtgagcctttcatctccgcgatttttcccattcaggtaaatgagcaaatggatttgaacactcatatctaaaacaagagagaaccagctggaaatgccctttgaatttctttctctatgtaaaccatttttctttctggtgcctcacctataaataacaggagttccaccttcctttatagactcttgctgaaagcatggtttggaacaagaccgtacaggtgcacacaaattacagttgggaaagaagcctgcagtgcatcttgtctctgaaggttatgaaatcctccttttagtaatggagctggcgtgatcaagccagcaggatgaaatttggcatttgtgagatcaccccccttctcacttgcccactgtacatagcatcccagccttactcttcaaatctccacattttttcttatctagctacaaaattcataggctgatttttttggggtgcgtgtgtggttttttttttgtttttttggtaaataaagacctgcatttttattttgatataggtggttgagttttgtctttaatttcatgacagagatttaactagtctcaacttttgaaaagacaacaatgatatttggggatcacacacttaaagttagatttctagatgattaataccaaagtagatgattttttagcctcagccatttataggtatgcccttctgtgaattttttatgacagtgaaaatcatggcacagataaaaattaaataaatacttctgttattttcctgaagaaaaaaaaaaaaagcttaaactatgagaatactgtctttgagcactttaaaataaaattgacttcagccagcaggattttgagcattacatcacaaataaaaaacaagattaacatcaaaaggagtcagttttcattcaattgtgcagcactgtgggctgtgaaatttaatattattttgactcatatgctaattgtagactgacagaggaaaatggattgtgtttaaataaaaggatacacagcatcacacgcagctgtatcaaatacaagttgaggtctttgggccaggaactgggggccctctagctctgttattgcagattcaagtttgacaaataaaactttcctttagactgtagtttaattactttttttcaaaggtatgcgtgatgaagaggcacaaatacacctcaccttgaagagttgctaaactggtttgtgtgccgatcagttcaccgtgtgtttgaatttctgtgcttctcatctttccttttcttgaaaagattttgcttgtcattggtgtgaattgtaccccccacccccacccatctagtctttgctctcagatttataacactttaatggttccaaattgtatagcctgctcttagaccccttttcttttccttgaataaatcaggttcatgttgcagacgatatttgttttaggaaagtgtgaaagaaggggcacctgtgaaaacacgcaattgttccaacacacatatacatccaaattaaagcagaaaatgtcaaagcctccaatcactaccttatttcttggaggtttaaagccgctgagaagatagtggtgccctcgctggaagttttaaggtaattactttttactctaagcagtagtatctggtaacctaattccgtataaacctgacaccctatcgctacaccccagtatttctctgatttcagaataagtctgcgtagaaacttgttctgatgttaaagtgcaaaagggggcagtaaagtgctatccacaaaaaaggaaaaacattttccaagtatttcttattactgcctgtgtctttcgtaggccctgcctttatttattcattttataacaaaactcttatgtttggggcattcagagaataccttattaagctgttgcagcaatctagcattaaatggaagacatgcaagactgaagatcctgcctgtttatgaagtgtgccatcaaattcacatgctcatgatgcagagtccttctttgggagtattcgtattcccaagtgcacagagcacttcggaaaggagccttggtctttggtgttaatgctctcctagctccgtatagatgtggcaggcccaaagtacatggtggggtgaagggtcaagggtttgggcttatccagagcagcgtgcatcctttgtcaggaggtgactggaaacaccagccaattacagcagaactgcagactgctcatctgcattcggaattgcagatgaaccagtttgtactcgacttctcttcttcactgtaggctttgacatttaattaaaaattaaagccttttatggaaaaagtacatgttttccaaaatggggtaaattcgaagtatacttgatacagaacactggcttgggaataaacctgtgatattacatgacttttggtttgcaactgctaggctgagcctctttgtaaagctgggatttagaatctttgaaatgtttgtacagttcaatgattaagcataaattgtatatattccctttttttcacttatttgagtaaacaagtttgttactacagcttctgtggactcagagatttatgtattaaataggccacaacttcaactaggataattttatttatctgcttgttagggaattgcatcaaaagtttaagtctgtaggcattaaatattttaaatgcttatttttaaagtcaattatgaaagatagcacaaagtttttctgaaactacattaaaaaaataatgttttaatcttatcacaaaagcattgactatttattgcaaagaaaacacagaaagctaaaaatcattctaagtccaccattcagtagcccaaagtggtctcaggtaaaggcggtgtgtgtgaccatttgtttatggttgtctccgtgcagtcagcaaaataaacagaacaacatgccatatattattgatgtgtatattttcaactgaaattagccatctgcttacaatgatcatatacactaatggtataattttgaaatgaaaagaaaaataaaataattctttgtggagagtaatgcgaattgacttatgaatctcgccctgcttggcagtttgctctagaggtagaagagctttatgtgtgggcctcctccccccccacacatttattctgctcacacttgcaccagcatccatgtcaggactcaccttgtcctgttacatgagtaacatggccctgattctcaagtgcatgataactgccataattacacataaatattaaatatttaaatagatctttacgtgtgtaatattaggtagaagtggctctggatcgaatctgatgctttttaaatagaagctttcccacaacatttccaagcactgtcatcgtgtctgtctcgatttggggtttacctggcctagttatctgtctgggtgtagaaactggtagttcctgtttgtatcttttttgttctgatctctttattctgtgtcagctaaatattcttgcagtcagttactaacatattaactcatccttgtttggaaactttggcatatccttccatggtttccttccgtggacctgtcgcgtctctcaggagagccaccaggtatattgtcacacatttcgcatgtattttcagagactacagcagcatcaagtggccccccagcgatttgggttttcttctcggttaatctacactctttggccaaccgtgagaaaacttgtaagaaggcatcagatgtttgtgctaaggtgcgtgtagtatggtcagaggaagaaagaagcagggaaaatggagtggccgtgggtgggaggggaagcagggagtgcaatttcgggttcactacacagctctccataaacttctccactgctggcttcccacggatcctcctattacactgggcaaagtgcagaaatagatcaggcgaccactgcctccgtccatttcccaggcaccctgtgagacccgataatgcaatacaggtcagcagaaaagtccagacttgacatcccaacgtgccatggtctggtctgtgaatgaaaatcacatgaggtgacctctgaactctaagtggctggtttatgttttcagtgtattaggcccgtgttttaaacaagcatgtgctcgtagtgtaggttaaaactttctgttgtcttcattaattatgctgtgttctagtctattaatattaaagaatattgtgttgcataatgactaatttttttattttttggagacggagtcttgctctgtcacccaggctggagtgcagtagtgcgatctcggctcactgcaacctccgcctctcggattcaagcaattctctgtctcagcctccgagtaactaggactacaggcgcccgccaccatgcccagctaagtgttgtatttttaatagagacggggttttaccatcttggccaggctggtcttgaactcctgacctcgtgatccacccgcctcagcctcccaaagtgctgggattataggcgtgagccaccacgcctggcaacataaggactattttttaaagtttttacaattatgactgtgaagttgaaatgtctaaattattagagatccagtttagattactaaatatttatgtctaattgagatgattagacttagccaaagtatccatgtagaagtattagagtctagattggtgaaaaacttgaaaaagcttggcttaagttcaataggtaatccaagagtaaaaacagattccaatatcagatcttttcaccatagtcatgttaagtttggaagccctacttgagtgtttccagttttttccacattatattgtgtctatatttgattcaaaggcagggcatctattgtcttgcttaggactgattcactgggaaaagccactggagttgcctatttccactcagtatgcctcactcttagagtagcttcccatggttcccaggcaggccctccagtgagaatgcaccaagccacacgccatggcctgggaagcagtcctgaacctggagattgtcttgatggaaaggaagaggcagccttcccctcccaggaagatagtagagagcctgctctgacttcgctcagggatggaactggtctggctcagttctctctcctgtgtgggacatgaatcactcttggtggtctttgctttttatttgggcttaaaatcagcagactttattaaatgacacctctctctaaccactctctgtctgggcgaagtttaacaagaacagcctccccccatgtggtatgggttgtaactgtggcggtttccctctgctgtttttggttacaagatgaacattatctgaacacacagaaagaaatctgtatttggcatccataatggaaagtcagtttagtaatttaaacttagccagttatcatcatcataattctttttaacactttcaaagtcagcataggagaagtgtattgttgaatattacaaaatatttagggcatagatagatgtgctgtgtagtttgatttgttaatgtgtctaagcaatcaaagcaacagaattcaaatataaaccccatcacttccaaaataggaactctgtttactgacttgattataacatatggaactcaattgttttccattaaaaaatgatactattaggaaactcaccccattttcttttcatatatattctgctatttgcataattgtctggagtccatatgtaatattaaatgtaaaacacaaatgccatgtagctggtctgtttcttcctcaccttttggttcctggcctcctggggaagggttgcacatctgagccgtggtctcagatgactgcctcggaagaagcctcttcccttcaggcaccactgatgtgtgcttggtgtggagctagactttccctggctctccatgtgacgctcacatgtgcgtgtcttgatttcccttaacttcatggcttatctatgaacagcttgatttgggggaaaaaaatgtgtttcccaatgctggagttataattgaatgtgctgcagtcaaaactgaaatgtgtgcagagaaagggggcttttcctgtcatgctcattgggcaccagtgtgtcttcacctgttttgtgtgttaggtccatgcgtcatgctgaaatgaagaacatgggatgtatggggctttggacagtgctgagccaaaagcaagtgctcaaaagcagctgtgtttgtattattagtggttctggaggtggctgattgccttgcattttaagtagagagggattgtagaagactgccaatacttagaactttttccagagaggaagggtcagaaactgcatctgcagggctccttgctctccagaaatgccagtgtgcctgggagggcatcttcagaaatccagtctctcctcctcagtgtgtcctgtaccgactcagtggttctgtcttcagaattcctatcatgtctgtgatctgcaaatagtggtatttaatttgacttcaatttgtataaatgttagcttctatttgttcattcctattttttgttcaattaatacattatttattgagcatctactctgtgtcagccccttgggtgtttaatactgaattagtcacatgtgggacttgcctgccctcagggagctagactataaattcctaatgatcagtggtctccacttttctgtcactcataatgtctggcacaacataggttacttgagttgttacactcacagtactgttgtttgctgccatggtgctttaggaagtgtgagagttcccgggaggcagagtcaataatgcagactacacgtagtgaaaacatggccaggagagctgtagttcaggctctcagctcaactgcactctgtccactgagaagccataatttcttcacttaaagtgactgtgcgctatggctgtttatatatacgcttaaaaagtaaaagctgctaaaccactcaaggattggggccttttgtattgatttaattaaaggaacaatcattgttttaatgagctctagaaacaattacttttgaagagccgaggatcaaattcttgcctcacgttttgccacagtgtgttctgaaaggtgaattaatgcttttggaatcatcaggaatagtgagctttgtcacgatttactttttacaagcgtatctaatatgcatattgaaatgtgagcctccccaccacacttccgctttgataagcatcccccggattgccgtcactgaccattatagatttttaacaaagttggacagtacacactgaatgaaaactttacatcaaggaaggcctggcgtgtttgtaaaatgaattaaaaggctcattaaatgatttatatgacttacgccttctgaaaatatggcctcaaacacagagatccccaaagccacaccgacccctgcgtcccatgttctcgacctcaccgcatcagcaccagcaagacctgtcgctgagacggtgagtgatgagagtcaagaggagtgacttgcatggcctgggaggaaacctcctgtgaatctttagttaagcaggaaaaaaaaaatcctcatgaaggaaacaggatcttgggagcattttgaatgaagaaggagcttagtgagccaaacttgagacatagggtgtaatgtgggagagttttaagatttgcagagatgtacagcttgggagggggtgtaatgcattttcttaaaagagctgaatgaatggttgaggaaatgggtacatctggtttggttaaggatcctaatctctgaagcctgggatgcccccagggcttgtaatttaggaatacttcccctaatagtagctaacccttatatagtgctgtctgtgcaggctacaaaaggagcagattaaggatagaaaaggtttggagtgtatgagaaaccctaggcaggaattgactcctggtgtttgtaaaccttaaagatgtcctaaaaaggtcaaggaataagacaggagaaaaaggaaatgtcaggaagatgatcaatttaatgtttatggaatttagtttgtacttactgcccggcatcttgcctgaggtttttaacctcagcagcacatcagaattactgtgtgtgtgttggaggggctgggggagataaagaaattagcctcatcccaaacattctgattcagtctgttacttgagaaactgaattgtgttttgtccataaagaagatgaaattgtctacagagaacacattgccattcacaaggttgaggggataccacagagaggctcccactgtgatttgcatttgtcaaaagttctagagaattcttcaacagtacacacatggttgttttaaatatatcattgttataaaaattcgttttgagttctgtttcacagaaagtttttttgaatgaatgaatgtcatatatccttgctaaaggagctcagttaaaaaaaaagggaccatccttctcttttgggggttgtacagtaacacattcccaagaaagaggtaacagccacatacatttttcttcccaataaagagtgtgggtttttaatatgaatccatagtatgatttctgttatgttttgtgctgcttcataaccacactcatgcacttttcagaaaattaataccattcattagcataaatcataaactattcccttggtatgggtttgaaattgggggtgccctatcatccttgctttatctcttagtgaattatgaccctgtagtcatcatggctggtgggcgtctctggttaaagaaagggttggattggaaggattcagaggcgattctttgttcttaggctttaatattttaatgagcctgcaggcttggctgcttacgaacgagctgagatttctaagtgtgttgttagtgttagcacttgtagaaggatgttcattaggaagttcttgtttcagtttttcagagaaactccccattaagaaagatcattcaggaacatggctaccaagaaagaggaaagggaggagggaggctttcagctataagcattaaggggatattgtatcagtagtcttagttctaaagatttgcttctgagaattaattggagcaaatacatctcaagggaagaaaaaaaaagatttatagggcagggacagtagttgtccttgcaagtagaggacacttcattttgcagctgaatcaataccacaactaattatttctggttatcttttacgcatttgtaagacattgcttttgttcagtgtaataaaaaacccattgtttgatcagtgactgactaattatgataagtaatttgaaacattcttgatgaaacttgtctgttaattaacatcaacagcacagggaaactaacaggacaacaaagtattagtggatccactgttccctccaattgacgagctttctctgtggcatgcccaataaactaaagctgccaatggttaaaaaataacaaacatgtgggagatctgactcaccacggaggaagagttatggtaaagttacacaaaggagtactgaaatattacaagcgagggggtggtaaagaaatgtcagcaggtagcctgatcctacagcttagagtaaggaaagtggtttctttctgtctttcctttttcttttaaagcttaattccaaaatacattcatcccatattgatctgaagtaagagacttttgataaattaaagtgtgaatctgaaaatgtgtagtttgggattatgggcattgcctggctatcttgtaactgtcattaatactgttaatttttatcaactcaatggcttttttttcttatgcttttagatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatgtaagtataattttattcatttattttatagaaattaagataagctatataggtttgtatcaattttttgtttccttaaaattattgtgacaaataatttgatgaaaatctatgtggaaaaattgtcccccccccctttttttttttcaaagaaaacttcattgaatttgggaccctgtgctaccagtattcattaagtatacatacccaaagagaaaaaaaaacactagaattcttaatagtattgaaataaatgtattatatgaatatattcagcatctctactgacaaaaccatttttaaggaccattggtggattttgataggtaaatcttgtgcattgccttttctcttcacccatccatccattcattcactcattcatttcgtatttattctgtgccagagactgtgcttaagggctagggattcagcagtgaaaggtggtaaaatagcatgttttcctcaagaagttaacagtctagagaagatggagctcataaattcgaaagatggggatgacaggtcacattaaaaccagattcagaagaaaaagacgaaacttggtttgcttagtacattactcttttttgcatacatatatataatttgacacgctgtttcaagaagagatggtacgtatcccttgggtcatatctgaggctgacttgtgaggatgtgaagtcagctgatgagcacatttggagcccacgcctactatgtgcagatctctcgtcagcgtcattcccagggccccaggtggtgttaaagtctaggtgactcagacagctgttcgcgtcattcaagcaatgaagtcttttttcttaatttctttggtttaaaattatactcataattaattgggttgaattttccagtggcttggttaccatagacttcagtttattagggaactgctatctgccactggtttattatttgccccaaggtggactctaaaactttaggtaggagactcttggtgatcaaactgaaactcttgcatctcaacctatgagccgcactttattgttattttatttttttagagacagggtctagctttgttgccgaggctggcgtgcagtggcatgatcacagctcactgtagccttgaactccagggctcaagtgatcctcccacctcagcctccaagtagctcggactacaggcatgtgccactgcacccagctcaagagctacacttcaaagcacagaatgaaaacctatttttaaagccaacttgatacatagagtagcttaccaagaattagtaacaacaacaacaagaaaaaaaagagagaatgtggtagagtatatacttagtaaggagtaattattataaaataaaagcattctgaaatgaaacaggtagatggggtggccaagtatgcagcatagtagggaaatctttgaaaatgtaaaatagttaccaggtaaaataaatggaaactttaagcttttggaagcctaacaatgtatttatattagtaaagactttatttttttattttattttattttatttttgagacggagtctctctctttcgtcaggctggagtgcagtggcgtgatctcggctcactgcaacctccacctcctgggttcaagtgattctcctgcctcagcctcccaagtagctgggactacaggtgtgcgctaatttttgtatttttagtcaagacggggtttcaccatgttggccaggatcatctggatctcttgaccttgtgatccttccgccttggcctcccaaagtactgggattccaggcgtgagccaccgcgcctggccttagtaaagacttttaaagtaagactttttcagtgaaagctactgttaggcatgacatttacaggcaactgaaactgatcagatgcatttattaagaaggttaatgcccctaggtggggtgggagaaagaaggtcgtggtacgggaagaggggacacactagagatgagatgccctagggcagtgaacgcatgtccctaatgcgtggatgcagcccacgtccaccgataatgccgacacacccagagtctctcttcttactttagcttatgacttcacgaagaatgctttgcaaattctaagttcgcactgggcgcaagtggaattttagtaaacattaagagtttaacctttagtgtgaaataatatgcaagatatgcaaataattgtttaccaacatctctttgcttaatgtggtgagcatttaataattgctttttattaatacatgagagatttgtatttagaagcagtttaatttataattataatattaatctacacaataacgacatctattattttctttttttggaaactcttcataccacactaacaggttcattgcagttactgaactactctggccatcagagctctccttagagttacgatttaccatgcaaaagcatatggtagcctgggataaatgaatctttcttaatacagaattgagggtctcaagtttgaaactacgagaggctatttgaatgttgctttgggggactgtcataagggctgggtggaggactcagggctaagaagtttgccaggaagtccagttgagactttcagcagagttgaaagacttccacgatggcgtaggcagaggaaggcgtttcagatacttgggaaaatatagaagccaatttctcacccaccctacagcaaagctcattgatctacaagtttccctagaaaggaaatgggaaatgcagagaacaaatgttaaaatagttttagaaattaatattgactttgtattgcttctgcataagttccaagacaccaaaacaatgaatggattttaaaaagtcactactttgcatatcagacaaatgcacacacacacacacacacacacacacacacacacacacacacagtcaagctctgtactggcttttttgagaaggaaagtgtttgaagttagtaatttttatatcagtacatttataaatagtgctaggtagcatgacggaaagtattaaaatttacatgtatatttttaacacttcaaatcgttggttcactttgagacagtaaataatattagcatttgagttcagctttaataaattctacatgggtttaaccccaaatctgagtgtctagttggtaagcgccttcagaacgagcagtgttataataaatatgttattgtgtgctggtttctttccatggagaggaaaaagagacctgatgctttggaggagtgcttgacttttccccagtgaggagtagtccagagggactgacttgcattggggagtaccctacatgaacagcatttcagaagaattaaaccaggaacctagagtcctacttgctagtcctgcttcctaagcttaatgagaaagtcaattttatttctttgaactttaatttatttccctaaaaaacgcttttagtattgtcattgttctggctaatgatggcggtctcctccagtttcaagccaccttagggctgggcatacaaatgcaatataggatcacttgttagtgtggtttcaaatggacatgatcctctgtaaattctttaaaaacatttaatttgatttgtggtgttacctgctttaaaatatagtcatcacacttgtgagtttcagacgtgaatatgaatttttaatttgaactgtatttttaaacacactaagtattaactaagtccccttaggagatatgtggcaaactgatatgcatcctcattcattcttctcatagatggttatttgttttttaacttgtggcaaaattatatatgaatggtcaccgacttaaaatagttccacttaaatttttcaactttctgatgggtttattggagtattaaatgtattttcaatttaatgatattttcagcttaccttgtgcttatcaagtatcaagacatagccccacctaagtcatggagcatctgtatatgggtttttattcttgtttagaattgactttttcaagtgacctatttcagtaattagccctgggcctgatttgcataatgagatctcctaatcttcaagtaatgcaaagatggagatattatggccatgtggtctgaagagaccttttctttattatgttcagatctttaattgccttaaaaatagagtagctaatttacctaacctctagttattttattattgtctttaaagttttttttaatgttcatgaaataactgttctgaaattgcctattttcaagggaagctgtgtcttagacttactaaatgctccagttgatactgggaaagccttcttgtgttcgtagcctttatccgtagagttttctttgcagcattttctgtgcctggtttagtttcttttcagaggcgacacccagagctgaatgagtcagcaggtttggtgtgtcgaccctttgcaacagctgtccttacgaaggttctgtgggctggttattctaccttcgcataaaaccttgcaaaataacccacaaagaggttttcgtcacactaccaaaatcatgtgagtcagagatggatgaaaaatgaatgccattgtgttcatacttttccagtgaacagtagctacagcagagctgttagacaaagaaaaccgtattaatgaagcgcctcccaatttagcttcatatggcttttgcattattttgctgcaaatccatagctaagacacatcttgtggcatagtccgtaagtcatctttccgaaggactgtttgattaaaggttgttctgtgagatccaccctgtgttgttcatggcatcctcttggaggcctccctcactctccatgccttggcaaagtcttccttaaggaacactgaacaagtctggagaagctgccatttcttagggccctcattggttcagttgtctatagctttttattttttattttttttttaataaagagtatgtaaaattggaaagcttcacaaacagctttgctattttttagacatgtactccacttctaagcaaaatcacaaaataaagtaaaatgcttccacaaatataatgaaacaatattcttaaagaatcaaagcagaagaacttcagagtctgttgcttatgttaagcatatatttgttttcttctctgcttttgatttacttatttctggggtgtaggtttggcaagtagtactgaaacgtactgaatgcactgttctttagcaagatagttacaggagctttcaaatgtcctcttaacatatagatttcttttagaatatagaataatgtgtgggctgtataaagcgattatgtgctttatttgatgaattatttatgtacgataaatgtagcaaaagccacatttccatcattaaatgtaatcccatttggtgatacagcaacatcagcctgtcatttgggtcctctgattgaggggtgaggatttctgtttgataccttgtgcataatggctgcgttcaagcatttaaactcatttttatttctaacctacagctgtcatctttgtaataggatattcatcagaatcttgccagagactgtgcatttgggatcttgggggatacagcaccaccaccaccctccccctgtccaagagaaacagatcaacatcttaggttgagagtctggggtctggaagacccgagttcctgagtgccctttgacaagtaacttaacccctgtctgcctcagtctcttcatctgtaaagtggggataatgacagcacctgcttcacagggttgatgggaatccagatgtggtgggatatagaaaatgcttattacttccacctttgacaccaaatacatataactaagagttaactttggagcaggggaggaagtgtgaggctccaggctggaggcagacctgtgttcggctgcaagctggagaggatggaccccaaaagcttggctgatttgaagtccatccataaaatggaactccagagagtttacacgtttcagtaatgctgcataacttaattataagatcttctctctttgtcttctttcagtgttataaaagctcttttgtccttgagcttcctttaccaagaaacatgcatttatgtatctttttgttcatggaattgcccaagcttgttagcagatcctttgtaagacccaaaagagacagacaggggaggagtcttcagatacatataatcatttttcccaatttccatgttaccagccttgccaggactttttctcagttccctgttacacaatgaaaatagtgtctctttattgataattttagtagcatcctaatgtggtataaatcgtcttccagagaagaaaatgtgtcagggttgcgttatcactgaggctagctgggaaagtagatcagcccattagtctgataattcgaagcgttgtttctgttatttctgaacatcatgtgaactccttttctgggtgtattaaaggttttcccagtgtgtgtcagtgagactcctgattgaatttaatatgaataaagataaattctttacatttaaggattaaagtctcagcttctgcttaacttgagattgcactgagaaactcctggctctcgggtatagcggagtcacgacctggggatgtctgtcccatatggctctgtgtgtaagaagaaaaagctgctgtggacggagactctgttcacattaaatgacatcacctaagccatcatgacagcaagaattatttaggaattgctcagaataaaactgccttcattatttcataaaatgtatcttggtatctttagcaccttatttatggctttttaaaggttcactgggatttataaataattggacaatgctagagacctagtacaagaatgaaagaggacaggcttctttcttaataacctttaaacattcatcaggaagataaaactttaaagcaaaataaaacacatgaaaatagccaagatgcacagaccagacaagcaaatactactttaacttatttgtatagttcttaagagtcacatttgttcctgaagtttcaaaatctcgggctgagtgtttgatcacttagggaagtgttgtggccttcacatactcttgtctcactttgaagtctagaaacacaggtcttagagcaatttttatcactgtgagaaagctgaaacttagtgtgagtagcttagtacaattcagttggccatcaaatgtcagaaacaaaactcagtccagggccgctggacccttaggccggcgttgttagtttacaacagtgcctcctgggtccaaacatctaagtgcacatgtagcaatagtaaagatagtatgtatgcatacataacacatatgtagagacagcagagtatacgtacacacatgttgcatacatagcaacagcagagaagctcatgaactataaaggatggactgtatgcttgtatcagacattttggtactgacgctttgtcatatattgtgtaacatataaccagcttgcaatcatctgcccccaaagttgaactaagaaaatcctacagggtactaggaaaggaaggccattgggaaaaggtggttatagtggcaatttgttagctcttatgaattttctttttctttttagacatactcttaattccattttttcaataaatctatactattttgtgtttttatgttagcaagtactttaagcccctcaatagaaagttgctacatcatatagtgattaaaaataaaaatctctcaaacatacaagtagaggtggtatgagacttcaaattcccttagccaagtacaagtgcagcagttttgttggctggctggctgcatagaaggactgatggattggcagaccctcaagctggagtgtaattgatctcattacagaggagccaggctgggtgacagttgtgctttgcaagtggttttttgcattggtgaagtagcccattttgttgttcctgatgttaaacaggggatgaaggtattcttttattggcacaaacgcgggaaattgctctggattcttagaggatagaacatgtcccctggacggaataaggttcatgtgtagggcaaatttagataggggcaccttattggggttactactggtctctagatggtcaaagcaaacaacatgtccatctaagctgtgatgtccatctaagctgtgtgtgtccatgagagtgacgcattttctcctctgcagtgttgttatattctaaactgtcagcagacattaattcggtcgctggtgaagtcccaccgcctagagatgaactctgcctccgatggatgttttccacttcagtgccactcgtctcgcaattactgggtcattaatatcattgcatgcaattagtgacagtagaaagagctagagggttgtgggatgtgcaccctccccaccatgaactttttactctgaccctttcccagctagaccttttcgtatcttggcaaggatattttaatgattgagactgtcagaatcttcagagcaggcactggattatgtgctggaaataattcactcaaacacctgcttctccatggttcagaatattttcattagatattatcactatcccttccctgggaagtttcatttttaaaaatctgatgcttaagtacagctaatatagacaatagggaattatgttttatctttagaactcttacattattcttttctttaaaaatgtgagctgagtcattgctattgcagtggtcatctggccgcctatttttaaaacacaattcctctatcttagtagattttggcccatattaagcatatcaagaatgactttttttttttcaagacatggggttttattgggggcttatatacaaggaaagagagagtccagtggcagtgggctggacaagatatccacatggccctgtggcagtgagctgggcaggaaaactgcaactgcttgcaaacagcatgtagttcatctatagcattttcacttaacaccacccagctaatgacttccacctggcaaccttcatttaatccagaacttaggacctcgagtccctgtacggcccatgttccacaggatgggccgagggctcagctgttcctcatagacaaggaatgactctccacattggccactcccggattccctagctcaggacacatattcaggtgtgtctaaggctggctcttctatgtgaagttacttattcttttaccattgactctcatgttcccactatattaagtttttctgaattactgtggcaataagaaacggtcccttaaattatactagaagaaaagctttttttttgttttgttttttattttgaaattatgttaaattttttttcttaactgagagattccacctgcataaatcgtcataacttttaacagtaagatcttagacttagaaagtgatgtttttcctcaacagaatttattaaaaatcaagacaccaagctgttccaaacaatagtttgaggggaaataaaataaacaactccataaataatcttatgttgttaaacatgtctctagcaaaacaaacaaacaaaaaagtcgggggttgggggaggtgcagtttattgccagtactgtctggtctttctcagaaaagcgtcagtgtacatcactgagcctggacggtatgttttcttgatctataccccctatgtgtacatgtgcttgcacgcacacacatgtagacacgcacacatgtgcacctgccatcactttctgctcttccgtcttttcactcttgagtgtctgtagccagtagctttccaggtctgtatagtcaaagatacctatggccctgaatgtcttcactgattgctatttgacattcatacggtttttaatggttaaaaggctttatgcgaaagctgtgatagaatttctcctgttctagatgtggtgtttattgctttattttgtgacttttctctcagtagattgaccttctccctcagtgtccaagcctcgcatagcatgatggcacctgtaaactcagttctgtatcctggtatcctttctcttcccaagtagaagcaattaagtaatatatgtcatcaaaaccttttaagtgcacatacaaacaaaatcaacttaccaaactgcttcaaagttgttccatgtttaacactcttctttctgagctctgggtagaatgtcctattattgttcatcatgaatatttgaaattaaagaaataaaactgtaccattttctttaagagcatccatttgtacttgataacatcttcagtcatatttcaatgctggcaaagaggaggggagttctaaactgtgactcaattttagaatctactttttccaaattattctgtttagtgcagaaaactaattaatagtgttgcatagaaaagtcactgaagctaagccagttattacttcttaatgcatgatttactgctttaagttttcaaaacacaaccatagcaatgtggtattaattcaagtgattcttcctatcatattgaacgatattttcacgggtgaaaaactcacacatcctacatcactgatagtttatacagtgttttagctgtggctccctgcatgcaaaataagagttaatcaaatgtcagtgagaaccatctcatcaagtagagggcttgttttgtttaaattaactttgctaagtataaatttcttcttgaaaataaattctgggccgggcgcggtggctcacgcctgtaatcctagcactttgggaggccgaggcgggcggatcacgaggtcaggagatcgagaccaaactggctaacactgtgaaaccccgtctctactaaaaatacaaaaaatgagccgggtgtggtggcgggctcctgtagtcccagctactcgggaggctgaggcaggagaatggcgtgaacctgggaggcagagcttgtggtgagccaagatcacaccactgcactccagcctgggtgacagagcgagactccgtctcaaaaaaaaaaaaaaggaaaataaattcttctgtatttttctttcttcaagtgaggccatttaggggaaagtataccataaaacttgctctaagataaggcaaatttggtattataggatgaagtgctatgtgatttgaagtaatgctgaattttttaaatatattaaactaaacaagaataatgaggccctcggaaagtcatgattatatttctcatttttctcattttaaagccacagtgaaaaacacataaaaggaagaagttagaaaaaaaaatgaatgaaattctttttttccttttggcaaattaaatagatgtttctgtttcagaagattttattaattaactttaaagaaacagtcatttatttttggcattcagtgaacactatcatttccatgtttagaacttttcttctaagttagcatcttaaaagataactgtgaaactcaaggcattcaactacattaatttgagtttcagaaattgaattcttgtttctagagtacatagtttgaattgatgtcagggtgttaaatagataaatcttagcttcctaggttgtatattcacactaattatttttttatcagccttcttatttttcaacttaccttattctttttgtttttttgacactcagatttgatagccctgtggtagaagaaaacagtaatacagtttggtttgttgttgtgtttgtgtttattttaaagtcacggctttgctttccatgttgttactggattatgctttttttaattcttcagtttgccaagataacagtcttccgatcttcagaagtctgtatcaagcttaaggaaactgatgtgtaggaagactcgcctaagaagtccaaattagcaaggctagcatgtgaggacatgctggaaaagaatagttcccatagatattgacagagaatgttcataaaatgctacttgttttgtggttacatgagagtaacttgtgtccagtgcagctgtatgtaagggcaacgtttttattctgacgactctgtggttttcatgaccctggatgcttatcatgtctctctgttggacttcttcaacggagttgatacaaatacttgcttccaagtgtccatctgccctctcctccatcctggccccatacaaatacgctacatttttaaataatttgaaataccctcaatagtatttatatttcctggtgcttcattctttccataagaactgtgataccattattctgtaggatttttttgtgcttccccgtttcacatctctgtgccagtgagacccatatatcggtgcaaatccagaagtttgattgtccatctgattagcacactgttagcaatgtggtggactaaacacagccaagatgtggggctggagcttagcctcctgggagcagagcggtgaacatcagatgaagacatgtgaaaatggagtactacttcctcttcctggggatgggctaaaaagcacagccagaaatattcttgcccttccagtctgctttacagttactcactggttctcttttttttcctactcagataaccagtatactcttcccagtgactaagaactgcagataagtataggtgcaaatagatggcaaaccgcagatggcagctgtgtggtttcagatgtgctgcagaacttttagacgatgtgaacgcaaggaacttttttgctgagcagtaatctctacccactggaaattaggccctggggggaacaatgtagtgacttctatatacttactacatgcagttagacccctgaagcaaaagcttttaaaaacaggctgtaaaatgcccatgtatctttattaagcctattttccaactggatagagaaattttctggtaatttttaaatttgtaaagtctatttttttcctgagccaagggaaaaaaaatatctgggccctaaaagcttagttataacaatgttattttttctatctctgaatgattaaatgtgatttcatttatgtagcaatactatgattgtggctgcattagatcacgctgatagaaagatacaaagaaaaactaagtataatgaactaacaatttattttcactctttctctaagttaaaaattcccagtacattcaaatgaacaatgaaaataattgcagaattgtctcctgaaatggaaatagattttttttcccaagcattagcaatttcttgttatttttcaaaatcagccactaagcctttcagagcttcttggtgactattgcaggagaaatcagaatattaatcttgtggttttatttcagagttcgctgccaggaaggaggtataattgggataggagactttttttttttagctgtgtcactgttcaaggaggggggtttggaacctcagcataagaattacactctgtgatgaggatgtagcaggggagaagaaaggtgattttcactatgggaagctatacttacatcaagtataaaatagactgaagtcattttgaattacgttatacttgtaaagtttacctcctggagtttcagttagtaccagtgtactaactgggttaaaacagttcatggcaccttagatcatttctaactcatggcaaaaatctttcctggtggaacgtgtaactgtattttaaatgcccctttataagcaaccaagtatttgggatgttattttgatattagtagtgaatttttcagtatcttccagtaccctttgcaagtcacaggttgacttaaaaggaaaagaagcaaaatgctgaatatagcagaaaaactgtctgcattcagactgttcagcccacttttgctccccacgtggcaagcacactcccccaaacaagcaatagcctgtggcttcagaggaacctacaaaggcagcatctgtagatttttccttcttcaactctaagacttgaatgtttccctcttccccacacactttttttttaaaccaagaaataaaaaagttttcactcttaaaggtgcaaagcagtttcattcttatgcaacacagccttcctcctactgtcttatagtctgtggatgttaaattatagattccaattgaattttaatactctagagattttacatttgtggttgtcaagaccccgttttggtaaacctagggagctccgcacaaaagcattgatattcagaaaaggcactgacctacaaattaaaagaaaaaaaaatcaaataatgtgcacctcttgtgcttccagtttgacaaagcagaagtcatcagcagtttctccctctgcagacgcagttctcaattctatttacaagtaactgctctactgtgcctgtttttctcttgctgatactcatttaattgtttttcttttggatctgaatctttgactgtcttttccccctcaagattaaaataaatacatctgtattcctcccctttctttctgtgcactgcccttcagatctcattttgtcatttttcagcttagtgttgaaacttttagcaacaaaaagtcagttacttactttgagtaagtaactcaaagtaagttaactttgagtttgagtgcacttttgcgtgtaggttcatttatgtgcttgtgaatttaaaaacattgggattccacctgaatgaagtaaaccaaacattttaaactatcagccagatagagacatcagcctttcacttctttctatatgcagacatatcctaattttttagaaaaatcaaataggaaaattctcaacaattaattgaagattatagctctgctctgaaatggtccagaaataggatctgctcatagaaactcatagtttgaagcctctgggaggaaaggatactttaaaatttagtcacatatttggaggagggaaaagggaaagagcagaatgaagaactgaaaaaaatcacacaccggggcctgtcgtgaggtgggggactgggggagggatagcattaggagatatacctaatgtaaatgacgagttaacaggcgcagcccaccaacatggcacacgtatacatatgtaacaaacctgcacgttgtgcacatgtaccctagaacttaaagtataataaaaaaaaattttaatagccccattaaataattaaaaagattttttttagattcacagaagtgtacaaaatttttaggtttttttttttttaagctgtctgctgaatagtttcttaatggtctacaatgtttgtatctacaaacagatactgtctgcttcttactacccttccaagacaagtattattatggcaattattgcccagtttcccgggaaaaatttatccacagttacagaagaatgagatgcaattgtgagactgtaaagtttaagcaagcactcagagaagcacagtgatatgtatgcacagaagaggcagtctttgttttgaggaaaacagtgaaagtaaagttaattcaagaccacaaagacaagtaaataagtgccttatttttgtagttaatataatttcagtggaatgcatatttctaccataaatgcatatagaacttgtttgctgacctactgtttggaaaacaaacaatcccattagaagaatgtctttgggatttatttttaccagaaaatcaatccttttttcagtcccttgcaaagtacagtgttacaagccaagactttgataatcaggtagaaaatggatttaaattgcagaaatgtatatgaaacacttttgttccttgccccttgaactttaggggaatgaaaatgtctagcactctccaccttcttttctctcctggaacttgaactgtaattcaaagcctgtttctcattaaagtacctggcagcctatctctttacagcttgagttacaaagctattcagagacctcgctggtctaaagagacagaacaaggatgtgtttaaatagagcataggctgttgaaaaaaaaaatgctgaaaatggtaaaatgattctgtccttccttccactcctcactgctgaggtggagagggaattcagttggtgaacaccagcaagtggctggtaaaagtccccactttctctccagggctgccacaggacccagaatgagtggtgggcatgtgtgtgaaccctctattcagccagagttttcccgcaacaggtagtttggttgaagaggttgactaaggttgacattggcagtaataacacgtatgttcttctgatttacaaaacgatggaggaaaaaggggagattttgaagacctgatttctggtatacttcttaagcatgcataaggctgaaaaaagaagacaagggttgtgggaggctcctggtctagtgtttacagaacttggatgcttgacaaacagagcgtcaagctaattgttcttgaagcaggaaatctgcagtggaggaagcaggtgtggggggatgattaccacgtttggaaatggctgcattaactattttgctcttctgagtttggccccaaaagagtccatagactttttgaaggatgccatcccttttatttatagactaacattaaatcagtcatttgtgaaggaaggagaaagtgcctaaataaatttggagtcagatagcatacgtgcggcagtgtttccgatatccatttctctttatttctttttctttttctttttggctttcagcatccccatactttcagaaaacttgtgactaagagtgaattcttatttttcaaattgttttcagacatttcatgttcatgtaaacttggcttattgatttcctgatttttctttatttttttgttttgtccattttatttttaatcagctacatcaaatgggtctttggagggcctggataaccaggagggaggggtgtgccagacaagagccatgaagatcctcatgaaagttggacaaggtaaagaccatctgctgcttcatgacgccactgtgacctggtgtagcccccagctagtatggtgctaatgttgccgatgcccaccttcattcgctcttctttttagttttcaaagcaaacccttctgcactttgagccactgacagatttcctcaagtcaatgtactaagcttttattggagatctaagagttaagatcagcaaggtagaatgtctattgccatagatagatagatagatagatagataatagatagatagatagatagatagatatttctttttaaaaagcaaaacactttggttcaaaatcaaaatatccagaatgaaaactaaaagcttgtgcagttttgctcatttctgaatcttgactacagaagagttttgttcattgtgacttttccaatatagataacctattgtgcagaaagaaataattattcttctaattaaaaattggtatagtagtcaatcaacttgctcagttaaattgaaatgtcatctgcaatgctttgcctgccaaatgcaagaatccctatagtttccacagatggcctcacgttctaaacctctgaaataactagtataaccattttgttttaaaagaaaaattatattcttgtatttcacagtactttgcataaagactcttatgttcattgctattcatgcctgttgaaatatatatgcagctcctaaagctagatattgtcagatgtctgtgccgtaattaatcatttgtttttcatatagatgcaagttctgctggatcaaccaggaataaagatccaacaagacgtccagaactagaagctggtacaaatggaagaagttcgacaacaagtccctttgtaaaaccaaatccaggtataacagcatgatctgtgtgtatggaggtctgtgggtaccacattcttagtagtatcttaaaaggtagggcagagtctaaagacttctaaccagttaggattagctggaagttacagtgatcaggaatctttgctgtcagtgagtcattattaattacactcaataagaacaaaataactcattccaatgaaagtcatatattcaaaggagtagagttcatgagctgtaagtgccagttattagaactactctgtcaggccaaaggtttcattggctgacattttatcaagctggttgtcaactccagcttaaagctgatgttaatgtatatgtaattaatgtgctaatccctcatctaattatatctaagccacagagggtttaattgatcctcttctaaattttaaatggtaacatttttaaatattgcataatagtattttttcaggtggttatcgttattttgtttcacattttccatgtaaaagaaaatattaaacaggtccctgacaaaagtgtagaataccagataaaattgtccgtcgttgaccttcgttttcttaacagtcttggaacaaatagttctgtatttgttaccatgctaatgaaggttttatagagtagctgttgagcagacatcagcagttttgtattaggattgttgtgtgcttgcttggtcgttgtgcaaatttatcgtctgcagcaatattccatccctttccaagagtcaaggagggaagttgttatttctaactttcaatgacaagatgtgtcaaattcttgtgacaaactgataaatggataatataatgatgccaggcagttttttagtgcttaacatttgggctggcagtctgttcggtgtgagagtttctgctgccttccaaatatattttaagtgtaaatcaaataatacagacgagttacgagctgaacattttcccaggccccctcactccttccgcgttcccgagctgttctgttctgccaggaggcagggctcttctttagaaggcaggccctttgaaggtttgcatgaaactccctttctcaaaggaggcggaagagcaataccacataaacgctcaccgctgacctggagaattggccacttccctttttcttccctgccgctgccccaggctggctgacacgggttagaagatgaagcaagatcaagggctggctgtcaccgacagtctgtgctcttgctggataatgatacaaaggaaaccctgtggcttgggagggtagggaagtccctcctagagatacctctcatttccttttgcgttgagctcttagacgaggtattggcgaggcaaagtccagcttctagttagtaataagcctggcttatttttcacatttttaagggtcataaaagcagtccgtctgcactgggacagcagtaactatctctgaccttttctgtctccgcgtctgcaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggcgactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctaga

SEQ ID NO: 330 - DNA, 4335 bp, Homo sapiens
gcgcggagctgggagtggcttcgccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctagaatccagcaggtcacactgggggcttcaggtgacgatttagctgtggctccctcctcctgtcctcccccgcaccccctcccttctgggaaacaagaagagtaaacaggaaacctactttttatgtgctatgcaaaatagacatctttaacatagtcctgttactatggtaacactttgctttctgaattggaagggaaaaaaaatgtagcgacagcattttaaggttctcagacctccagtgagtacctgcaaaaatgagttgtcacagaaattatgatcctctatttcctgaacctggaaatgatgttggtccaaagtgcgtgtgtgtatgtgtgagtgggtgcgtggtatacatgtgtacatatatgtataatatatatctacaatatatattatatatatctatatcatatttctgtggagggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagttccctgcaaccaattgaaaagtgttctgtgcgtctgttttgtgtctggtgcagaatatgacaatctaccaactgtccctttgtttgaagttggtttagctttggaaagttactgtaaatgccttgcttgtatgatcgtccctggtcacccgactttggaatttgcaccatcatgtttcagtgaagatgctgtaaataggttcagattttactgtctatggatttggggtgttacagtagccttattcacctttttaataaaaatacacatgaaaacaagaaagaaatggcttttcttacccagattgtgtacatagagcaatgttggttttttataaagtctaagcaagatgttttgtataaaatctgaattttgcaatgtatttagctacagcttgtttaacggcagtgtcattcccctttgcactgtaatgaggaaaaaatggtataaaaggttgccaaattgctgcatatttgtgccgtaattatgtaccatgaatatttatttaaaatttcgttgtccaatttgtaagtaacacagtattatgcctgagttataaatatttttttctttctttgttttattttaatagcctgtcataggttttaaatctgctttagtttcacattgcagttagccccagaaaatgaaatccgtgaagtcacattccacatctgtttcaaactgaatttgttcttaaaaaaataaaatatttttttcctatggaaaaaaaaaaaaaaaaaa

SEQ ID NO: 331 - protein, 987 aa, Homo sapiens
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 332 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 333 - DNA, 29 bp, Unknown
  oligonucleotide primer
ggatccgccatggagctccgggtgctgct

SEQ ID NO: 334 - DNA, 27 bp, Unknown
  oligonucleotide primer
gcggccgctcagtactgcggggccggt

SEQ ID NO: 335 - DNA, 2981 bp, Homo sapiens
ggatccgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgaagtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtccggacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggcgtggctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctgattgcgggcacggcagtcgtgggtgtggtcctggtcctggtggtcattgtggtcgcagttctctgcctcaggaagcagagcaatgggagagaagcagaatattcggacaaacacggacagtatctcatcggacatggtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcggctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctgggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcggggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgaggacctgctccgaatcggagtcactctggcgggacaccagaagaaaatcttggccagtgtccagcacatgaagtcccaggccaagccgggaaccccgggtgggacaggaggaccggccccgcagtactgagcggccgc

SEQ ID NO: 336 - DNA, 27 bp, Unknown
  oligonucleotide primer
gcggccgctcagtactgcggggccggt

SEQ ID NO: 337 - DNA, 29 bp, Unknown
  oligonucleotide primer
gcggccgcagttcctgcaggtcaagtact

SEQ ID NO: 338 - DNA, 32 bp, Unknown
  oligonucleotide primer
tactagtccgccatggagctccgggtgctgct

SEQ ID NO: 339 - DNA, 50 bp, Unknown
  oligonucleotide primer
gcggccgcttaatggtgatggtgatgatgagccgaaggaggggtggtgca

SEQ ID NO: 340 - DNA, 1018 bp, Homo sapiens
actagtccgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgaagtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctcatcatcaccatcaccattaagcggccgc

SEQ ID NO: 341 - protein, 332 aa, Homo sapiens
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAHHHHHH

SEQ ID NO: 342 - DNA, 6860 bp, Unknown
  plasmid vector sequence
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattgccctttactagtccgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctcatcatcaccatcaccattaagcggccgcaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgtcgacaggtgcttctcgatctgcatcctgggatcaaagcgatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 343 - DNA, 32 bp, Unknown; identical to SEQ ID NO: 338
  oligonucleotide primer

SEQ ID NO: 344 - DNA, 53 bp, Unknown
  oligonucleotide primer
agcggccgcttaatggtgatggtgatgatggacattgacaggctcaaatggga

SEQ ID NO: 345 - DNA, 1323 bp, Homo sapiens
tactagtccgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgaagtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtccggacttcacctatacctttgaggtcactgcattgaacggggtatcctccttagccacggggcccgtcccatttgagcctgtcaatgtccatcatcaccatcaccattaagcggccgct

SEQ ID NO: 346 - protein, 431 aa, Homo sapiens
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVHHHHHH

SEQ ID NO: 347 - DNA, 7164 bp, Unknown
  plasmid vector sequence
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattgccctttactagtccgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgcctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgtcgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtccatcatcaccatcaccattaagcggccgctaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgtcgacaggtgcttctcgatctgcatcctgggatcaaagcgatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 348 - protein, 570 aa, Homo sapiens; identical to SEQ ID NO: 322

SEQ ID NO: 349 - DNA, 7429 bp, Unknown
  plasmid vector sequence
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattgcccttataagcttccgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgaagtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtccggacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggcgtggctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagggatccaaaagggcaattctgcagatcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgtcgacaggtgcttctcgatctgcatcctgggatcaaagcgatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 350 - DNA, 32 bp, Unknown; identical to SEQ ID NO: 338
  oligonucleotide primer

SEQ ID NO: 351 - DNA, 25 bp, Unknown
  oligonucleotide primer
cagctgagtttccaattttgtgttc

SEQ ID NO: 352 - DNA, 39 bp, Unknown
  oligonucleotide primer
gaacacaaaattggaaactcagctgactgtgaacctgac

SEQ ID NO: 353 - DNA, 30 bp, Unknown
  oligonucleotide primer
gcggccgccctgctcccgccagccctcgct

SEQ ID NO: 354 - DNA, 1119 bp, Homo sapiens
tactagtccgccatggagctccgggtgctgctctgctgggcttcgttggccgcagctttggaagagaccctgctgaacacaaaattggaaactcagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtccggacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggcgtggctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagggcggccgc

SEQ ID NO: 355 - protein, 401 aa, Homo sapiens
MELRVLLCWASLAAALEETLLNTKLETQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQGGRSSLEGPRFEGKPIPNPLLGLDSTRTGHHHHHH

SEQ ID NO: 356 - DNA, 6921 bp, Unknown
  plasmid vector sequence
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattgccctttactagtccgccatggagctccgggtgctgctctgctgggcttcgttggccgcagctttggaagagaccctgctgaacacaaaattggaaactcagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgtcgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgtcgacaggtgcttctcgatctgcatcctgggatcaaagcgatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 357 - DNA, 20 bp, Unknown
  oligonucleotide primer
cattggatcagccgtctgcc

SEQ ID NO: 358 - DNA, 44 bp, Homo sapiens
tgtttaaacttactgctcccgccagccctcgctctcatccagtt

SEQ ID NO: 359 - DNA, 1633 bp, Homo sapiens
aagcttccgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgaagtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtccggacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggcgtggctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagtaagtttaaac

SEQ ID NO: 360 - protein, 537 aa, Homo sapiens
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQ

SEQ ID NO: 361 - protein, 522 aa, Homo sapiens
LEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQ

SEQ ID NO: 362 - DNA, 20 bp, Unknown
  oligonucleotide primer
tcctgcaaggagaccttcac

SEQ ID NO: 363 - DNA, 20 bp, Unknown
  oligonucleotide primer
cagaggcctcgcaactacat

SEQ ID NO: 364 - DNA, 20 bp, Unknown
  oligonucleotide primer
gaggggtgatgtggggagta

SEQ ID NO: 365 - DNA, 20 bp, Unknown
  oligonucleotide primer
gagcttcccgttcagctcag

SEQ ID NO: 366 - protein, 1124 aa, Unknown
  Recombinant HSA-EphB4 precursor protein
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQSRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 367 - protein, 1109 aa, Unknown
  Recombinant HSA-EphB4 mature protein
LEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQSRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 368 - DNA, 9244 bp, Artificial Sequence
  PEF6-GCF2 plasmid sequence
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattgcccttcaagcttgccgccaccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgcctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgtcgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagtctagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgtagctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaaatgtgccagtctccaaaaatttggagaaagagctttcaaagcatgggcagtggctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaactgtgagctttttaagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataatagcggccgcttaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgtcgacaggtgcttctcgatctgcatcctgggatcaaagcgatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc